(19) United States
(12) Patent Application Publication
(10) Pub. No.: US 2026/0034252 A1
(43) Pub. Date: Feb. 5, 2026

(54) FIBROBLAST ACTIVATION PROTEIN-TARGETED COMPOSITIONS AND METHODS OF USE THEREOF

(71) Applicant: Ratio Therapeutics, Inc., Boston, MA (US)

(72) Inventors: John W Babich, Brightwaters, NY (US); Stephen DiMagno, Syosset, NY (US); Shashikanth Ponnala, Tenafly, NJ (US)

(21) Appl. No.: 18/998,265

(22) PCT Filed: Jul. 28, 2023
(86) PCT No.: PCT/US2023/028945

§ 371 Date: Jan. 24, 2025

Related U.S. Application Data
(60) Provisional application No. 63/392,899, filed on Jul. 28, 2022.

(51) Int. Cl. A61K 51/04; A61K 49/00; C07D 401/14

(52) U.S. Cl. CPC A61K 51/0497; A61K 49/0032; A61K 49/0041; A61K 49/0052; A61K 51/0455; C07D 401/14

(57) ABSTRACT

Disclosed are compounds, compositions, and methods useful for imaging and treating tumor cells in a subject. In particular, the compounds have the structure of Formula I is useful in the disclosed imaging methods: (I). The variables in Formula (I) are defined herein.

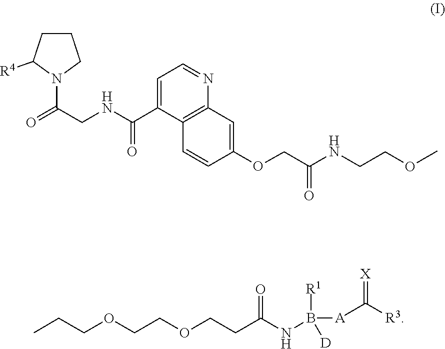

### RELATED APPLICATIONS

This application claims the benefit of U.S. Provisional Application Ser. No. 63/392,899, filed on Jul. 28, 2022, the entire teachings of which are incorporated herein by reference.

### FIELD OF THE INVENTION

The present technology relates to targeted imaging and therapy agents, more particularly compounds useful in the diagnosis and treatment of disease. For example, the compositions described herein may be used as radiopharmaceutical agents, or conjugated to optical dyes or fluorophores, or drug/toxin conjugates, useful for the diagnosis and treatment of cancers and fibrotic disease in tissues.

### BACKGROUND OF THE INVENTION

Fibroblast activation protein alpha (“FAP”) is a 170 kDa type-II membrane-bound enzyme that demonstrates serine protease activity. A soluble form of FAP is present in blood plasma, lacking the intracellular and transmembranal sequences of the full-length protein. Other common names for FAP include Prolyl Endopeptidase and Surface Expressed Protease (seprase).

FAP is one of several members of the S9B prolyl oligopeptidase subfamily, which includes among other proteins, DPP4, DPP8 and DPP9. Substrates for FAP include neuropeptide Y, peptide YY, substance P, B-type natriuretic peptide, fibroblast growth factor 21 (FGF-21), alpha2 antiplasmin, and denatured collagen I and III.

FAP is actively expressed in tissues that are undergoing wound healing and remodeling, but is otherwise not expressed, or expressed at extremely low levels in healthy, mature tissues. Tumors are localized regions of histological injury to the host, and are active at remodeling local vasculature and endothelium, as well as several other phenomena that serve to hide the tumor from immune surveillance and promote wound healing—a microenvironment that allows the neoplastic cells to proliferate. Accordingly, FAP expression is correlated with regions of tumorigenic tissues, particularly the tumor stroma and therefore presents an excellent molecular target for the diagnosis and treatment of various cancers. Its expression has been confirmed in numerous cancers, such as pancreatic, liver, gall bladder, neuroblastoma, breast, ovarian, esophageal, kidney, melanoma and many other deadly and aggressive tumors and cancer types. FAP expression is also detected in fibrotic tissues and can also be a marker for a wide spectrum of clinical conditions including systemic fibrotic diseases such as systemic sclerosis (SSc), sclerodermatous graft vs. host disease, and nephrogenic systemic fibrosis, as well as numerous organ-specific disorders including radiation-induced fibrosis and cardiac, pulmonary, liver (such as NAFLD: non-alcoholic fatty liver disease and NASH: non-alcoholic steatohepatitis), and kidney fibrosis.

While several researchers have explored FAP as a cancer target, the development of agents that can serve as diagnostics or even therapeutics have proven only marginally useful, due to their pharmacokinetic limitations. To date FAP has proven to be a difficult target due to the low and limited expression of the target and the limited residence time the prior art compounds display in vivo. What is needed are improved FAP-binding agents that demonstrate better binding kinetics and biodistribution, thereby providing the basis for improved FAP-targeted diagnostic and therapeutic agents, i.e., compounds that can accumulate to a greater degree in tumors without unacceptable uptake in normal non-target tissues and organs.

### SUMMARY OF THE INVENTION

Disclosed herein are a series of compounds that bind to the extracellular domain of FAP, displaying high-to-very high affinities for the human FAP protein (see Example 12). They can be attached to chelating groups for radionuclide binding and are therefore suitable for radioimaging and/or radiotherapy applications, for example the disclosed compounds can be radiolabeled with a positron emitter such as ¹⁸F, ⁶⁸Ga or ⁶⁴Cu, and used for positron emission tomography (PET) (see Example 11). Alternatively, the compounds can be radiolabeled with an alpha particle emitter such as ²²⁵Ac, a beta particle emitter such as ⁶⁷Cu or ¹⁷⁷Lu, or an Auger electron emitter (e.g. ¹¹¹In, ⁶⁷Ga, ⁹⁹mTc, ¹⁹⁵mPt, ¹²⁵I and ¹²³I). The compounds can also be attached to a cytotoxic agent for targeted delivery of the cytotoxic agent to tumors, for example conjugated to gemcitabine or doxycycline, or a venom. Likewise, the compounds can be conjugated to compounds having physiological effects, such as TLR agonists to stimulate the immune response of a recipient. The disclosed compounds have the advantage of increased circulatory residence time, which has the effects of increasing target loading while reducing accumulation of the compound in non-target tissues. (See Example 15) Accordingly, the favorable binding kinetics of the compounds disclosed herein reduce the “wash-out” effect (i.e., low residence time) seen with prior art FAP-targeted compounds.

One embodiment of the invention is a compound represented by the following structural formula (I):

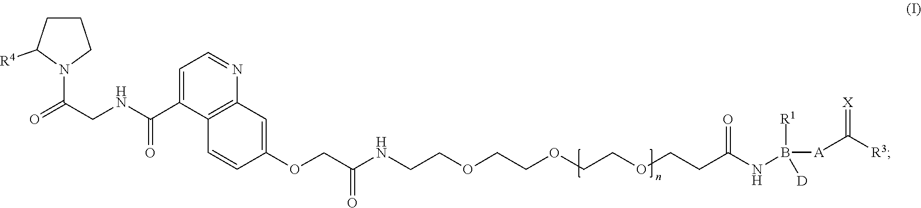

or a pharmaceutically acceptable salt thereof, wherein:
n is 0 or 1;
   A is NH, O, S or CR⁶R⁷;
   B comprises or is a branched, unbranched or cyclic aliphatic group of up to 30 carbon atoms optionally interrupted by up to 10 heteroatoms or a peptidyl chain of up to 20 amino acid residues (for example, 3-20 or 3-15 carbon atoms optionally interrupted by up to 6 heteroatoms or up to 5 amino acid residues); wherein B is optionally substituted with 1-5 groups selected from F, Cl, Br, I, ═O, OR⁶, OCOR⁶, COOR⁶, CN, ═NR⁶, NR⁶R⁷, ═S, and SR⁶, provided that B comprises at least 3 atoms in a chain between group D and the group A;
   D is selected from the group consisting of OPO₃H₂, PO₃H₂, OSO₃H, SO₃H and COOH or a C₁-C₄ alkyl ester thereof;
   X is O or S;
   R¹ is a chelating group, an optical dye or fluorophore, a cytotoxic agent, an immune stimulant, or a benzoyl group optionally substituted by one or more groups represented by R⁵;
   R³ is C₁-C₈ alkyl or C₁-C₄ aralkyl, wherein:
      the alkyl and aryl portions of the aralkyl are each optionally and independently substituted with F, Cl, Br, I, branched, unbranched or cyclic C₁-C₆ aliphatic group, OR⁶, OCOR⁶, COOR⁶, CHO, COR⁶, CH₂OR⁶, NR⁶R⁷, CH₂NR⁶ R⁷, SR⁶, ═O, ═S and ═NH;
   R⁴ is CN or B(OH)₂;
   each R⁵ is independently selected from halo, cyano, halomethyl, N⁺(CH₃)₃W⁻ wherein W⁻ is a pharmaceutically acceptable anion; and
   R⁶ and R⁷ are independently selected from the group consisting of H or a C₁-C₆ alkyl.

Another embodiment of the invention is a pharmaceutical composition comprising: i) a compound disclosed herein or a pharmaceutically acceptable salt thereof; and ii) a pharmaceutically acceptable carrier or diluent. For compounds comprising a chelating group, the chelating group is preferably chelated with a radionuclide.

Another embodiment of the invention is a method of treating a subject with diseased tissue that expresses fibroblast activation protein alpha. The diseased tissue in one aspect can be a cancer or a fibrotic tissue. The method comprises administering an effective amount of the compound disclosed herein or pharmaceutically acceptable salt to the subject. Preferably, the compound used for therapy comprises a cytotoxic agent, such as a chelating group having a radionuclide that emits beta, alpha, Auger or other cytotoxic radiation which can kill the diseased tissue.

Yet another embodiment of the invention is a method of imaging a region in a subject having or suspected of having a cancer or a fibrotic tissue disease which expresses fibroblast activation protein alpha or fibrotic tissue, comprising:
a. administering to the subject a diagnostically effective amount of a compound disclosed herein or pharmaceutically acceptable salt thereof or a pharmaceutical composition disclosed herein;
b. exposing a region in the subject to an imaging device, the region suspected of having diseased tissue; and
c. obtaining an image of diseased tissue in the region.

Preferably, the compound used for imaging comprises a chelating group having a radionuclide that emits gamma-rays or positrons or other detectible radiation. In another aspect, the compound comprises an optical dye or a fluorophore, the emissions of which can be detected.

Yet another embodiment of the invention is a method of imaging tumors. The method comprises:
a. administering to a subject, a compound disclosed herein, or a pharmaceutically acceptable salt thereof, or a pharmaceutical composition thereof, in an amount effective to contact and bind to the tumor and/or surrounding tissue;
b. irradiating the tumor and/or surrounding tissue at a wavelength absorbed by the bound compound;
c. and detecting a signal from the irradiated bound compound, thereby imaging the tumor and/or surrounding tissue.

Preferably, the compound used for imaging comprises a chelating group having a radionuclide that emits gamma-rays or positrons, or an optical dye or a fluorophore, or other detectible radiation.

Still another embodiment of the invention is a method of treating diseased tissue. The method comprises:
a. administering to a subject, a compound disclosed herein, or a pharmaceutically acceptable salt thereof, or a pharmaceutical composition thereof, in an amount effective to contact and bind to the diseased tissue;
b. using the compound as a fiducial, irradiating the region of the bound compound with one or more doses of external beam radiation,

thereby treating the diseased tissue with radiation.

Preferably, the compound used for the fiducial comprises a chelating group having a radionuclide that emits gamma-rays or positrons, or an optical dye or a fluorophore, or other detectible radiation.

Even still another embodiment of the invention is a method of treating diseased tissue. The method comprises:
a. administering to a subject, a compound disclosed herein, or a pharmaceutically acceptable salt thereof, or a pharmaceutical composition thereof, in an amount effective to contact and bind to the diseased tissue;
b. using the compound as a fiducial for guided surgery applications, to resect the region of the diseased tissue

thereby excising the diseased tissue.

Preferably, the compound used for the fiducial comprises a chelating group having a radionuclide that emits gamma-rays or positrons, or an optical dye or a fluorophore, or other detectible radiation.

### BRIEF DESCRIPTION OF THE FIGURES

FIG. 1A-B show compound biodistribution of [¹⁸F]RTX-1312S in BALB/c nude mice previously transplanted with U-87 (human glioblastoma) cells, which express FAP.

FIG. 2 shows the organ biodistribution of [Cu-67]RTX-1363S in BALB/C nude mice previously transplanted with U-87MG cells.

FIG. 3 shows the organ biodistribution of [Lu-177]RTX-1354S in BALB/C nude mice previously transplanted with U-87MG cells.

FIG. 4 shows the organ biodistribution of [Lu-177]RTX-1359R in BALB/C nude mice previously transplanted with U-87MG cells.

FIG. 5 shows a comparison of the biodistribution of [Ac-225]RTX-1399R in the tumor compared to the blood in tumor-bearing JAX nude mice.

### DETAILED DESCRIPTION

Disclosed herein are a series of compounds that bind with high affinity to the extracellular domain of FAP and are capable of delivering a payload to a tissue expressing FAP. Compounds of the invention are described herein below.

A first embodiment of the invention is a compound represented by structural formula (I) or a pharmaceutically acceptable salt thereof. The variables in structural formula (I) are defined above in the Summary.

A second embodiment of the invention is compound represented by structural formula (II):

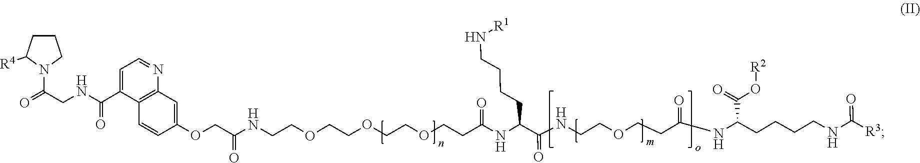

or a pharmaceutically acceptable salt thereof, wherein m is an integer from 0 to 12; o is 0 or 1; and R² is H or C₁-C₄ alkyl; and the remainder of the variables are as described in the first embodiment.

A third embodiment of the invention is a compound represented by structural formula (III):

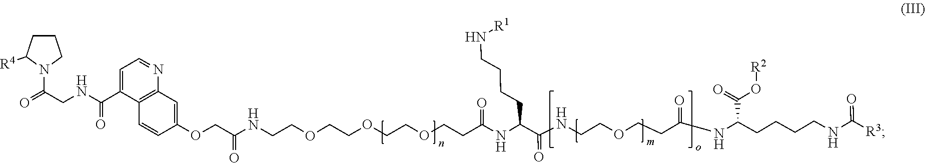

or a pharmaceutically acceptable salt thereof; wherein the variables are as described in the second embodiment.

A fourth embodiment of the invention is compound represented by structural formula (IV):

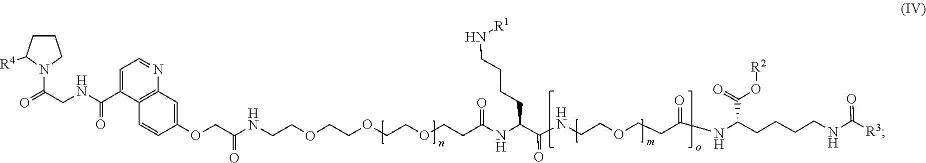

or a pharmaceutically acceptable salt thereof; wherein the variables are as described in the second embodiment.

A fifth embodiment of the invention is a compound represented by structural formula (I), (II), (III) or (IV), or a pharmaceutically acceptable salt thereof, wherein R³ is C₁-C₈ alkyl or C₁-C₄ aralkyl optionally substituted with C₁-C₄ alkyl, and the remainder of the variables are as described in the first or second embodiment. Alternatively, R³ is methyl, propyl, pentyl, heptyl, (4-isobutylphenyl)methyl, (4-isobutylphenyl)propyl.

A sixth embodiment of the invention is a compound represented by structural formula (I), (II), (III) or (IV), or a pharmaceutically acceptable salt thereof, wherein m is 3 to 12 and o is 1. Alternatively, m is 8 and o is 1. In another alternative, o is 0. The remainder of the variables are as described in the first, second or fifth embodiment.

A seventh embodiment of the invention is a compound represented by structural formula (I), (II), (III) or (IV), or a pharmaceutically acceptable salt thereof, wherein n is 1 and o is 0; and the remainder of the variables are as described for the first embodiment, second, fifth or sixth embodiment.

An eighth embodiment of the invention is a compound represented by structural formula (I), (II), (III) or (IV), or a pharmaceutically acceptable salt thereof, wherein R¹ is a fluorophore or optical dye; and the remainder of the variables are as described in the first second, fifth, sixth or seventh embodiment. In one aspect, the fluorophore is

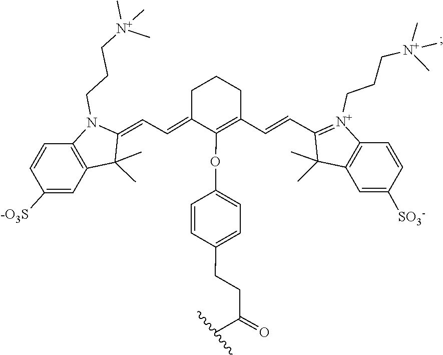

and the optical dye is carbocyanin, indocarbocyanin, oxacarbocyanin, thiacarbocyanin, merocyanin, polymethine, coumarin, rhodamine, xanthene, fluorescein, Borodipyrromethane (BODIPY), VivoTag-680, VivoTag-S750, AlexaFluor dyes (e.g., AlexaFluor660, AlexaFluor680, AlexaFluor700, AlexaFluor750, AlexaFluor790) and DylightFluor dyes.

A ninth embodiment of the invention is a compound represented by structural formula (I), (II), (III) or (IV), or a pharmaceutically acceptable salt thereof, wherein R¹ is a chelating group that is the residue of a chelating agent; and the remainder of the variables are as described in the first, second, fifth, sixth or seventh embodiment. Suitable chelating agents and residues of chelating agents are described below.

A tenth embodiment of the invention is a compound represented by structural formula (I), (II), (III) or (IV), or a pharmaceutically acceptable salt thereof, wherein R¹ is a benzoyl group optionally substituted by one or more groups represented by R⁵; each R⁵ is independently selected from halo, cyano, halomethyl, N⁺(CH₃)₃W⁻; and W⁻ is a pharmaceutically acceptable anion; and the remainder of the variables are as described in the first, second, fifth, sixth or seventh embodiment. In one aspect, the halo group represented by R⁵ is ¹⁸F.

An eleventh embodiment of the invention is a compound represented by structural formula (I), (II), (III) or (IV), or a pharmaceutically acceptable salt thereof, wherein each R⁵ is independently selected from fluoro, cyano, trifluoromethyl, N⁺(CH₃)₃W⁻; and the remainder of the variables are as described in the first, second, fifth, sixth, seventh or tenth embodiment. In one aspect, the fluoro group represented by R⁵ is F¹⁸.

A twelfth embodiment of the invention is a compound represented by structural formula (I), (II), (III) or (IV), or a pharmaceutically acceptable salt thereof, R² is H and R⁴ is CN; and the remainder of the variables are as described in the first, second, fifth, sixth, seventh, eighth, ninth, tenth or eleventh embodiment.

Also included in the invention are the compounds whose preparation is described in the Exemplification and shown in the Figures, both pharmaceutically acceptable salts thereof and the neutral form. For those compounds comprising a chelating group, chelation with a radionuclide is also included in the invention.

Also included in the invention are the compounds shown below, both pharmaceutically acceptable salts thereof and the neutral form. Chelation with a radionuclide is also included in the invention.

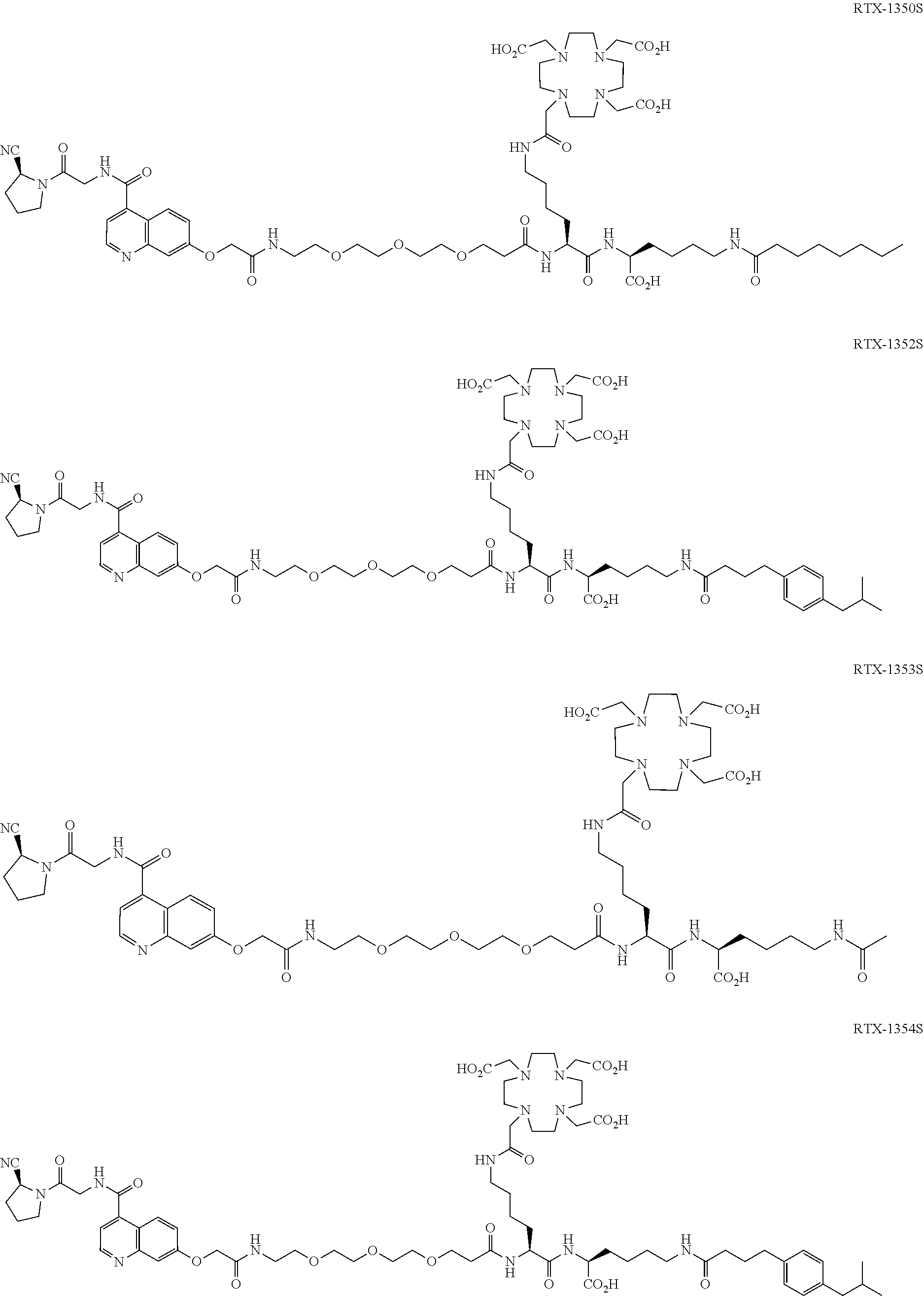

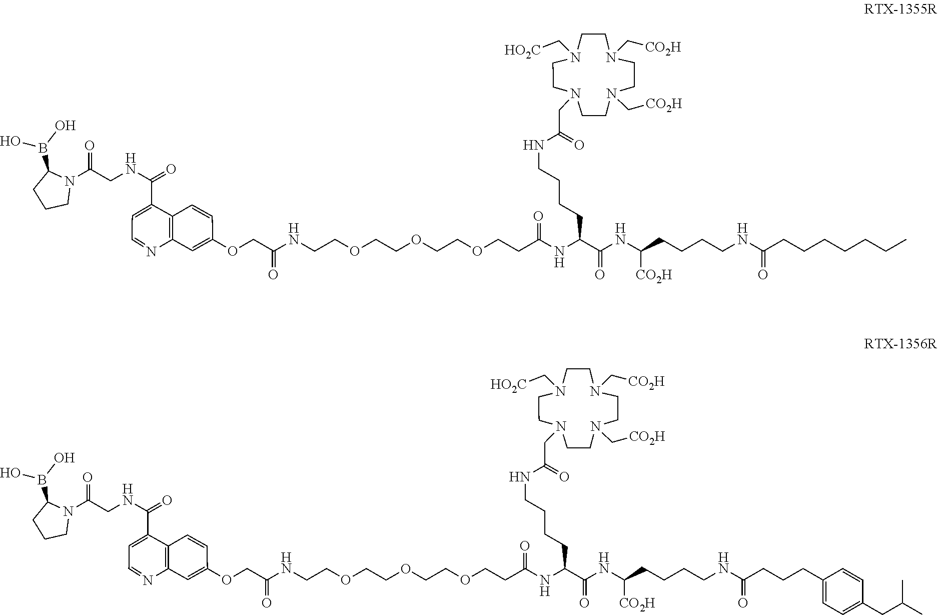 [SMILES: CCCCCCCC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3B(O)O)ccnc2c1)C(=O)O ; CC(C)Cc1ccc(CCCC(=O)NCCCC[C@H](NC(=O)[C@H](CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B(O)O)ccnc3c2)C(=O)O)cc1]

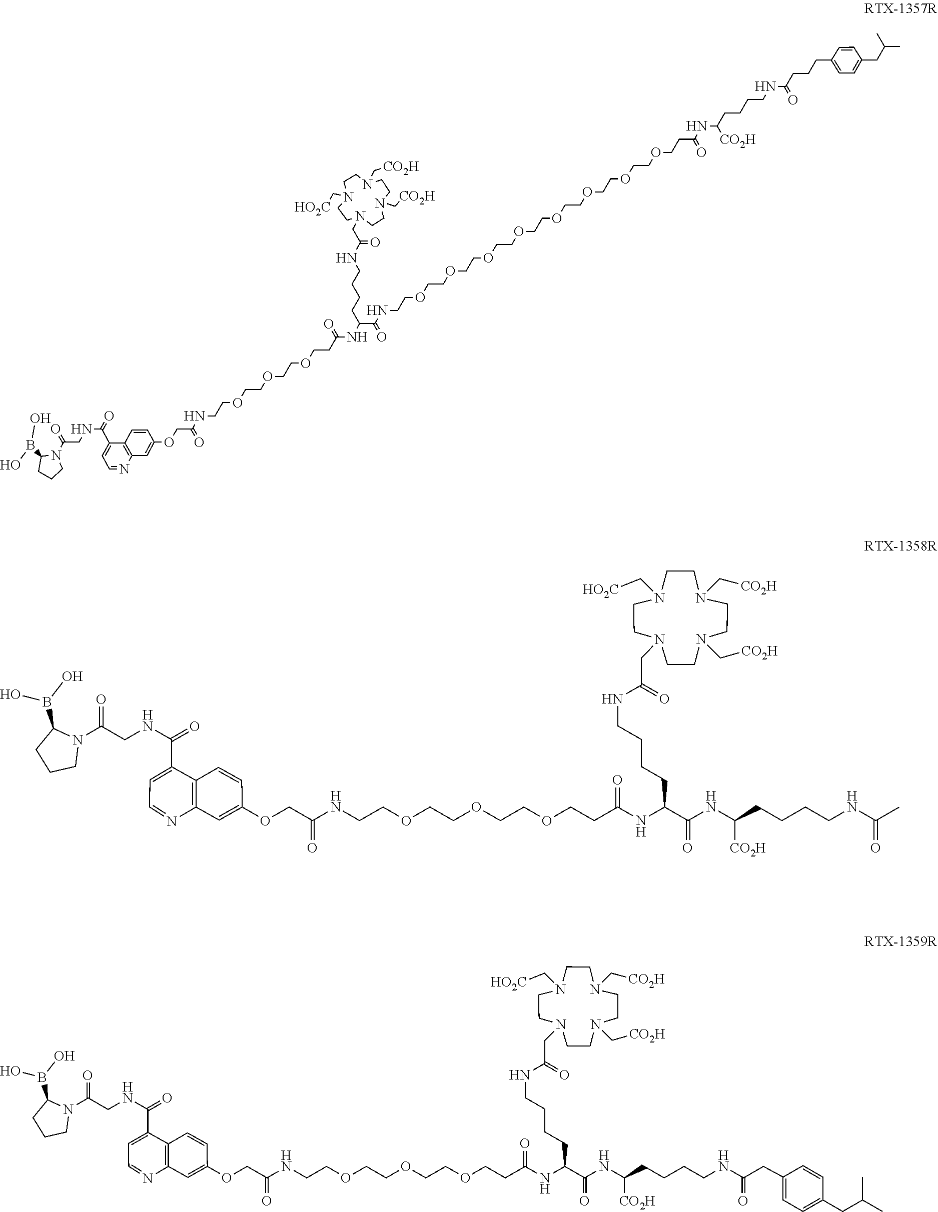

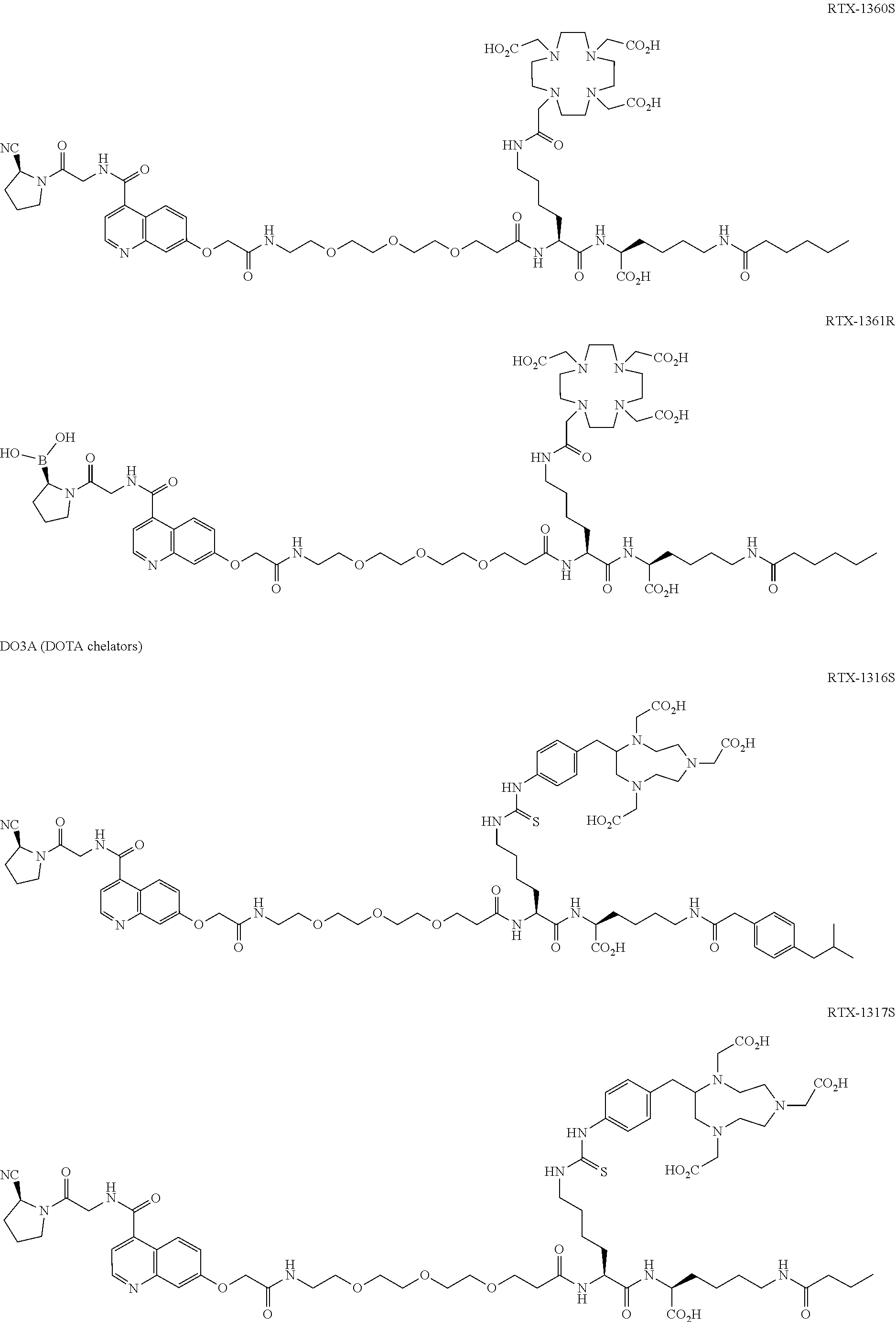

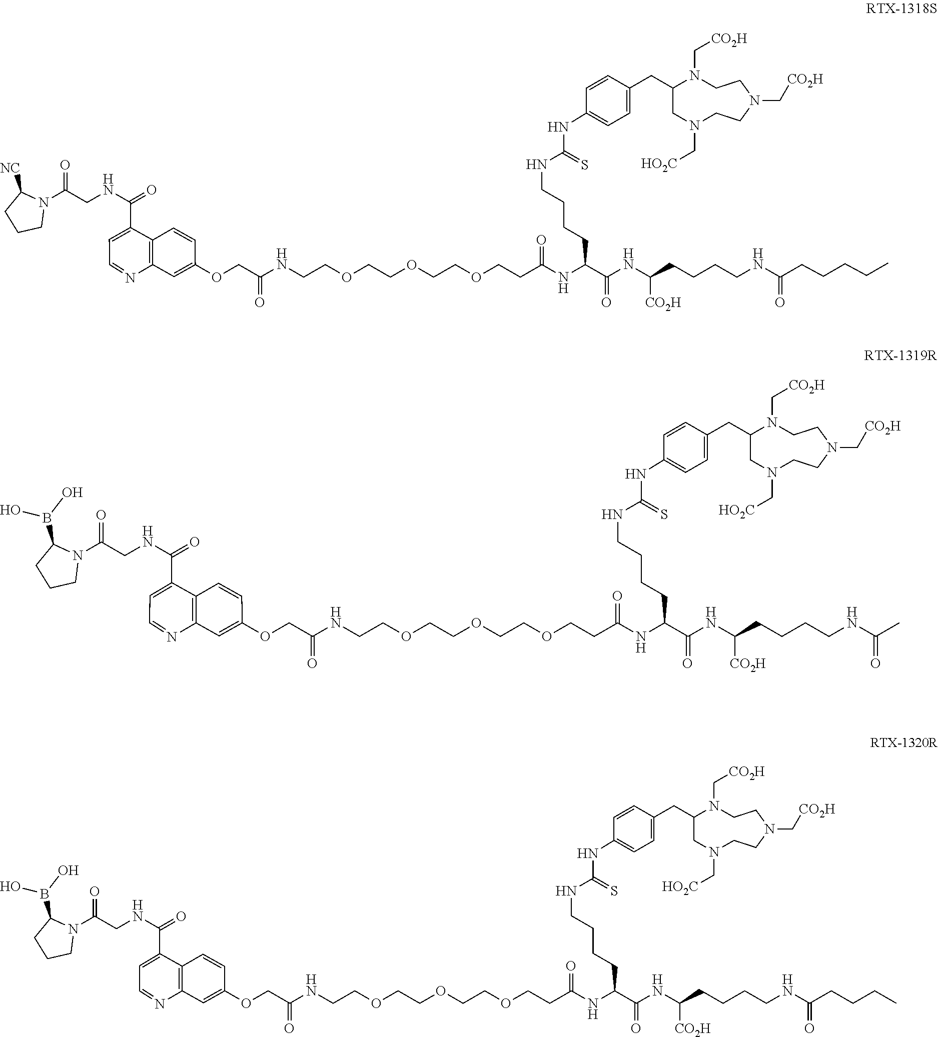

### Examples of NOTA Chelators

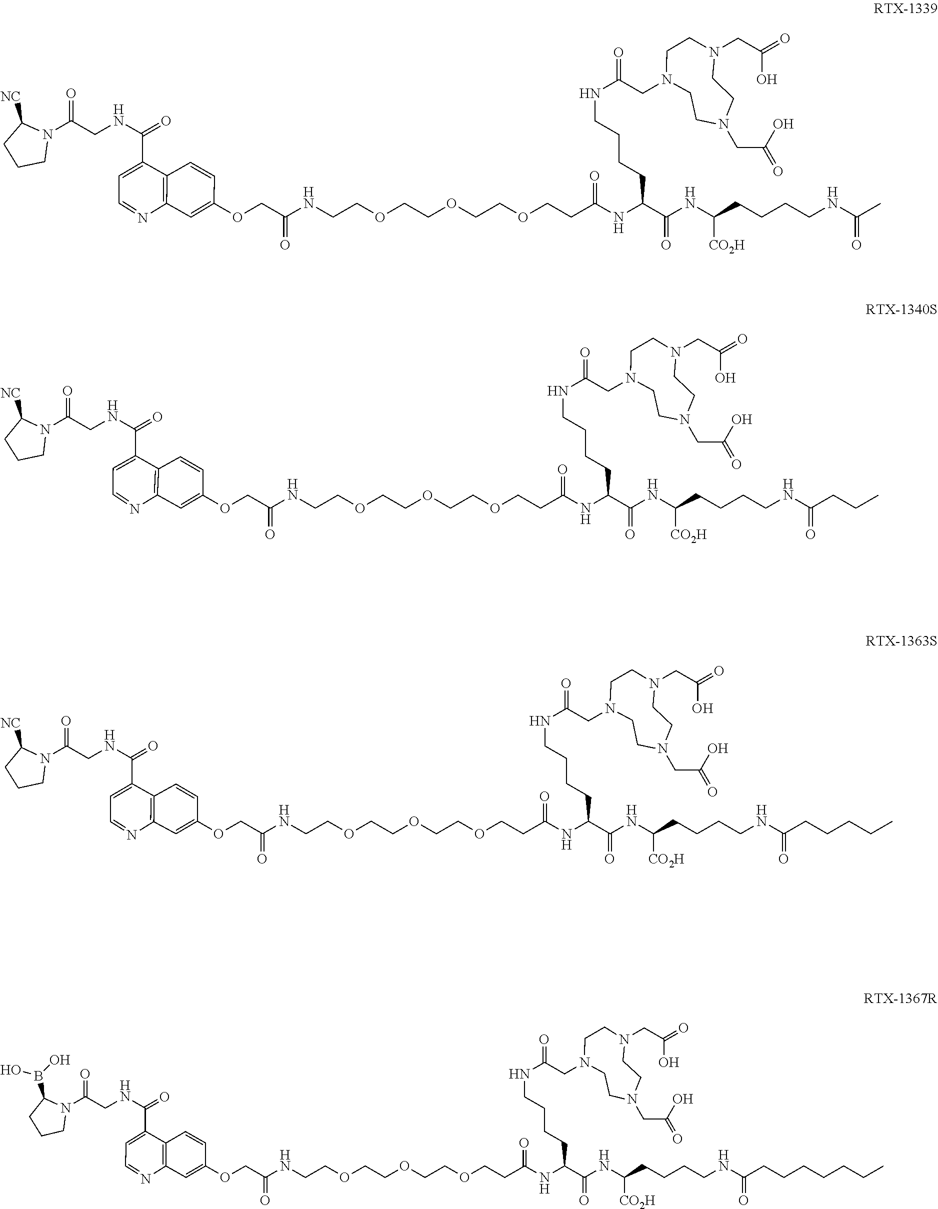

### Examples of N3O2 (NOTA) Chelators

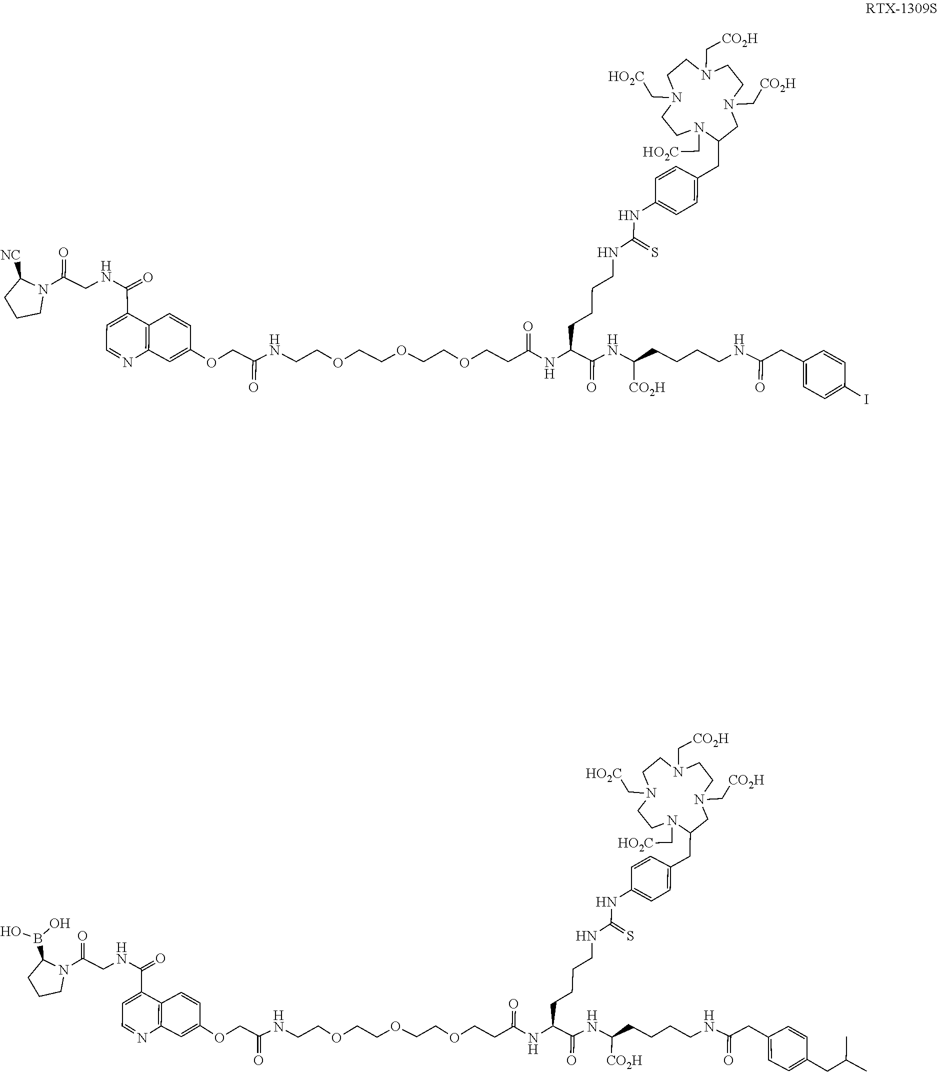 [SMILES: N#C[C@@H]1CCCN1C(=O)CNC(=O)c1ccnc2cc(OCC(=O)NCCOCCOCCOCCC(=O)N[C@@H](CCCCNC(=S)Nc3ccc(CC4CN(CC(=O)O)CCN(CC(=O)O)CCN(CC(=O)O)CCN4CC(=O)O)cc3)C(=O)N[C@@H](CCCCNC(=O)Cc3ccc(I)cc3)C(=O)O)ccc12 ; CC(C)Cc1ccc(CC(=O)NCCCC[C@H](NC(=O)[C@H](CCCCNC(=S)Nc2ccc(CC3CN(CC(=O)O)CCN(CC(=O)O)CCN(CC(=O)O)CCN3CC(=O)O)cc2)NC(=O)CCOCCOCCOCCNC(=O)COc2ccc3c(C(=O)NCC(=O)N4CCC[C@H]4B(O)O)ccnc3c2)C(=O)O)cc1]

### Examples of DOTA Chelators

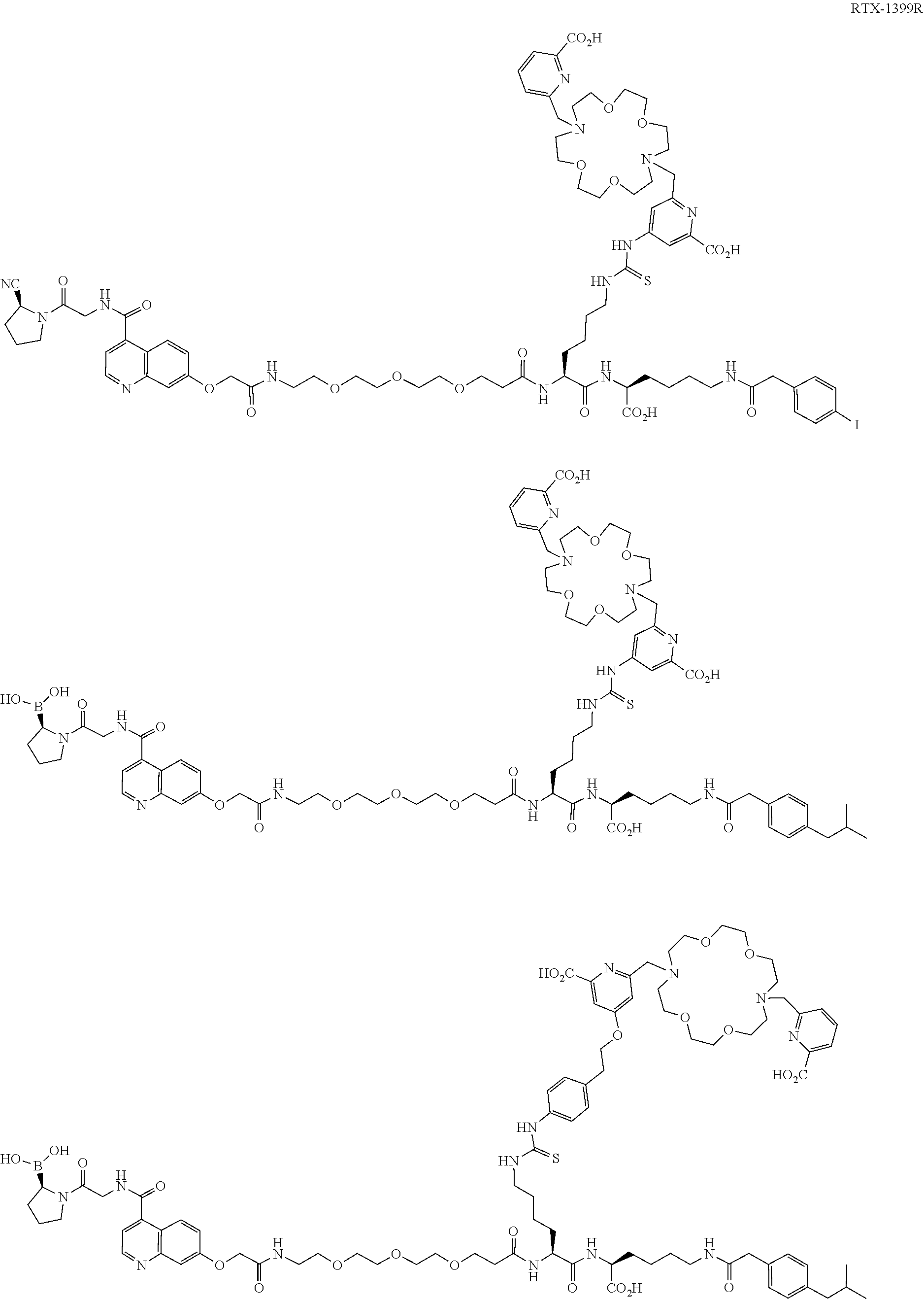

### Examples of NCS-Macropa Chelators

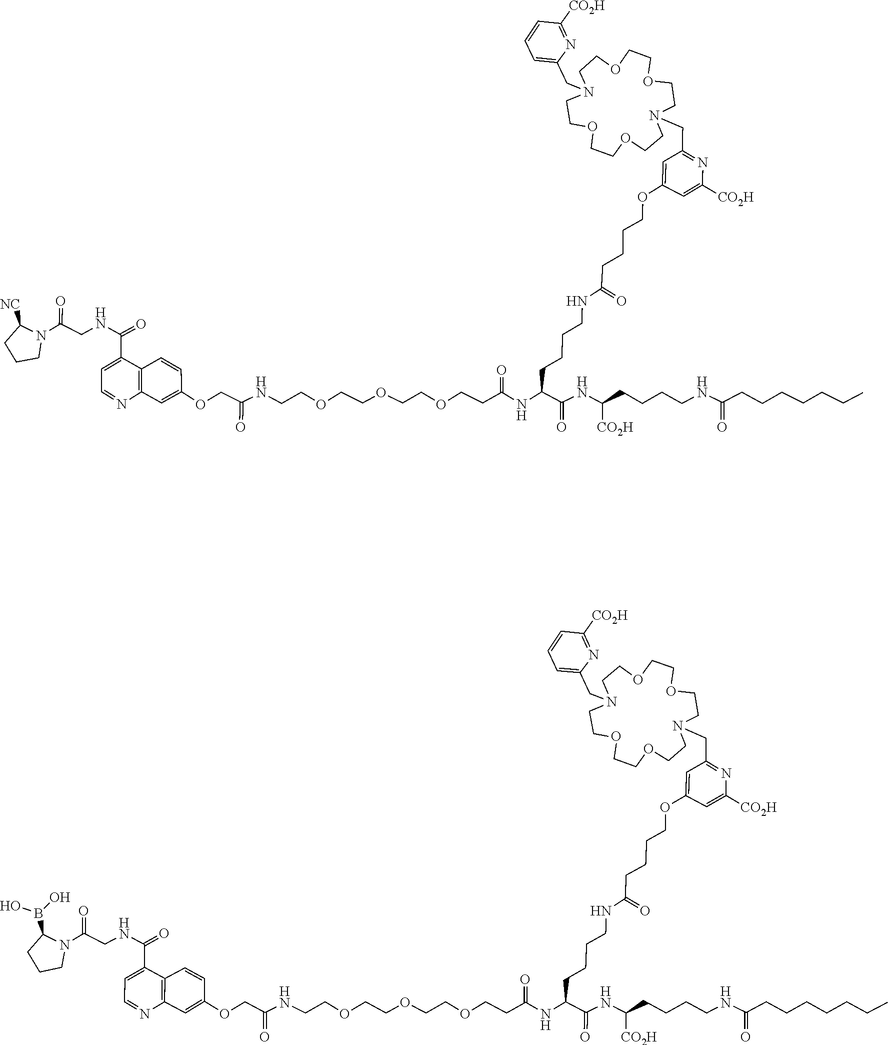 [SMILES: CCCCCCCC(=O)NCCCC[C@H](NC(=O)[C@H](CCCCNC(=O)CCCCOc1cc(CN2CCOCCOCCN(Cc3cccc(C(=O)O)n3)CCOCCOCC2)nc(C(=O)O)c1)NC(=O)CCOCCOCCOCCNC(=O)COc1ccc2c(C(=O)NCC(=O)N3CCC[C@H]3C#N)ccnc2c1)C(=O)O ; CCCCCCCC(=O)NCCCC[C@H](NC(=O)[C@H](CCCCNC(=O)CCCCOc1cc(CN2CCOCCOCCN(Cc3cccc(C(=O)O)n3)CCOCCOCC2)nc(C(=O)O)c1)NC(=O)CCOCCOCCOCCNC(=O)COc1ccc2c(C(=O)NCC(=O)N3CCC[C@H]3B(O)O)ccnc2c1)C(=O)O]

### Examples of NCO-Macropa Chelators

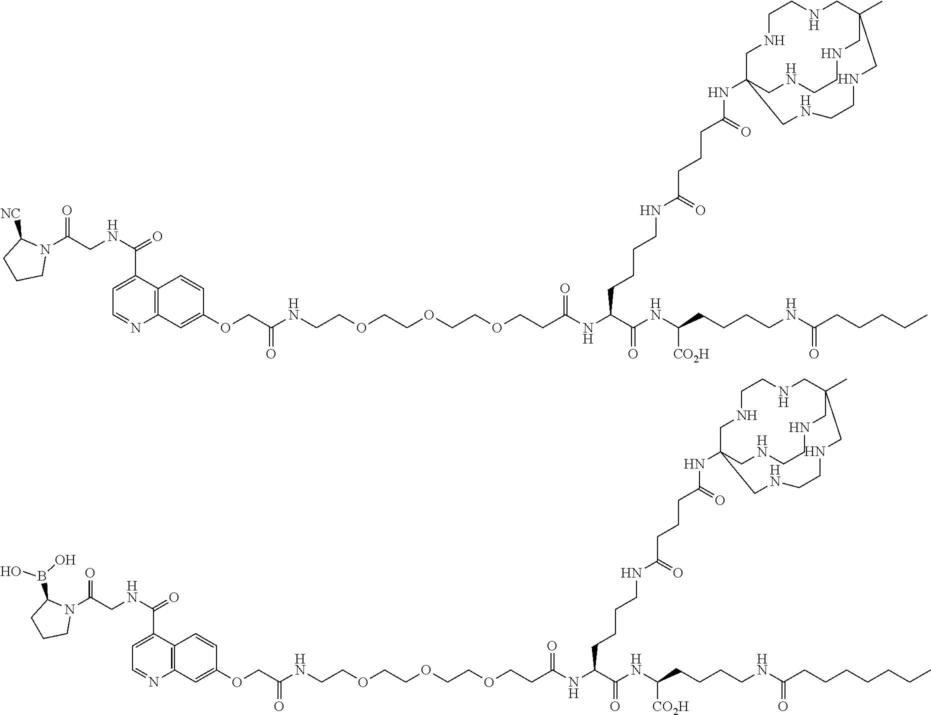

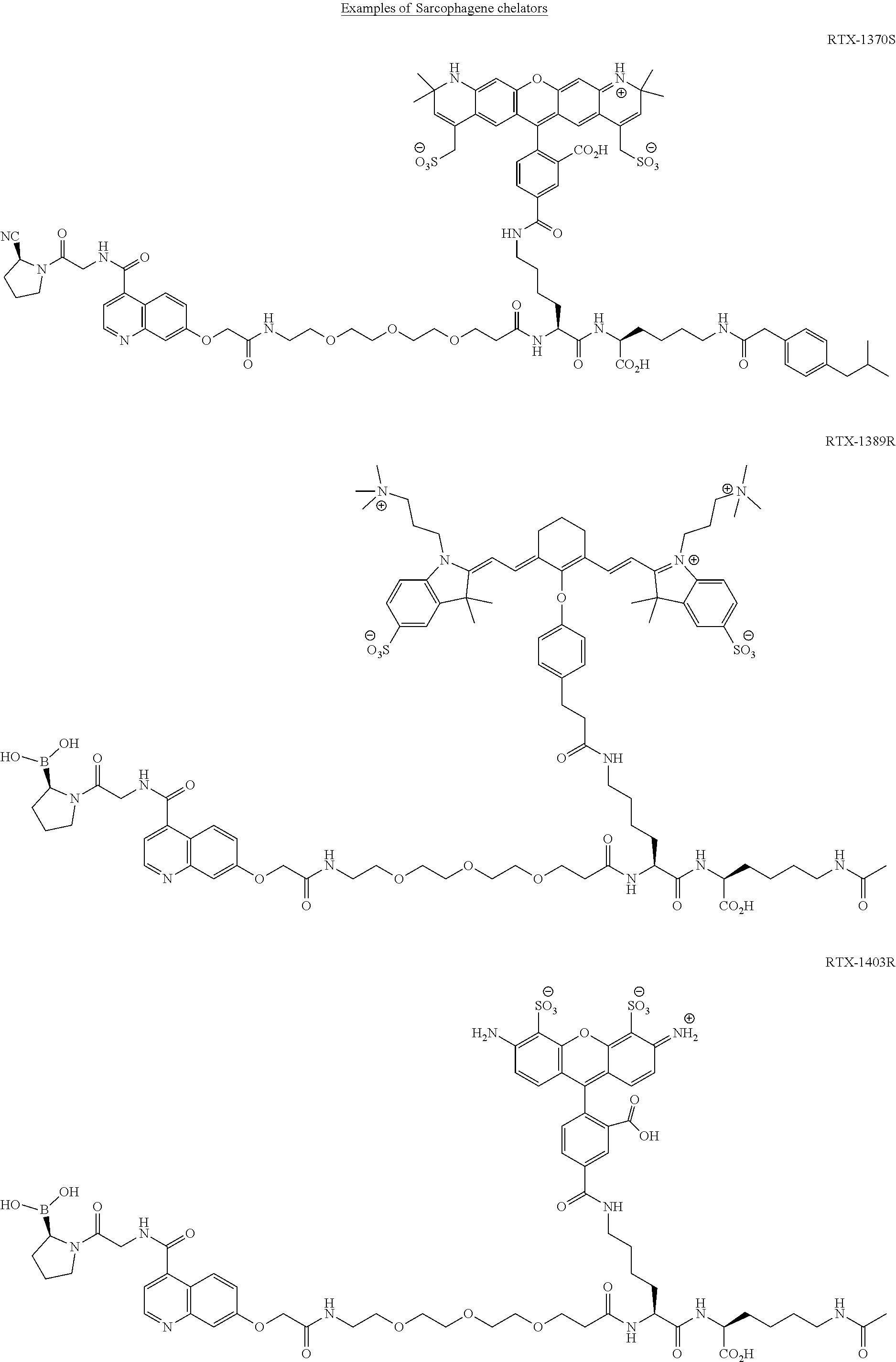

### Examples of Optical and Near-IR Imaging Compounds

The nomenclature in which a compound name is preceded by an isotope indicates that the isotope is chelated to the chelating group of the compound. For example, “[Cu-67]RTX-1363S” refers to RTX-1363S in which its chelating group is chelated with 67Cu.

Exemplary compounds of the invention (with their chelating group) include [68Ga]RTX-1339S; [68Ga]RTX-1340S; [68Ga]RTX-1363S; [68Ga]RTX-1367S; [64Cu]RTX-1339S; [64Cu]RTX-1340S; [64Cu]RTX-1363S; [64Cu]RTX-1367S; [67Cu]RTX-1339S; [67Cu]RTX-1340S; [67Cu]RTX-1363S; [67Cu]RTX-1367S; [Al18F]RTX-1339S; [Al18F]RTX-1340S; [Al18F]RTX-1363S; and [Al18F]RTX-1367S.

Other exemplary compounds of the invention (with their chelating group) include [68Ga]RTX-1350S; [68Ga]RTX-1352S; [68Ga]RTX-1353S; [68Ga]RTX-1354S; [68Ga]RTX-1355S; [68Ga]RTX-1356R; [68Ga]RTX-1357R; [68Ga]RTX-1358R; [68Ga]RTX-1359R; [68Ga]RTX-1360S; [68Ga]RTX-1360R; [177Lu]RTX-1350S; [177Lu]RTX-1352S; [177Lu]RTX-1353S; [177Lu]RTX-1354S; [177Lu]RTX-1355S; [177Lu]RTX-1356R; [177Lu]RTX-1357R; [177Lu]RTX-1358R; [177Lu]RTX-1359R; [177Lu]RTX-1360S; [177Lu]RTX-1360R; [225Ac]RTX-1350S; [225Ac]RTX-1352S; [225Ac]RTX-1353S; [225Ac]RTX-1354S; [225Ac]RTX-1355S; [225Ac]RTX-1356R; [225Ac]RTX-1357R; [225Ac]RTX-1358R; [225Ac]RTX-1359R; [225Ac]RTX-1360S; and [225Ac]RTX-1360R.

Another exemplary compound of the invention (with their chelating group) includes [AL18F]RTX-1312S.

Compounds with Macropa chelators are commonly chelated with 225Ac. Accordingly, another compound of the invention (with their chelating group) includes [Ac225]RTX-1399R.

“Aliphatic” means a saturated or unsaturated straight-chain or branched monovalent or bivalent hydrocarbon radical. Unless otherwise specified, an aliphatic group typically has 1 to 10 carbon atoms. “Alkyl” means a saturated aliphatic straight-chain or branched monovalent aliphatic radical. Unless otherwise specified, an alkyl group typically has 1 to 10 carbon atoms (C₁₋₁₀ alkyl), alternatively, 1 to 6 carbon atoms (C₁₋₃ alkyl) (i.e., 1, 2 or 3).

“Cyclic aliphatic” means a saturated or unsaturated, monovalent or bivalent, cyclic hydrocarbon ring radical. Unless otherwise specified, a cyclic aliphatic has 3 to 8 ring carbon atoms (C₃₋₈ cycloalkyl). “Cycloalkyl” means a saturated aliphatic cyclic aliphatic. Unless otherwise specified, a cycloalkyl has 3 to 8 ring carbon atoms.

“Aryl”, alone or part or a larger moiety such as “aralkyl” is carbocyclic aromatic group such as phenyl or napthyl.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are non-superimposable mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

The FAP-targeted compounds of the present invention are useful imaging agents for diagnostic applications. For example, these can be conjugated to various metals for magnetic resonance imaging applications or conjugated to an optical dye or a fluorophore or other detectable moiety (i.e., dyes, quantum dots, etc.) for histochemistry applications and luminescence imaging applications. Likewise, the compounds can be radiolabeled and used in nuclear medicine applications. Radionuclides that can be used for imaging applications are referred to herein as “imaging radionuclides”. Nonlimiting examples of imaging radionuclides include ¹⁸F, ⁶⁴Cu or ⁶⁸Ga, which are suitable for use in PET imaging applications, and ⁶⁷Cu or ¹⁷⁷Lu, which are typically therapy nucleotides but are also suitable for use in SPECT imaging applications.

The FAP-targeting compounds of the present invention are useful therapy compounds. Such a therapy compound includes a FAP-targeted compound of the invention with a suitable therapeutic moiety. The FAP-targeted compound may be separated from the therapeutic moiety by a covalent linker. The separation between these (on the basis of a contiguous atom count) may be from about 4 atoms to about 100 atoms. Furthermore, by including additional targeting structures on the compound, the pharmacokinetics of the compound can be altered. For example, using a blood-targeting moiety it is possible to increase circulatory residence time, which has the effects of increasing tumor perfusion and loading while reducing accumulation of the radiotherapy compound in non-target tissues. See, for example U.S. Pat. No. 11,285,277 which describes a trifunctional (“Trillium”) compound having a tumor-targeting domain, a blood protein binding domain and a third domain constituting a cytocidal or cytostatic therapeutic agent. In a currently preferred embodiment, the FAP-binding compounds of the present invention are capable of being adapted to the Trillium scaffold by covalent linkage, to constitute the tumor-targeting domain for such FAP-targeted Trillium agents. Exemplary constructs include drug conjugates having a toxin, a venom, a metabolic poison, or a chemotherapy drug, as well as radiotherapy compounds having an alpha-emitting radionuclide, a beta-emitting radionuclide, an Auger electron emitting radionuclide or one that emits a spectrum of radiation upon decay (including positron emissions, which are also suitable for diagnostic uses).

For radiotherapy, the FAP-targeted compound is conjugated to a chelator, which is selected based on its suitability to hold an appropriate therapeutic radionuclide. A “therapeutic radionuclide” is a radionuclide that can be used for therapeutic purposes, e.g., for treating cancer or fibrotic tissue due to their radioactive emissions, which have cytotoxic effects on targeted tissues (i.e., FAP-expressing cancers and tumor microenvironments, malignancies, and fibrotic cells). Although targeted radiotherapy has been practiced for some time using macrocyclic complexes of radionuclides, the macrocycles currently in use (e.g., DOTA) generally form complexes with many therapeutic radionuclide metals, such as actinium, radium, bismuth, astatine, lutetium, and lead isotopes among others. Instability of many known macrocyclic-containing compounds can result in some dissociation of the radionuclide from the macrocycle, and this results in a lack of selective delivery to the intended targeted tissue, which can also result in toxicity to non-targeted tissue. Alpha-emitting radionuclides such as ²²⁵Ac can provide much greater cytotoxic effects, and thus, for therapy are considered substantially more potent than beta-emitting radionuclides. But this toxicity requires a chelator with increased retention of the chelated metal. U.S. Pat. No. 11,279,698 (see also, PCT/US2018/025488, and PCT/US2019/062479) describes a novel chelator (“Macropa”) and its' use as a chelator for ²²⁵Ac, including as a component of a Trillium PK-tuned, targeted radiotherapy agent. The ratio of tumor activity to kidney activity of 1 or greater may persist up to about 36 hours after administration of the radiotherapeutic, and in the case of an 225Ac Trillium-based therapeutic may persist for 72, or even 128 hours, or longer, maximizing the therapeutic effects of the radiation on the target tissues.

Accordingly, an exemplary preferred FAP-targeted Trillium compound will have in its' third (non-targeting) domain, a chelator. Macropa is the currently preferred chelator for ²²⁵Ac-FAP-targeted Trillium compounds. (see also, PCT/CA2021/050226).

The radionuclide usable with the compounds disclosed herein depends on the application, radiation type desired and half-life as will be apparent to those of skill in the art. Exemplary radionuclides include: ¹⁷⁷Lu, ¹⁷⁵Lu, ⁴⁵Sc, ⁶⁴Cu, ⁶⁷Cu, ⁶⁸Cu, ⁶⁶Ga, ⁶⁷Ga, ⁶⁸Ga, ⁶⁹Ga, ⁷¹Ga, ⁹⁰Y, ⁸⁹Y, ⁸⁶Y, ⁸⁹Zr, ⁹⁰Y, ⁹⁹mTc, ¹¹¹In, ¹¹³In, ¹¹⁵In, ¹³⁹La, ¹³⁴Ce, ¹³⁶Ce, ¹³⁸Ce, ¹⁴⁰Ce, ¹⁴²Ce, ¹⁵¹Eu, ¹⁵³Eu, ¹⁵²Dy, ¹⁴⁹Tb, ¹⁵⁹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th, ²²⁵Ac, ²³³Ra, ¹⁵²Dy, ²¹³Bi, ²¹²Bi, ²¹¹Bi, ²⁰³Pb, ²¹²Pb, ²⁵⁵Fm, and uranium-230. The radionuclide of any embodiment herein may be both a therapeutic radionuclide, and a diagnostic radionuclide depending on its' decay profile. Currently preferred alpha-emitting radionuclides for therapy applications include ²²⁵Ac, ²³³Ra, and ²¹²Pb. Currently preferred beta-emitting radionuclides for therapy applications include ¹⁷⁷Lu, ⁹⁰Y, and ⁶⁷Cu.

Chelating groups and polyaza polycarboxylic macrocycles useful in the present technology include, and refer to a group that can chelate, bind or otherwise deliver a radionuclide to a therapeutic or diagnostic target. A chelating group is the residue of a chelating agent after the chelating agent reacts with a nucleophilic group in a compound to form a targeted bivalent radio pharmaceutical or radio diagnostic agent that can bind and deliver a radionuclide. In the case of the disclosed compounds, the reactive group is the side chain amine of the lysyl group in the penultimate precursor that reacts with the chelating agent to form the disclosed compounds. Examples of chelating agents include, but are not limited to, a covalently conjugated substituted or unsubstituted member of the following group: 1,4,7-triazacyclononane-1,4,7-triacetic acid (NOTA), p-SCN-Bn-NOTA, 1,4,7,10-tetraazacyclododecane-1,4,7,10-tetraacetic acid (DOTA), p-SCN-Bn-DOTA (also known as 2B-DOTA-NCS), PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl-1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA, {4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2-(bis(carboxymethyl)amino)ethyl)-1,4,7,10-tetraazacyclododecane-1,7-diyl)diacetic acid, N,N′-bis[(6-carboxy-2-pyridil)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 3,9-carboxymethyl-6-(2-methoxy-5-isothiocyanatophenyl)carboxymethyl-3,6,9,15-tetraazabicyclo-[9.3.1]pentadeca-1(15), 11,13-triene and 2-[4,7,10-tris(2-amino-2-oxoethyl)-1,4,7,10-tetrazacyclododec-1-yl]acetamide (TCMC or DOTAM). In a currently preferred embodiment, the chelator is the residue of a polyaza polycarboxylic macrocycle, such as Macropa NCS or NCO-Macropa. In another aspect, the chelator is the residue of a siderophores, In one aspect, ²²⁵Ac is the radionuclide for Macropa NCS or NCO-Macropa. In another aspect, the chelating agent is the residue of p-SCN-Bn-DOTA, p-SCN-Bn-NOTA, NOTA or DOTA. In another embodiment, the chelating agent is sarcophagene chelator (compounds of the invention with sarcophagene chelators can be prepared according to procedures disclosed in WO2021225760, the entire teachings of which are incorporated herein by reference). In another aspect, the chelating agent is the residue of p-SCN-Bn-DOTA, p-SCN-Bn-NOTA, NOTA or DOTA chelated with ⁶⁸Ga.

As noted above, complexes of the disclosed compounds or pharmaceutically acceptable salts thereof may contain one or more radionuclides which are suitable for use as radio-imaging agents. Imaging methods include positron emission tomography (PET) or single photon emission computed tomography (SPECT). Accordingly in another aspect, the invention provides for theranostic applications, i.e., methods where a subject with a cancer, a tumor or a fibrotic disease is administered an effective amount of a disclosed compound (or a pharmaceutically acceptable salt thereof) having a chelator, which is complexed to an imaging radionuclide for imaging applications, and administered an effective amount of the compound complexed to a therapeutic radionuclide for treatment. Exemplary cancers which can be imaged and/or treated with the disclosed compounds or a pharmaceutically acceptable salt thereof include pancreatic cancer, liver cancer, gall bladder cancer, neuroblastoma, breast cancer, ovarian cancer, esophageal cancer, kidney cancer, prostate cancer, colorectal cancer, soft tissue sarcoma, bone sarcoma or melanoma.

A “subject” is a mammal in need of medical treatment or diagnosis, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The disclosed compounds or pharmaceutically acceptable salts thereof (including chelation with a radionuclide) or pharmaceutical compositions thereof, may be administered orally or via a parenteral route, usually injection or infusion. A “parenteral administration route” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

“Effective amount” of the disclosed compounds or pharmaceutically acceptable salts thereof (including chelation with a radionuclide) means that amount of therapeutic agent alone, or in combination with other therapies, that provides a therapeutic benefit in the treatment of prevention of a disease, that improves overall therapy, reduces or avoids symptoms or causes of disease, or enhances the therapeutic efficacy of or synergies with another therapeutic agent. With respect to imaging, “diagnostically effective amount” refers to the amount which will result in a useful image for diagnosing, e.g., the presence of a tumor. “Effective amount” of the disclosed compounds or pharmaceutically acceptable salt thereof, is determined by the physician on the basis of the patient-specific parameters, such as age, weight, sex, severity of the disease, etc. The dosage is preferably from 0.00001 mg/kg to 100 mg/kg body weight.

Corresponding to the kind of administration, the medicament is suitably formulated, e.g. in the form of solutions or suspensions, simple tablets or dragees, hard or soft gelatine capsules, suppositories, ovules, preparations for injection, which are prepared according to common galenic methods.

When solutions for infusion or injection are used, they are preferably aqueous solutions or suspensions, it being possible to produce them prior to use, e.g. from lyophilized preparations which contain the active substance as such or together with a carrier, such as mannitol, lactose, glucose, albumin and the like. The ready-made solutions are sterilized and, where appropriate, mixed with excipients, e.g. with preservatives, stabilizers, emulsifiers, solubilizers, buffers and/or salts for regulating the osmotic pressure. The sterilization can be obtained by sterile filtration using filters having a small pore size according to which the composition can be lyophilized, where appropriate. Small amounts of antibiotics can also be added to ensure the maintenance of sterility.

According to another aspect, a pharmaceutical composition is provided, which is suitable for in vivo imaging and/or radiotherapy of a target tissue. Suitable pharmaceutical compositions may contain a radioimaging agent that has a radionuclide either as an element, (i.e., ¹⁸F), or a diagnostic radioactive metal chelate complex (e.g., with ⁶⁴Cu or ⁶⁸Ga), or a radiotherapeutic agent which is radioactive metal chelate complex, in an amount sufficient for binding to the target tissue, together with a pharmaceutically acceptable radiological vehicle. The radiological vehicle should be suitable for injection or aspiration, such as human serum albumin; aqueous buffer solutions, e.g., tris(hydromethyl) aminomethane (and its salts), phosphate, citrate, bicarbonate, etc; sterile water physiological saline; and balanced ionic solutions containing chloride and or dicarbonate salts or normal blood plasma cautions such as calcium potassium, sodium and magnesium.

The concentration of the radiopharmaceutical agent in the radiological vehicle should be sufficient to provide reasonable binding to the target tissue, such as about 4% to 40% ID/gram. For example, when using an aqueous solution, the human dosage can range from about 1.0 to 500 millicuries of activity. The actual dose administered to a patient for imaging or therapeutic purposes, however, is determined by the physician administering treatment. The imaging agent or therapeutic agent should be administered so as to remain in the patient for about 1 hour to 10 days, although both longer and shorter time periods are acceptable. Therefore, convenient ampoules containing 1 to 10 mL of aqueous solution may be prepared.

Imaging may be carried out in the normal manner, for example by injecting a sufficient amount of the imaging composition to provide adequate imaging and then scanning with a suitable imaging or scanning machine, such as a tomograph or gamma camera. In certain embodiments, a method of imaging a region in a patient includes the steps of: (i) administering to a patient a diagnostically effective amount of a compound complexed with a radionuclide; exposing a region of the patient to the scanning device; and (ii) obtaining an image of the region of the patient. Accordingly, the invention provides a method for obtaining an image of a mammalian subject following administration of the compound. Likewise, imaging can be performed after administration of a therapeutic drug or radiotherapy cycle to assess efficacy. Thus, obtaining an image after administration of the radiotherapeutic may occur after about 1 hour, about 4 hours, about 9 hours, about 12 hours, about 16 hours, about 20 hours, about 24 hours, about 36 hours, about 48 hours, about 72 hours, about 96 hours, about one week, about two weeks, about four weeks, or after completion of a cycle of therapeutic treatments. Thus, in some embodiments, a method of imaging tissue such as FAP-expressing tumor tissue is provided including contacting the tissue with a complex synthesized by contacting an imaging radionuclide with a disclosed compound.

According to another aspect, complexes of the disclosed compounds or pharmaceutically acceptable salts thereof may contain one or more radionuclides which are suitable for use as radio-imaging agents in the field of image guided radiation therapy (IGRT). As described in U.S. Pat. No. 10,688,320B2, IGRT uses images acquired before a treatment session to guide the application of therapeutic radiation during a treatment session. The concentration of the imaging agent or the therapeutic agent in the radiological vehicle should be sufficient to provide satisfactory imaging. For example, when using an aqueous solution, the dosage is about 1.0 to 100 millicuries. Imaging can be performed to provide a fiducial, for guidance for the target region to receive a calculated radiation fluence from a therapeutic radiation source. Similar uses of the compounds as fiducials can be used in guided surgery applications.

The amount of the compound of the present invention, or a formulation comprising a complex of a metal and a compound or pharmaceutically acceptable salt thereof that is administered to a patient depends on several physiological factors that are routinely used by the physician, including the nature of the procedure to be carried out, the volume of tissue to be targeted for imaging or therapy and the body weight and medical history of the patient to be imaged or treated using the compounds.

The examples herein are provided to illustrate advantages of the present technology and to further assist a person of ordinary skill in the art with preparing or using the compounds of the present technology or salts, pharmaceutical compositions, derivatives, prodrugs, or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### EXEMPLIFICATION

### Example 1—Synthesis of RTX-1312S: (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy)ethoxy]ethoxy}propionylamino)-6-[3-(p-{[1,4,7-tris(carboxymethyl)-1,4,7-triazonan-2-yl]methyl}phenyl)thioureido]hexanoylamino]-6-acetylaminohexanoic Acid

### Synthesis of RTX-1312S from Fragments

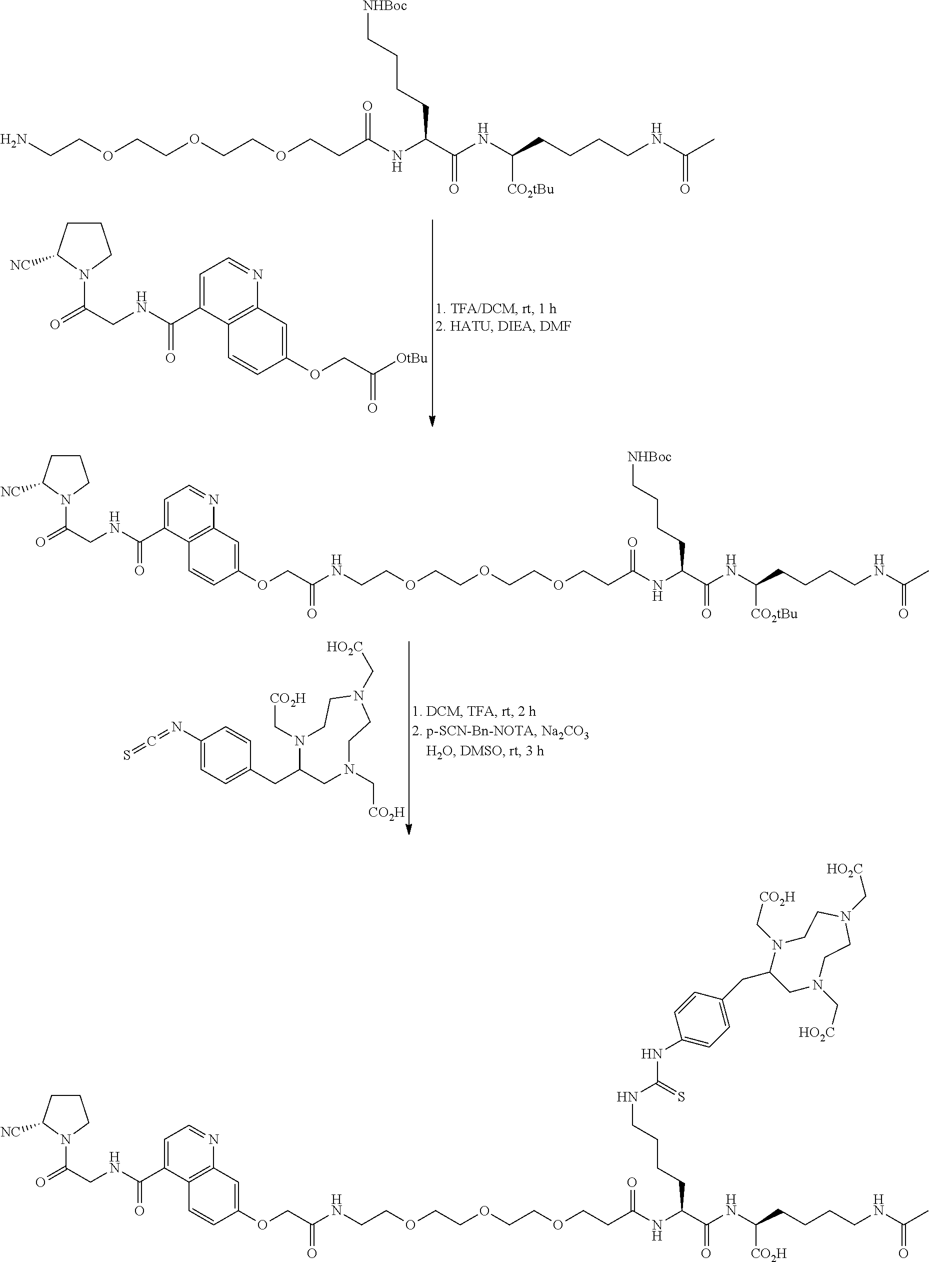 [SMILES: CC(=O)NCCCC[C@H](NC(=O)[C@H](CCCCNC(=O)OC(C)(C)C)NC(=O)CCOCCOCCOCCN)C(=O)OC(C)(C)C ; CC(C)(C)OC(=O)COc1ccc2c(C(=O)NCC(=O)N3CCC[C@H]3C#N)ccnc2c1 ; CC(=O)NCCCC[C@H](NC(=O)[C@H](CCCCNC(=O)OC(C)(C)C)NC(=O)CCOCCOCCOCCNC(=O)COc1ccc2c(C(=O)NCC(=O)N3CCC[C@H]3C#N)ccnc2c1)C(=O)OC(C)(C)C ; O=C(O)CN1CCN(CC(=O)O)CC(Cc2ccc(N=C=S)cc2)N(CC(=O)O)CC1 ; CC(=O)NCCCC[C@H](NC(=O)[C@H](CCCCNC(=S)Nc1ccc(CC2CN(CC(=O)O)CCN(CC(=O)O)CCN2CC(=O)O)cc1)NC(=O)CCOCCOCCOCCNC(=O)COc1ccc2c(C(=O)NCC(=O)N3CCC[C@H]3C#N)ccnc2c1)C(=O)O]

### Synthetic Scheme of Intermediate-1:

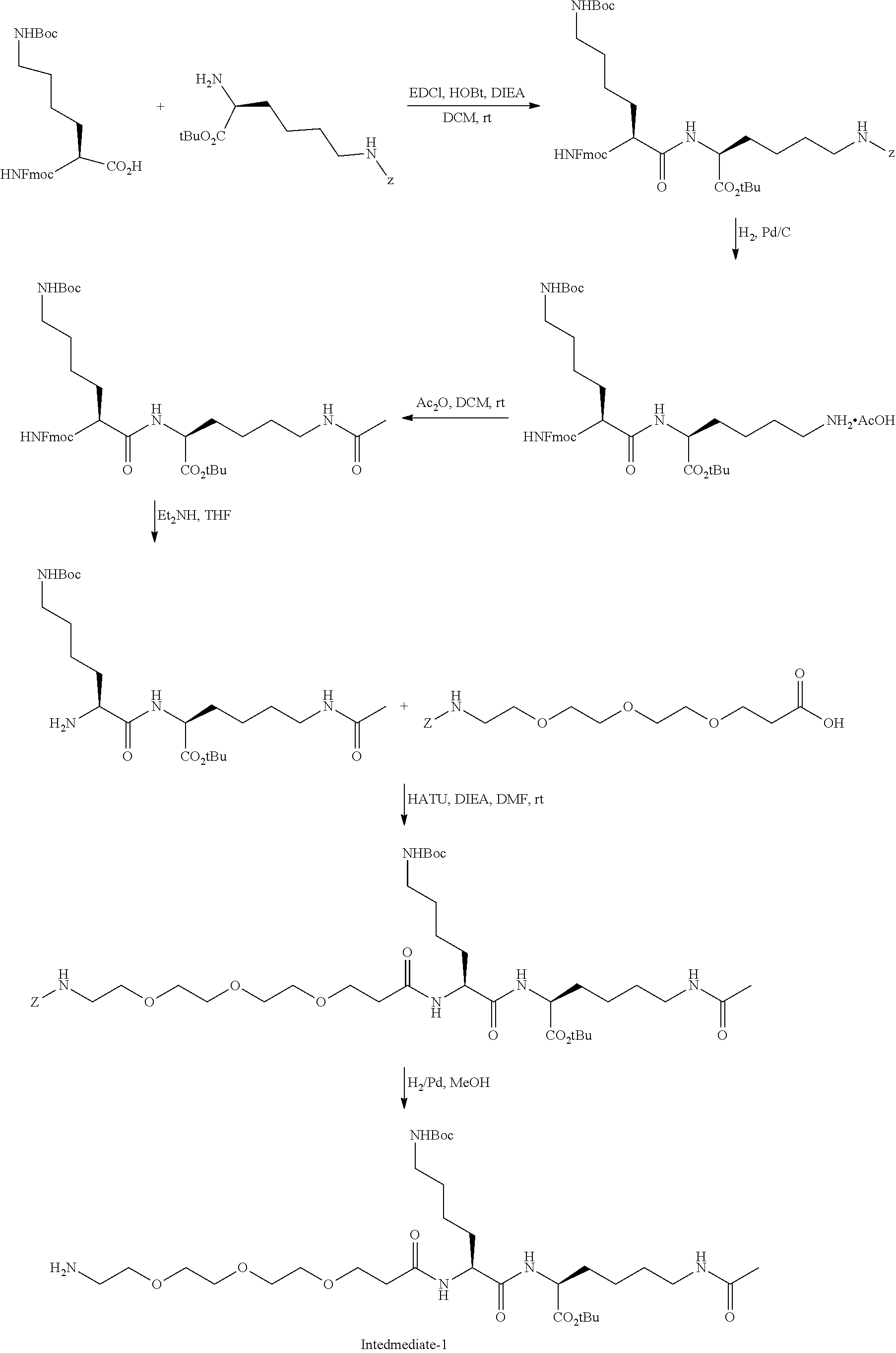

### Preparation of Intermediate 1: (S)-6-acetylamino-2-[(S)-2-(3-{2-[2-(2-amino-ethoxy)-ethoxy]-ethoxy}-propionylamino)-6-tert-butoxycarbonyl aminohexanoylamino]-hexanoic Acid Tert-Butyl Ester

Procedure 1: Preparation of (S)-tert-butyl 2-((S)-2-((((9H-fluoren-9-yl)methoxy)carbonyl)amino)-6-((tert-butoxycarbonyl)amino)hexanamido)-6-(((benzyloxy)carbonyl)amino)hexanoate:

A solution of Fmoc-Lys(Boc)-OH (4.69 g), H-Lys(Z)—OtBu, HCl (4.48 g, 1.2 equiv.), EDC·HCl (3.82 g, 2.0 equiv.), and HOBt·H2O (1.84 g, 1.2 equiv.) in DCM (100 mL) was stirred for 10 min and DIEA (4 equiv.) was added slowly. The stirred reaction mixture was monitored by LC-MS RM for 4 h at room temperature. The mixture was diluted with DCM and the organic layer was washed sequentially with water and brine, dried over MgSO₄, filtered, and the volatiles were removed under reduced pressure to afford the crude product, which was purified by combi-flash silica gel chromatography (DCM and EtOAc eluant) to obtain the pure product (6.0 g, 77% yield).

Procedure 2: Preparation of (S)-tert-butyl 2-((S)-2-((((9H-fluoren-9-yl)methoxy)carbonyl)amino)-6-((tert-butoxycarbonyl)amino)hexanamido)-6-aminohexanoate acetate:

Activated palladium on carbon (5%, 1.0 g) was suspended in a solution (S)-tert-butyl 2-((S)-2-((((9H-fluoren-9-yl)methoxy)carbonyl)amino)-6-((tert-butoxycarbonyl)amino)hexanamido)-6-(((benzyloxy)carbonyl)amino)hexanoate (7.8 g, 10 mmol) in MeOH (100 mL) and acetic acid (1 mL). The suspension was subjected to hydrogenation (50 psi) for 2 h at room temperature. The mixture was then filtered through celite, and the filtrate was concentrated under reduced pressure to afford the product (6.05 g, 85% yield as acetate), Fmoc deprotection was also observed during hydrogenation (˜5%). The crude product was sufficiently pure to be used in the next step without further purification.

Procedure 3: Preparation of (S)-6-acetylamino-2-[(S)-6-tert-butoxycarbonylamino-2-(9H-fluoren-9-ylmethoxycarbonylamino)-hexanoylamino]-hexanoic acid tert-butyl ester:

To a stirred solution of (S)-6-amino-2-[(S)-6-tert-butoxycarbonylamino-2-(9H-fluoren-9-ylmethoxy carbonylamino)-hexanoylamino]-hexanoic acid tert-butyl ester (3 g, 4.6 mmol) and DIEA (1.76 ml, 10.12 mmol) in DCM (24 ml) was added acetic anhydride (524 ul, 5.51 mmol). The mixture was stirred at room temperature for 90 min, at which time LCMS indicated complete conversion. The reaction mixture was diluted with EtOAc, washed with water and brine, dried over anhydrous MgSO₄ and filtered. Evaporation of the volatiles under reduced pressure left the target compound (2.9 g, 91%) as off-white solid.

Procedure 4: Preparation of (S)-6-acetylamino-2-((S)-2-amino-6-tert-butoxycarbonylamino-hexanoylamino)-hexanoic acid tert-butyl ester:

To a stirred solution of (S)-6-acetylamino-2-[(S)-6-tert-butoxycarbonyl amino-2-(9H-fluoren-9-ylmethoxycarbonylamino)-hexanoylamino]-hexanoic acid tert-butyl ester (2.9 g, 4.2 mmol) in DCM (12 ml) was added diethylamine (6.51 ml, 62.59 mmol) at ambient temperature. Full conversion was detected by LCMS after 45 min. The solvents were evaporated under reduced pressure and the residue was dissolved in DCM purified on a CombiFlash apparatus (220 g silica gel column, gradient 0-50% MeOH/DCM over 100 minutes). The desired fractions were combined and evaporated to produce target material (1.66 g, 84%) as amorphous colorless solid.

Procedure 5: Preparation of (S)-6-acetylamino-2-[(S)-2-(3-{2-[2-(2-benzyloxycarbonylamino-ethoxy)-ethoxy]-ethoxy}-propionylamino)-6-tert-butoxycarbonylamino-hexanoylamino]-hexanoic acid tert-butyl ester:

DIEA (1.34 ml, 1.72 mmol) was added to a stirred solution of (S)-6-acetylamino-2-((S)-2-amino-6-tert-butoxycarbonylamino-hexanoylamino)-hexanoic acid tert-butyl ester (1.66 g, 3.51 mmol), 3-{2-[2-(2-benzyloxy-carbonylamino-ethoxy)-ethoxy]-ethoxy}-propionic acid (1.25 g, 3.51 mmol) and HATU (1.47 g, 3.86 mmol) in DMF (10 ml) at ambient temperature. The reaction mixture was stirred at room temperature for 14 h, at which time full conversion was detected by LCMS. The mixture was diluted with EtOAc, washed with water (×2) and brine, dried over anhydrous MgSO₄ and filtered. The filtrate was evaporated to obtain target material (2.84 g, 100%) as a colorless amorphous solid.

Procedure 6: Preparation of (S)-6-acetylamino-2-[(S)-2-(3-{2-[2-(2-amino-ethoxy)-ethoxy]-ethoxy}-propionyl amino)-6-tert-butoxycarbonylamino-hexanoylamino]-hexanoic acid tert-butyl ester:

To a solution of (S)-6-acetylamino-2-[(S)-2-(3-{2-[2-(2-benzyloxycarbonylamino-ethoxy)-ethoxy]-ethoxy}-propionylamino)-6-tert-butoxycarbonylamino-hexanoyl-amino]-hexanoic acid tert-butyl ester (2.84 mg, mmol) in methanol (50 ml) was added Pd/C (5%, 500 mg, 0.235 mmol). The mixture was subjected to hydrogenation on Parr apparatus (60 PSI H2) at ambient temperature for 1 h. The supported catalyst was removed by filtration through the pad of Celite, and the filtrate was evaporated to obtain the target material, Intermediate 1, (2.075 g, 88%) as a colorless amorphous solid.

### Preparation of Intermediate-2: {4-[2-((S)-2-Cyano-pyrrolidin-1-yl)-2-oxo-ethylcarbamoyl]-quinolin-7-yloxy}-acetic Acid

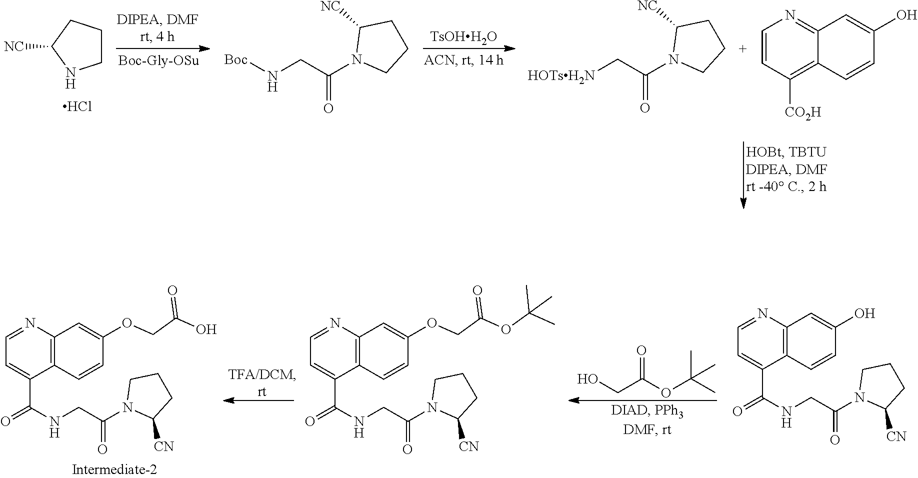

Procedure 7: Preparation of [2-((S)-2-cyano-pyrrolidin-1-yl)-2-oxo-ethyl]-carbamic acid tert-butyl ester:

To a solution of (S)-pyrrolidine-2-carbonitrile hydrochloride (1 g, 7.5 mmol) in DCM (35 ml) was added DIEA (3.9 ml, 22.5 mmol) and Boc-Gly-OSu (2.26 g, 8.3 mmol). The reaction mixture was stirred at ambient temperature for 14 h. The solvents were evaporated and the residue was dissolved in DCM, washed with water, dried over anhydrous MgSO₄ and filtered. The filtrate was concentrated to 20% of the initial volume and loaded onto a CombiFlash™ apparatus and eluted (gradient 0-10% MeOH/DCM). The desired fractions were combined and evaporated to obtain target product (1.1 g, 58%) as an amorphous solid.

Procedure 8: Preparation of (S)-1-(2-amino-acetyl)-pyrrolidine-2-carbonitrile:

p-Toluenesulfonic acid monohydrate (1.25 g, 6.6 mmol) was added to a stirred solution of (2-((S)-2-cyano-pyrrolidin-1-yl)-2-oxo-ethyl)-carbamic acid tert-butyl ester (1.1 g, 4.4 mmol) in acetonitrile (20 ml) and the mixture was stirred at ambient temperature for 14 h. The solvent was removed at reduced pressure to obtain the tosylate salt of the target product (1.8 g, yield exceeded theoretical). The compound was sufficiently pure to carry through to the next chemical transformation.

Procedure 9: Preparation of (S)—N-(2-(2-cyanopyrrolidin-1-yl)-2-oxoethyl)-7-hydroxyquinoline-4-carboxamide:

A solution of 7-hydroxyquinoline-4-carboxylic acid (307 mg, 1.62 mmol), HOBt (221 mg, 1.62 mmol), and TBTU (521 mg, 1.62 mmol) in DMF (15 mL) was stirred at room temperature for 5 min. A solution of (S)-1-(2-amino-acetyl)-pyrrolidine-2-carbonitrile (1.5 tosylate) (1.78 mmol) and DIEA (0.74 mL, 4.86 mmol) in DMF (5 mL) was added to the activated ester solution and the resulting mixture was stirred at room temperature for 2 h while the reaction progress was monitored by LCMS. Upon full conversion, the reaction mixture was concentrated under reduced pressure, dissolved in a small amount of dichloromethane, and filtered. The filtrate was evaporated, dissolved in DCM, loaded onto a CombiFlash™ silica gel column, and purified (gradient 0-20% MeOH/DCM) to obtain the target product (422 mg, 80% yield) as an off-white solid.

Procedure 10: Preparation of (S)-tert-butyl 2-((4-((2-(2-cyanopyrrolidin-1-yl)-2-oxoethyl) carbamoyl)quinolin-7-yl)oxy)acetate:

A flask containing 7-hydroxyquinoline-4-carboxylic acid [2-((S)-2-cyanopyrrolidin-1-yl)-2-oxo-ethyl]-amide (407 mg, 1.25 mmol), 2-tert-butyl glycolate (249 mg, 1.88 mmol), and triphenylphosphine (395 mg, 1.51 mmol) in DMF (15 mL) was chilled in an ice water bath. Di-isopropyl azodicarboxylate (300 uL, 1.51 mmol) was added dropwise to the chilled reaction mixture. The ice water bath was removed, and the resulting solution was stirred at room temperature and monitored by LCMS. Upon completion, the solvent was removed under reduced pressure and the residue was dissolved in DCM, loaded onto a CombiFlash™ silica gel column (gradient 0->10% MeOH/DCM) and purified to obtain the target product (404 mg, 61% yield) as a glassy solid.

Procedure 11: Preparation of {4-[2-((S)-2-Cyano-pyrrolidin-1-yl)-2-oxo-ethylcarbamoyl]-quinolin-7-yloxy}-acetic acid:

To a solution of {4-[2-((S)-2-cyano-pyrrolidin-1-yl)-2-oxo-ethylcarbamoyl]-quinolin-7-yloxy}-acetic acid tert-butyl ester (141 mg, 0.322 mmol) in DCM (1.5 ml) was added TFA (1.51 ml, 19.64 mmol) dropwise at 0° C. The temperature of the stirred reaction mixture was allowed to rise to room temperature over the course of 1 h, at which time full conversion was detected by LCMS. The solvent was evaporated at rt and residue was co-evaporated with toluene (×3) at 40° C. The crude compound, Intermediate 2, was sufficiently pure to use for subsequent transformations, but it was unstable to hydrolysis upon long standing. For this reason, the compound was prepared immediately before use.

### Preparation of RTX-1312S from Fragments

Procedure 12: Preparation of tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-(tert-butoxycarbonylamino)hexanoylamino]-6-acetylaminohexanoate:

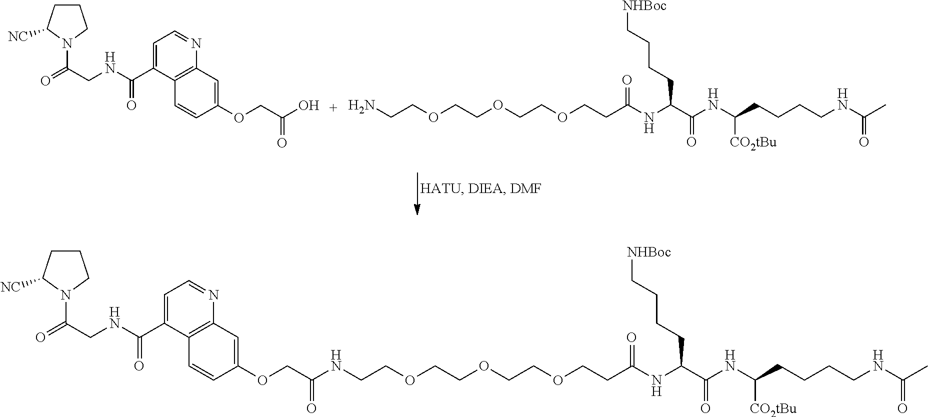 [SMILES: N#C[C@@H]1CCCN1C(=O)CNC(=O)c1ccnc2cc(OCC(=O)O)ccc12 ; CC(=O)NCCCC[C@H](NC(=O)[C@H](CCCCNC(=O)OC(C)(C)C)NC(=O)CCOCCOCCOCCN)C(=O)OC(C)(C)C ; CC(=O)NCCCC[C@H](NC(=O)[C@H](CCCCNC(=O)OC(C)(C)C)NC(=O)CCOCCOCCOCCNC(=O)COc1ccc2c(C(=O)NCC(=O)N3CCC[C@H]3C#N)ccnc2c1)C(=O)OC(C)(C)C]

DIEA (1.64 ml, 9.43 mmol) was added to a stirred mixture of {4-[2-((S)-2-cyano-pyrrolidin-1-yl)-2-oxo-ethylcarbamoyl]-quinolin-7-yloxy}-acetic acid (1.57 mmol), (S)-6-acetylamino-2-[(S)-2-(3-{2-[2-(2-amino-ethoxy)-ethoxy]-ethoxy}-propionylamino)-6-tert-butoxycarbonylamino-hexanoylamino]-hexanoic acid tert-butyl ester (1.17 g, 1.73 mmol) and HATU (717 mg, 1.89 mmol) in DMF (10 ml). The stirred mixture was held at room temperature for 30 minutes, when full conversion was detected by LCMS. The mixture was diluted with EtOAc, washed with water and brine, and the collected aqueous phase was back-extracted with EtOAc. The combined organic extracts were washed with brine, dried over anhydrous MgSO₄, filtered, and evaporated under reduced pressure. The residue was dissolved in DCM, loaded onto a CombiFlash silica gel column (220 g, gradient 0->30% MeOH/DCM in 60 minutes) and purified. The desired fractions were combined and evaporated to produce target material (950 mg, 58%) as glassy solid.

Procedure 13: Preparation of (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy) ethoxy]ethoxy}propionylamino)-6-aminohexanoylamino]-6-acetylaminohexanoic acid:

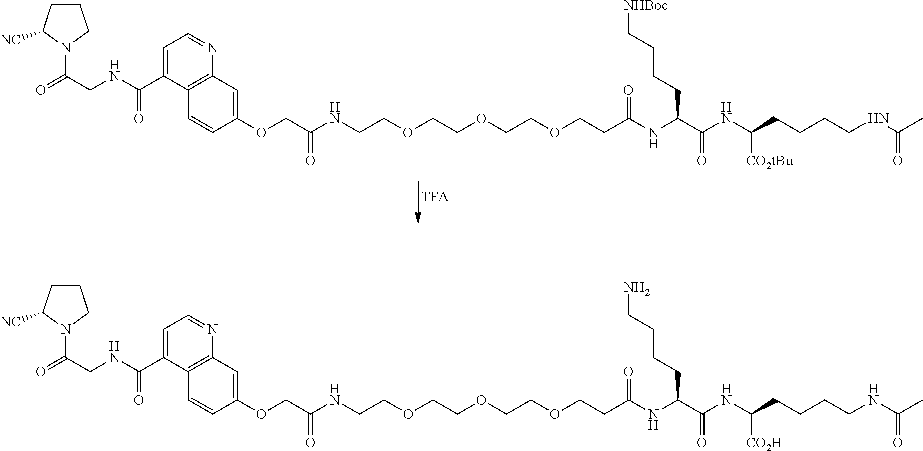 [SMILES: CC(=O)NCCCC[C@H](NC(=O)[C@H](CCCCNC(=O)OC(C)(C)C)NC(=O)CCOCCOCCOCCNC(=O)COc1ccc2c(C(=O)NCC(=O)N3CCC[C@H]3C#N)ccnc2c1)C(=O)OC(C)(C)C ; CC(=O)NCCCC[C@H](NC(=O)[C@H](CCCCN)NC(=O)CCOCCOCCOCCNC(=O)COc1ccc2c(C(=O)NCC(=O)N3CCC[C@H]3C#N)ccnc2c1)C(=O)O]

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-(tert-butoxycarbonylamino)hexanoylamino]-6-acetylaminohexanoate (74 mg, 0.071 mmol) in anhydrous DCM (1 ml) was added anhydrous trifluoroacetic acid (1 ml, 13 mmol) dropwise at 0° C. The temperature of the reaction mixture was allowed to rise to ambient and stirring was continued for 90 minutes, at which time full conversion was detected by LCMS. The solvent was evaporated at room temperature and residue was co-evaporated with toluene. The compound so obtained compound was used immediately in the next chemical transformation.

Procedure 14: Preparation of RTX-1312S: (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy) ethoxy]ethoxy}propionylamino)-6-[3-(p-{[1,4,7-tris(carboxymethyl)-1,4,7-triazonan-2-yl]methyl}phenyl)thioureido]hexanoylamino]-6-acetylaminohexanoic acid.

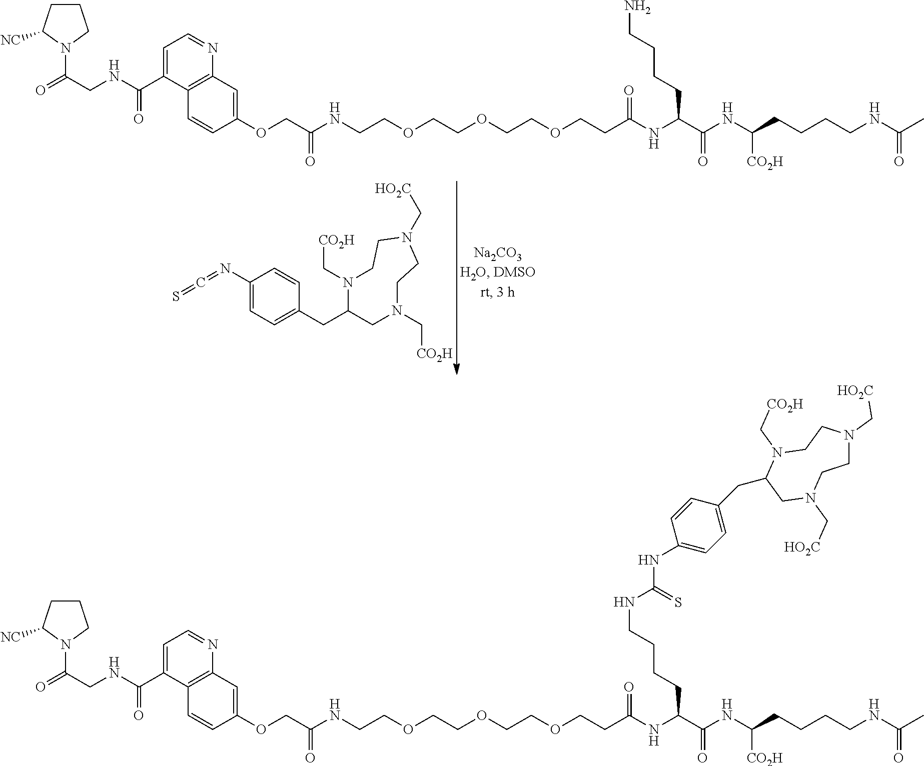 [SMILES: CC(=O)NCCCC[C@H](NC(=O)[C@H](CCCCN)NC(=O)CCOCCOCCOCCNC(=O)COc1ccc2c(C(=O)NCC(=O)N3CCC[C@H]3C#N)ccnc2c1)C(=O)O ; O=C(O)CN1CCN(CC(=O)O)CC(Cc2ccc(N=C=S)cc2)N(CC(=O)O)CC1 ; CC(=O)NCCCC[C@H](NC(=O)[C@H](CCCCNC(=S)Nc1ccc(CC2CN(CC(=O)O)CCN(CC(=O)O)CCN2CC(=O)O)cc1)NC(=O)CCOCCOCCOCCNC(=O)COc1ccc2c(C(=O)NCC(=O)N3CCC[C@H]3C#N)ccnc2c1)C(=O)O]

A solution of p-SCN-Bn-Nota 3HCl (Intermediate 3; 40 mg, 0.071 mmol) in water (500 ul) was added to a solution of (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-aminohexanoylamino]-6-acetylaminohexanoic acid×TFA (0.071 mmol) in DMSO (1300 μl) followed by the addition a solution of sodium carbonate (188 mg, 1.78 mmol) in water (1 ml) at ambient temperature. The pH of the resulting solution mixture was approximately 9. The reaction mixture was stirred at ambient temperature for 14 h and subjected to HPLC purification with a Clipeus C18, 10 μn, 250×20 mm column (CS-2520-C181 Higgins Analytical, Inc.); MP A: 0.1% TFA in H₂O; MP B: 0.1% TFA in Acetonitrile 5% of buffer B over 5 minutes, then 5% to 55% of buffer B in 40 minutes. Flow rate: 25 mL/min. Detection UV: UV254. Desired fractions were combined and lyophilized to obtain the target compound RTX-1312S (84 mg, 88%) as an off-white solid.

### Synthesis of RTX-1317S

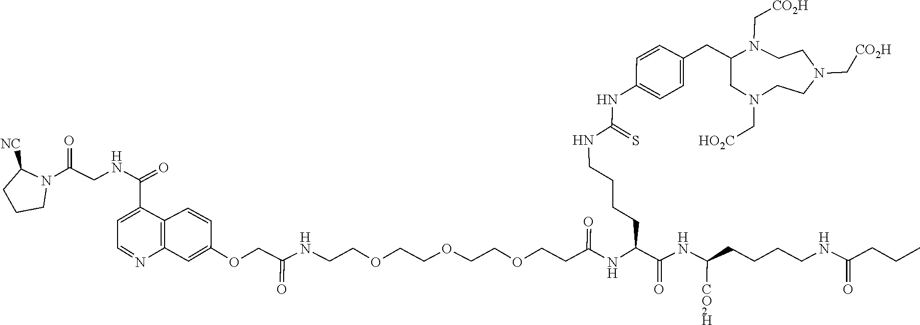 [SMILES: CCCC(=O)NCCCC[C@H](NC(=O)[C@H](CCCCNC(=S)Nc1ccc(CC2CN(CC(=O)O)CCN(CC(=O)O)CCN2CC(=O)O)cc1)NC(=O)CCOCCOCCOCCNC(=O)COc1ccc2c(C(=O)NCC(=O)N3CCC[C@H]3C#N)ccnc2c1)C(=O)O]

Prepared according to the procedures of Example 1 with the following changes:
Procedure 40 was used in lieu of Procedure 3.
In Procedure 40, butyric acid was used in lieu of hexanoic acid.

### Synthesis of RTX-1318S

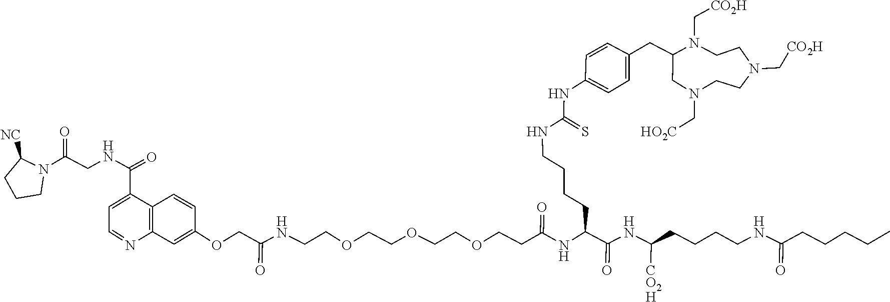 [SMILES: CCCCCC(=O)NCCCC[C@H](NC(=O)[C@H](CCCCNC(=S)Nc1ccc(CC2CN(CC(=O)O)CCN(CC(=O)O)CCN2CC(=O)O)cc1)NC(=O)CCOCCOCCOCCNC(=O)COc1ccc2c(C(=O)NCC(=O)N3CCC[C@H]3C#N)ccnc2c1)C(=O)O]

Prepared according to the procedures of Example 1 with the following changes:
Procedure 40 was used in lieu of Procedure 3.

### Synthesis of RTX-1319R

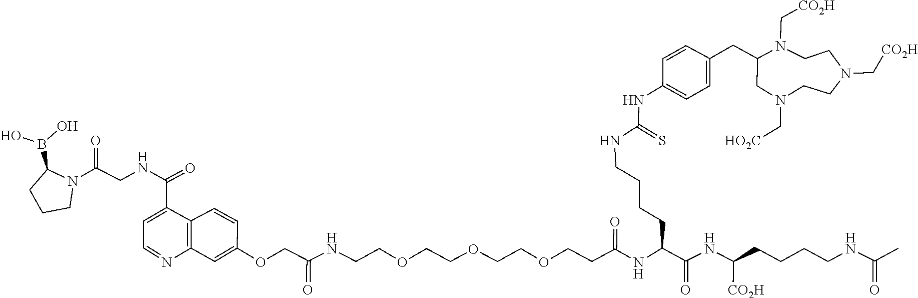 [SMILES: CC(=O)NCCCC[C@H](NC(=O)[C@H](CCCCNC(=S)Nc1ccc(CC2CN(CC(=O)O)CCN(CC(=O)O)CCN2CC(=O)O)cc1)NC(=O)CCOCCOCCOCCNC(=O)COc1ccc2c(C(=O)NCC(=O)N3CCC[C@H]3B(O)O)ccnc2c1)C(=O)O]

Prepared according to the procedures of Example 1 with the following changes:
Procedures 52-54 were used in lieu of Procedure 12.
The chelator was installed using Procedure 13 & 14 prior to subjecting the intermediate to procedure 52-54.

### Synthesis of RTX-1341S

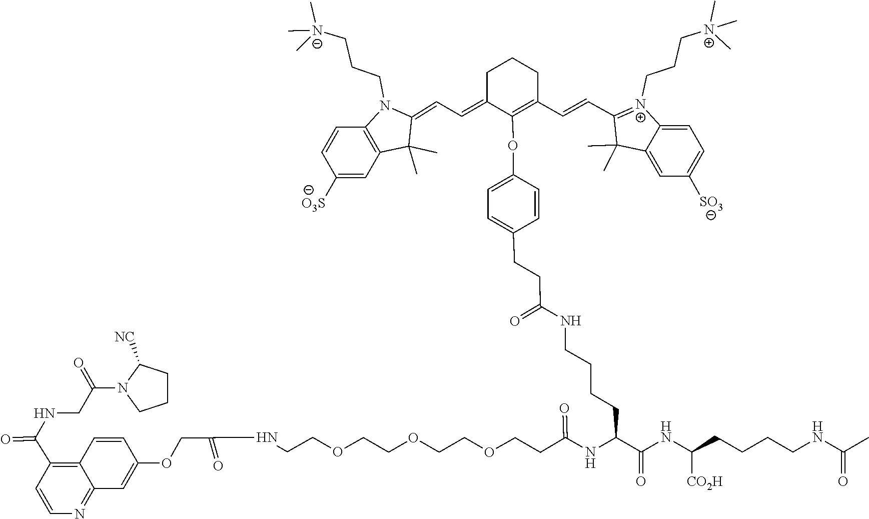

Prepared according to the procedures of Example 1 with the following changes:
Procedures 15 & 16 were used in lieu of Procedure 14 for conjugation of the optical dye or fluorophore.

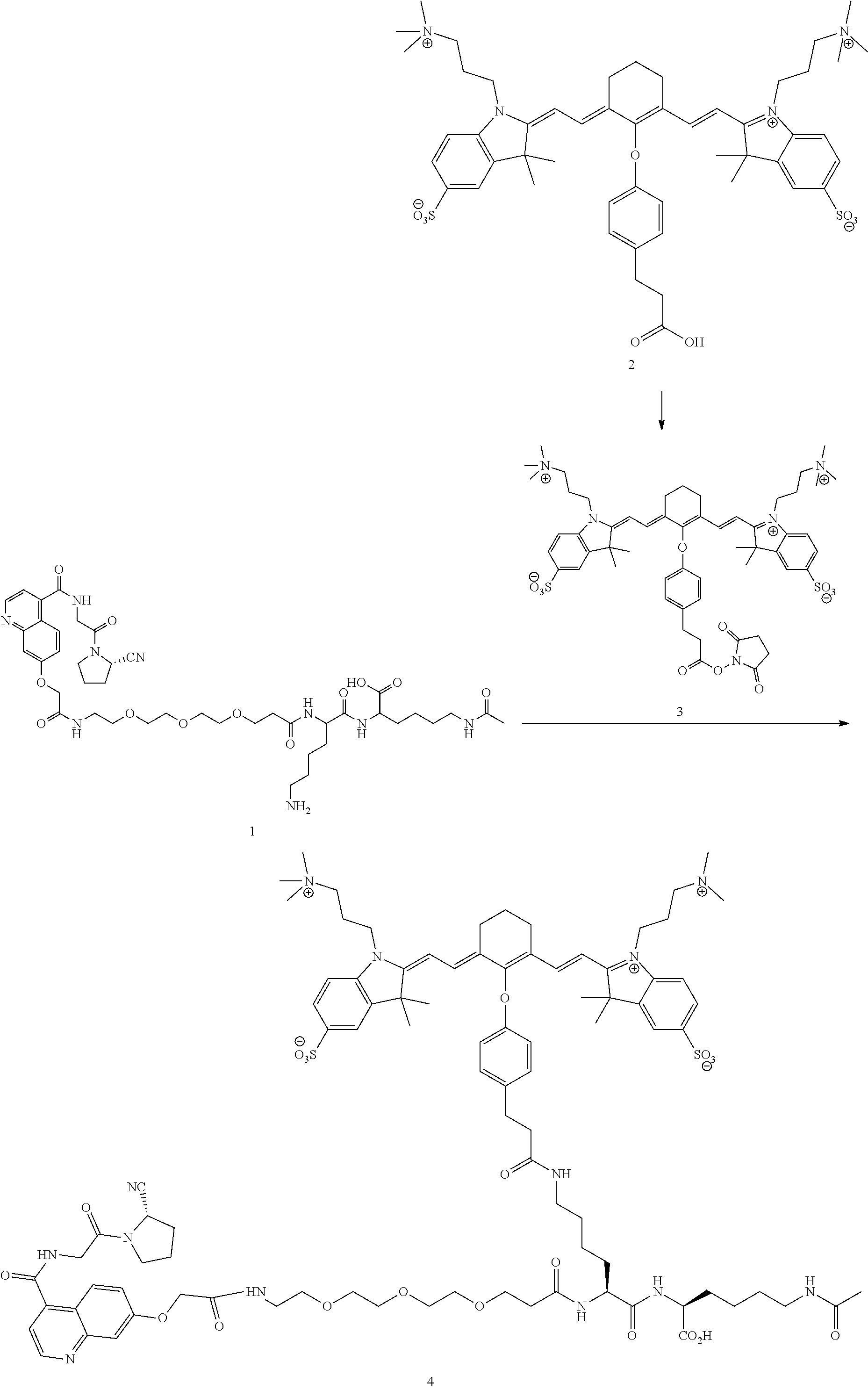

Procedure 15: Synthesis of 2-((E)-2-((E)-3-((E)-2-(3,3-dimethyl-5-sulfonato-1-(3-(trimethylammonio)-propyl)-indolin-2-ylidene)-ethylidene)-2-(4-(3-((2,5-dioxopyrrolidin-1-yl)oxy)-3-oxopropyl)phenoxy)cyclohex-1-en-1-yl)vinyl)-3,3-dimethyl-1-(3-(trimethylammonio)propyl)-3H-indol-1-ium-5-sulfonate:

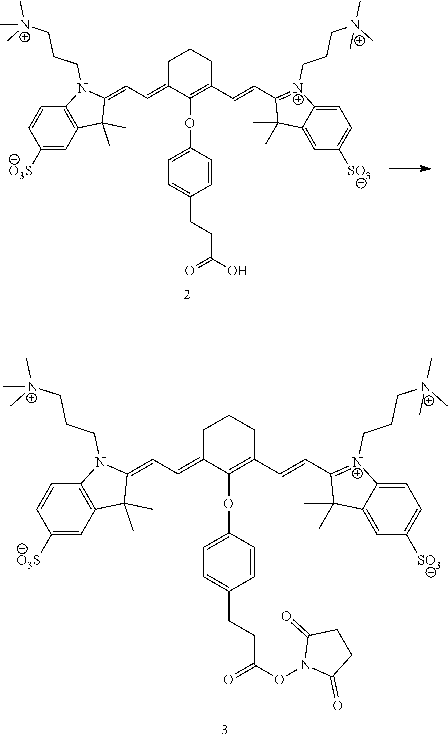

To a solution of ZW800-1 (100 mg, 0.105 mmol) in anhydrous DMSO (10 ml) was added dipyrrolidino(N-succinimidyloxy)carbenium hexafluorophosphate (130 mg, 0.3 mmol) followed by the addition of N,N-diisopropylethylamine (0.2 mL, 1.1 mmol) dropwise at room temperature. The reaction mixture was stirred at ambient temperature for 16 hours, at which time full conversion was detected by LCMS. The mixture was treated with a solution of 1:1:1 ethanol:ethylacetate:acetone (150 mL) and 0.1% trifluoroacetic acid (0.9 mL) and was allowed to mix and sit for 30 min. The solids were filtered and dried under vacuum to obtain 2-((E)-2-((E)-3-((E)-2-(3,3-dimethyl-5-sulfonato-1-(3-(trimethylammonio)-propyl)indolin-2-ylidene)ethylidene)-2-(4-(3-((2,5-dioxo-pyrrolidin-1-yl)oxy)-3-oxopropyl)phenoxy)cyclohex-1-en-1-yl)vinyl)-3,3-dimethyl-1-(3-(trimethylamm-onio)propyl)-3H-indol-1-ium-5-sulfonate as a green powder (51 mg, 46%) which was used directly for the next step without further purification. LCMS: C₄₆H₆₁N₉O₁₂: m/z: 1040.32, observed m/z=1040.5 [M]⁺.

Procedure 16: Synthesis of Final step of RTX-1341S:

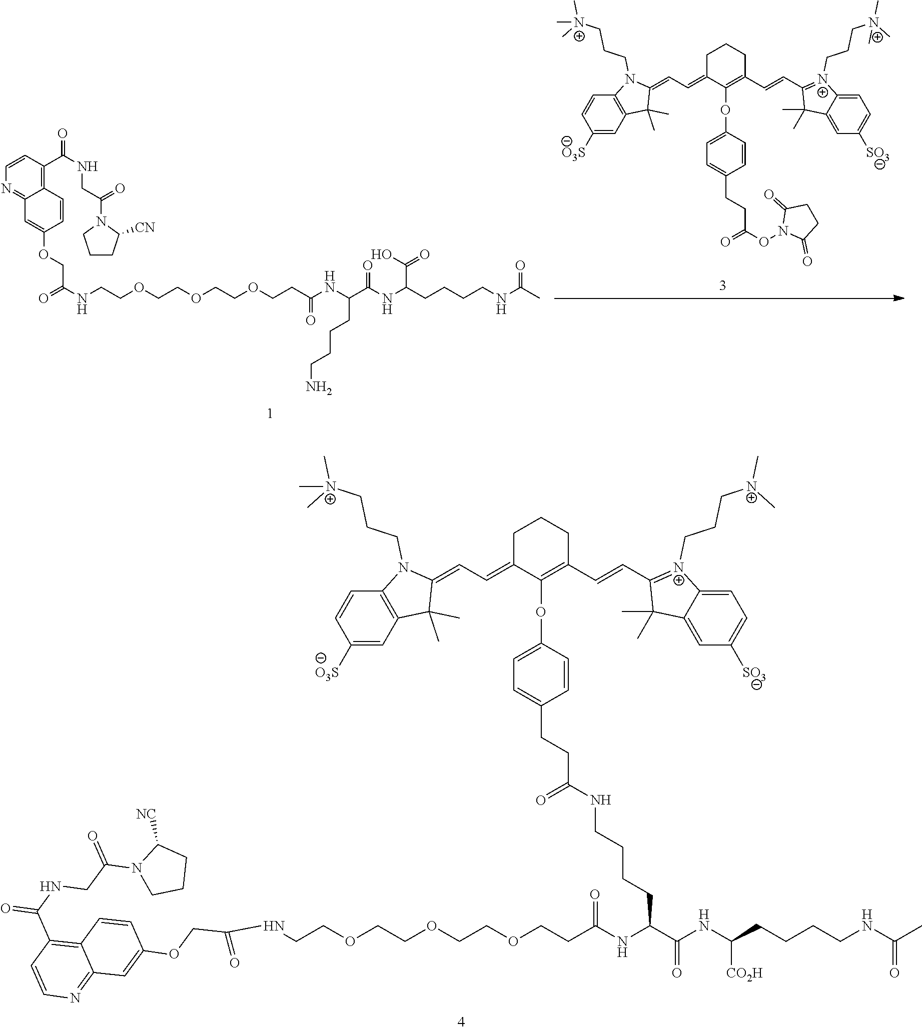

To the solution of 3 (0.070 g, 0.067 mmol) in DMSO (1 mL) was added a solution of (S)-2-[(S)-2-(3-{2-[2-(2-{2-[4-({2-[(R)-2-cyano-1-pyrrolidinyl]-2-oxoethylamino}carbonyl)-7-quinolyloxy]acetylamino}etho-xy)ethoxy]ethoxy}propionylamino)-6-aminohexanoylamino]-6-acetylaminohexanoic acid (0.040 g, 0.045 mmol) in DMSO (1 mL). N,N-Diisopropylethylamine (0.070 g, 0.067 mmol) was added at room temperature, and the mixture was allowed to stir for 16 h. Full conversion was detected by LCMS. After the reaction completion, the volatiles were removed and the residue was purified by HPLC to yield RTX-1341S (14.4 mg, 14%) as a green solid. LCMS: C₄₆H₆₁N₉O₁₂: m/z: 1809.9, observed m/z=1811.4 [M+H]⁺.

### Synthesis of RTX-1370S

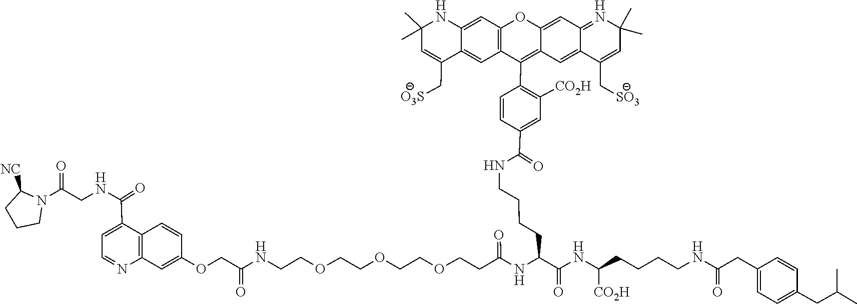

Prepared according to the procedures of Example 1 with the following changes:
Procedure 40 was used in lieu of Procedure 3.
In Procedure 40, 2-(4-isobutylphenyl)acetic acid was used in lieu of hexanoic acid.
Alexa Fluor 568 was installed using a procedure analogous to Procedure 16.

### Synthesis of RTX-1354S

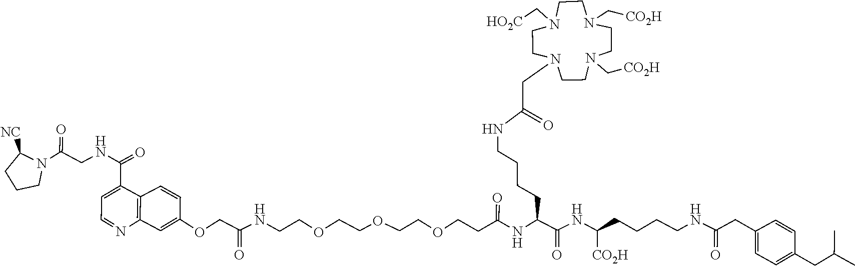 [SMILES: CC(C)Cc1ccc(CC(=O)NCCCC[C@H](NC(=O)[C@H](CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4C#N)ccnc3c2)C(=O)O)cc1]

Prepared according to the procedures of Example 1 with the following changes:
Procedure 40 was used in lieu of Procedure 3.
In Procedure 40, 2-(4-isobutylphenyl)acetic acid was used in lieu of hexanoic acid.
Procedure 17 & 18 were used in lieu of Procedure 14 for the installation of the DOTA chelator.

### Synthesis of RTX-1354S DOTA Chelators

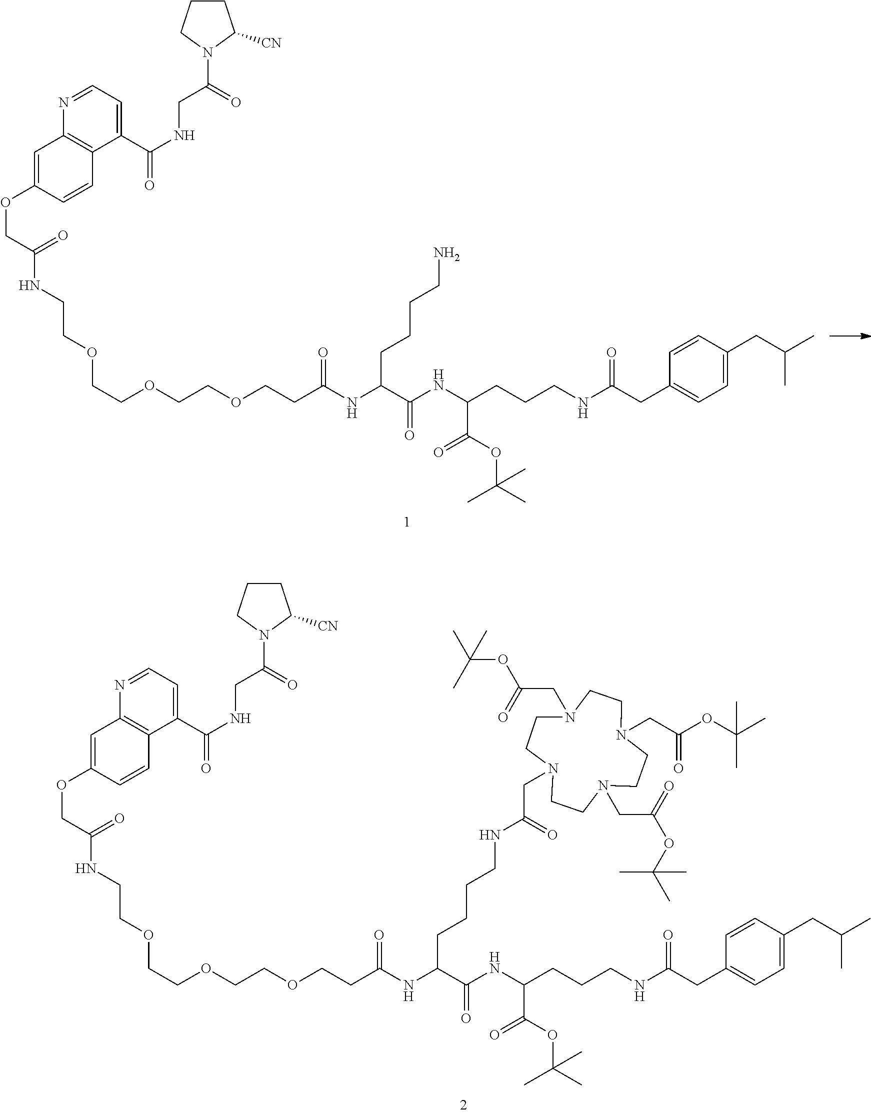 [SMILES: CC(C)Cc1ccc(CC(=O)NCCCC(NC(=O)C(CCCCN)NC(=O)CCOCCOCCOCCNC(=O)COc2ccc3c(C(=O)NCC(=O)N4CCC[C@@H]4C#N)ccnc3c2)C(=O)OC(C)(C)C)cc1 ; CC(C)Cc1ccc(CC(=O)NCCCC(NC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCNC(=O)COc2ccc3c(C(=O)NCC(=O)N4CCC[C@@H]4C#N)ccnc3c2)C(=O)OC(C)(C)C)cc1]

Procedure 17: To a solution of (S)-2-[(S)-2-(3-{2-[2-(2-{2-[4-({2-[(S)-2-cyano-1-pyrrolidinyl]-2-oxoethylamino}carbon-yl)-7-quinolyloxy]acetylamino}ethoxy)ethoxy]ethoxy}propionylamino)-6-aminohexanoylamino]-6-[4-(p-isobutylphenyl)butyrylamino]hexanoic acid trifluoroacetate (0.035 mmol) and DIEA (0.031 ul, 0.18 mmol) in DMSO (1 ml) was added DOTA-mono-NHS-tris(tBu ester) (31 mg, 0.046 mmol) and H₂O (1 ml). The pH of the reaction mixture was adjusted to 9 with DIEA (250 ul). The reaction mixture was stirred at ambient temperature o/n, at which time full conversion was detected by LCMS. The reaction mixture was subjected to HPLC purification, the desired fractions were collected and evaporated to obtain 2 as a glassy solid. LCMS: C₈₀H₁₂₃N₁₃O₁₉: m/z: 1569.91, observed m/z=1571.5 [M+H]⁺.

### Synthesis of 1354S

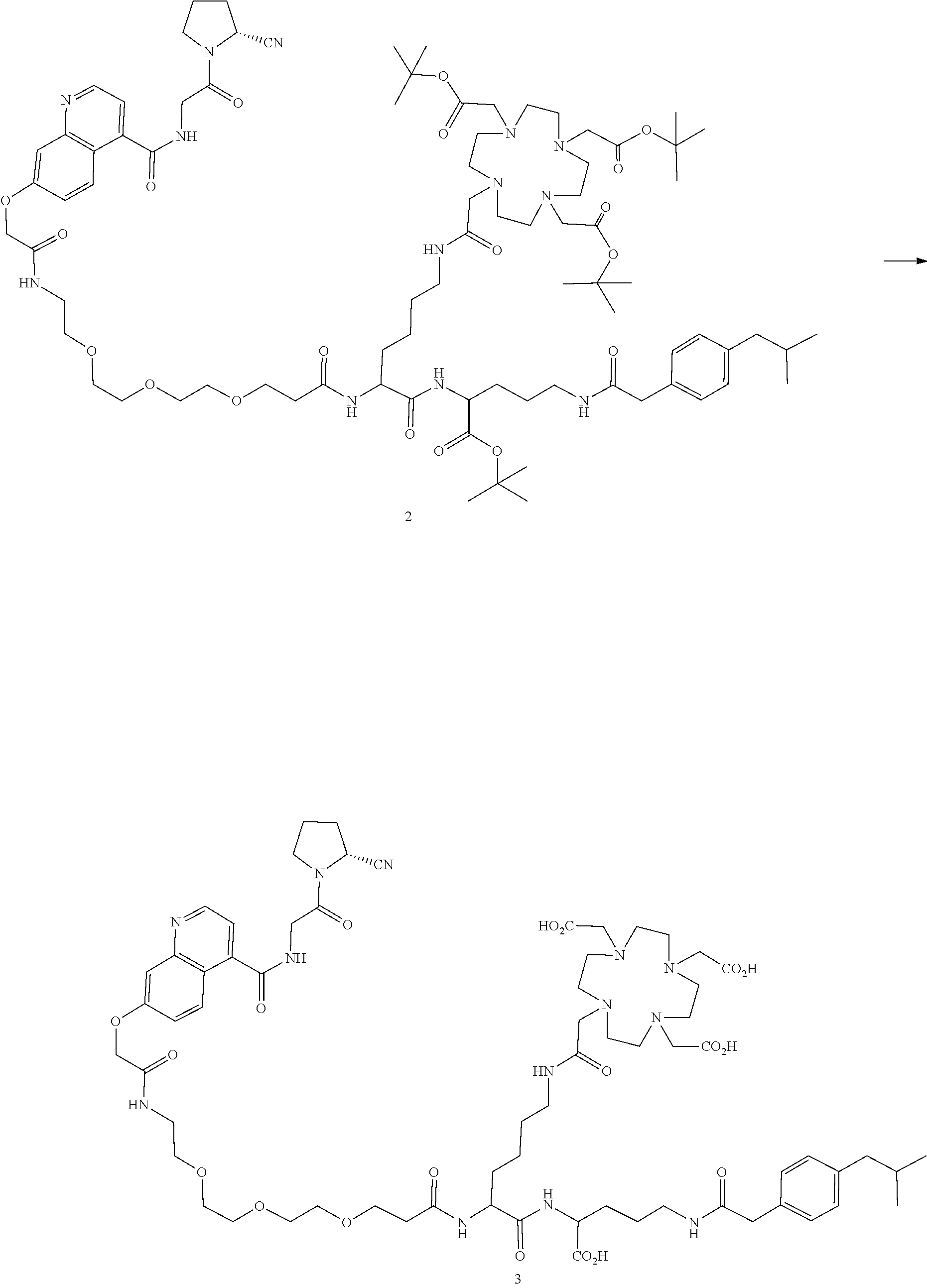 [SMILES: CC(C)Cc1ccc(CC(=O)NCCCC(NC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCNC(=O)COc2ccc3c(C(=O)NCC(=O)N4CCC[C@@H]4C#N)ccnc3c2)C(=O)OC(C)(C)C)cc1 ; CC(C)Cc1ccc(CC(=O)NCCCC(NC(=O)C(CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@@H]4C#N)ccnc3c2)C(=O)O)cc1]

Procedure 18: To a solution of 2 (55 mg, 0.041 mmol) in DCM (1 ml) was added TFA (1 ml, 13 mmol) at 0° C. The reaction mixture was warmed to ambient temperature and stirring was continued for 1 hour. Full conversion was detected by LCMS. The mixture was evaporated and the residue was dissolved in DMSO (2 ml) and subjected to HPLC purification. The desired fractions were lyophilized to obtain 3 (16 mg, 33%) as an off-white solid. LCMS: C₆₈H₉₉N₁₃O₁₉: m/z: 1402.59, observed m/z=1403.0 [M+H]⁺.

### Synthesis of 1350S

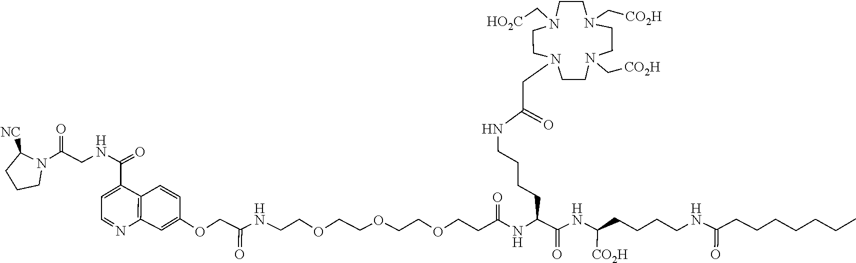 [SMILES: CCCCCCCC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Prepared according to the procedures of Example 1 with the following changes:
Procedure 40 was used in lieu of Procedure 3.
In Procedure 40, octanoic acid was used in lieu of hexanoic acid.
Procedure 17 & 18 were used in lieu of Procedure 14 for the installation of the DOTA chelator.

### Synthesis of RTX-1352S

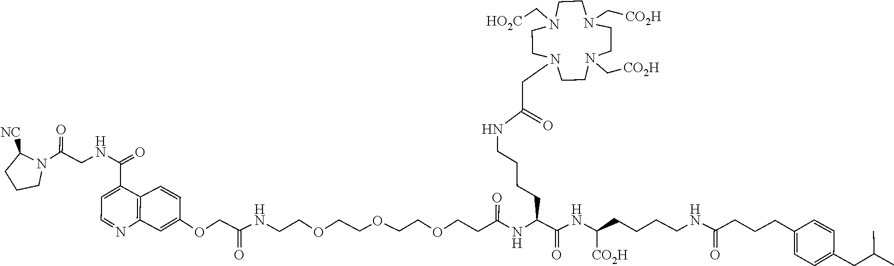 [SMILES: CC(C)Cc1ccc(CCCC(=O)NCCCC[C@H](NC(=O)[C@H](CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4C#N)ccnc3c2)C(=O)O)cc1]

Prepared according to the procedures of Example 1 with the following changes:
Procedure 40 was used in lieu of Procedure 3.
In Procedure 40, 2-(4-isobutylphenyl)butanoic acid was used in lieu of hexanoic acid.
Procedure 17 & 18 were used in lieu of Procedure 14 for the installation of the DOTA chelator.

### Synthesis of RTX-1353S

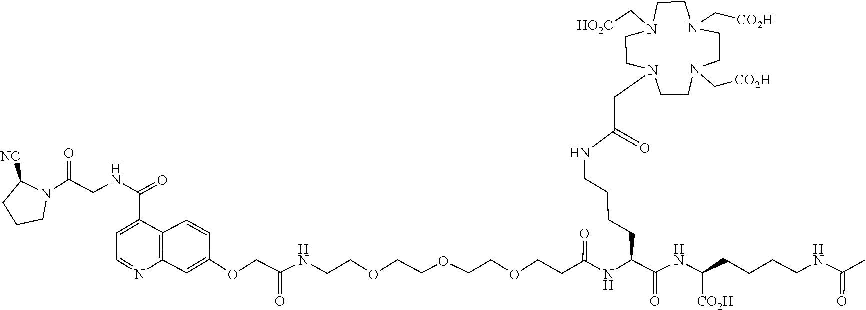 [SMILES: CC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Prepared according to the procedure of Example 1 with the following changes:
Procedure 17 & 18 were used in lieu of Procedure 14 for the installation of the DOTA chelator.

### Synthesis of 1360S

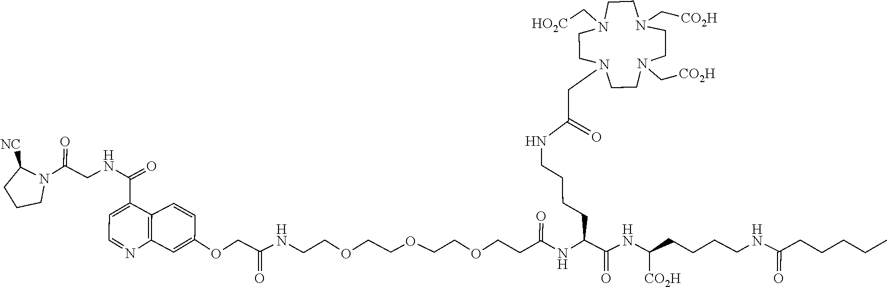 [SMILES: CCCCCC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Prepared according to the procedures of Example 1 with the following changes:
Procedure 40 was used in lieu of Procedure 3.
Procedure 17 & 18 were used in lieu of Procedure 14 for the installation of the DOTA chelator.

### Example 2—Synthesis of RTX-1324S: (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy)ethoxy]ethoxy}propionylamino)-6-(3-cyano-4-fluorobenzoylamino)hexanoylamino]-6-acetylaminohexanoic Acid

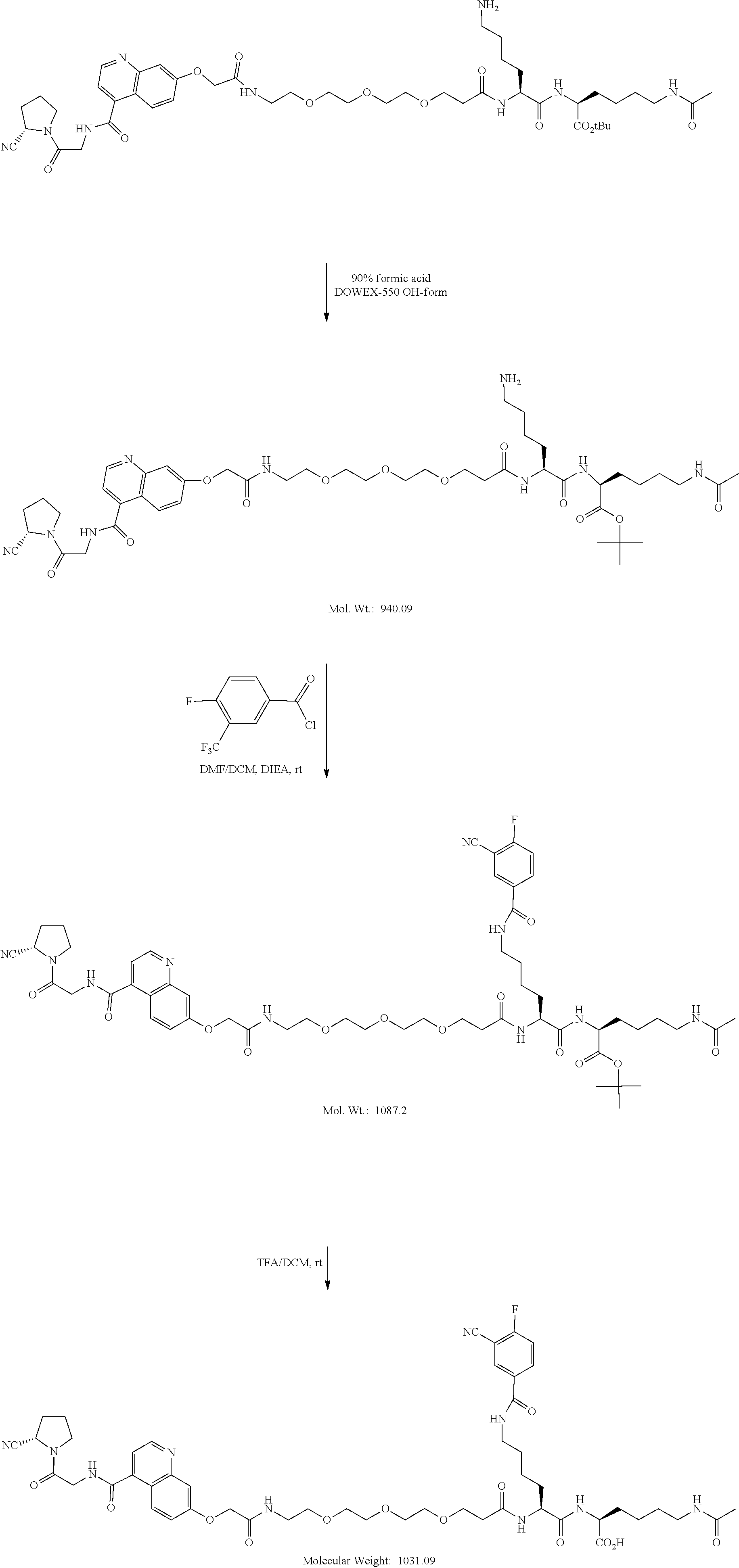

Procedure 19: Preparation of 3-Cyano-4-fluoro-benzoyl chloride

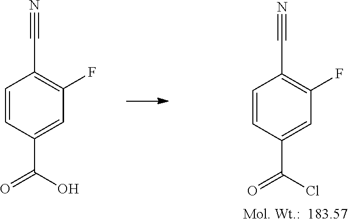 [SMILES: N#Cc1ccc(C(=O)O)cc1F ; N#Cc1ccc(C(=O)Cl)cc1F]

To a suspension of 3-cyano-4-fluorobenzoic acid (500 mg, 3.03 mmol) in DCM (10 ml) at ambient temperature was added oxalyl chloride (339 ul, 3.94 mmol) followed by DMF (12 ul, 0.151 mmol). After 1 h the solvent was evaporated and the residue was co-evaporated with toluene to give target compound (607 mg, yield exceeded theoretical) as an orange solid. This material was used in the next step without further purification.

Procedure 20: Preparation of tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy) ethoxy]ethoxy}propionylamino)-6-aminohexanoylamino]-6-acetylaminohexanoate:

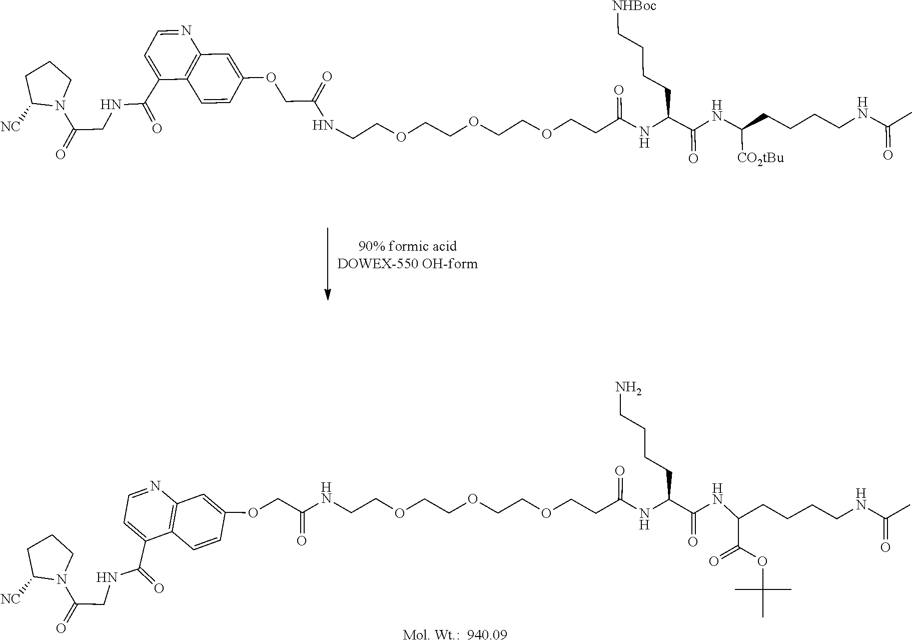 [SMILES: CC(=O)NCCCC[C@H](NC(=O)[C@H](CCCCNC(=O)OC(C)(C)C)NC(=O)CCOCCOCCOCCNC(=O)COc1ccc2c(C(=O)NCC(=O)N3CCC[C@H]3C#N)ccnc2c1)C(=O)OC(C)(C)C ; CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C]

A solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionyl amino)-6-(tert-butoxycarbonylamino)hexanoylamino]-6-acetylaminohexanoate (198 mg, 0.19 mmol) in 90% aq. formic acid (2.17 ml, 57.17 mmol) was stirred at ambient temperature for 2.5 h, at which time no starting material was detectable by LCMS. The solvents were removed under reduced pressure and the residue was co-evaporated with ACN (×3) and toluene (×2). The remainder was dissolved in the mixture MeOH/water (1:1, 50 ml) and treated with ion-exchange resin (Dowex 550A, OH-form, 15 ml) at ambient temperature for 15 mins. The resin was removed by filtration and washed with MeOH and water. The filtrate was evaporated to obtain target material (166 mg, 93%) as an off-white solid (in the form of the free base).

Procedure 21: Preparation of tert-Butyl (S)-2-[(S)-2-(3-{2-[2-(2-{2-[4-({2-[(S)-2-cyano-1-pyrrolidinyl]-2-oxoethyl amino}carbonyl)-7-quinolyloxy]acetylamino}ethoxy)ethoxy]ethoxy}propionylamino)-6-(3-cyano-4-fluorophenylamino)hexanoylamino]-6-acetylaminohexanoate:

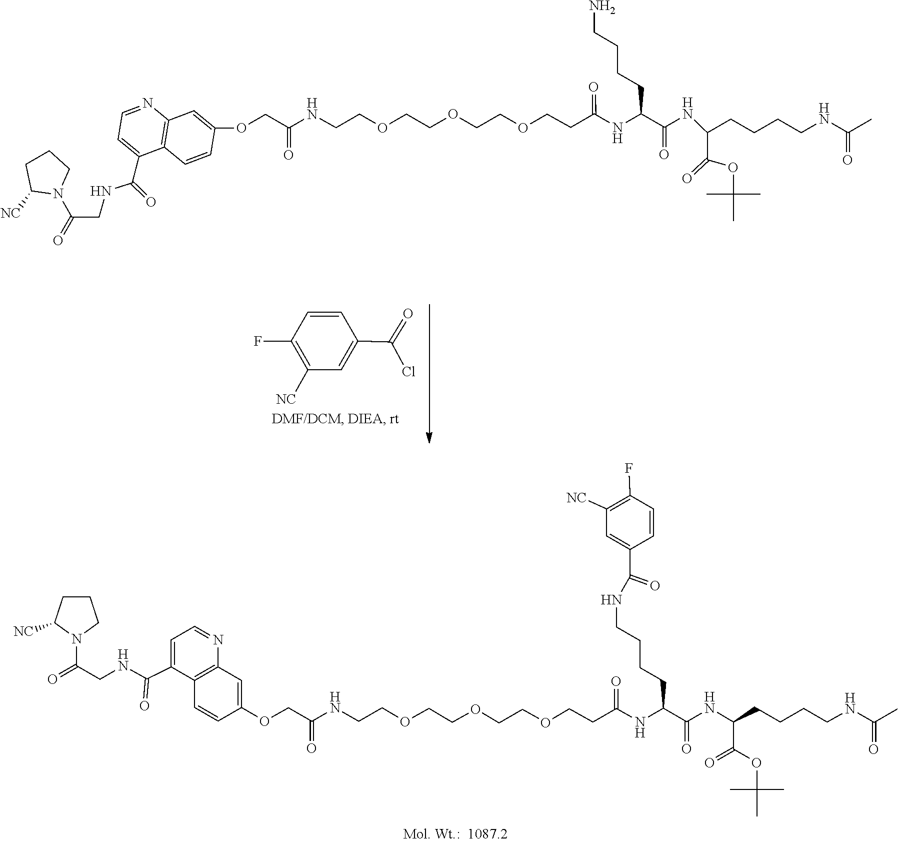 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C ; N#Cc1cc(C(=O)Cl)ccc1F ; CC(=O)NCCCC[C@H](NC(=O)[C@H](CCCCNC(=O)c1ccc(F)c(C#N)c1)NC(=O)CCOCCOCCOCCNC(=O)COc1ccc2c(C(=O)NCC(=O)N3CCC[C@H]3C#N)ccnc2c1)C(=O)OC(C)(C)C]

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy] ethoxy} propionyl amino)-6-aminohexanoylamino]-6-acetylaminohexanoate (83 mg, 0.088 mmol) and DIEA (31 ul, 0.176 mmol) in a mixture of DCM/DMF (1:1, 1 ml) was added 4-fluoro-3-trifluoromethyl-benzoyl chloride (16 mg, 0.088 mmol) in DCM (500 ul) at 0° C. under nitrogen. The temperature was allowed to rise to ambient and the reaction mixture was stirred for 10 min. Full conversion was detected by LCMS. The mixture was diluted with EtOAc, washed with water (×2) and brine, dried over anhydrous MgSO₄ and filtered. The filtrate was evaporated to provide the target material (37 mg, 39%) as a colorless glassy solid.

Procedure 22: Preparation of RTX-1324S: (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy) ethoxy]ethoxy}propionylamino)-6-(3-cyano-4-fluorobenzoylamino) hexanoylamino]-6-acetylaminohexanoic acid:

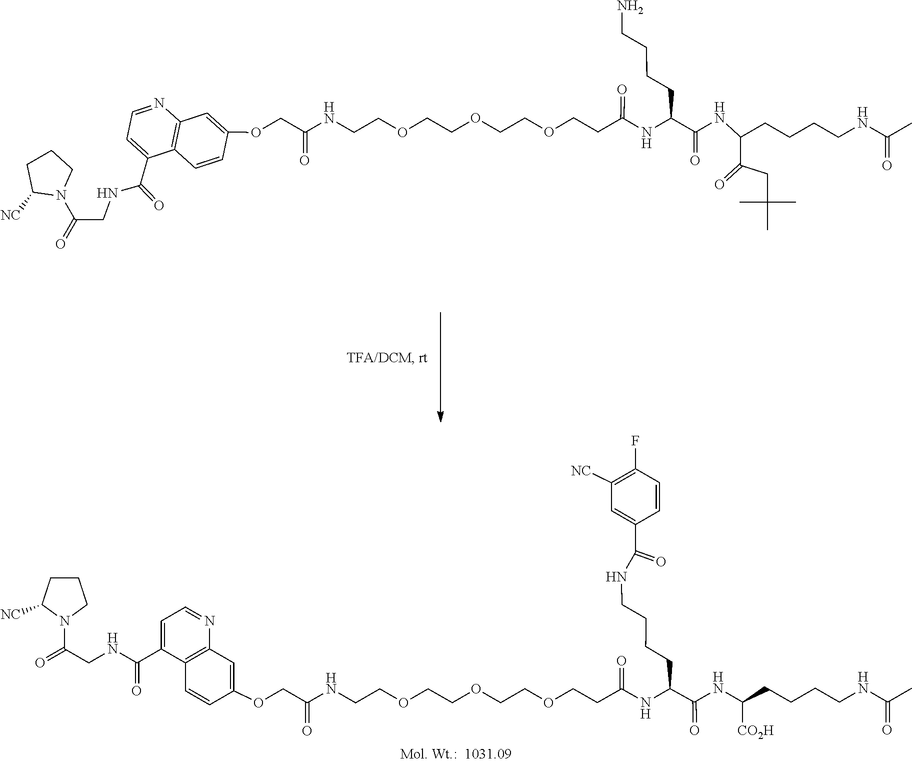 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)CC(C)(C)C ; CC(=O)NCCCC[C@H](NC(=O)[C@H](CCCCNC(=O)c1ccc(F)c(C#N)c1)NC(=O)CCOCCOCCOCCNC(=O)COc1ccc2c(C(=O)NCC(=O)N3CCC[C@H]3C#N)ccnc2c1)C(=O)O]

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-(3-cyano-4-fluorophenylamino)hexanoylamino]-6-acetylaminohexanoate (37 mg, 0.034 mmol) in anhydrous DCM (1 ml) was added anhydrous TFA (1 ml, 13 mmol) dropwise at 0° C. The temperature of the reaction mixture was allowed to rise to ambient and stirring was continued for 90 mins. Full conversion was detected by LCMS. The solvent was evaporated at room temperature and residue was co-evaporated with toluene. The residue so obtained residue was subjected to HLPC purification. The desired fractions were combined and lyophilized to obtain the target material, RTX-1324S (15.8 mg, 45%) as an off-white solid.

### Example 3—Synthesis of RTX-1325S: ((S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino} ethoxy) ethoxy]ethoxy}propionylamino)-6-[4-fluoro-3-(trifluoromethyl) benzoylamino]hexanoylamino]-6-acetylaminohexanoic Acid

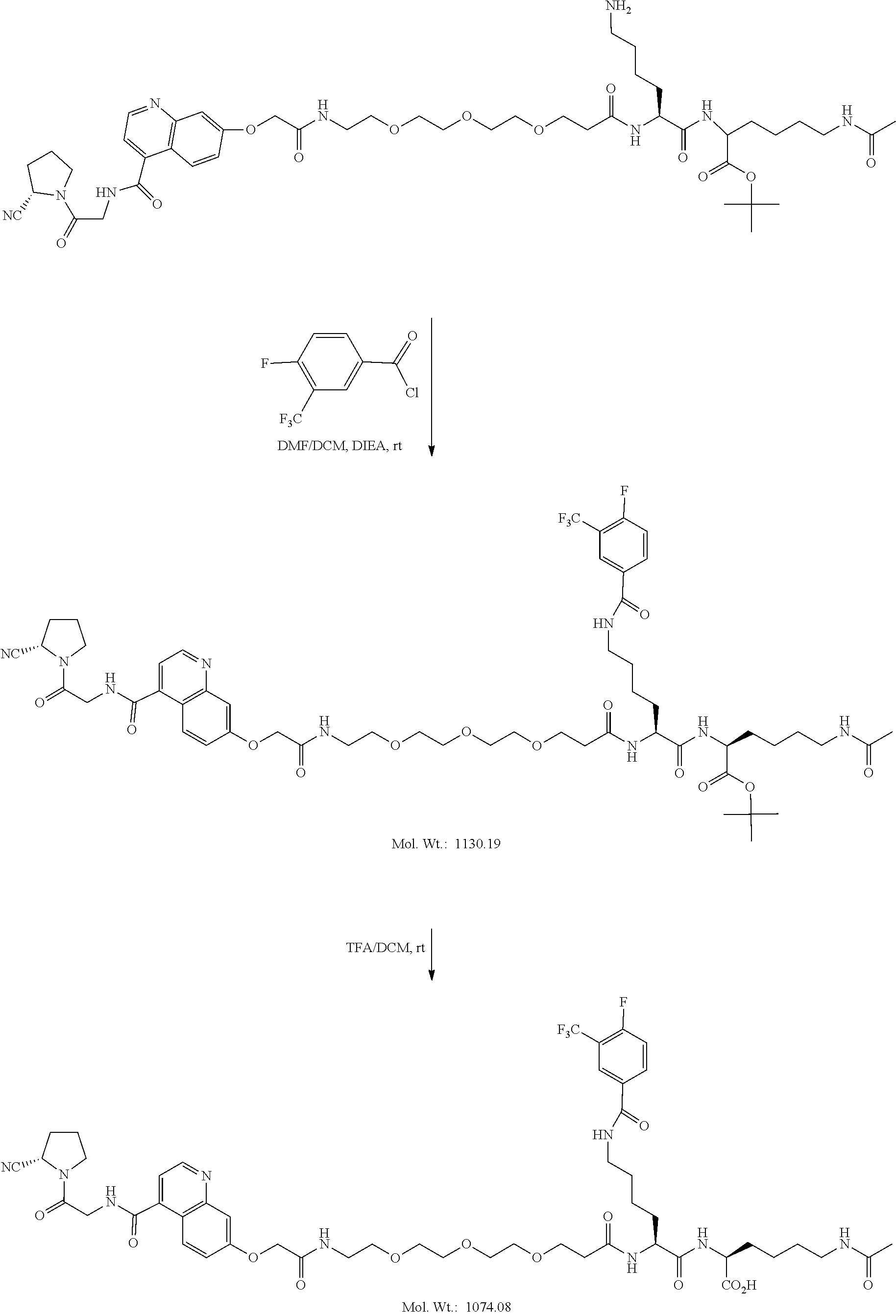 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C ; O=C(Cl)c1ccc(F)c(C(F)(F)F)c1 ; CC(=O)NCCCC[C@H](NC(=O)[C@H](CCCCNC(=O)c1ccc(F)c(C(F)(F)F)c1)NC(=O)CCOCCOCCOCCNC(=O)COc1ccc2c(C(=O)NCC(=O)N3CCC[C@H]3C#N)ccnc2c1)C(=O)OC(C)(C)C ; CC(=O)NCCCC[C@H](NC(=O)[C@H](CCCCNC(=O)c1ccc(F)c(C(F)(F)F)c1)NC(=O)CCOCCOCCOCCNC(=O)COc1ccc2c(C(=O)NCC(=O)N3CCC[C@H]3C#N)ccnc2c1)C(=O)O]

Procedure 23: Preparation of tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy] ethoxy} propionyl amino)-6-[4-fluoro-3-(trifluoromethyl)benzoylamino]hexanoylamino]-6-acetylaminohexanoate:

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy] ethoxy} propionyl amino)-6-aminohexanoylamino]-6-acetylaminohexanoate (83 mg, 0.088 mmol) and DIEA (31 ul, 0.176 mmol) in the mixture of DCM/DMF (1:1, 1 ml) was added 4-fluoro-3-trifluoromethyl-benzoyl chloride (20 mg, 0.088 mmol) in DCM (500 ul) at 0° C. under nitrogen. The temperature of the reaction mixture was allowed to rise to ambient and stirring was continued for 10 min. Full conversion was detected by LCMS. The reaction mixture was diluted with EtOAc, washed with water (×2) and brine, dried over anhydrous MgSO₄ and filtered. The filtrate was evaporated to provide target material (57 mg, 58%) as colorless glassy solid.

Procedure 24: Preparation of RTX-1325S: (S)-2-[(S)-2-(3-{2-[2-(2-{2-[4-({2-[(S)-2-Cyano-1-pyrrolidinyl]-2-oxoethylamino}carbonyl)-7-quinolyloxy]acetylamino}ethoxy)ethoxy] ethoxy}propionylamino)-6-[4-fluoro-3-(trifluoromethyl) benzoylamino]hexanoylamino]-6-acetylaminohexanoic acid (RTX-1325S):

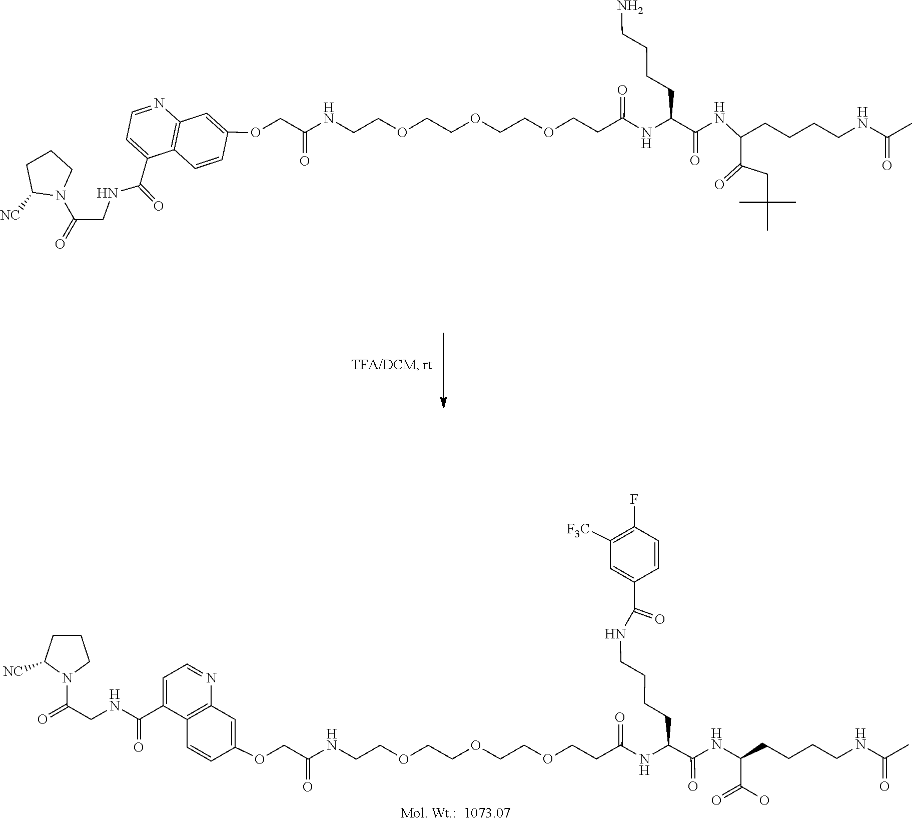 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)CC(C)(C)C ; CC(=O)NCCCC[C@H](NC(=O)[C@H](CCCCNC(=O)c1ccc(F)c(C(F)(F)F)c1)NC(=O)CCOCCOCCOCCNC(=O)COc1ccc2c(C(=O)NCC(=O)N3CCC[C@H]3C#N)ccnc2c1)C(=O)O]

To a solution of tert-butyl (S)-2-[(S)-2-(3-{2-[2-(2-{2-[4-({2-[(S)-2-cyano-1-pyrrolidinyl]-2-oxoethyl amino} carbonyl)-7-quinolyloxy]acetylamino}ethoxy)ethoxy]ethoxy} propionyl amino)-6-(3-trifluoromethyl-4-fluorophenylamino)hexanoylamino]-6-acetylaminohexanoate (57 mg, 0.051 mmol) in anhydrous DCM (1 ml) was added anhydrous TFA (1 ml, 13 mmol) dropwise at 0° C. The temperature of the reaction mixture was allowed to rise to ambient and stirring was continued for 90 min. Full conversion was detected by LCMS. The solvent was evaporated at rt and residue was co-evaporated with toluene. The residue so obtained was subjected to HLPC purification. The desired fractions were combined and lyophilized to obtain target material RTX-1325S (21.8 mg, 40%) as off-white solid.

### Example 4—Synthesis of RTX-1326S: 7-Methoxy-quinoline-4-carboxylic Acid [2-((S)-2-cyano-pyrrolidin-1-yl)-2-oxo-ethyl]-amide

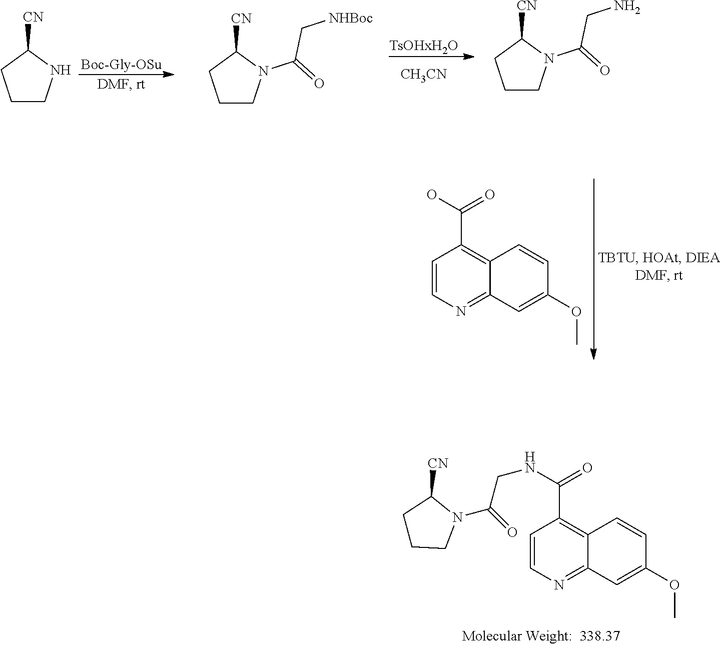 [SMILES: N#C[C@@H]1CCCN1 ; CC(C)(C)OC(=O)NCC(=O)N1CCC[C@H]1C#N ; "CH3CN" ; N#C[C@@H]1CCCN1C(=O)CN ; COc1ccc2c(C(=O)O)ccnc2c1 ; COc1ccc2c(C(=O)NCC(=O)N3CCC[C@H]3C#N)ccnc2c1]

Procedure 25: Preparation of [2-((S)-2-Cyano-pyrrolidin-1-yl)-2-oxo-ethyl]-carbamic acid tert-butyl ester:

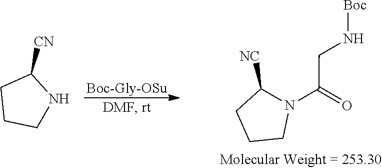 [SMILES: N#C[C@@H]1CCCN1 ; CC(C)(C)OC(=O)NCC(=O)N1CCC[C@H]1C#N]

To a solution of (S)-pyrrolidine-2-carbonitrile hydrochloride (1 g, 7.5 mmol) in DCM (35 ml) was added DIEA (3.9 ml, 22.5 mmol) and Boc-Gly-OSu (2.26 g, 8.3 mmol). The resulting solution was stirred at ambient temperature for 14 h. The reaction mixture was concentrated under vacuum and the residue was dissolved DCM, washed with water, dried over anhydrous MgSO₄ and filtered. The filtrate was evaporated and subjected to flash purification on CombiFlash (silica gel, gradient 0-10% MeOH/DCM in 30 min). The desired fractions were collected and evaporated to obtain the target material (1.1 g, 58% yield) as an amorphous solid.

Procedure 26: Preparation of (S)-1-(2-Amino-acetyl)-pyrrolidine-2-carbonitrile hydrochloride:

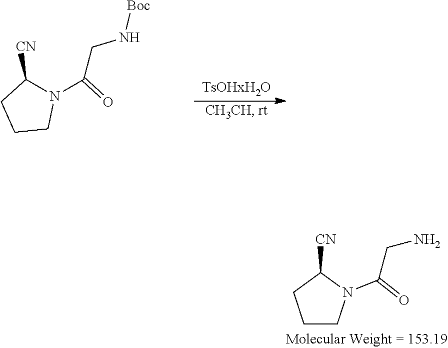 [SMILES: CC(C)(C)OC(=O)NCC(=O)N1CCC[C@H]1C#N ; N#C[C@@H]1CCCN1C(=O)CN]

To a solution (1.1 g, 4.4 mmol) of (2-((S)-2-cyano-pyrrolidin-1-yl)-2-oxo-ethyl)-carbamic acid tert-butyl ester (1.1 g, 4.4 mmol) in acetonitrile (20 ml) was added p-toluenesulfonic acid monohydrate (1.25 g, 6.6 mmol). The reaction mixture was stirred at ambient temperature for 14 h. The solvent was evaporated to dryness to obtain the target product (1.8 g, yield exceeded theoretical). This material was used without further purification.

Procedure 27: Preparation of 7-Methoxy-quinoline-4-carboxylic acid [2-((S)-2-cyano-pyrrolidin-1-yl)-2-oxo-ethyl]-amide:

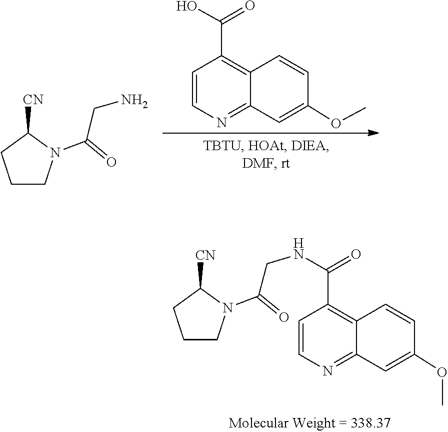 [SMILES: N#C[C@@H]1CCCN1C(=O)CN ; COc1ccc2c(C(=O)O)ccnc2c1 ; COc1ccc2c(C(=O)NCC(=O)N3CCC[C@H]3C#N)ccnc2c1]

To a solution of 7-methoxy-4-quinolinecarboxylic acid (303 mg, 1.5 mmol) in DMF (5 ml) was added HOAt (205 mg, 1.5 mmol) and TBTU (482 mg, 1.5 mmol) followed by the addition of (S)-1-(2-amino-acetyl)-pyrrolidine-2-carbonitrile tosylate (789 mg, 1.8 mmol) and DIEA (0.7 ml, 4.5 mmol). The reaction mixture was stirred at ambient temperature for 4 hours before the solvents were evaporated. The residue was suspended in DCM and insoluble material was removed by centrifugation. The DCM solution was loaded onto a CombiFlash silica gel column and purified by flash chromatography (gradient 0-10% MeOH/DCM in 30 min). The desired fractions were collected and evaporated to obtain the target material (400 mg, 75% yield) as an off-white solid.

### Example 5—Synthesis of RTX-1335S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-(3-cyano-4-fluorobenzoylamino)hexanoylamino]-6-acetylaminohexanoate

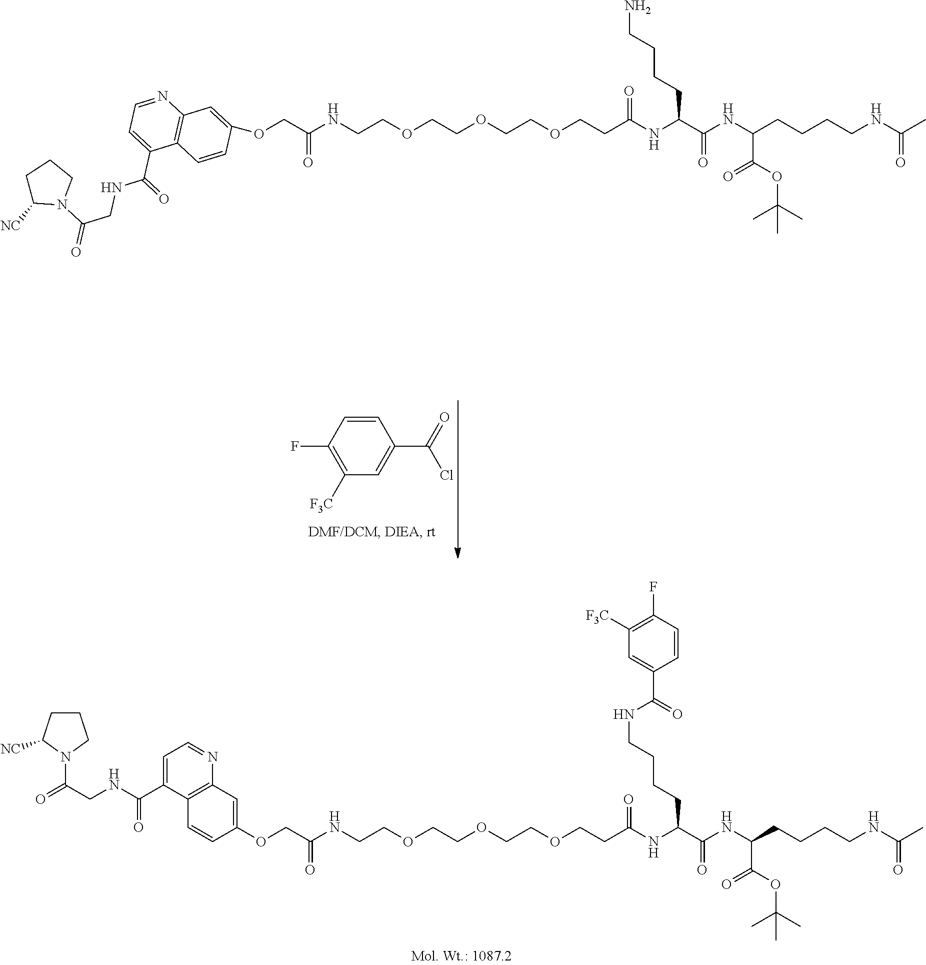 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C ; O=C(Cl)c1ccc(F)c(C(F)(F)F)c1 ; CC(=O)NCCCC[C@H](NC(=O)[C@H](CCCCNC(=O)c1ccc(F)c(C(F)(F)F)c1)NC(=O)CCOCCOCCOCCNC(=O)COc1ccc2c(C(=O)NCC(=O)N3CCC[C@H]3C#N)ccnc2c1)C(=O)OC(C)(C)C]

Procedure 28: To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy] ethoxy} propionyl amino)-6-aminohexanoylamino]-6-acetylaminohexanoate (0.072 mmol) and DIEA (25 ul, 0.144 mmol) in the mixture of DCM/DMF (1:1, 1 ml) was added 4-fluoro-3-cyano-benzoyl chloride (13 mg, 0.072 mmol) in DCM (500 ul) at 0° C. under nitrogen. The temperature of the reaction mixture was allowed to rise to ambient and reaction was stirred for 10 min. Full conversion was detected by LCMS. The reaction mixture was diluted with EtOAc, washed with water (×2) and brine, dried over anhydrous MgSO₄ and filtered. The filtrate was evaporated and the residue was subjected to HPLC purification. The desired fractions were collected and lyophilized to obtain the target material, RTX-1335S (25.4 mg, 33%) as an off-white solid.

### Example 6—Synthesis of RTX-1336S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy] acetylamino}ethoxy)ethoxy]ethoxy}propionyl amino)-6-(3-cyano-4-trimethylammoniumbenzoylamino)hexanoylamino]-6-acetylaminohexanoate

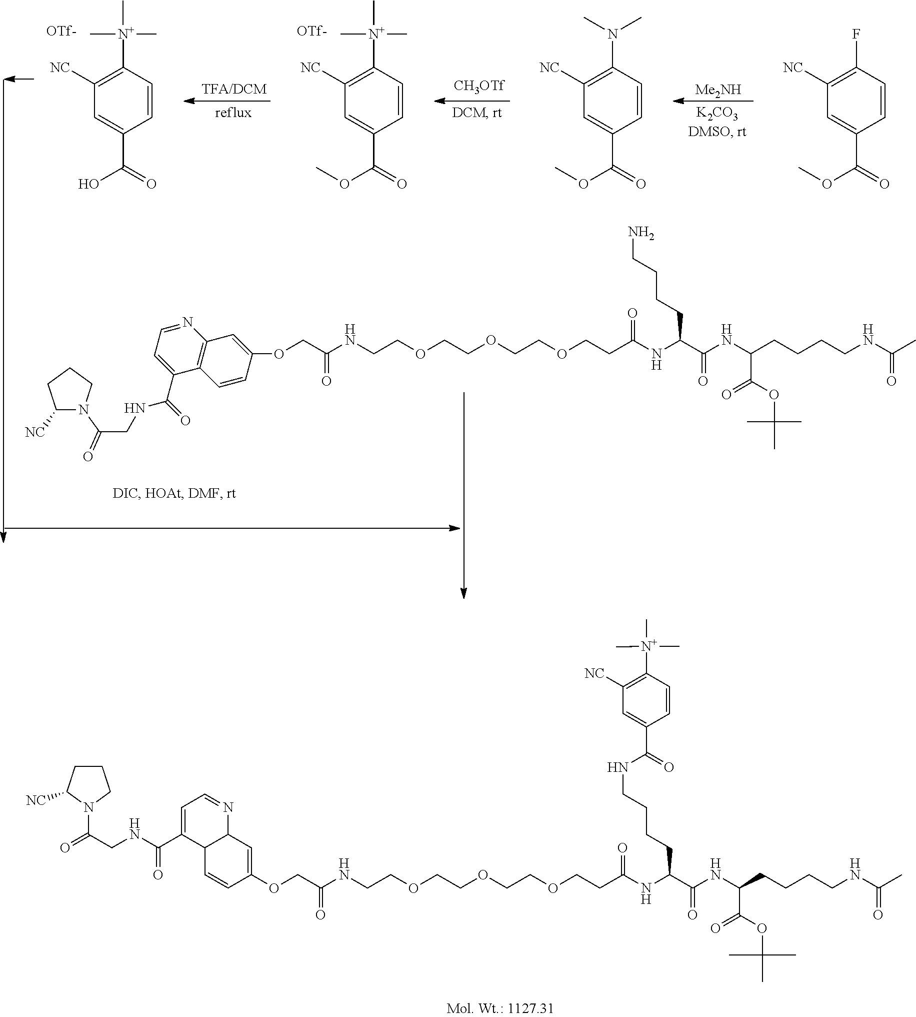

Procedure 29: Preparation of 3-Cyano-4-dimethylamino-benzoic acid methyl ester:

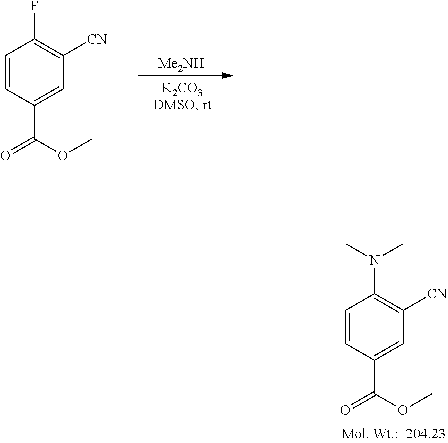

To a solution of methyl 3-cyano-4-fluorobenzoate (1 g, 5.58 mmol) and dimethylamine hydrochloride (546 mg, 6.7 mmol) in DMSO (12 ml) was added potassium carbonate (1.62 g, 11.72 mmol) and the resulting mixture was stirred for 14 h at ambient temperature. The mixture was concentrated at 65° C. using a high vacuum rotary evaporator. The residual solution was diluted with DCM and extracted with water (×2). The combined aqueous layers were extracted back with DCM. The combined organic layers were washed with dilute sodium bicarbonate solution, dried over anhydrous MgSO₄, filtered, and evaporated to obtain target compound (1.13 g, 99%) as an off-white solid.

Procedure 30: Preparation of (2-Cyano-4-methoxycarbonylphenyl)-trimethylammonium trifluoromethanesulfonate:

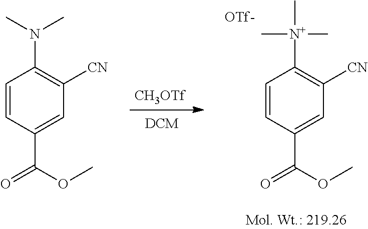 [SMILES: COC(=O)c1ccc(N(C)C)c(C#N)c1 ; COC(=O)c1ccc([N+](C)(C)C)c(C#N)c1 ; "CH3OTf"]

Methyl triflate (6.26 ml, 55.3 mmol) was added slowly (dropwise) to a stirred solution of 3-cyano-4-dimethylamino-benzoic acid methyl ester (1.13 g, 5.53 mmol) in DCM (20 ml). The reaction mixture was stirred for 14 h before diethyl ether was added. After evaporation of one third of the solvent volume, the desired compound precipitated from solution and the remaining solvent was decanted. The solid was washed extensively with a large amount of ether. Residual ether was removed in vacuo and the solid was purified by HPLC. The desired fraction was collected and evaporated, and the residue was dried under vacuum to obtain the target compound (1.033 g, 51%) as an off-white solid.

Procedure 31: Preparation of (4-Carboxy-2-cyanophenyl)-trimethylammonium trifloromethanesulfonate:

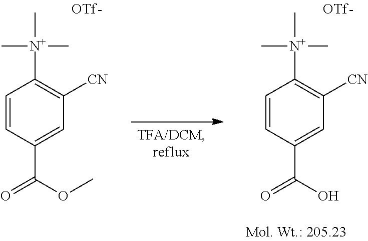 [SMILES: COC(=O)c1ccc([N+](C)(C)C)c(C#N)c1 ; C[N+](C)(C)c1ccc(C(=O)O)cc1C#N]

A solution of (2-cyano-4-methoxycarbonylphenyl)-trimethylammonium trifluoromethanesulfonate (680 mg, 1.85 mmol) in water (20 ml) and TFA (20 ml, 260 mmol) was heated at reflux for 2 days. The solvents were evaporated, and the residue was dried in vacuo at ambient temperature for 14 h. The residue was treated with diethyl ether, and the precipitate that formed was filtered, washed with ether, and dried in vacuo to the obtain target compound (562 mg, 83%) as an off-white solid.

Procedure 32: Preparation of RTX-1336S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy] acetylamino}ethoxy)ethoxy]ethoxy}propionyl amino)-6-(3-cyano-4-trimethylammoniumbenzoylamino)hexanoylamino]-6-acetylaminohexanoate

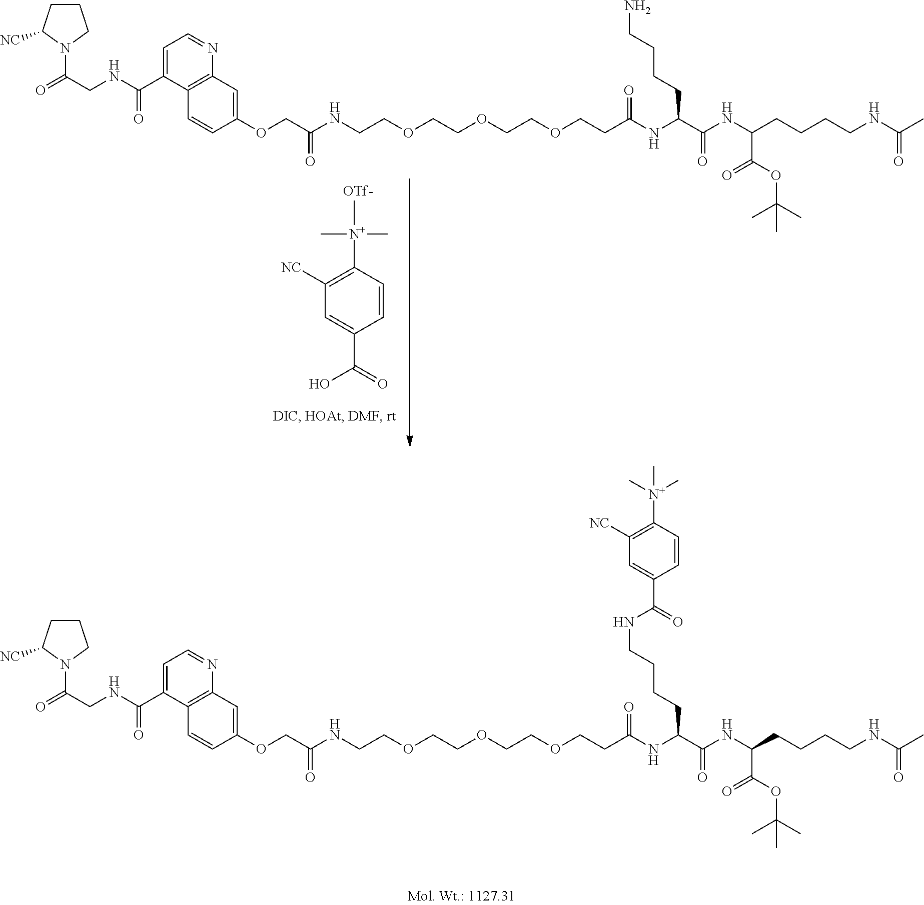 [SMILES: CC(=O)NCCCCC(NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C ; C[N+](C)(C)c1ccc(C(=O)O)cc1C#N ; CC(=O)NCCCC[C@H](NC(=O)[C@H](CCCCNC(=O)c1ccc([N+](C)(C)C)c(C#N)c1)NC(=O)CCOCCOCCOCCNC(=O)COc1ccc2c(C(=O)NCC(=O)N3CCC[C@H]3C#N)ccnc2c1)C(=O)OC(C)(C)C]

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-aminohexanoylamino]-6-acetylaminohexanoate (0.125 mmol), (4-carboxy-2-cyano-phenyl)-trimethylammonium trifloromethanesulfonate (0.51 mg, 0.150 mmol) and HOAt (1-hydroxy-7-azabenzotriazole, 24 mg, 0.175 mmol) in DMSO (3 ml) was added DIC (24 ul, 0.150 mmol). The reaction mixture was stirred 14 h at ambient temperature. Full conversion was observed by LCMS. The reaction mixture was subjected to HPLC purification, and the desired fractions were isolated, combined and lyophilized to obtain the trifluoroacetate salt of the target compound (56 mg, 35%) as an off-white solid.

### Example 7—Synthesis of RTX-1337S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-[4-fluoro-3-(trifluoromethyl)benzoylamino]hexanoylamino]-6-acetylaminohexanoate

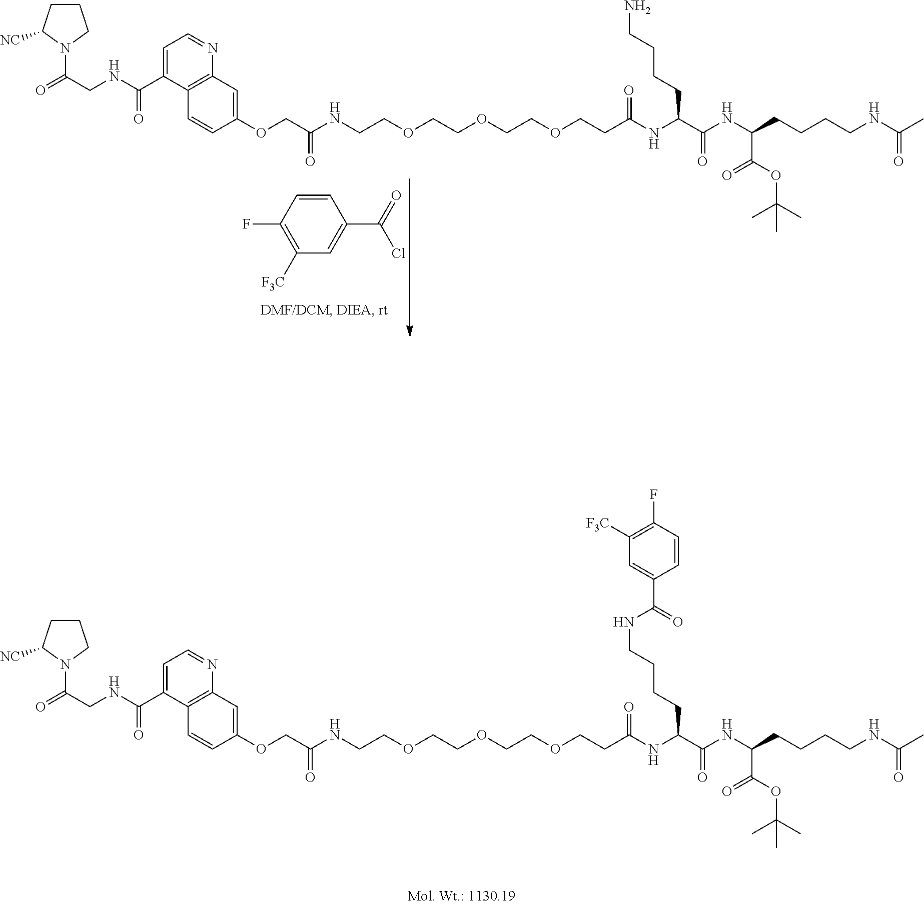

Procedure 33: To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-aminohexanoylamino]-6-acetylaminohexanoate (0.072 mmol) and DIEA (25 ul, 0.144 mmol) in the mixture of DCM/DMF (1:1, 1 ml) was added 4-fluoro-3-trifluoromethyl-benzoyl chloride (16 mg, 0.072 mmol) in DCM (500 ul) at 0° C. under nitrogen. The temperature of the reaction mixture was allowed to rise to ambient and reaction was stirred for 10 min. Full conversion was detected by LCMS. The reaction mixture was diluted with EtOAc, washed with water (×2) and brine, dried over anhydrous MgSO₄ and filtered. The filtrate was evaporated and the residue was purified by HPLC (Method 4). The desired fractions were collected and lyophilized to obtain the target material, RTX-1337S (19.5 mg, 24%) as an off-white solid.

### Example 8—Synthesis of RTX-1338S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy] acetylamino}ethoxy)ethoxy]ethoxy}propionyl amino)-6-(3-trifluoromethyl-4-trimethylammoniumbenzoylamino) hexanoylamino]-6-acetylaminohexanoate

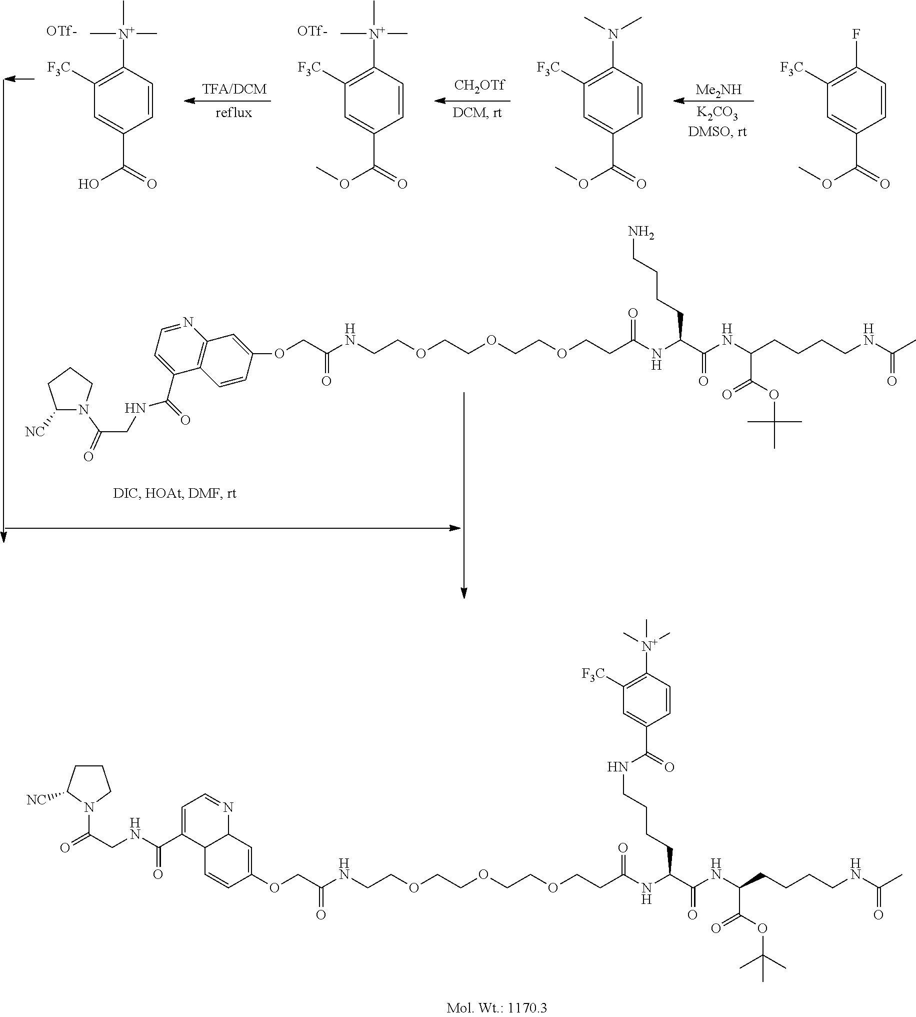

Procedure 34: Preparation of 3-Trifluoromethyl-4-dimethylamino-benzoic acid methyl ester.

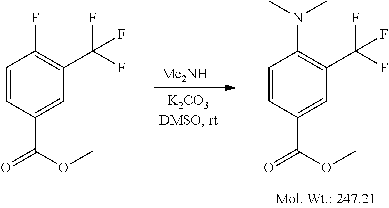 [SMILES: "Me2NH" ; COC(=O)c1ccc(F)c(C(F)(F)F)c1 ; COC(=O)c1ccc(N(C)C)c(C(F)(F)F)c1]

To a solution of methyl 4-fluoro-3-(trifluoromethyl)benzoate (1.24 g, 5.58 mmol) and dimethylamine hydrochloride (546 mg, 6.7 mmol) in DMSO (12 ml) was added potassium carbonate (1.62 g, 11.72 mmol) and reaction mixture was stirred for 14 h at ambient temperature. The reaction mixture was concentrated at 65° C. using a high vacuum rotary evaporator. The residual solution was diluted with DCM and extracted with water (×2). The combined aqueous layers were extracted back with DCM. The combined organic layers were washed with dilute aqueous sodium bicarbonate, dried over anhydrous MgSO4, filtered, and evaporated to the obtain target compound (1.38 g, quant.) as colorless amorphous solid.

Procedure 35: Preparation of (2-Trifluoromethyl-4-methoxycarbonylphenyl)-trimethylammonium trifluoromethanesulfonate:

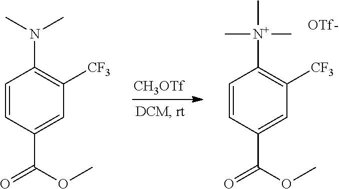 [SMILES: COC(=O)c1ccc(N(C)C)c(C(F)(F)F)c1 ; COC(=O)c1ccc([N+](C)(C)C)c(C(F)(F)F)c1 ; "CH3OTf"]

Methyl triflate (9.51 ml, 84.2 mmol) was added slowly (dropwise) to a stirred solution of 4-dimethylamino-3-trifluoromethylbenzoic acid methyl ester (1.38 g, 5.59 mmol) in DCM (25 ml). The reaction mixture was heated reflux for 2 days, cooled to ambient temperature and treated with diethyl ether. The desired compound precipitated and the solvent was removed by decanting it. The solid was washed repeatedly with diethyl ether and dried in vacuo. The remaining solid was purified by HPLC. The desired fraction was collected and evaporated, and the residue residue was dried in vacuo to obtain the target compound, isolated as the trifluoromethanesulfonate salt (1.35 g, 59%) as an off-white solid.

Procedure 36: Preparation of (4-Carboxy-2-trifluoromethylphenyl)-trimethylammonium trifluoromethanesulfonate:

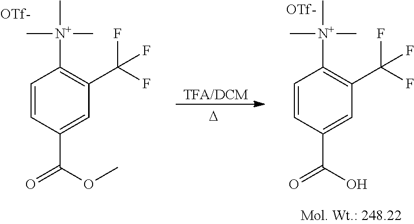

A solution of (2-trifluoromethyl-4-methoxycarbonyl-phenyl)trimethylammonium trifluoromethanesulfonate (760 mg, 2.9 mmol) in a mixture of water (20 ml) and TFA (20 ml, 260 mmol) was heated at reflux for 2 days. The solvents were evaporated and the residue was dried in vacuo at ambient temperature for 14 h. The residue was treated with diethyl ether and the resulting precipitate was filtered, washed with diethyl ether and dried in vacuo to obtain the target compound (668 mg, 58%) as a white solid.

Procedure 37: Preparation of RTX-1338S: tert-Butyl (S)-2-[(S)-2-(3-{2-[2-(2-{2-[4-({2-[(S)-2-cyano-1-pyrrolidinyl]-2-oxoethylamino}carbonyl)-7-quinolyloxy] acetylamino}ethoxy)ethoxy]ethoxy}propionylamino)-6-(3-trifluoromethyl-4-trimethylammoniumbenzoylamino)hexanoylamino]-6-acetylaminohexanoate

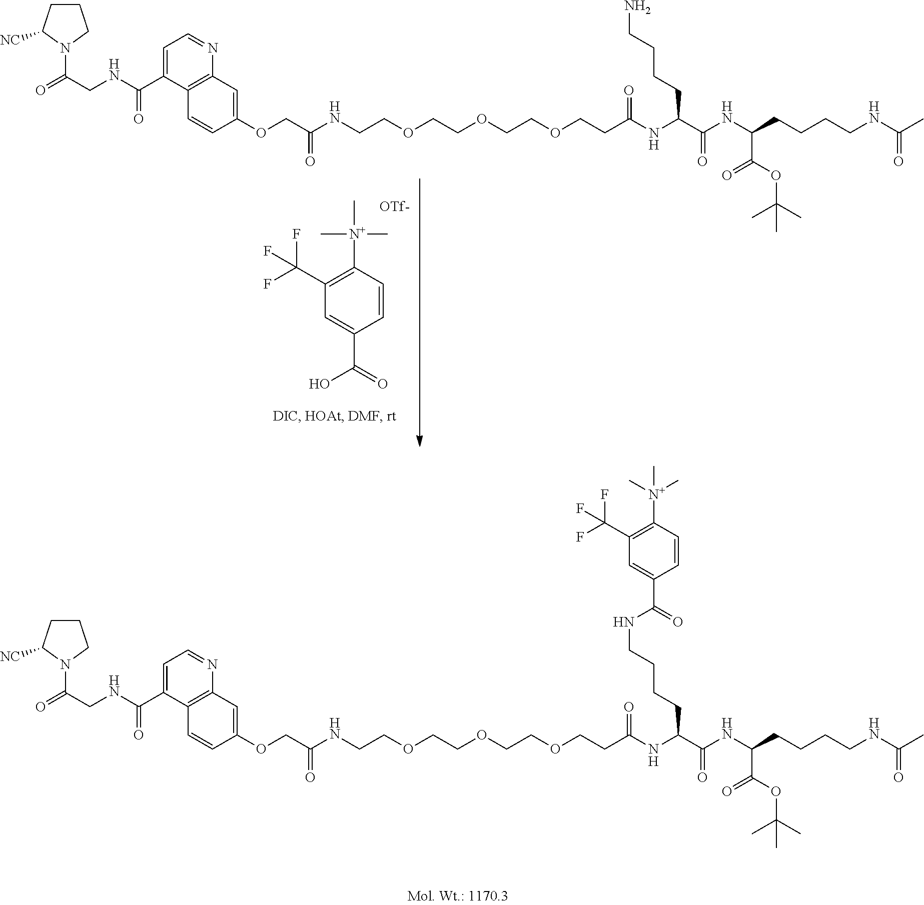 [SMILES: CC(=O)NCCCC[C@H](NC(=O)[C@H](CCCCN)NC(=O)CCOCCOCCOCCNC(=O)COc1ccc2c(C(=O)NCC(=O)N3CCC[C@H]3C#N)ccnc2c1)C(=O)OC(C)(C)C ; C[N+](C)(C)c1ccc(C(=O)O)cc1C(F)(F)F ; CC(=O)NCCCC[C@H](NC(=O)[C@H](CCCCNC(=O)c1ccc([N+](C)(C)C)c(C(F)(F)F)c1)NC(=O)CCOCCOCCOCCNC(=O)COc1ccc2c(C(=O)NCC(=O)N3CCC[C@H]3C#N)ccnc2c1)C(=O)OC(C)(C)C]

To a solution of tert-butyl (S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-aminohexanoylamino]-6-acetylaminohexanoate (77 mg, 0.082 mmol), (4-carboxy-2-trifluoromethylphenyl)-trimethylammonium trifloromethanesulfonate (35 mg, 0.098 mmol) and HATU (62 mg, 0.164 mmol) in DMF (2 mL) was added 2,6-lutidine (22 μL, 0.205 mmol). The reaction mixture was stirred at ambient temperature for 14 h, at which point full conversion was detected by LCMS. The reaction mixture was subjected to HPLC purification, and the desired fractions were combined and lyophilized to obtain the trifluoroacetate salt of the target compound RTX-1338S (56 mg, 35%) as an off-white solid.

### Example 9—Synthesis of RTX 1363S: 2,2′-(7-((S)-17-(((S)-1-carboxy-5-hexanamidopentyl)carbamoyl)-1-((4-((2-((S)-2-cyanopyrrolidin-1-yl)-2-oxoethyl)carbamoyl)quinolin-7-yl)oxy)-2,15,23-trioxo-6,9,12-trioxa-3,16,22-triazatetracosan-24-yl)-1,4,7-triazonane-1,4-diyl)diacetic Acid

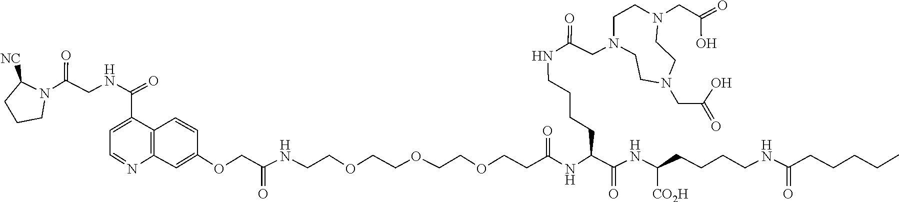 [SMILES: CCCCCC(=O)NCCCC[C@H](NC(=O)[C@H](CCCCNC(=O)CN1CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

For the purpose of synthesis, RTX-1363S molecule was broken down into three fragments as follows:

### Preparation of Int #1

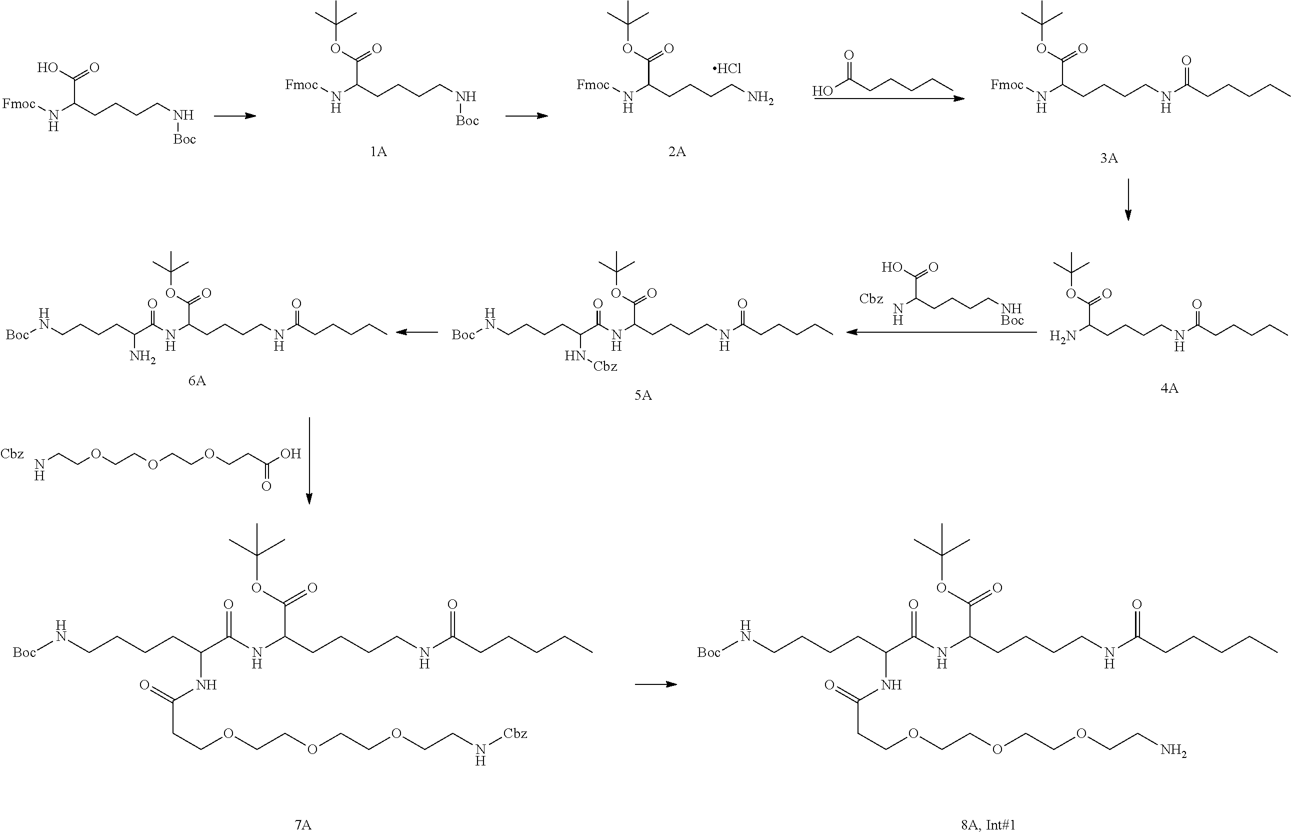

### Synthesis of RTX-1363S from Fragments

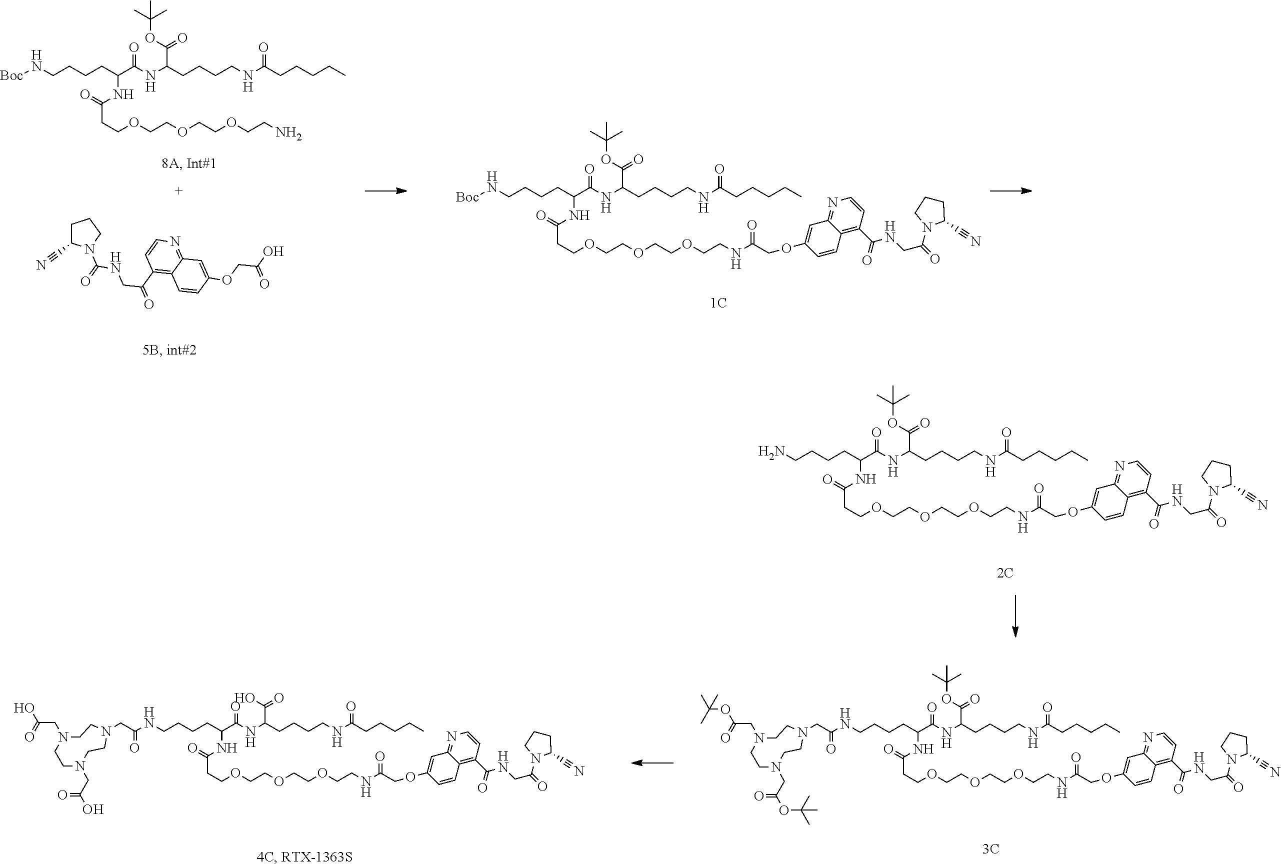

Procedure 38: Preparation of Int #1. Tert-Butyl-(S)-2-[(S)-2-(3-{2-[2-(2-aminoethoxy)ethoxy]ethoxy} propionylamino)-6-(tert-butoxycarbonylamino)hexanoylamino]-6-(hexanoylamino)hexanoate: Int 1A, Fmoc-L-Lys (Boc)-OtBu, SCR448-17

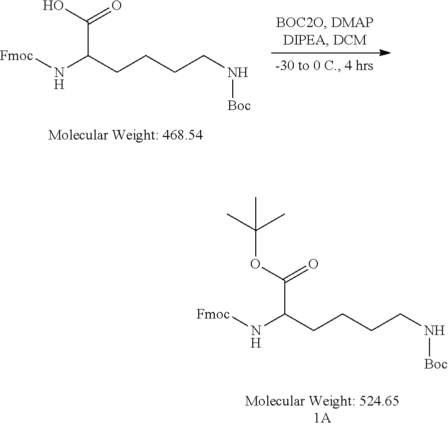

Fmoc-L-Lys(Boc)-OH (47 g, 100 mmol) was dissolved in DCM (470 ml), and diisopropylethylamine (25 ml) was added at −30° C. The reaction mixture was stirred at −30° C. for 5 min, followed by slow addition of Boc anhydride (30.1 g, 140 mmol) at −30° C. The reaction mixture was stirred at −30° C. for a further 30 mins, then N,N-dimethylaminopyridine (1.7 g, 14 mmol) was added at −30° C. The reaction mixture was slowly warmed to 0° C. over the course of 2 h and was stirred at 0° C. for another 2 h at which time LCMS analysis of the reaction mixture showed complete consumption of the starting carboxylic acid). The reaction mixture was cooled to −30° C. and 1M HCl aqueous solution was added slowly to obtain a pH˜3 (the temperature of mixture was kept below −20° C.). The resulting biphasic mixture was separated and the organic layer was washed with water (200 ml) and 0.1M NaHCO₃ (200 ml), dried with MgSO4, filtered and evaporated to dryness. The residue was diluted with 30 ml of ethyl acetate, heated to reflux and diluted with 300 ml of hexane. The mixture was stirred at room temperature for 2 h and filtered. The precipitate was washed with 100 ml of 20% ethyl acetate in hexane and the residual solvent was removed in vacuo overnight. Yield=23 g (44%).

Procedure 39: Int 2A, Fmoc-L-Lys (Boc)-OtBu

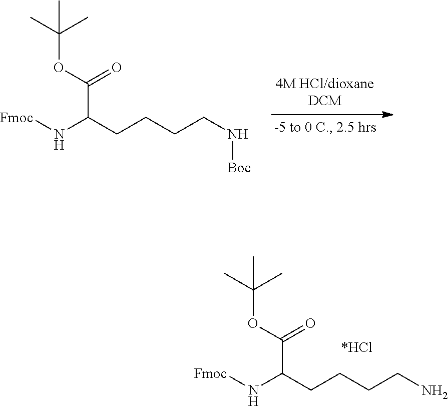

To a solution of Fmoc-L-Lys(Boc)-OtBu (23 g, 44 mmol) in 130 ml DCM was added 4M HCl in dioxane (275 ml) at −5° C. The mixture was stirred for 2.5 hour at −5° C. to 0° C. (until LCMS showed no starting material). The solvents were evaporated in vacuo without heating, re-evaporated with 150 ml of DCM, and residual solvents were removed under vacuum at room temperature overnight. The product was obtained as a white amorphous solid (22.6 g, 110%).

Procedure 40: Int #3A, tert-Butyl-(S)-2-[(9H-fluoren-9-yl)methoxycarbonylamino]-6-(hexanoylamino) hexanoate

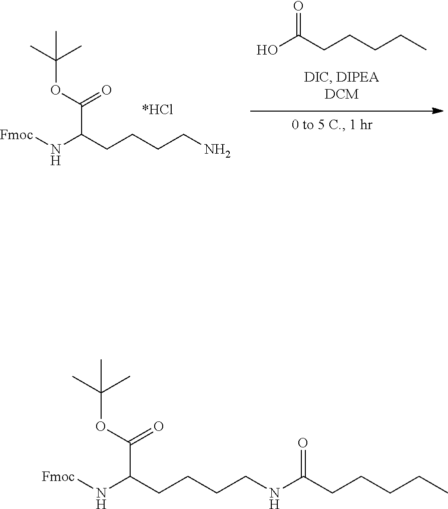

Diisopropylcarbodiimide (DIC, 16.7 g, 132 mmol) was added dropwise to a solution of hexanoic acid (30.7 g, 264 mmol) in DCM (120 ml) at 0° C. and the reaction mixture was stirred for 30 minutes at 0° C. The precipitated solid was removed by filtration. The filtrate was added to a suspension of Fmoc-L-Lys-OtBu hydrochloride (20.3 g, 44 mmol) in DCM (30 ml), followed by the addition of DIPEA (7.7 ml, 1 eq); the temperature was held at 0° C. to 5° C. during the addition. The reaction mixture was stirred for 30 minutes at 5° C., at which time LCMS showed no starting material remaining. The mixture was diluted with 150 ml of DCM, and the organic layer was washed with 0.5 M HCl solution (100 ml), washed with 0.1 M NaHCO₃ solution (100 ml), dried over MgSO₄ and evaporated. The residue was purified by flash chromatography (330 g silica gel column, 0% to 50% ethyl acetate in hexane) to obtain the target compound (16.8 g, 73%) as a white glass.

Procedure 41: Int #4A, tert-Butyl (S)-2-amino-6-(hexanoylamino)hexanoate

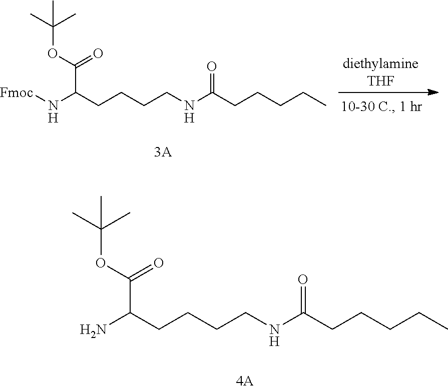

To a solution of tert-butyl-(S)-2-[(9H-fluoren-9-yl)methoxycarbonylamino]-6-(hexanoylamino) hexanoate (16 g, 30 mmol) in THF (200 ml) was added diethylamine (92 ml, 900 mm0l) at 10° C. The reaction mixture was stirred for 1 hour and evaporated to dryness under vacuum without heating. The residue was re-evaporated with toluene (100 ml) at 25-30° C. and subjected to silica gel purification (220 g column, 0% to 15% MeOH in DCM) to obtain the product (7.1 g, 79%) as a viscous yellow oil.

Procedure 42: Int #5A, tert-Butyl-(S)-2-[(S)-2-amino-6-(tert-butoxycarbonylamino)hexanoylamino]-6-(hexanoylamino)hexanoate

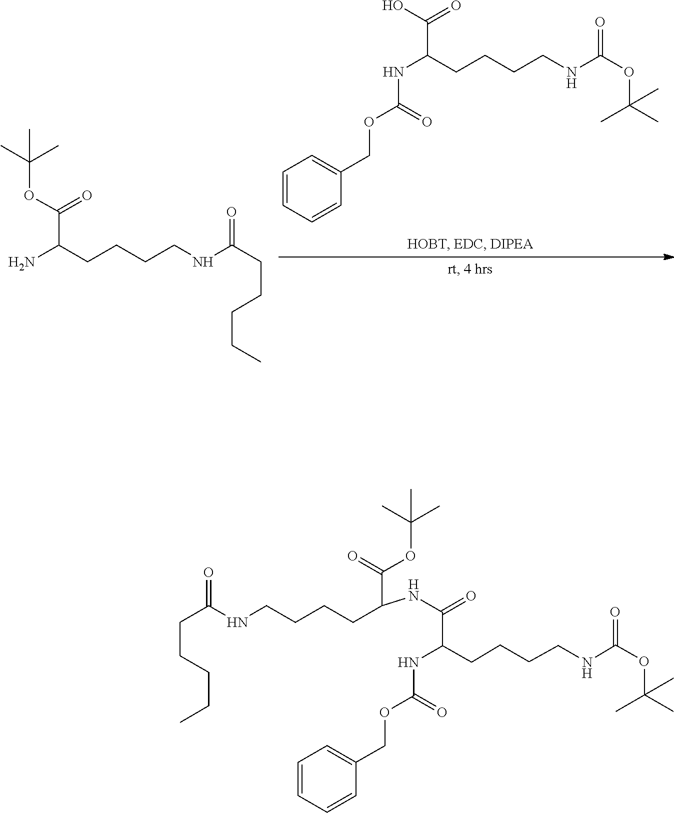 [SMILES: CCCCCC(=O)NCCCCC(N)C(=O)OC(C)(C)C ; CC(C)(C)OC(=O)NCCCCC(NC(=O)OCc1ccccc1)C(=O)O ; CCCCCC(=O)NCCCCC(NC(=O)C(CCCCNC(=O)OC(C)(C)C)NC(=O)OCc1ccccc1)C(=O)OC(C)(C)C]

To a solution of CBZ-Lys(Boc)-OH (9.7 g, 25.5 mmol) in dry DCM (100 ml), HOBt (3.45 g, 25.5 mmol) was added and the mixture was stirred for 10 min at ambient temperature. A solution of tert-butyl (S)-2-amino-6-(hexanoylamino)hexanoate (6.9 g, 23 mmol) in DCM, DIPEA (12.2 ml) and EDC (5.35 g) were added sequentially. The reaction mixture was stirred at ambient temperature for 4 hours (until LCMS showed tert-butyl (S)-2-amino-6-(hexanoylamino)hexanoate was consumed), diluted with DCM (200 ml), washed with water (150 ml) and saturated NaCl solution (100 ml), and dried over anhydrous MgSO₄. The organic solution was concentrated under reduced pressure and the residue was purified by silica gel chromatography (220 g column, 15% to 80% ethyl acetate in hexane) to obtain the product (11.4 g, 75%) as an amorphous yellow solid.

Procedure 43: Int #6A, tert-Butyl-(S)-2-[(S)-2-amino-6-(tert-butoxycarbonylamino)hexanoylamino]-6-(hexanoylamino)hexanoate

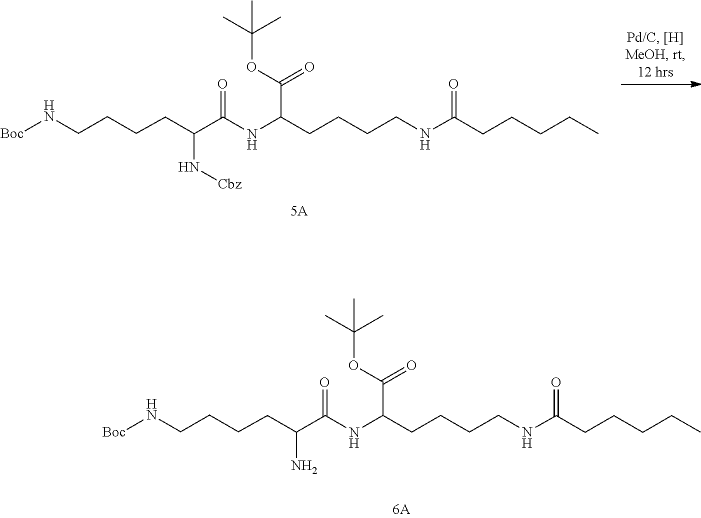 [SMILES: CCCCCC(=O)NCCCCC(NC(=O)C(CCCCNC(=O)OC(C)(C)C)NC(=O)OCc1ccccc1)C(=O)OC(C)(C)C ; CCCCCC(=O)NCCCCC(NC(=O)C(N)CCCCNC(=O)OC(C)(C)C)C(=O)OC(C)(C)C]

To a solution of tert-butyl-(S)-2-[(S)-2-amino-6-(tert-butoxycarbonylamino)hexanoylamino]-6-(hexanoylamino)hexanoate (11 g, 16.6 mmol) in methanol (400 ml) was added 10% palladium (50% wet) on activated carbon (2 g). The suspension was subjected to catalytic hydrogenation (H₂, 1 atm, overnight) at ambient temperature (after 12 h LCMS indicated complete consumption of starting material). The reaction mixture was filtered through Celite. The filtrate was concentrated under reduced pressure, re-evaporated twice with 200 ml of acetonitrile, and dried for 4 hours under high vacuum to give the product (8.96 g, 100%) as a colorless glass.

Procedure 44: Int #7A, tert-Butyl-(S)-2-[(S)-2-[3-(2-{2-[2-(benzyloxycarbonylamino)ethoxy]ethoxy} ethoxy)propionylamino]-6-(tert-butoxycarbonylamino)hexanoylamino]-6-(hexanoylamino)hexanoate

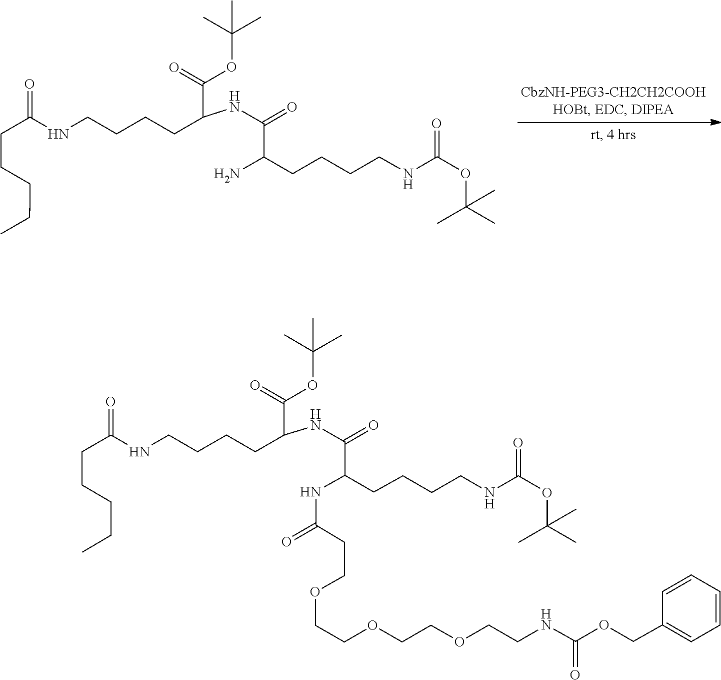 [SMILES: CCCCCC(=O)NCCCCC(NC(=O)C(N)CCCCNC(=O)OC(C)(C)C)C(=O)OC(C)(C)C ; CCCCCC(=O)NCCCCC(NC(=O)C(CCCCNC(=O)OC(C)(C)C)NC(=O)CCOCCOCCOCCNC(=O)OCc1ccccc1)C(=O)OC(C)(C)C]

To a solution of 3-(2-{2-[2-(benzyloxycarbonylamino)ethoxy]ethoxy}ethoxy)propionic acid (5.8 g, 16.6 mmol) in dry DCM (50 ml), HOBt (2.53 g, 18.7 mmol) was added and the mixture was stirred for 10 min at ambient temperature. A solution of tert-butyl-(S)-2-[(S)-2-amino-6-(tert-butoxycarbonylamino) hexanoylamino]-6-(hexanoylamino)hexanoate (8.9 g, 16.6 mmol) in DCM (50 ml) EDC (5.35 g) and DIPEA (12.2 ml) were added subsequently. The reaction mixture was stirred 4 h (LCMS monitoring), diluted with DCM (200 ml), washed with water (150 ml) and saturated NaCl solution (100 ml), and dried over MgSO₄. The organic layer was concentrated under reduced pressure and the residue was purified by silica gel chromatography (220 g column, 0% to 10% MeOH in DCM) to give target product (9.63 g, 67%) as a colorless glass.

Procedure 45: (Int #1), tert-Butyl-(S)-2-[(S)-2-(3-{2-[2-(2-aminoethoxy)ethoxy]ethoxy} propionylamino)-6-(tert-butoxycarbonylamino)hexanoylamino]-6-(hexanoylamino) hexanoate

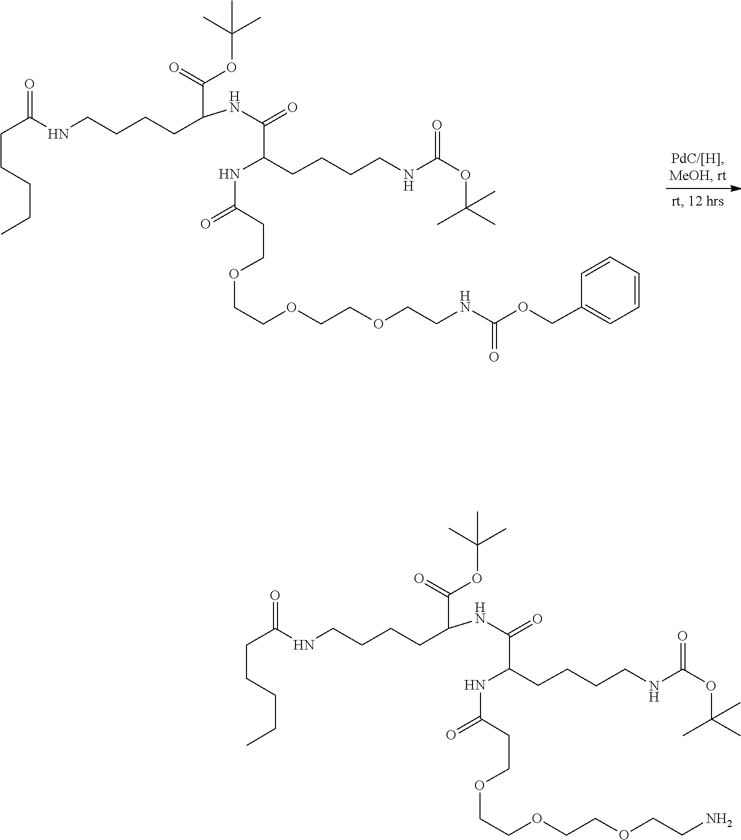 [SMILES: CCCCCC(=O)NCCCCC(NC(=O)C(CCCCNC(=O)OC(C)(C)C)NC(=O)CCOCCOCCOCCNC(=O)OCc1ccccc1)C(=O)OC(C)(C)C ; CCCCCC(=O)NCCCCC(NC(=O)C(CCCCNC(=O)OC(C)(C)C)NC(=O)CCOCCOCCOCCN)C(=O)OC(C)(C)C]

To a solution of tert-butyl-(S)-2-[(S)-2-[3-(2-{2-[2-(benzyloxycarbonylamino)ethoxy]ethoxy} ethoxy)propionylamino]-6-(tert-butoxycarbonylamino)hexanoylamino]-6-(hexanoylamino)hexanoate (9.5 g) in methanol (100 ml) was added 10% palladium on activated carbon (50% wet, 2 g). The suspension was subjected to catalytic hydrogenation at room temperature overnight (1 atm of H₂, then LCMS control absence of starting material). The reaction mixture was filtered through Celite. The filtrate was concentrated under reduced pressure, re-evaporated twice with 100 ml of acetonitrile and dried for 4 hrs in high vacuum to give target product (8.15 g, 100%) as a colorless glass.

### Preparation of RTX-1363S from Fragments

Procedure 46: Int #1C, scale up, tert-Butyl-(S)-2-[(S)-2-(3-{2-[2-(2-{2-[4-({2-[(S)-2-cyano-1-pyrrolidinyl]-2-oxoethyl amino}carbonyl)-7-quinolyloxy]acetylamino}ethoxy)ethoxy]ethoxy}propionyl-amino)-6-(tert-butoxycarbonylamino)hexanoylamino]-6-(hexanoylamino)hexanoate

To a mixture of {4-[2-((S)-2-cyano-pyrrolidin-1-yl)-2-oxo-ethylcarbamoyl]-quinolin-7-yloxy}-acetic acid (2.7 g, 5.2 mmol, crude TFA salt), (S)-2-[(S)-2-(3-{2-[2-(2-amino-ethoxy)-ethoxy]-ethoxy}-propionylamino)-6-tert-butoxycarbonylamino-hexanoylamino]-6-hexanoylamino-hexanoic acid tert-butyl ester (4.20 g, 5.72 mmol) and HATU (2.40 g, 6.24 mmol) in DMF (85 ml) was added DIEA (5.45 ml, 31.2 mmol) at ambient temperature. The mixture was stirred for 30 minutes (LCMS check for absence of SM), then diluted with EtOAc (500 ml), washed with water (2*100 ml) and brine (100 ml). The aqueous phase was back-extracted with EtOAc (250 ml). The EtOAc extract was washed with brine (100 ml). The organics were combined, dried over anhydrous MgSO₄ and filtered. The filtrate was evaporated to give ˜7.2 g of a yellow glass. The crude was dissolved in DCM (40 ml) and subjected to flash purification on CombiFlash (0% to 15% of MeOH in DCM, 2*220 g silica gel column). The desired fractions were combined and evaporated to give target material (5.42 g, 95%) as a yellow glass.

### Int #2C, tert-Butyl-(S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionyl amino)-6-aminohexanoylamino]-6-(hexanoylamino)hexanoate, and

### Int #3C, tert-Butyl-(S)-2-[(S)-2-(3-{2-[2-(2-{2-[4-({2-[(S)-2-cyano-1-pyrrolidinyl]-2-oxoethyl amino}carbonyl)-7-quinolyloxy]acetylamino}ethoxy)ethoxy]ethoxy}propionylamino)-6-{2-[4,7-bis(tert-butoxycarbonylmethyl)-1,4,7-triazonan-1-yl]acetylamino} hexanoylamino]-6-(hexanoylamino)hexanoate

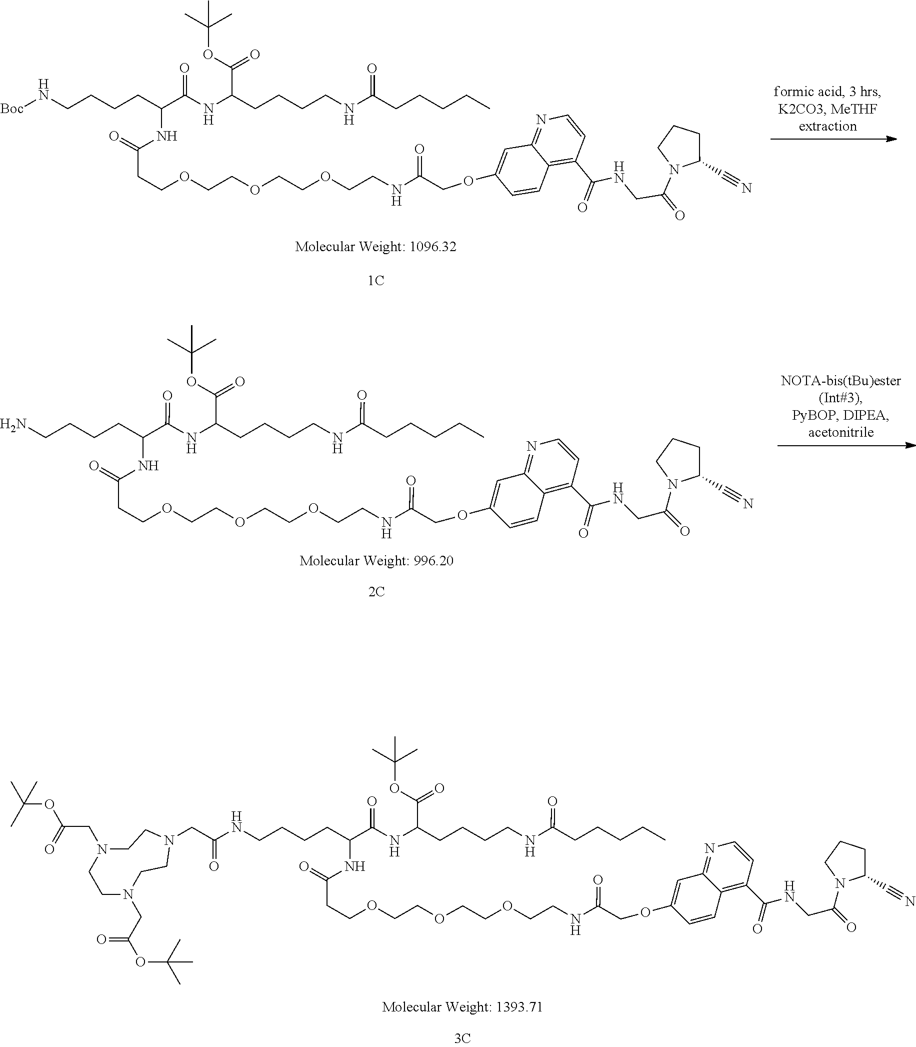 [SMILES: CCCCCC(=O)NCCCCC(NC(=O)C(CCCCNC(=O)OC(C)(C)C)NC(=O)CCOCCOCCOCCNC(=O)COc1ccc2c(C(=O)NCC(=O)N3CCC[C@@H]3C#N)ccnc2c1)C(=O)OC(C)(C)C ; CCCCCC(=O)NCCCCC(NC(=O)C(CCCCN)NC(=O)CCOCCOCCOCCNC(=O)COc1ccc2c(C(=O)NCC(=O)N3CCC[C@@H]3C#N)ccnc2c1)C(=O)OC(C)(C)C ; CCCCCC(=O)NCCCCC(NC(=O)C(CCCCNC(=O)CN1CCN(CC(=O)OC(C)(C)C)CCN(CC(=O)OC(C)(C)C)CC1)NC(=O)CCOCCOCCOCCNC(=O)COc1ccc2c(C(=O)NCC(=O)N3CCC[C@@H]3C#N)ccnc2c1)C(=O)OC(C)(C)C]

Procedure 47: A solution of tert-butyl-(S)-2-[(S)-2-(3-{2-[2-(2-{2-[4-({2-[(S)-2-cyano-1-pyrrolidinyl]-2-oxoethylamino}carbonyl)-7-quinolyloxy]acetylamino}ethoxy)ethoxy]ethoxy}propionylamino)-6-(tert-butoxycarbonylamino)hexanoylamino]-6-(hexanoylamino) hexanoate (5.4 g, 5.9 mmol) in 90% aq. formic acid (180 ml) was stirred at ambient temperature for 2.5 h, then solvents were evaporated. The residue was co-evaporated with acetonitrile (60 ml×3) and toluene (60 ml×2), dissolved in 180 ml of 2-methyltetrahydrofuran and treated with 1% K₂CO₃ aqueous solution (30 ml). The organic layer was separated, dried with MgSO₄ and evaporated to obtain target material in the form of free base. This material was used as obtained for the next step.

Procedure 48: The material from previous step (˜5.96 mmol), NOTA-bis(tBu-ester) (3.66 g, 8.82 mmol) and PyBop (3.10 g, 5.96 mmol) in acetonitrile (120 ml) was added DIPEA (2.3 ml, 12.3 mmol) and reaction mixture was stirred at ambient temperature for 2 hours (LCMS control). The reaction mixture was evaporated, diluted with ethyl acetate (600 ml) and extracted with water (2*120 ml). The organic fraction was evaporated and purified by silica-gel column chromatography (220 g column, 3% to 40% MeOH in DCM, 5% DIPEA added). Desired fractions were combined and evaporated to give 6.2 g (4.63 mmol, 75%) of target product as a light-yellow glass.

### RTX-1363S, (S)-2-[(S)-2-(3-{2-[2-(2-{2-[4-({2-[(S)-2-Cyano-1-pyrrolidinyl]-2-oxoethylamino} carbonyl)-7-quinolyloxy]acetylamino}ethoxy)ethoxy]ethoxy}propionylamino)-6-{2-[4,7-bis(carboxymethyl)-1,4,7-triazonan-1-yl]acetylamino}hexanoylamino]-6-(hexanoylamino)hexanoic Acid

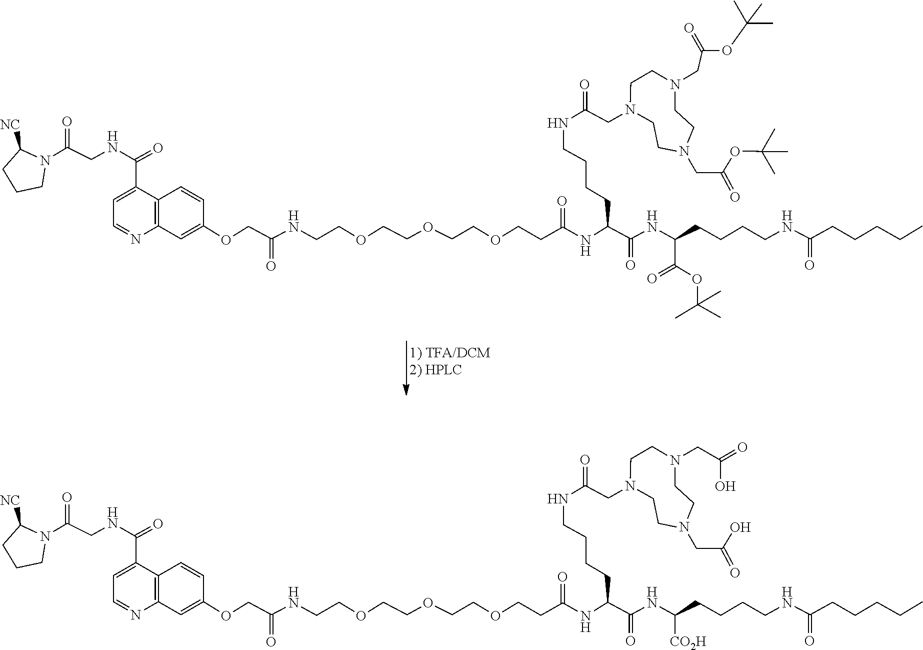 [SMILES: CCCCCC(=O)NCCCC[C@H](NC(=O)[C@H](CCCCNC(=O)CN1CCN(CC(=O)OC(C)(C)C)CCN(CC(=O)OC(C)(C)C)CC1)NC(=O)CCOCCOCCOCCNC(=O)COc1ccc2c(C(=O)NCC(=O)N3CCC[C@H]3C#N)ccnc2c1)C(=O)OC(C)(C)C ; CCCCCC(=O)NCCCC[C@H](NC(=O)[C@H](CCCCNC(=O)CN1CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Procedure 49: To a solution of tert-butyl-(S)-2-[(S)-2-(3-{2-[2-(2-{2-[4-({2-[(S)-2-cyano-1-pyrrolidinyl]-2-oxoethylamino}carbonyl)-7quinolyloxy]acetylamino}ethoxy)ethoxy]ethoxy}propionylamino)-6-{2-[4,7-bis(tert-butoxycarbonylmethyl)-1,4,7-triazonan-1-yl]acetylamino}hexanoylamino]-6-(hexanoylamino) hexanoate (1 g, 0.717 mmol) in DCM (10 ml) was added TFA (15 ml, 195 mmol) dropwise at 0° C. The temperature of the reaction mixture was allowed to rise to ambient and stirring was continued for 1 h (˜60% conversion). TFA (10 ml) was added and reaction mixture was stirred at ambient temperature for an additional hour. Full conversion was detected by LCMS. The solvents were evaporated at ambient temperature. The residue was dissolved in DMSO (10 ml) and subjected to HPLC purification (Nanosyn-Pack, Silicycle, C-18, 100-10, 50×300 mm; 0.1% TFA/ACN/water). Desired fractions were combined and lyophilized to obtain target the compound (55 0 mg, 59%; 96% purity) as an off-white powder. The compound was re-purified (5 mM NH₄OAc/ACN) by HPLC and lyophilized to obtain two fractions of target material (fr #23: 243 mg, 98.5% purity and fr #24: 80 mg, 96.8% purity) as white powders (36.8% yield in total).

### Synthesis of RTX-1339

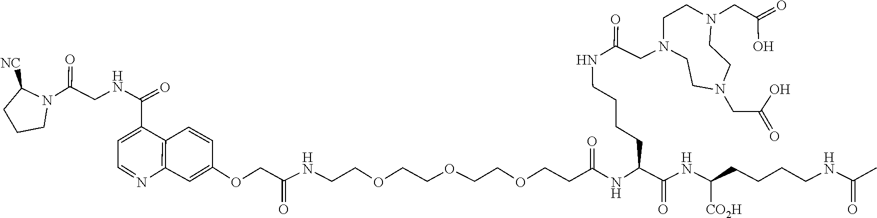 [SMILES: CC(=O)NCCCC[C@H](NC(=O)[C@H](CCCCNC(=O)CN1CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Prepared according to the procedures of Example 9 with the following changes:
Procedure 3 was used in lieu of Procedure 40.

### Synthesis of RTX-1340S

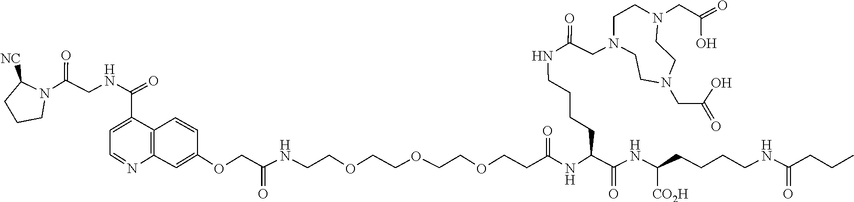 [SMILES: CCCC(=O)NCCCC[C@H](NC(=O)[C@H](CCCCNC(=O)CN1CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

Prepared according to procedure in Example 9 using butyric acid in lieu of hexanoic acid in Procedure 40.

### Synthesis of RTX-1363S-Cu

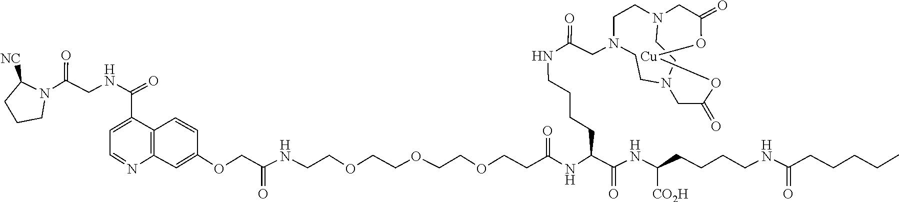

Prepared according to the procedures of Example 9. The resulting product was subjected in Procedure 50 for the installation of the radionuclide.

Procedure 50: 20 μg of precursor (unless otherwise noted) are combined with the indicated radioisotopes The radioisotopes are either eluted from a generator (for ⁶⁸Ga) or obtained commercially in HCl solutions that were buffered with varying amounts of 3 N NaOAc to obtain a final pH of 4-6. Reactions with a C18 Sep-Pak Lite cartridge as needed for additional purity and/or reformulation for injection.

### Synthesis of RTX-1359

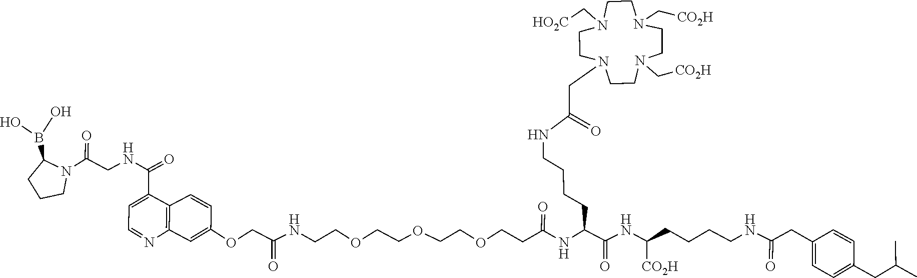 [SMILES: CC(C)Cc1ccc(CC(=O)NCCCC[C@H](NC(=O)[C@H](CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B(O)O)ccnc3c2)C(=O)O)cc1]

Prepared according to the procedures of Example 9 with the following changes:
In Procedure 40, 2-(4-isobutylphenyl)acetic acid was utilized in lieu of hexanoic acid.
The product of Procedure 44 was subjected the Procedures 13, 17, and 18 for the installation of the DOTA chelator.
The resulting product was subjected to Procedures 51-54 for the installation of the borylated proline derivative.

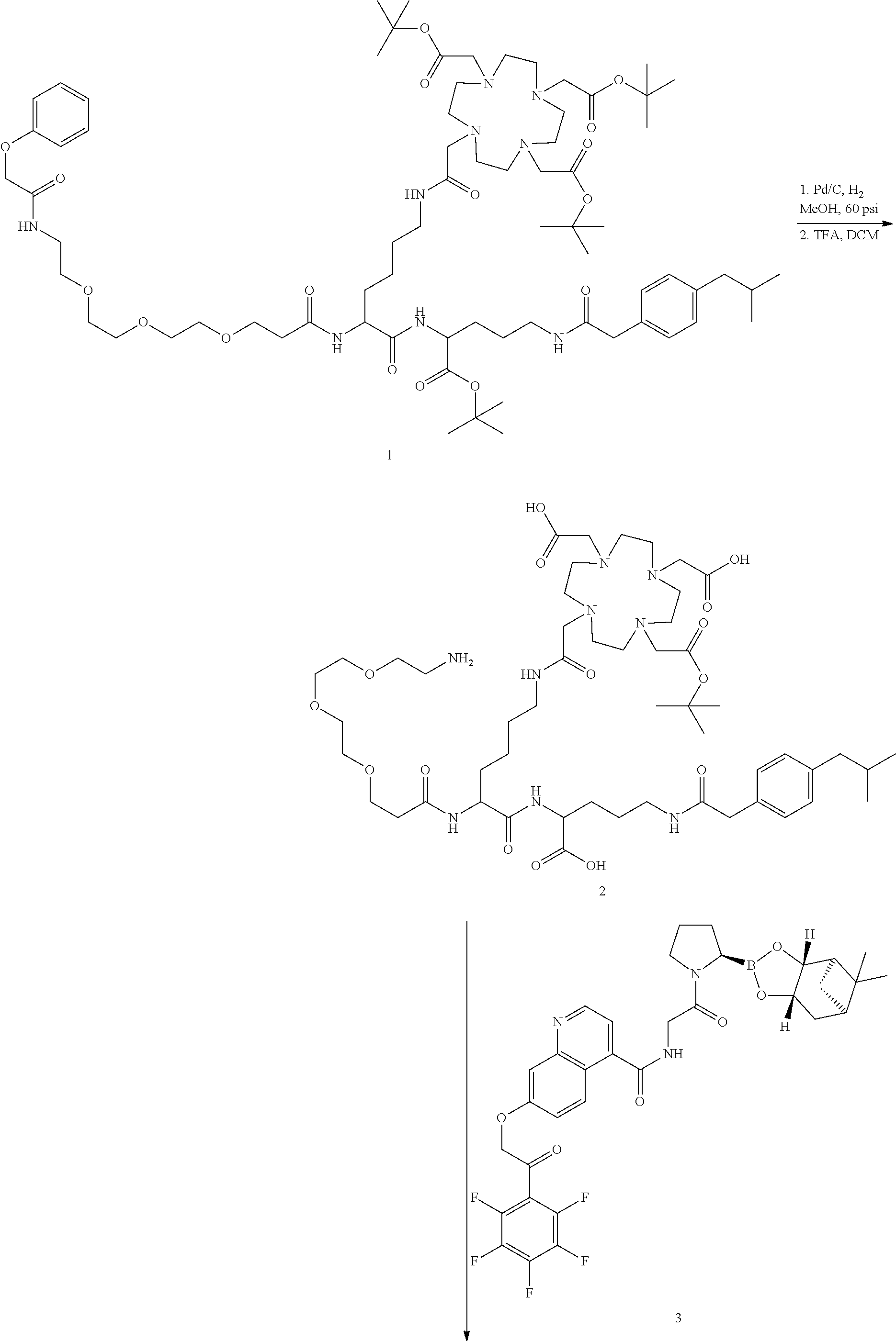

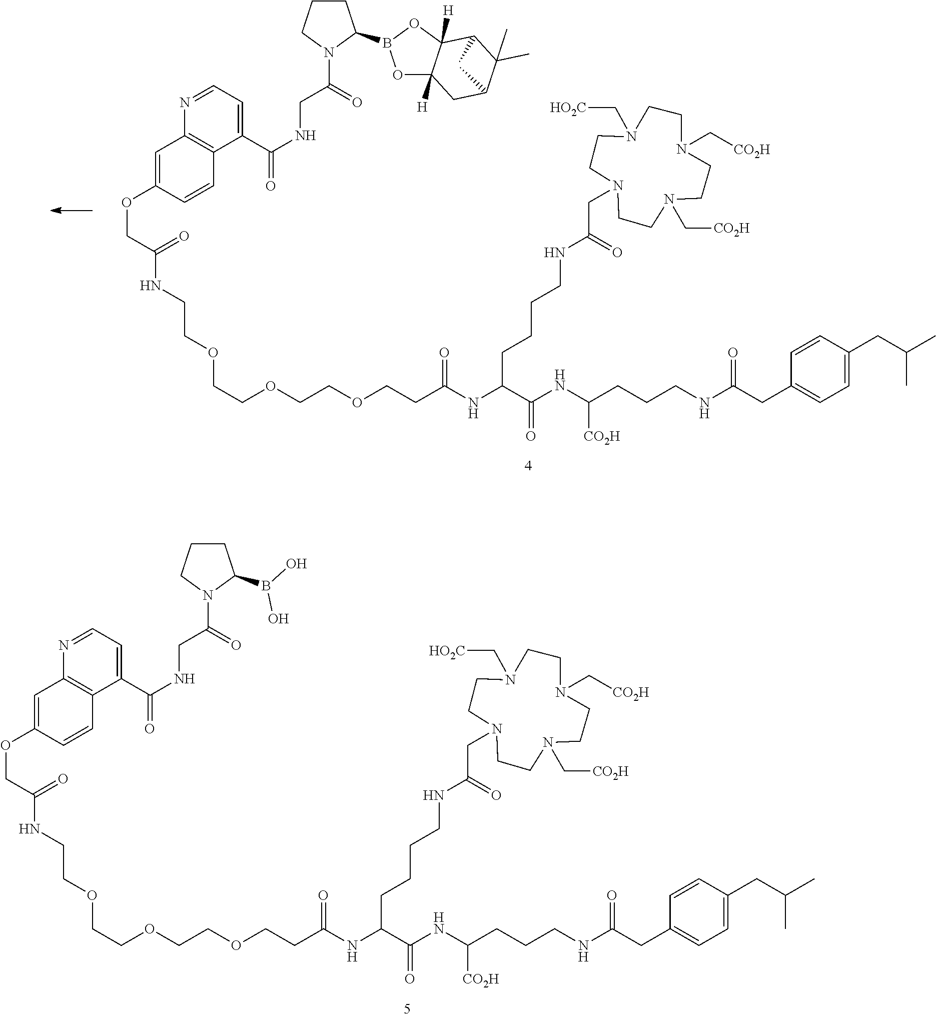

### Synthesis of 2-[2-(3-{2-[2-(2-aminoethoxy)ethoxy]ethoxy}propionylamino)-6-{2-[4,7,10-tris(carb-oxymethyl)-1,4,7,10-tetraaza-1-cyclododecyl]acetylamino}hexanoylamino]-6-[2-(p-isobutylphenyl)-acetylamino]hexanoic Acid

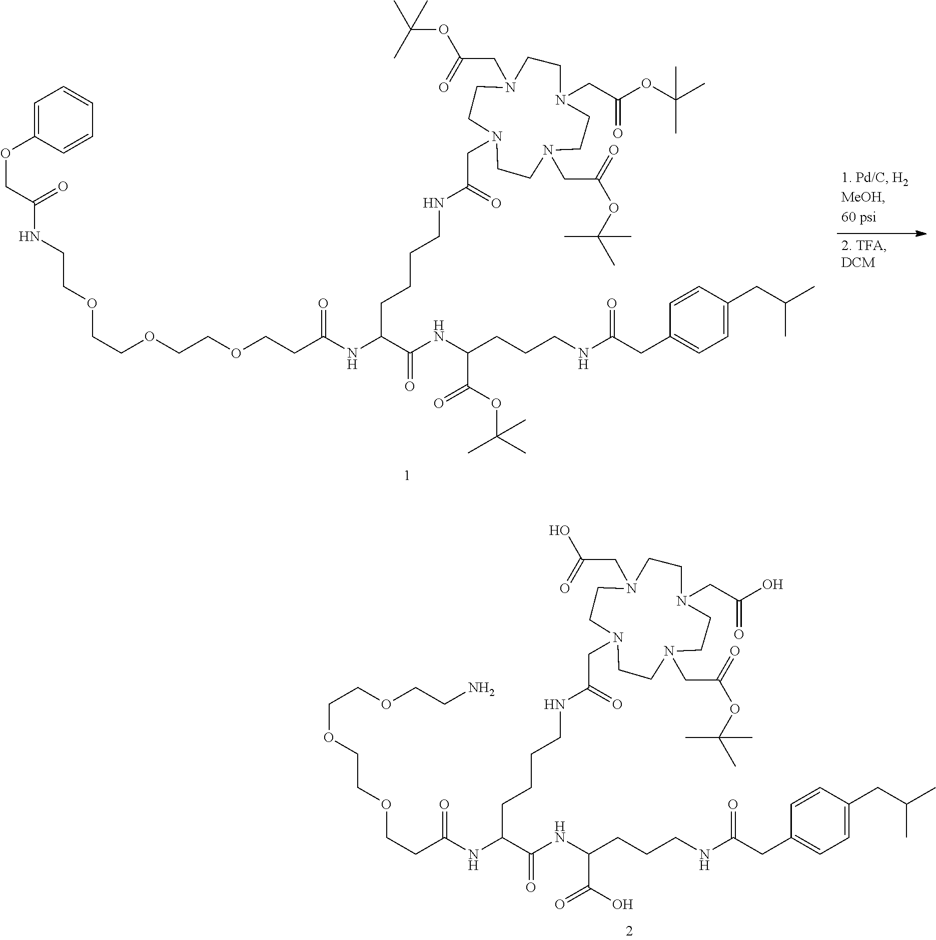 [SMILES: CC(C)Cc1ccc(CC(=O)NCCCC(NC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCNC(=O)COc2ccccc2)C(=O)OC(C)(C)C)cc1 ; CC(C)Cc1ccc(CC(=O)NCCCC(NC(=O)C(CCCCNC(=O)CN2CCN(CC(=O)O)CCN(CC(=O)O)CCN(CC(=O)OC(C)(C)C)CC2)NC(=O)CCOCCOCCOCCN)C(=O)O)cc1]

Procedure 51: To a solution of tert-butyl 2-{2-[3-(2-{2-[2-(benzyloxycarbonylamino)ethoxy]ethoxy}ethoxy)-propionylamino]-6-{2-[4,7,10-tris(tert-butoxycarbonylmethyl)-1,4,7,10-tetraaza-1-cyclododecyl-]acetylamino}hexanoylamino}-6-[2-(p-isobutylphenyl)acetylamino]hexanoate 1 (180 mg, 0.129 mmol) in MeOH (2 mL) was added Pd/C (10% wet) (23 mg, 0.217 mmol). This heterogeneous mixture was stirred under H₂ balloon (1 atm) for 2 h. The reaction was filtered through celite and concentrated. The obtained solid was dried in vacuo for 16 h to yield tert-butyl 2-[2-(3-{2-[2-(2-aminoeth-oxy)ethoxy]ethoxy}propionylamino)-6-{2-[4,7,10-tris(tert-butoxycarbonylmethyl)-1,4,7,10-tetr-aaza-1-cyclododecyl]acetylamino}hexanoylamino]-6-[2-(p-isobutylphenyl)acetylamino]hexano-ate as a viscous oil. The isolated material was used without further purification. LCMS: C₆₅H₁₁₅N₉O₁₅: m/z: 1262.66, observed m/z=1263.5 [M+H]⁺.

Procedure 52: To a solution of tert-butyl 2-[2-(3-{2-[2-(2-aminoethoxy)ethoxy]ethoxy}propionylamino)-6-{2-[4,7,10-tris(tert-butoxycarbonylmethyl)-1,4,7,10-tetraaza-1-cyclododecyl]acetylamino}hexanoylamino]-6-[2-(p-isobutylphenyl)acetylamino]hexanoate (0.129 mmol) in DCM (4 mL), was added TFA (2 mL) at 0° C. and the reaction mixture was stirred at ambient temperature for 2 h. The solvents were evaporated under reduced pressure and the residue was washed with hexane (2×) and ether (2×) to obtained 2 (120 mg) as a colorless powder. The crude product 2-[2-(3-{2-[2-(2-aminoethoxy)ethoxy]ethoxy}propionylamino)-6-{2-[4,7,10-tris(carboxymethyl)-1,4,7,10-tetraaza-1-cyclododecyl]acetylamino}hexanoylamino]-6-[2-(p-isobutylphenyl)acetylamino]hexanoic acid was used for the next step without further purification. LCMS: C₄₉H₈₃N₉O₁₅: m/z: 1038.23, observed m/z=1039.2 [M+H]⁺.

### Synthesis of 2-[2-(3-{2-[2-(2-{2-[4-({2-[(R)-2-{(1R,2R,6S,8R)-6,9,9-trimethyl-3,5-dioxa-4-boratricy-clo[6.1.1.0^{2,6}]dec-4-yl}-1-pyrrolidinyl]-2-oxoethylamino}carbonyl)-7-quinolyloxy]acetylamino}etho-xy)ethoxy]ethoxy}propionylamino)-6-{2-[4,7,10-tris(carboxymethyl)-1,4,7,10-tetraaza-1-cyclodode-cyl]acetylamino}hexanoylamino]-6-[2-(p-isobutylphenyl)acetylamino]hexanoic Acid

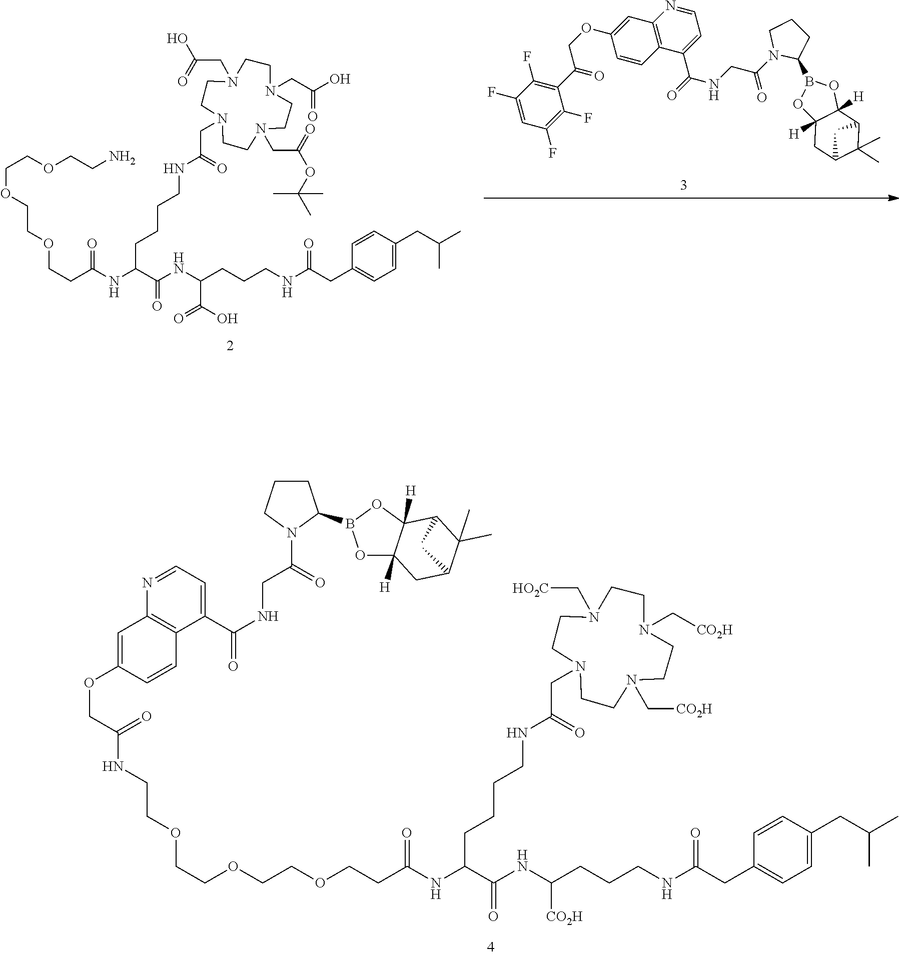

Procedure 53: To a solution of 2-[2-(3-{2-[2-(2-aminoethoxy)ethoxy]ethoxy}propionylamino)-6-{2-[4,7,10-tris(carb-oxymethyl)-1,4,7,10-tetraaza-1-cyclododecyl]acetylamino}hexanoylamino]-6-[2-(p-isobutylphenyl)acetylamino]hexanoic acid 2 (120 mg, 0.115 mmol) in DMF (0.3 mL), was added DIPEA (82 uL) at −78° C. and the reaction mixture was stirred at −78° C. for 5 min. Next, ((R)-1-((R)-2-(7-(2-oxo-2-(2,3,5,6-tetrafluorophenoxy)ethoxy)quinoline-4-carboxamido)propano-yl)pyrrolidin-2-yl)boronic acid 3 (158 mg, 0.230 mmol) in DMF (0.2 mL) was added at −78° C. and the temperature of the mixture was allowed to rise to 20° C. and stirred for 15 min. After complete consumption of the amine was evident by LCMS, the crude product was submitted for HPLC purification. The desired fractions were combined and lyophilized to obtain 2-[2-(3-{2-[2-(2-{2-[4-({2-[(R)-2-{(1R,2R,6S,8R)-6,9,9-trimethyl-3,5-dioxa-4-boratricyclo[6.1.1.0^{2,6}]dec-4-yl}-1-pyrrolidinyl]-2-oxoethylamino}carbonyl)-7-quinolyloxy]acetylamino}ethoxy)ethoxy]ethoxy}propion-ylamino)-6-{2-[4,7,10-tris(carboxymethyl)-1,4,7,10-tetraaza-1-cyclododecyl]acetylamino}hexanoylami-no]-6-[2-(p-isobutylphenyl)acetylamino]hexanoic acid 4 (55 mg, 31%) as an off-white solid. LCMS: C₇₇H₁₁₅BN₁₂O₂₁: m/z: 1554.84, observed m/z=778.2 [(½)M+H]⁺.

### Synthesis of 2-[2-(3-{2-[2-(2-{2-[4-({2-[(R)-2-(1,2-dihydroxaboro)-1-pyrrolidinyl]-2-oxoethylamino}-carbonyl)-7-quinolyloxy]acetylamino}ethoxy)ethoxy]ethoxy}propionylamino)-6-{2-[4,7,10-tris(carb-oxymethyl)-1,4,7,10-tetraaza-1-cyclododecyl]acetylamino}hexanoylamino]-6-[2-(p-isobutylphenyl)-acetylamino]hexanoic Acid

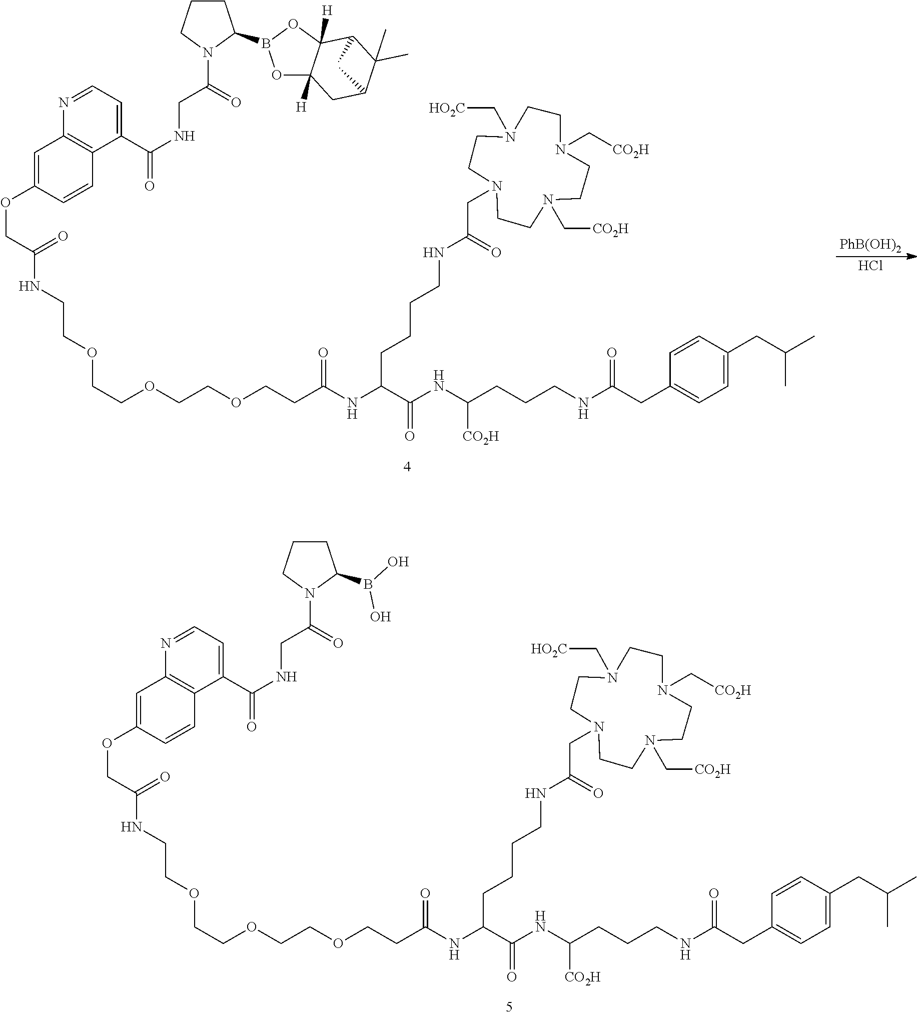

Procedure 54: To a solution of 2-[2-(3-{2-[2-(2-{2-[4-({2-[(R)-2-{(1R,2R,6S,8R)-6,9,9-trimethyl-3,5-dioxa-4-boratricyclo[6.1.1.0^{2,6}]dec-4-yl}-1-pyrrolidinyl]-2-oxoethylamino}carbonyl)-7-quinolyloxy]acetylamino-}ethoxy)ethoxy]ethoxy}propionylamino)-6-{2-[4,7,10-tris(carboxymethyl)-1,4,7,10-tetraaza-1-cyclodo-decyl]acetylamino}hexanoylamino]-6-[2-(p-isobutylphenyl)acetylamino]hexanoic acid (55 mg, 0.035 mmol) in acetonitrile (1 mL), a mixture of water and diethyl ether (1 mL) containing phenyl boronic acid (8.6 mg, 0.070 mmol) at rt was added, followed by 2 drops of 1 M HCl. The reaction mixture was stirred at ambient temperature for 10 min. The crude reaction mixture was purified by HPLC. The desired fractions were combined and lyophilized to obtain 2-[2-(3-{2-[2-(2-{2-[4-({2-[(R)-2-(1,2-dihydroxaboro)-1-pyrrolidinyl]-2-oxoethylamino}carbonyl)-7-quinolyloxy]acetylamino}ethoxy)ethoxy]ethoxy}propiony-lamino)-6-{2-[4,7,10-tris(carboxymethyl)-1,4,7,10-tetraaza-1-cyclododecyl]acetylamino}hexanoylami-no]-6-[2-(p-isobutylphenyl)acetylamino]hexanoic acid 5 (26.3 mg, 27%) as a white solid. LCMS: C₆₇H₁₀₁BN₁₂O₂₁: m/z: 1420.73, observed m/z=1403.8 [M-OH]⁺.

### Synthesis of RTX-1355R

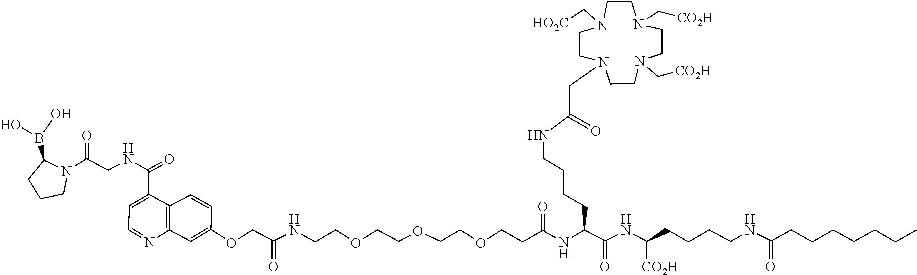 [SMILES: CCCCCCCC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3B(O)O)ccnc2c1)C(=O)O]

Prepared according to the procedures of Example 9 with the following changes:
In Procedure 40, octanoic acid was utilized in lieu of hexanoic acid.
The product of Procedure 44 was subjected the Procedures 13, 17, and 18 for the installation of the DOTA chelator.
The resulting product was subjected to Procedures 51-54 for the installation of the borylated proline derivative.

### Synthesis of RTX-1356R

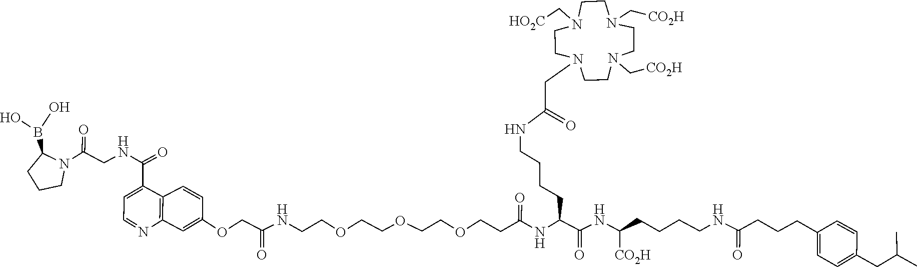 [SMILES: CC(C)Cc1ccc(CCCC(=O)NCCCC[C@H](NC(=O)[C@H](CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B(O)O)ccnc3c2)C(=O)O)cc1]

Prepared according to the procedures of Example 9 with the following changes:
In Procedure 40, 2-(4-isobutylphenyl)butanoic acid was utilized in lieu of hexanoic acid.
The product of Procedure 44 was subjected the Procedures 13, 17, and 18 for the installation of the DOTA chelator.
The resulting product was subjected to Procedures 51-54 for the installation of the borylated proline derivative.

### Synthesis of 1357R

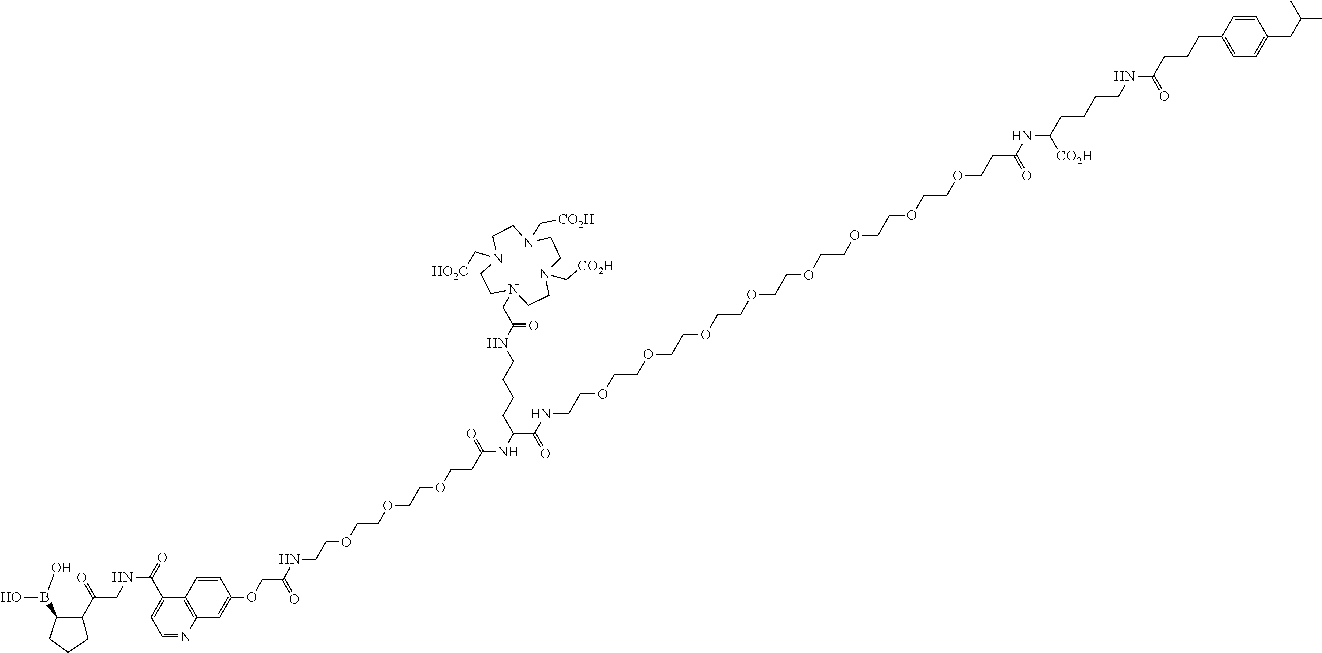 [SMILES: CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)C4CCC[C@H]4B(O)O)ccnc3c2)C(=O)O)cc1]

Prepared according to the following procedures:

Procedure 55: Synthesis of tert-butyl 2-((((9H-fluoren-9-yl)methoxy)carbonyl)amino)-6-aminohexanoate (2):

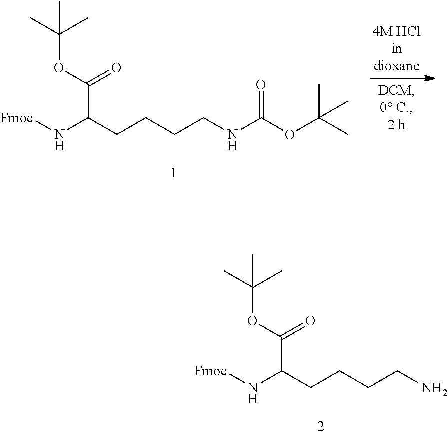

To a solution of tert-butyl 2-((((9H-fluoren-9-yl)methoxy)carbonyl)amino)-6-((tert-butoxycarbonyl)-amino)hexanoate (2.0 g, 3.81 mmol) in DCM (10 ml), was added, dropwise, HCl in dioxane (4 M, 20 ml) at 0° C., and the reaction mixture was stirred at 0° C. for 2 h. The solvents were evaporated and the residue was co-evaporated with hexane and dried in vacuo to obtain HCl salt of the product (1.62 g, 100%) as a white solid. The crude product was used for the next step without further purification. LCMS: C₂₅H₃₂N₂O₄: m/z: 424.53, observed m/z=425.3 [M+H]⁺.

Procedure 56: Synthesis of tert-butyl 2-((((9H-fluoren-9-yl)methoxy)carbonyl)amino)-6-(4-(4-isobutylphenyl)-butanamido)hexanoate (4):

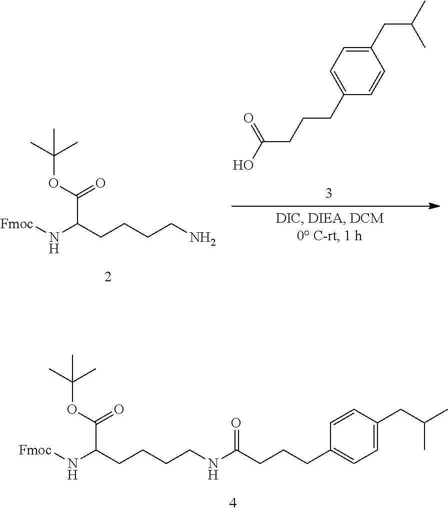

To a chilled solution of 4-(4-isobutylphenyl)butanoic acid (1.0 g, 4.57 mmol) in 10 ml of DCM was added DIC (0.71 ml, 4.47 mmol), dropwise, in an ice bath. The mixture was stirred for 30 mins and filtered. The filtrate was added to a solution of tert-butyl 2-((((9H-fluoren-9-yl)methoxy)carbonyl)amino)-6-aminohexanoate (1.62 g, 3.81 mmol) in 5 ml of DCM, and followed by a dropwise addition of DIEA (0.66 ml, 3.81 mmol). The mixture was stirred for another 30 mins. The solvents were evaporated under reduced pressure, and the crude product was purified by flash chromatography (EtOAc/hexane, eluted at 50% EtOAc in hexane) to afford the product (1.93 g, 82% yield) as a white solid. LCMS: C₃₉H₅₀N₂O₅: m/z: 626.82, observed m/z=627.4 [M+H]⁺.

Procedure 57: Synthesis of tert-butyl 2-amino-6-(4-(4-isobutylphenyl)butanamido)hexanoate (5):

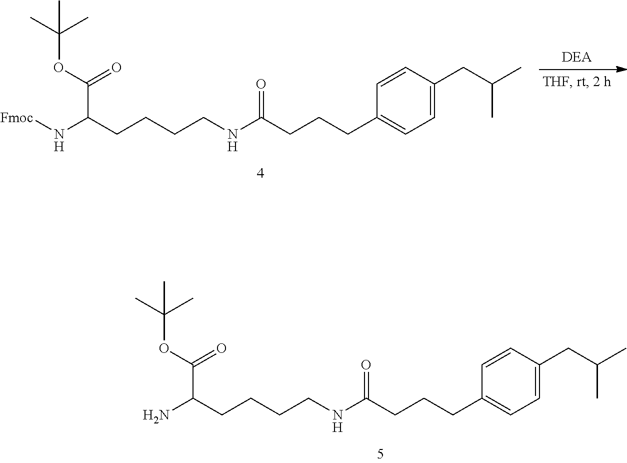

To a solution of tert-butyl 2-((((9H-fluoren-9-yl)methoxy)carbonyl)amino)-6-(4-(4-isobutylphenyl)butanamido)-hexanoate (0.95 g, 1.51 mmol) in THF (8 ml) was added diethylamine (8 ml) at room temperature. The reaction mixture was stirred at ambient temperature for 2 h. The solvent was evaporated, and the residue was washed with hexane (2×) and dried in vacuo to obtain the product (613 mg, 100%). The compound was used as is for the next chemical transformation. LCMS: C₂₄H₄₀N₂O₃: m/z: 404.59, observed m/z=405.4 [M+H]⁺.

Procedure 58: Synthesis of tert-butyl 33-(4-(4-(4-isobutylphenyl)butanamido)butyl)-3,31-dioxo-1-phenyl-2,7,10,13,16,19,22,25,28-nonaoxa-4,32-diazatetratriacontan-34-oate (7):

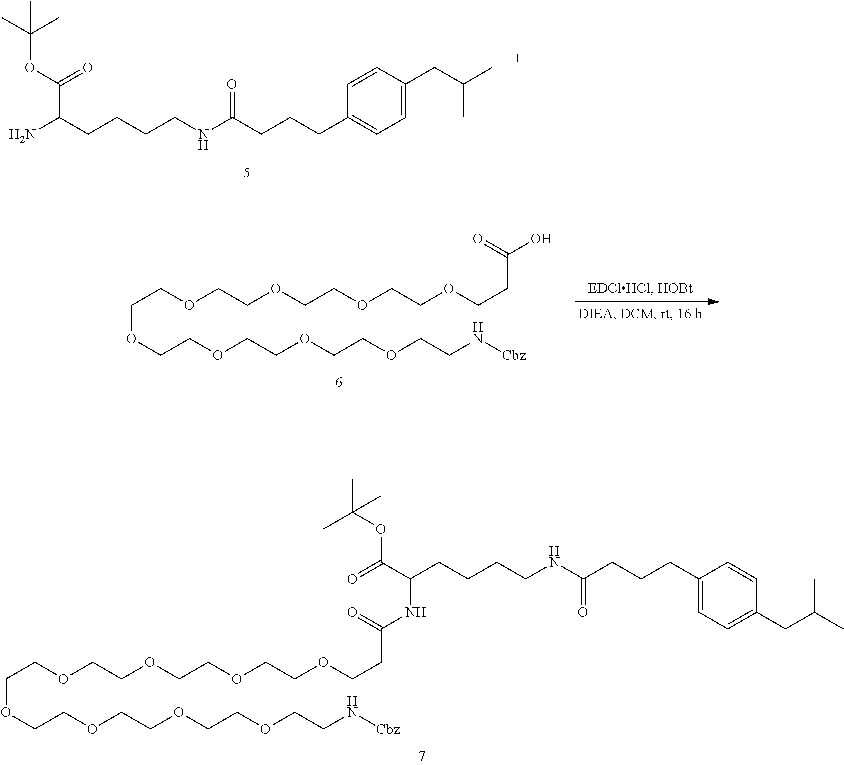 [SMILES: CC(C)Cc1ccc(CCCC(=O)NCCCCC(N)C(=O)OC(C)(C)C)cc1 ; O=C(O)CCOCCOCCOCCOCCOCCOCCOCCOCCNC(=O)OCc1ccccc1 ; CC(C)Cc1ccc(CCCC(=O)NCCCCC(NC(=O)CCOCCOCCOCCOCCOCCOCCOCCOCCNC(=O)OCc2ccccc2)C(=O)OC(C)(C)C)cc1]

To a solution of 3-oxo-1-phenyl-2,7,10,13,16,19,22,25,28-nonaoxa-4-azahentriacontan-31-oic acid (872 mg, 1.51 mmol) in 10 ml of DCM was added EDCI·HCl (434 mg, 2.27 mmol) and HOBt (203 mg, 1.51 mmol) and the reaction mixture was stirred at rt for 5 min. Tert-butyl 2-amino-6-(4-(4-isobutylphenyl)butanamido)hexanoate (613 mg, 1.51 mmol) in DCM (10 ml) and DIEA (0.8 ml, 4.53 mmol) were added sequentially and the mixture was stirred at room temperature for 16 hours. The reaction mixture was diluted with 20 ml of DCM and then washed with water. The organic layer was dried over Na₂SO₄ and concentrated under reduced pressure. The crude product was purified by flash chromatography (MeOH/DCM, eluted at 3-4% MeOH in DCM) to afford the product (1.0 g, 69% yield) as a colorless liquid. LCMS: C₅₁H₈₃N₃O₁₄: m/z: 962.22, observed m/z=962.9 [M+H]⁺.

Procedure 59: Synthesis of tert-butyl 1-amino-29-(4-(4-(4-isobutylphenyl)butanamido)butyl)-27-oxo-3,6,9,12,15,18,21,24-octaoxa-28-azatriacontan-30-oate (8):

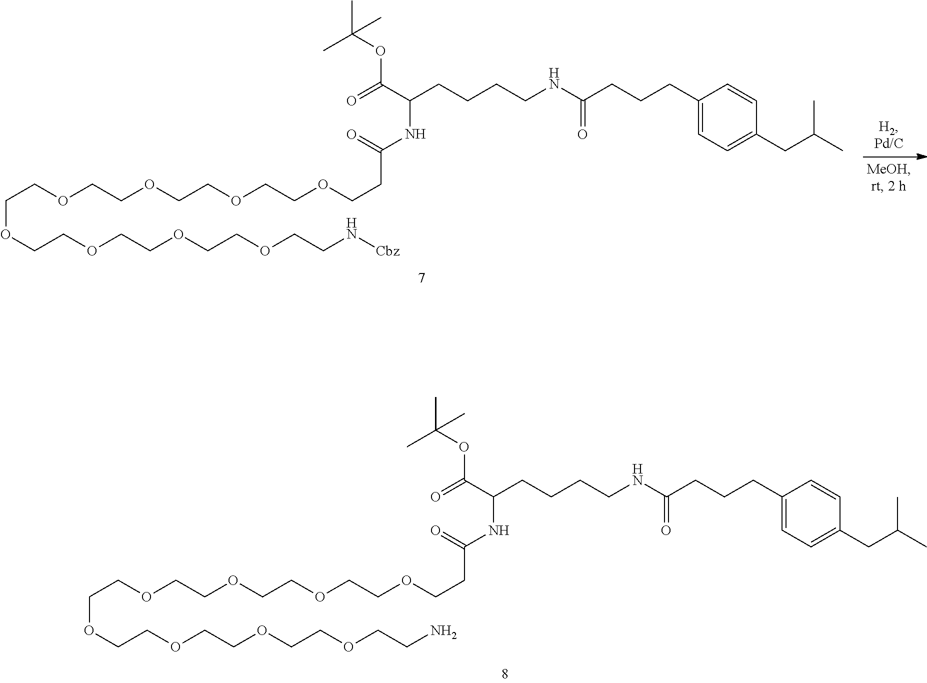 [SMILES: CC(C)Cc1ccc(CCCC(=O)NCCCCC(NC(=O)CCOCCOCCOCCOCCOCCOCCOCCOCCNC(=O)OCc2ccccc2)C(=O)OC(C)(C)C)cc1 ; CC(C)Cc1ccc(CCCC(=O)NCCCCC(NC(=O)CCOCCOCCOCCOCCOCCOCCOCCOCCN)C(=O)OC(C)(C)C)cc1]

Palladium on carbon (10%, 0.2 g, wet, 67%) was suspended in a solution of tert-butyl 33-(4-(4-(4-isobutyl-phenyl)butanamido)butyl)-3,31-dioxo-1-phenyl-2,7,10,13,16,19,22,25,28-nonaoxa-4,32-diazatetratriaco-ntan-34-oate (1.0 g, 1.03 mmol) in MeOH (25 ml). The suspension was stirred for 2 h at room temperature under H₂ at balloon pressure. The mixture was filtered through celite, and the filtrate was evaporated to afford the product (850 mg, 100%) as a white solid. The crude product was used for the next step without further purification. LCMS: C₄₃H₇₇N₃O₁₂: m/z: 828.08, observed m/z=828.7 [M+H]⁺.

Procedure 60: Synthesis of tert-butyl 10-((((9H-fluoren-9-yl)methoxy)carbonyl)amino)-41-(4-(4-(4-isobutylphen-yl)butanamido)butyl)-2,2-dimethyl-4,11,39-trioxo-3,15,18,21,24,27,30,33,36-nonaoxa-5,12,40-triaza-dotetracontan-42-oate (10):

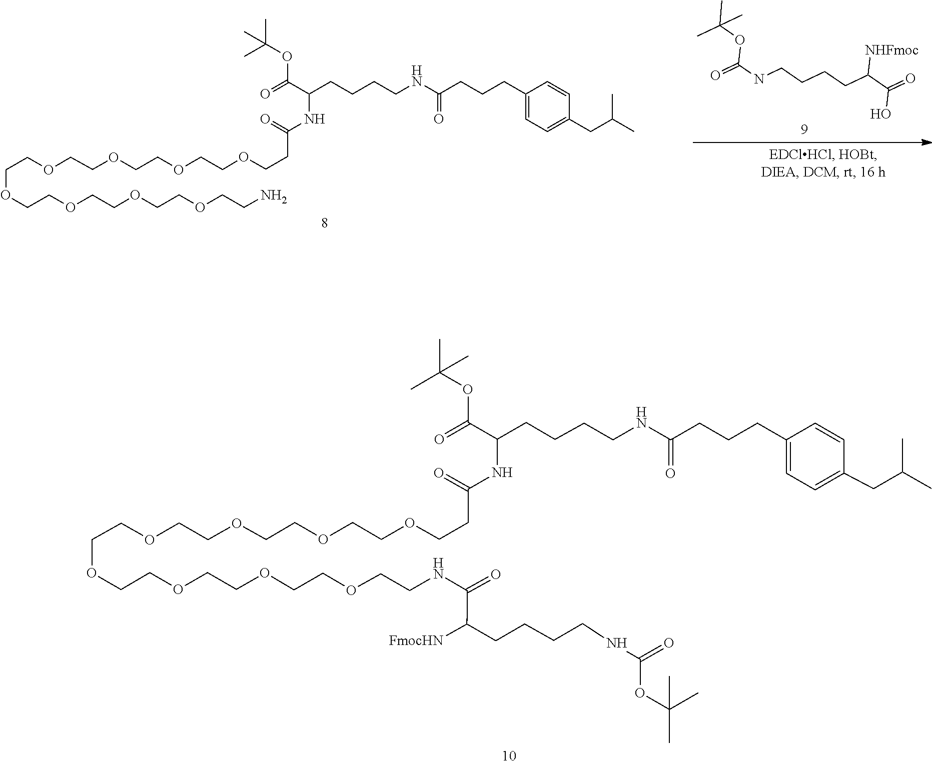

To a solution of 2-((((9H-fluoren-9-yl)methoxy)carbonyl)amino)-6-((tert-butoxycarbonyl)amino)hexanoic acid (0.48 g, 1.02 mmol) in 10 ml of DCM was added EDCI·HCl (0.29 g, 1.53 mmol) and HOBt (0.14 g, 1.02 mmol) and the reaction mixture was stirred at rt for 5 min. Tert-butyl 1-amino-29-(4-(4-(4-isobutylphenyl)butanamido)butyl)-27-oxo-3,6,9,12,15,18,21,24-octaoxa-28-azatriacontan-30-oate (0.85 g, 1.02 mmol) in DCM (10 ml) and DIEA (0.53 ml, 3.07 mmol) were added sequentially and the mixture was stirred at room temperature for 16 hours. The reaction mixture was diluted with 20 ml of DCM and washed with water. The organic layer was dried over Na₂SO₄ and concentrated under reduced pressure. The crude product was purified by flash chromatography (MeOH/DCM, eluted at 5% MeOH in DCM) to afford the product (0.66 g, 50% yield) as a white solid. LCMS: C₆₉H₁₀₇N₅O₁₇: m/z: 1278.61, observed m/z=1279.1 [M+H]⁺.

Procedure 61: Synthesis of tert-butyl 10-amino-41-(4-(4-(4-isobutylphenyl)butanamido)butyl)-2,2-dimethyl-4,11,39-trioxo-3,15,18,21,24,27,30,33,36-nonaoxa-5,12,40-triazadotetracontan-42-oate (11):

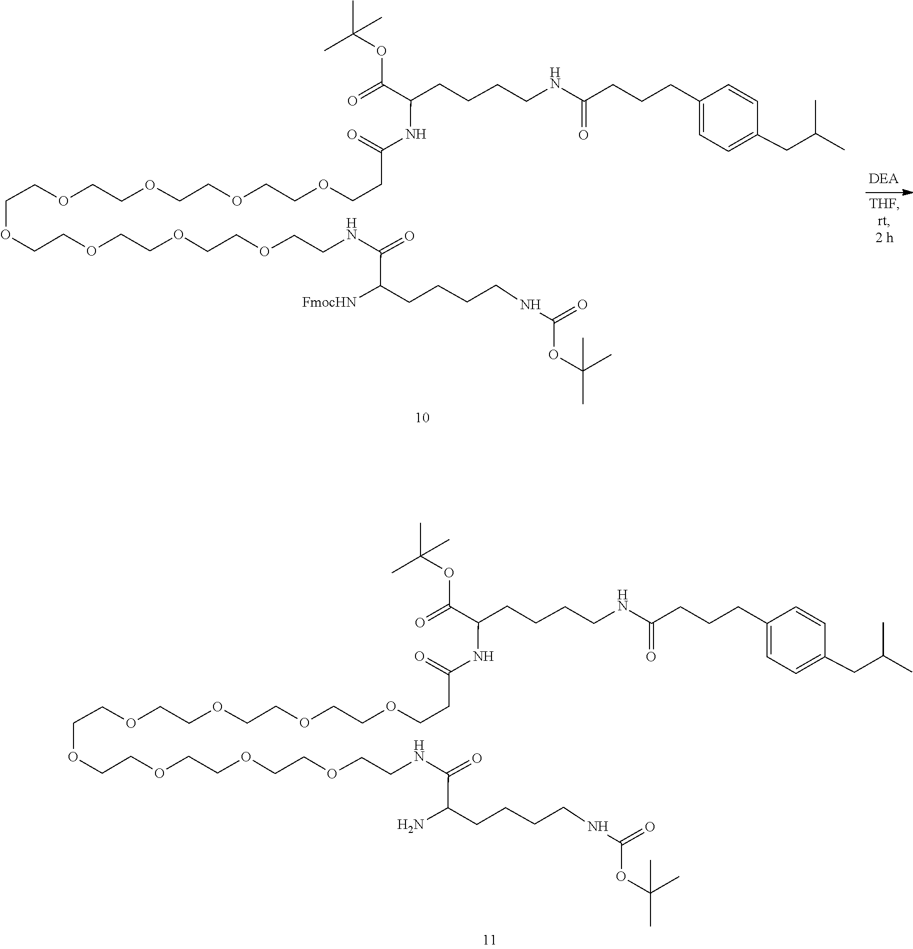

To a solution of tert-butyl 10-((((9H-fluoren-9-yl)methoxy)carbonyl)amino)-41-(4-(4-(4-isobutylphenyl)-butanamido)butyl)-2,2-dimethyl-4,11,39-trioxo-3,15,18,21,24,27,30,33,36-nonaoxa-5,12,40-triazadotetra-contan-42-oate (0.66 g, 0.51 mmol) in THF (7 ml) was added diethylamine (7 ml) at room temperature. The reaction mixture was stirred at ambient temperature for 2 hours, and the solvents were evaporated. The residue was washed with hexane (2×) and dried in vacuo to obtain the product (700 mg, 100%). The compound was used for the next step without further purification. LCMS: C₅₄H₉₇N₅O₁₅: m/z: 1056.37, observed m/z=1057.1 [M+H]⁺.

Procedure 62: Synthesis of tert-butyl 18-(4-((tert-butoxycarbonyl)amino)butyl)-49-(4-(4-(4-isobutylphenyl)-butanamido)butyl)-3,16,19,47-tetraoxo-1-phenyl-2,7,10,13,23,26,29,32,35,38,41,44-dodecaoxa-4,17,20,48-tetraazapentacontan-50-oate (13):

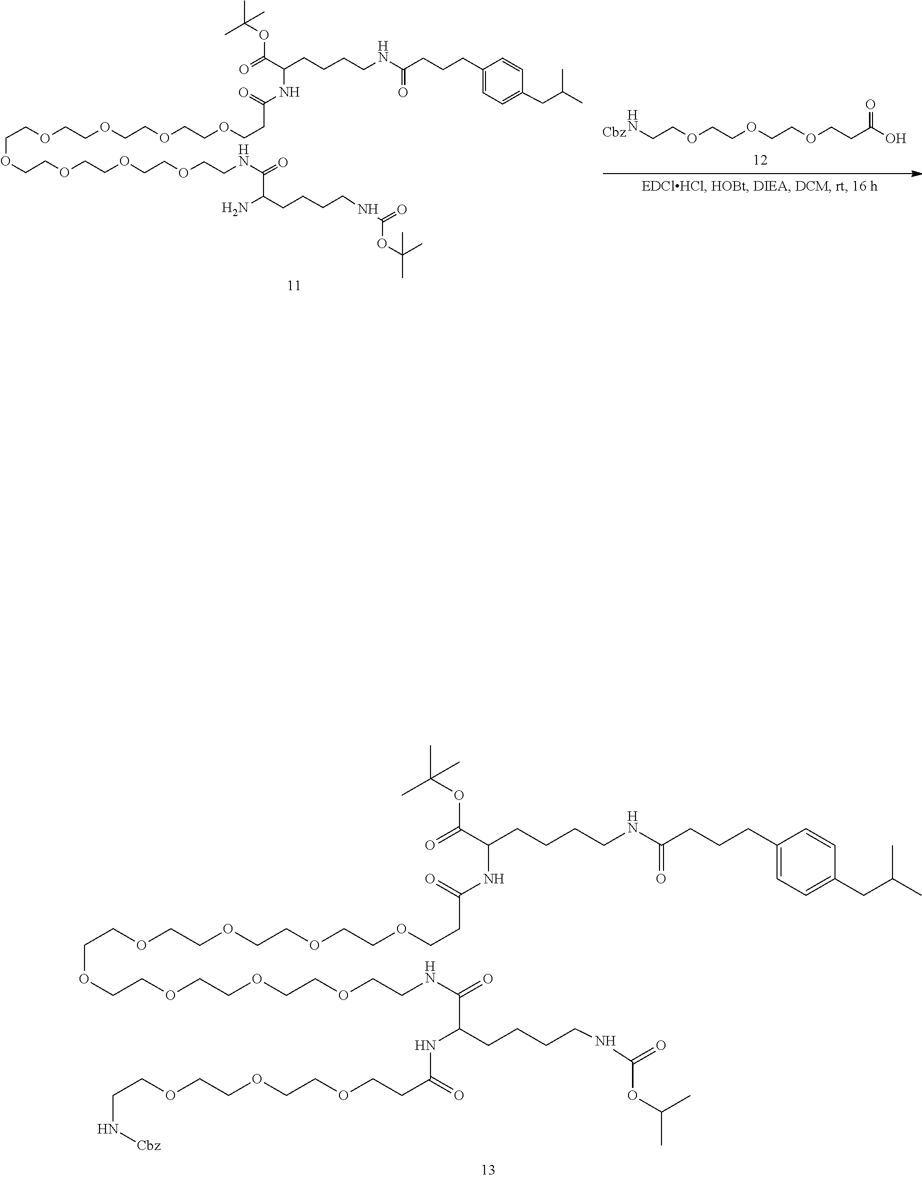

To a solution of 3-oxo-1-phenyl-2,7,10,13-tetraoxa-4-azahexadecan-16-oic acid (0.22 g, 0.62 mmol) in DCM (8 ml) was added EDCI·HCl (0.18 g, 0.93 mmol) and HOBt (0.08 g, 0.62 mmol) and the reaction mixture was stirred at rt for 5 min. Tert-butyl 10-amino-41-(4-(4-(4-isobutylphenyl)-butanamido)butyl)-2,2-dimethyl-4,11,39-trioxo-3,15,18,21,24,27,30,33,36-nonaoxa-5,12,40-triazadotetra-contan-42-oate (0.66 g, 0.62 mmol) in DCM (5 ml) and DIEA (0.32 ml, 1.87 mmol) were added sequentially and the mixture was stirred at room temperature for 16 hours. The reaction mixture was diluted with 20 ml of DCM and washed with water. The organic layer was dried over Na₂SO₄ and concentrated under reduced pressure. The crude product was purified by flash chromatography (MeOH/DCM, eluted at 8-10% MeOH in DCM) to afford the product (0.70 g, 50% yield) as a white solid. LCMS: C₇₁H₁₂₀N₆O₂₁: m/z: 1393.74, observed m/z=1394.7 [M+H]⁺.

Procedure 63: Synthesis of tert-butyl 18-(4-aminobutyl)-49-(4-(4-(4-isobutylphenyl)butanamido)butyl)-3,16,19,47-tetraoxo-1-phenyl-2,7,10,13,23,26,29,32,35,38,41,44-dodecaoxa-4,17,20,48-tetraazapentacontan-50-oate (14):

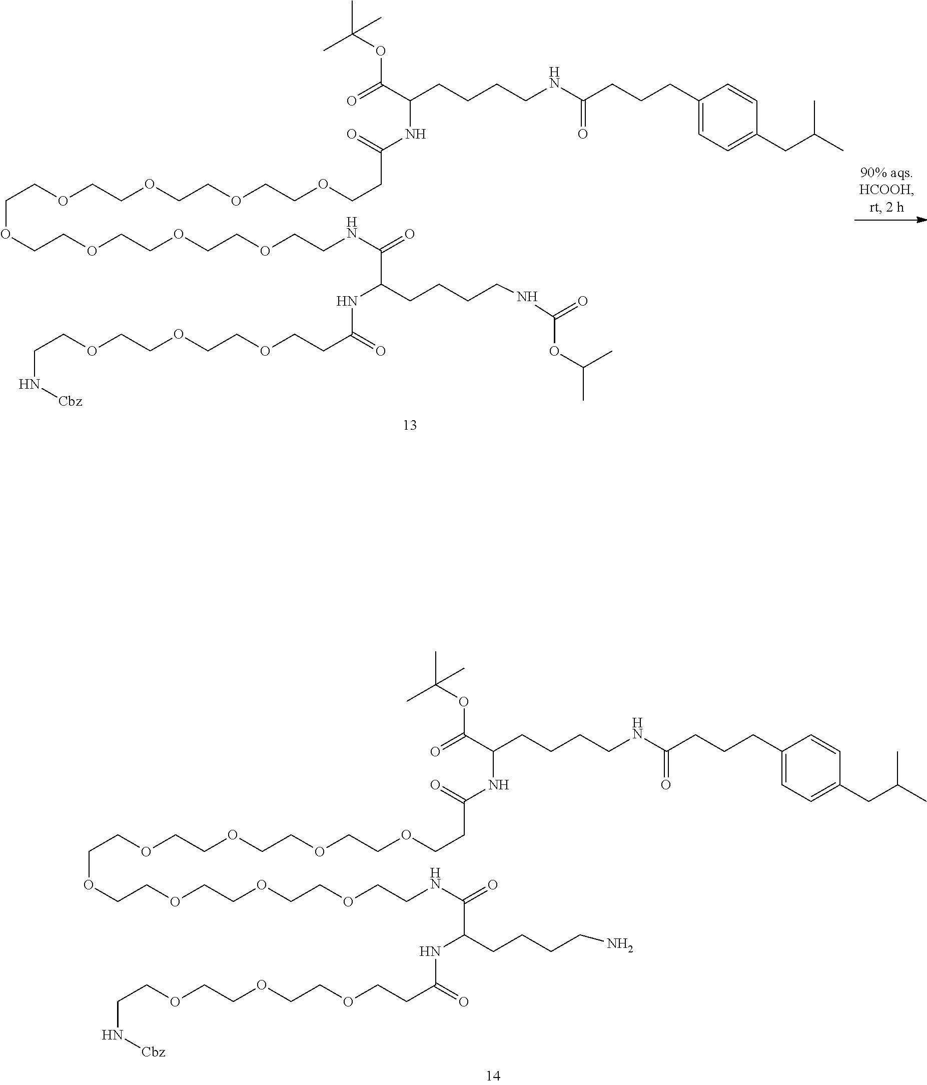 [SMILES: CC(C)Cc1ccc(CCCC(=O)NCCCCC(NC(=O)CCOCCOCCOCCOCCOCCOCCOCCOCCNC(=O)C(CCCCNC(=O)OC(C)C)NC(=O)CCOCCOCCOCCNC(=O)OCc2ccccc2)C(=O)OC(C)(C)C)cc1 ; CC(C)Cc1ccc(CCCC(=O)NCCCCC(NC(=O)CCOCCOCCOCCOCCOCCOCCOCCOCCNC(=O)C(CCCCN)NC(=O)CCOCCOCCOCCNC(=O)OCc2ccccc2)C(=O)OC(C)(C)C)cc1]

A solution of tert-butyl 18-(4-((tert-butoxycarbonyl)amino)butyl)-49-(4-(4-(4-isobutylphenyl)butanamido)butyl)-3,16,19,47-tetraoxo-1-phenyl-2,7,10,13,23,26,29,32,35,38,41,44-dode-caoxa-4,17,20,48-tetraazapentacontan-50-oate (0.70 g, 1.22 mmol) in 90% aq. formic acid (10 ml) was stirred at ambient temperature for 2 h. Full conversion was detected by LCMS. The solvents were evaporated, and the residue was co-evaporated with ACN (3×) and toluene (3×). The residue was dissolved in a mixture MeOH/water (1:1, 25 ml) and treated with ion-exchange resin AmberLite HPR550 (OH-form) at ambient temperature for 15 minutes. The resin was filtered and washed with MeOH and water. The filtrate was evaporated to obtain target material (free base, 0.62 g, 95% yield) as a colorless liquid. The crude amine was used for the step without further purification. LCMS: C₆₆H₁₁₂N₆O₁₉: m/z: 1293.62, observed m/z=1294.1 [M+H]⁺.

Procedure 64: Synthesis of tri-tert-butyl 2,2′,2″-(10-(39-(tert-butoxycarbonyl)-48-(4-isobutylphenyl)-2,9,37,45-tetraoxo-8-(3-oxo-1-phenyl-2,7,10,13-tetraoxa-4-azahexadecanamido)-13,16,19,22,25,28,31,34-octaoxa-3,10,38,44-tetraazaoctatetracontyl)-1,4,7,10-tetraazacyclododecane-1,4,7-triyl)triacetate (15):

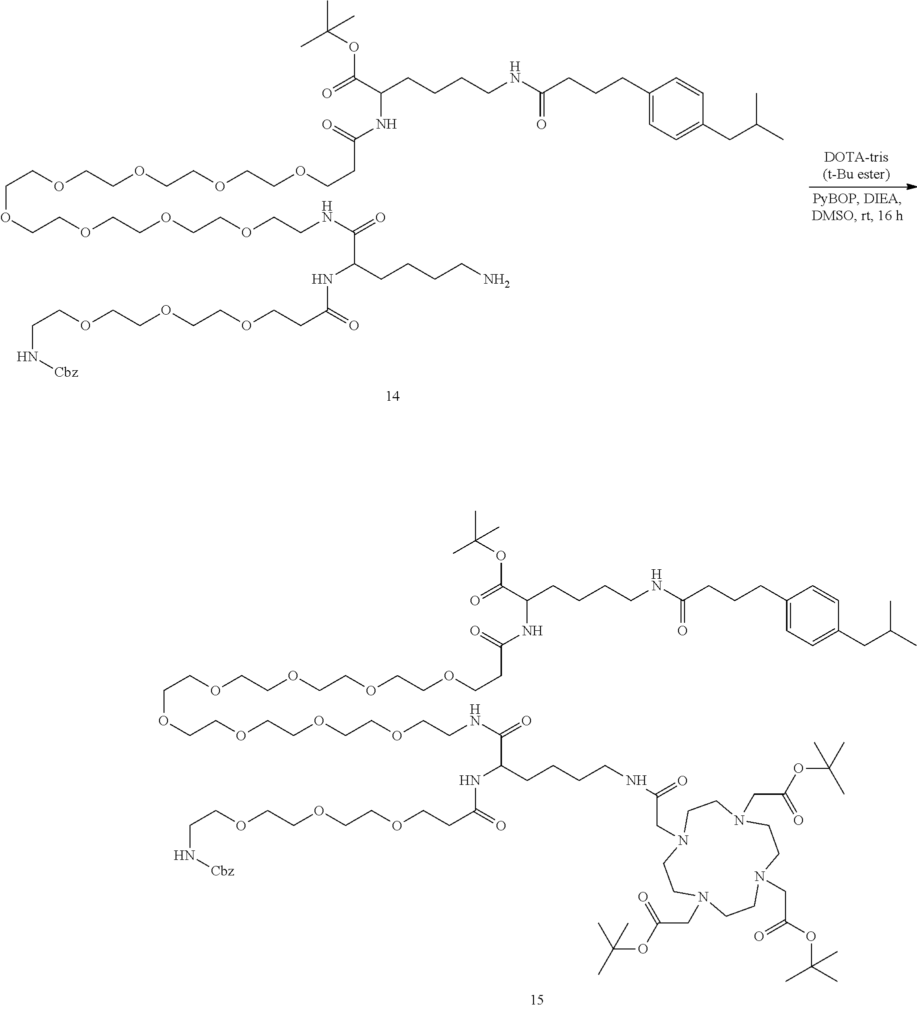 [SMILES: CC(C)Cc1ccc(CCCC(=O)NCCCCC(NC(=O)CCOCCOCCOCCOCCOCCOCCOCCOCCNC(=O)C(CCCCN)NC(=O)CCOCCOCCOCCNC(=O)OCc2ccccc2)C(=O)OC(C)(C)C)cc1 ; CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCNC(=O)OCc2ccccc2)C(=O)OC(C)(C)C)cc1]

To a solution of tert-butyl 18-(4-aminobutyl)-49-(4-(4-(4-isobutylphenyl)butanamido)butyl)-3,16,19,47-tetraoxo-1-phenyl-2,7,10,13,23,26,29,32,35,38,41,44-dodecaoxa-4,17,20,48-tetraazapentacontan-50-oate (0.62 g, 0.48 mmol), DOTA-tris(t-Bu-ester) (0.27 g, 0.48 mmol) and PyBop (0.30 g, 0.56 mmol) in DMSO (8 ml) was added DIEA (0.20 ml, 1.15 mmol) and the reaction mixture was stirred at ambient temperature for 16 h. Water (20 ml) was added, and the mixture was extracted with EtOAc (30 ml). The separated organic layer was washed with water (7×) and brine (8×) to remove excess PyBOP. The organic layer was dried over Na₂SO₄ and concentrated under reduced pressure obtained target product (820 mg, 93% yield) as colorless liquid. The compound was used for the nest step without further purification. LCMS: C₉₄H₁₆₂N₁₀O₂₆: m/z: 1848.34, observed m/z=1849.7 [M+H]⁺.

Procedure 65: Synthesis of tri-tert-butyl 2,2′,2″-(10-(8-(3-(2-(2-(2-aminoethoxy)ethoxy)ethoxy)propanamido)-39-(tert-butoxycarbonyl)-48-(4-isobutylphenyl)-2,9,37,45-tetraoxo-13,16,19,22,25,28,31,34-octaoxa-3,10,38,44-tetraazaoctatetracontyl)-1,4,7,10-tetraazacyclododecane-1,4,7-triyl)triacetate (16):

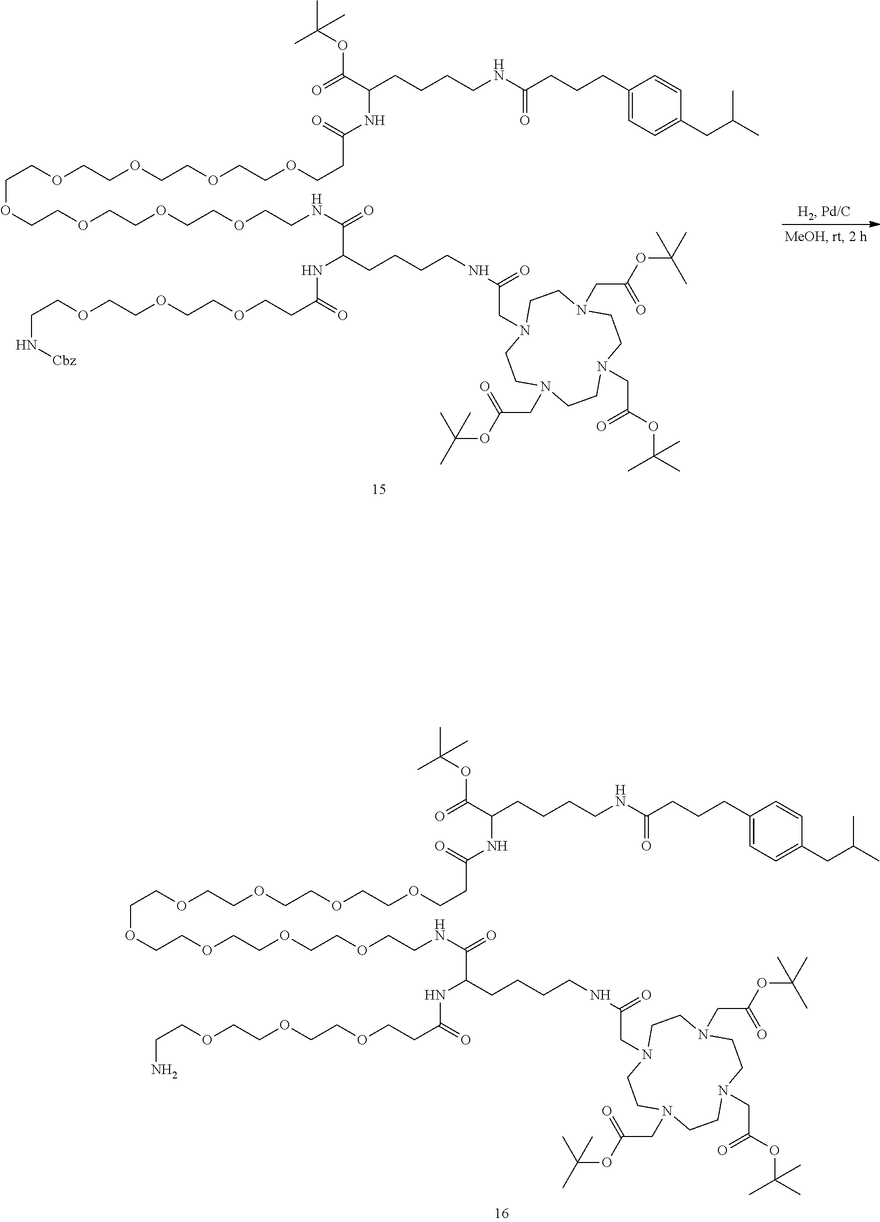 [SMILES: CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCNC(=O)OCc2ccccc2)C(=O)OC(C)(C)C)cc1 ; CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCN)C(=O)OC(C)(C)C)cc1]

Palladium on carbon (10%, 0.12 g, wet, 67%) was suspended in a solution of tri-tert-butyl 2,2′,2″-(10-(39-(tert-butoxycarbonyl)-48-(4-isobutylphenyl)-2,9,37,45-tetraoxo-8-(3-oxo-1-phenyl-2,7,10,13-tetraoxa-4-azahexadecanamido)-13,16,19,22,25,28,31,34-octaoxa-3,10,38,44-tetraazaoctatetracontyl)-1,4,7,10-tetra-azacyclododecane-1,4,7-triyl)tri acetate (500 mg, 0.270 mmol) in MeOH (15 ml). The suspension was stirred for 2 h at room temperature under H₂ at balloon pressure. The mixture was filtered through celite, and the filtrate was evaporated to afford target compound (387 mg, 830% yield) as a colorless liquid. The crude product was used for the next step without further purification. LCMS: C₈₆H₁₅₆N₁₀O₂₄: m/z: 1714.21, observed m/z=1714.7 [M+H]⁺.

Procedure 66: Synthesis of 2,2′,2″-(10-(8-(3-(2-(2-(2-aminoethoxy)ethoxy)ethoxy)propanamido)-39-carboxy-48-(4-isobutylphenyl)-2,9,37,45-tetraoxo-13,16,19,22,25,28,31,34-octaoxa-3,10,38,44-tetraazaoctatetracon-tyl)-1,4,7,10-tetraazacyclododecane-1,4,7-triyl)triacetic acid (17):

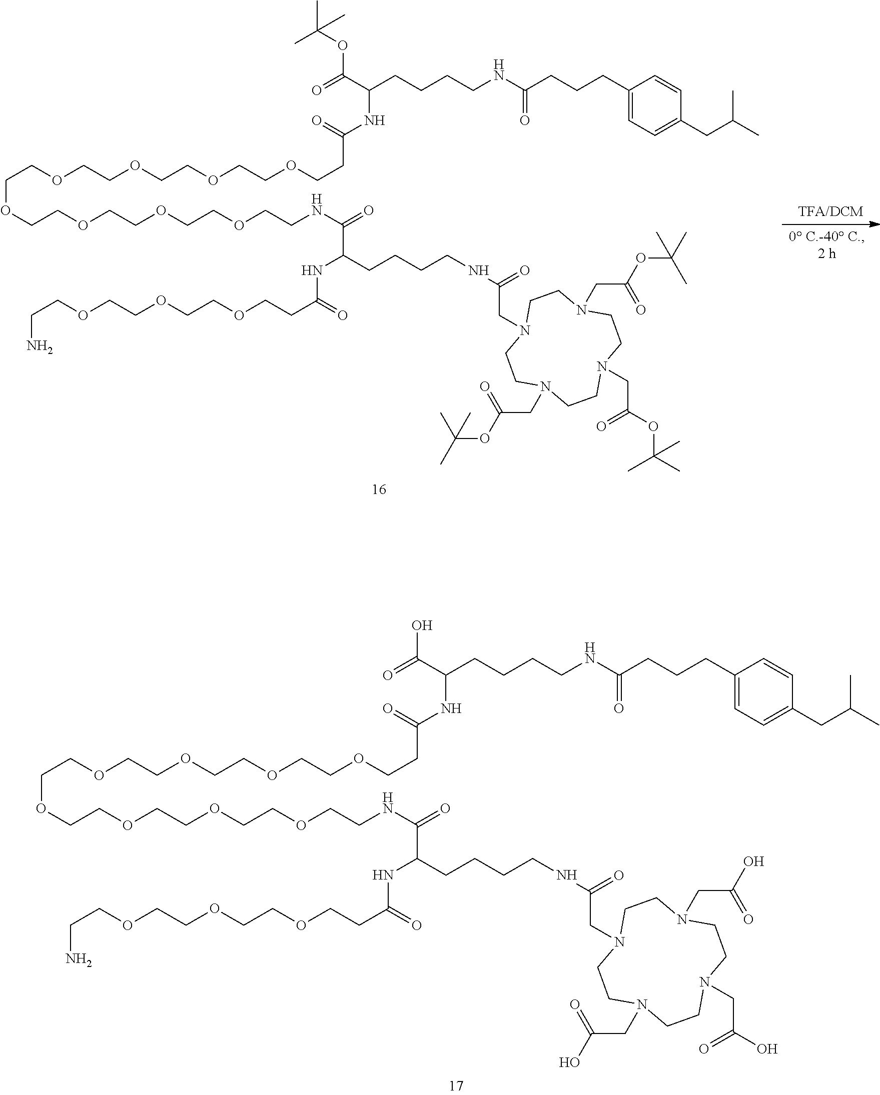 [SMILES: CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)OC(C)(C)C)CCN(CC(=O)OC(C)(C)C)CCN(CC(=O)OC(C)(C)C)CC2)NC(=O)CCOCCOCCOCCN)C(=O)OC(C)(C)C)cc1 ; CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCN)C(=O)O)cc1]

To a 0° C. solution of tri-tert-butyl 2,2′,2″-(10-(8-(3-(2-(2-(2-aminoethoxy)ethoxy)ethoxy)propanamido)-39-(tert-butoxycarbonyl)-48-(4-isobutylphenyl)-2,9,37,45-tetraoxo-13,16,19,22,25,28,31,34-octaoxa-3,10,38,44-tetraazaoctatetracontyl)-1,4,7,10-tetraazacyclododecane-1,4,7-triyl)triacetate (387 mg, 0.225 mmol) in DCM (4 ml), was added TFA (2 ml). The reaction mixture was stirred at 40° C. for 2 h and the solvents were evaporated under reduced pressure. The residue was washed with hexane (2×) and ether (2×) to obtain the product (340 mg, 100%) as a white powder. The crude product was used for the next step without further purification. LCMS: C₇₀H₁₂₄N₁₀O₂₄: m/z: 1489.79, observed m/z=1490.5 [M+H]⁺.

Procedure 67: Synthesis of 2,2′,2″-(10-(39-carboxy-48-(4-isobutylphenyl)-2,9,37,45-tetraoxo-8-(2-oxo-1-((4-((2-oxo-2-((R)-2-((3aR,4R,6R,7aS)-5,5,7a-trimethylhexahydro-4,6-methanobenzo[d][1,3,2]dioxaborol-2-yl)-pyrrolidin-1-yl)ethyl)carbamoyl)quinolin-7-yl)oxy)-6,9,12-trioxa-3-azapentadecanamido)-13,16,19,22,25,28,31,34-octaoxa-3,10,38,44-tetraazaoctatetracontyl)-1,4,7,10-tetraazacyclododecane-1,4,7-triyl)triacetic acid (4):

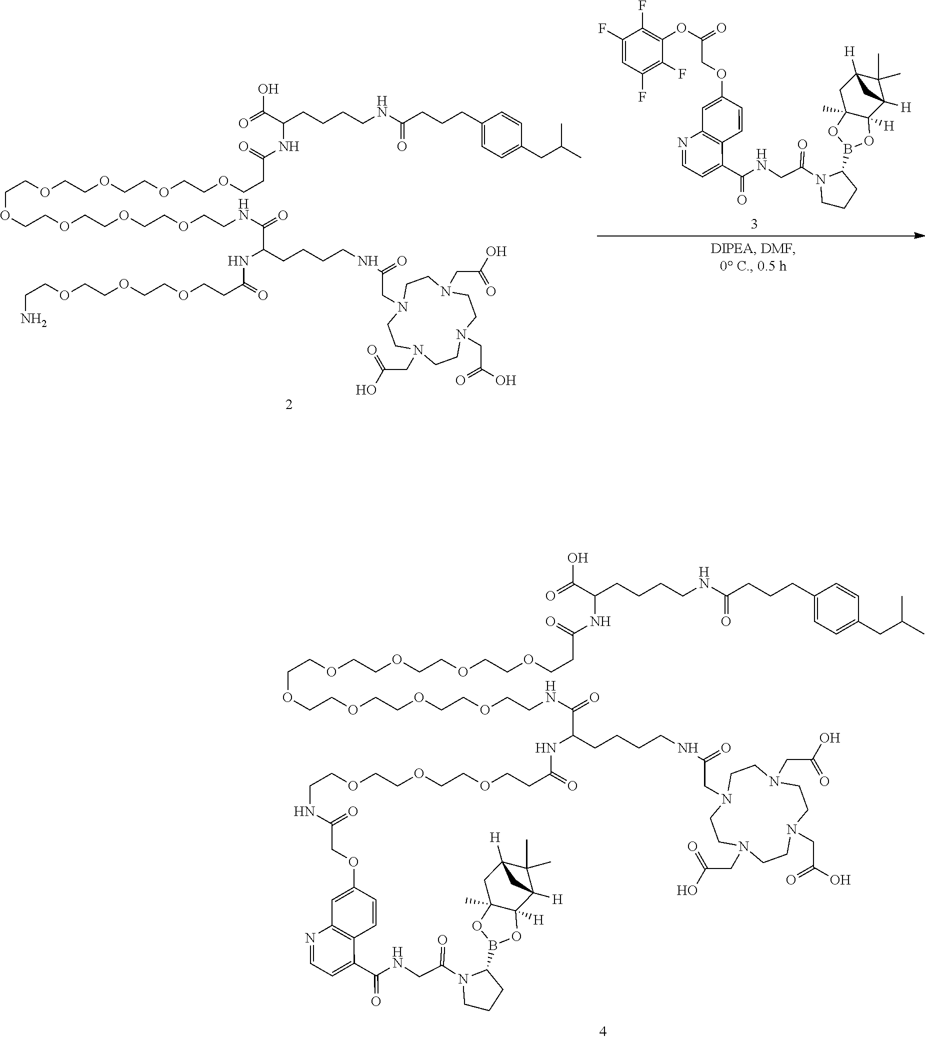 [SMILES: CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCN)C(=O)O)cc1 ; CC1(C)[C@@H]2C[C@H]1[C@H]1OB([C@@H]3CCCN3C(=O)CNC(=O)c3ccnc4cc(OCC(=O)Oc5c(F)c(F)cc(F)c5F)ccc34)O[C@@]1(C)C2 ; CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B4O[C@@H]5[C@@H]6C[C@H](C[C@]5(C)O4)C6(C)C)ccnc3c2)C(=O)O)cc1]

To a solution of 2,2′,2″-(10-(8-(3-(2-(2-(2-aminoethoxy)ethoxy)ethoxy)propanamido)-39-carboxy-48-(4-isobutylphenyl)-2,9,37,45-tetraoxo-13,16,19,22,25,28,31,34-octaoxa-3,10,38,44-tetraazaoctatetracontyl)-1,4,7,10-tetraazacyclododecane-1,4,7-triyl)tri acetic acid (120 mg, 0.080 mmol) in DMF (2 mL), was added DIPEA (0.11 mL, 0.645 mmol) at 0° C. and the reaction mixture was stirred at 0° C. for 5 min. Next, 2,3,5,6-tetrafluorophenyl 2-((4-((2-oxo-2-((R)-2-((3aR,4R,6R,7aS)-5,5,7a-trimethylhexahydro-4,6-methanobenzo[d][1,3,2]dioxaborol-2-yl)pyrrolidin-1-yl)ethyl)carbamoyl)quinolin-7-yl)oxy)acetate (66 mg, 0.096 mmol) in DMF (0.5 mL) was added at 0° C. and the reaction mixture was stirred at the same temperature for 0.5 h. After complete consumption of the amine by LCMS, the crude product was submitted for HPLC purification. Desired fractions were combined and lyophilized to obtain 4 (50 mg, 31%) as an off-white solid. LCMS: C₉₈H₁₅₆BN₁₃O₃₀: m/z: 2006.12, observed m/z=1004.80 [M/2+H]⁺.

Procedure 68: Synthesis of 2,2′,2″-(10-(8-(1-((4-((2-((R)-2-boronopyrrolidin-1-yl)-2-oxoethyl)carbamoyl)quinolin-7-yl)oxy)-2-oxo-6,9,12-trioxa-3-azapentadecanamido)-39-carboxy-48-(4-isobutylphenyl)-2,9,37,45-tetraoxo-13,16,19,22,25,28,31,34-octaoxa-3,10,38,44-tetraazaoctatetracontyl)-1,4,7,10-tetraazacyclo-dodecane-1,4,7-triyl)triacetic acid (RTX-1357R (5)):

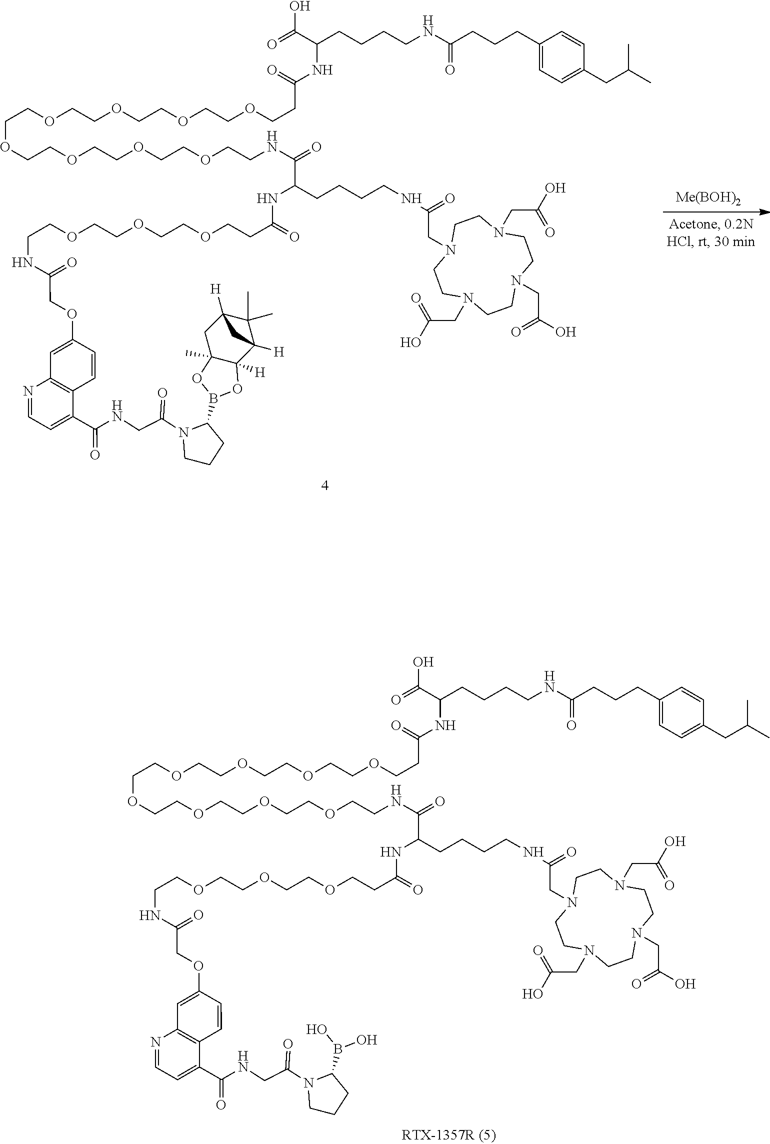 [SMILES: CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B4O[C@@H]5[C@@H]6C[C@H](C[C@]5(C)O4)C6(C)C)ccnc3c2)C(=O)O)cc1 ; CC(C)Cc1ccc(CCCC(=O)NCCCCC(NC(=O)CCOCCOCCOCCOCCOCCOCCOCCOCCNC(=O)C(CCCCNC(=O)CN2CCN(CC(=O)O)CCN(CC(=O)O)CCN(CC(=O)O)CC2)NC(=O)CCOCCOCCOCCNC(=O)COc2ccc3c(C(=O)NCC(=O)N4CCC[C@H]4B(O)O)ccnc3c2)C(=O)O)cc1]

To a solution of 2,2′,2″-(10-(39-carboxy-48-(4-isobutylphenyl)-2,9,37,45-tetraoxo-8-(2-oxo-1-((4-((2-oxo-2-((R)-2-((3aR,4R,6R,7aS)-5,5,7a-trimethylhexahydro-4,6-methanobenzo[d][1,3,2]dioxaborol-2-yl)pyrrolidin-1-yl)ethyl)carbamoyl)quinolin-7-yl)oxy)-6,9,12-trioxa-3-azapentadecanamido)-13,16,19,22,25,28,31,34-octaoxa-3,10,38,44-tetraazaoctatetracontyl)-1,4,7,10-tetraazacyclododecane-1,4,7-triyl)triacetic acid (35 mg, 0.017 mmol) in acetone (0.5 mL) and 0.2N HCl (0.5 mL) mixture was added MeB(OH)2 (5 mg, 0.087 mmol) at rt and the reaction mixture was stirred at ambient temperature for 30 min. After complete consumption of the starting material by LCMS, volatiles were evaporated under reduced pressure and the crude product was submitted for HPLC purification. Desired fractions were combined and lyophilized to obtain target compound (15 mg, 46%, HPLC purity 95%) as an off-white solid. LCMS: C₈₈H₁₄₂BN₁₃O₃₀: m/z: 1872.95, observed m/z=1873.80 [M+H]⁺.

### Synthesis of RTX-1358R

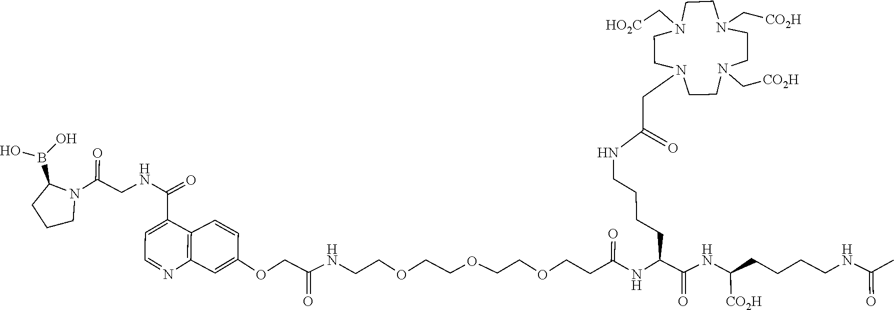 [SMILES: CC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3B(O)O)ccnc2c1)C(=O)O]

Prepared according to the procedures of Example 9 with the following changes:
Procedure 3 was used in lieu of Procedure 40.
The product of Procedure 44 was subjected the Procedures 13, 17, and 18 for the installation of the DOTA chelator.
The resulting product was then subjected to Procedures 51-54 for the installation of the borylated proline derivative.

### Synthesis of RTX-1361R

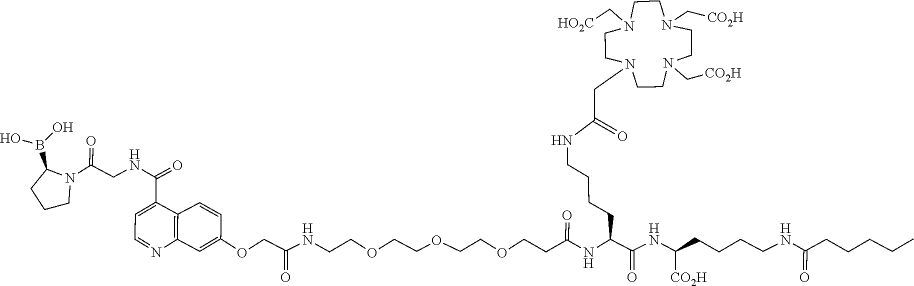 [SMILES: CCCCCC(=O)NCCCC[C@H](NC(=O)[C@H](CCCCNC(=O)CN1CCN(CC(=O)O)CCN(CC(=O)O)CCN(CC(=O)O)CC1)NC(=O)CCOCCOCCOCCNC(=O)COc1ccc2c(C(=O)NCC(=O)N3CCC[C@H]3B(O)O)ccnc2c1)C(=O)O]

Prepared according to the procedures of Example 9 with the following changes:
The product of Procedure 44 was subjected the Procedures 13, 17, and 18 for the installation of the DOTA chelator.

The resulting product was subjected to Procedures 51-54 for the installation of the borylated proline derivative.

### Example 10—Synthesis of RTX-1389

### Synthesis of Compound 10

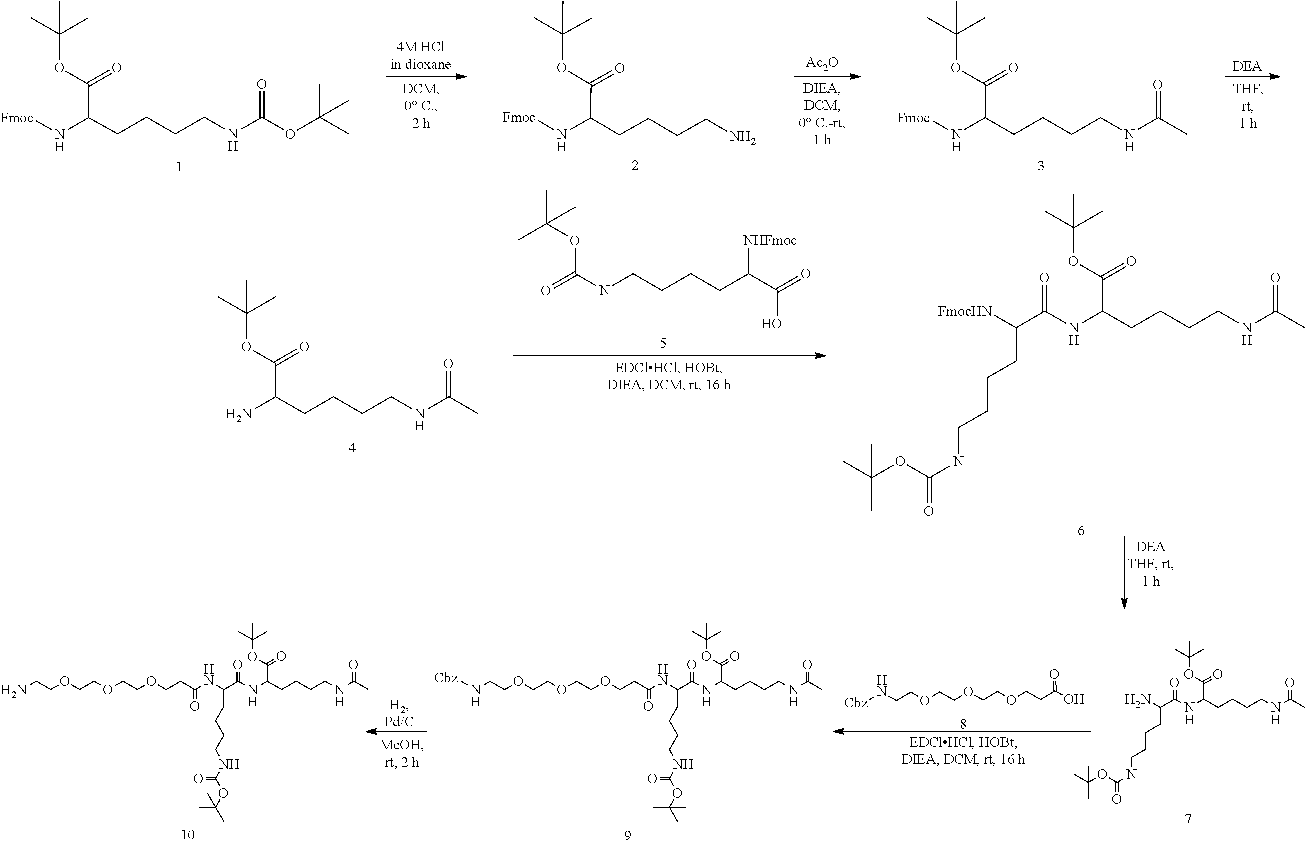

Procedure 69: Synthesis of tert-butyl 2-((((9H-fluoren-9-yl)methoxy)carbonyl)amino)-6-aminohexanoate (2):

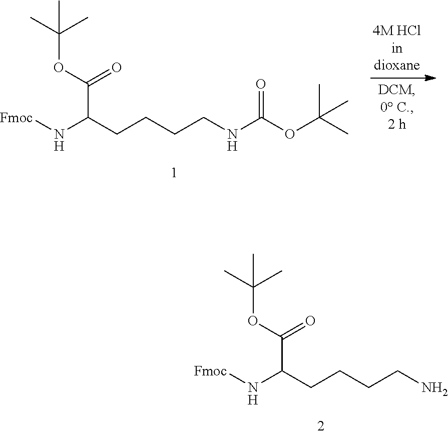

To a solution of tert-butyl 2-((((9H-fluoren-9-yl)methoxy)carbonyl)amino)-6-((tert-butoxycarbonyl)amino)hexanoate (2.5 g, 4.77 mmol) in DCM (10 ml), was added HCl in dioxane (4 M, 20 ml) at 0° C. dropwise and the reaction mixture was stirred at 0° C. for 2 h. The solvents were evaporated and the residue was co-evaporated with hexane and dried in vacuo to obtain HCl salt of target material (2.02 g, 100%) as a white solid. The crude product was used for the next step without further purification. LCMS: C₂₅H₃₂N₂O₄: m/z: 424.53, observed m/z=425.3 [M+H]⁺.

Procedure 70: Synthesis of tert-butyl 2-((((9H-fluoren-9-yl)methoxy)carbonyl)amino)-6-acetamidohexanoate (3):

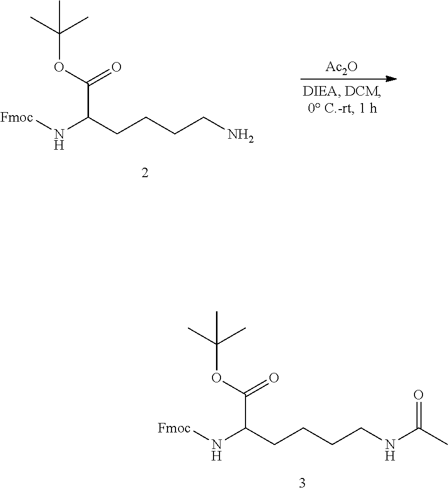

To a solution of tert-butyl 2-((((9H-fluoren-9-yl)methoxy)carbonyl)amino)-6-aminohexanoate (2.02 g, 4.71 mmol) and acetic anhydride (2.22 ml, 23.58 mmol) in DCM (20 ml), was added DIEA (1.64 ml, 9.43 mmol) at 0° C. The reaction mixture was stirred at rt for 1 h. The reaction mixture was diluted with 20 ml of DCM and then washed with water. The organic layer was dried over Na₂SO₄ and concentrated under reduced pressure. The crude product was purified by flash chromatography (EtOAc/hexane, eluted at 50% EtOAc in hexane) to afford the product (1.60 g, 73% yield) as a white solid. LCMS: C₂₇H₃₄N₂O₅: m/z: 466.57, observed m/z=467.3 [M+H]⁺.

Procedure 71: Synthesis of tert-Butyl 6-acetamido-2-aminohexanoate (4):

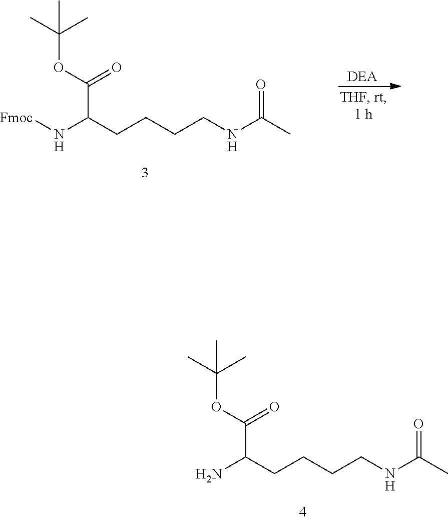

To a solution of tert-butyl 2-((((9H-fluoren-9-yl)methoxy)carbonyl)amino)-6-acetamidohexanoate (1.60 g, 3.43 mmol) in THF (10 ml) was added diethylamine (10 ml) at room temperature. The reaction mixture was stirred at ambient temperature for 2 hours. The solvent was evaporated and the residue was washed (2×) with hexane and dried in vacuum to obtain the product (900 mg, 99%). The compound was used for the next step without further purification. LCMS: C₁₂H₂₄N₂O₃: m/z: 244.33, observed m/z=245.4 [M+H]⁺.

Procedure 72: Synthesis of tert-butyl 2-(2-((((9H-fluoren-9-yl)methoxy)carbonyl)amino)-6-((tert-butoxycarbonyl)amino)hexanamido)-6-acetamidohexanoate (6):

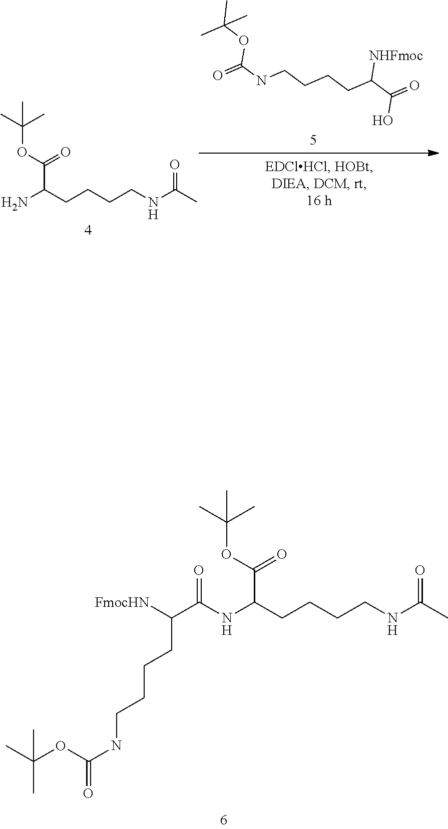

To a solution of 2-((((9H-fluoren-9-yl)methoxy)carbonyl)amino)-6-((tert-butoxycarbonyl)amino)hexanoic acid (1.72 g, 3.68 mmol) in 15 ml of DCM was added EDCI·HCl (1.05 g, 5.53 mmol) and HOBt (0.50 g, 3.68 mmol), and the reaction mixture was stirred at rt for 5 min. Next, tert-butyl 6-acetamido-2-aminohexanoate (0.90 g, 3.68 mmol) in DCM (10 ml) and DIEA (1.92 ml, 11.06 mmol) were added in sequentially and the mixture was stirred at room temperature for 16 hours. The reaction mixture was diluted with 20 ml of DCM and then washed with water. The organic layer was dried over Na₂SO₄ and concentrated under reduced pressure. The crude product was purified by flash chromatography (EtOAc/hexane, eluted at 75% EtOAc in hexane) to afford the product (2.20 g, 77% yield) as a white solid. LCMS: C₃₈H₅₄N₄O₈: m/z: 694.86, observed m/z=695.3 [M+H]⁺.

Procedure 73: Synthesis of tert-butyl 6-acetamido-2-(2-amino-6-((tert-butoxycarbonyl)amino)hexanamido)-hexanoate (7):

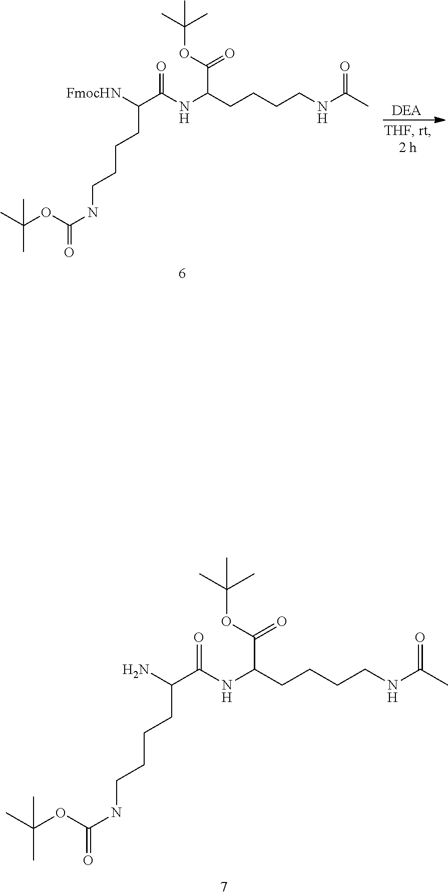

To a solution of tert-butyl 2-(2-((((9H-fluoren-9-yl)methoxy)carbonyl)amino)-6-((tert-butoxycarbonyl)-amino)hexanamido)-6-acetamidohexanoate (1.60 g, 2.30 mmol) in THF (10 ml) was added diethylamine (10 ml) at room temperature. The reaction mixture was stirred at ambient temperature for 2 hours. The solvent was evaporated and the residue was washed (2×) with hexane and dried under vacuum to obtain the product (1.0 g, 92% yield). The compound was used for the next step without further purification. LCMS: C₂₃H₄₄N₄O₆: m/z: 472.62, observed m/z=473.5 [M+H]⁺.

Procedure 74: Synthesis of tert-butyl 21-(4-acetamidobutyl)-18-(4-((tert-butoxycarbonyl)amino)butyl)-3,16,19-trioxo-1-phenyl-2,7,10,13-tetraoxa-4,17,20-triazadocosan-22-oate (9):

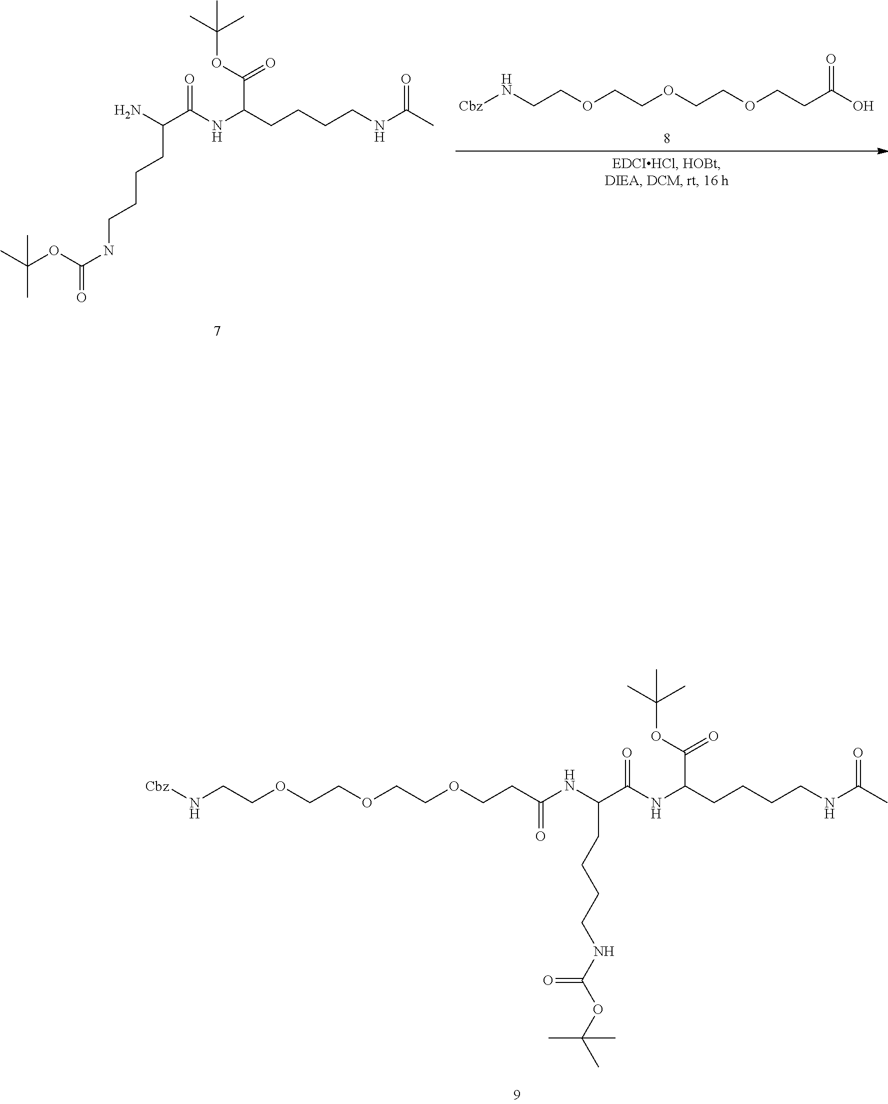 [SMILES: CC(=O)NCCCCC(NC(=O)C(N)CCCCNC(=O)OC(C)(C)C)C(=O)OC(C)(C)C ; O=C(O)CCOCCOCCOCCNC(=O)OCc1ccccc1 ; CC(=O)NCCCCC(NC(=O)C(CCCCNC(=O)OC(C)(C)C)NC(=O)CCOCCOCCOCCNC(=O)OCc1ccccc1)C(=O)OC(C)(C)C]

To a solution of 3-oxo-1-phenyl-2,7,10,13-tetraoxa-4-azahexadecan-16-oic acid (0.83 g, 2.33 mmol) in 10 ml of DCM was added EDCI·HCl (0.67 g, 3.49 mmol) and HOBt (0.31 g, 2.33 mmol) and the reaction mixture was stirred at rt for 5 min. Next, tert-butyl 6-acetamido-2-(2-amino-6-((tert-butoxycarbonyl)amino)hexanamido)hexanoate (1.0 g, 2.33 mmol) in DCM (10 ml) and DIEA (1.2 ml, 6.99 mmol) were added sequentially, and the mixture was stirred at room temperature for 16 hours. The mixture was diluted with 20 ml of DCM and washed with water. The organic layer was dried over Na₂SO₄ and concentrated under reduced pressure. The crude product was purified by flash chromatography (MeOH/DCM, eluted at 3-4% MeOH in DCM) to afford the product (1.20 g, 70% yield) as a white solid. LCMS: C₁₂H₂₄N₂O₃: m/z: 809.99, observed m/z=810.7 [M+H]⁺.

Procedure 75: Synthesis of tert-butyl 17-(4-acetamidobutyl)-1-amino-14-(4-((tert-butoxycarbonyl)amino)butyl)-12,15-dioxo-3,6,9-trioxa-13,16-diazaoctadecan-18-oate (10):

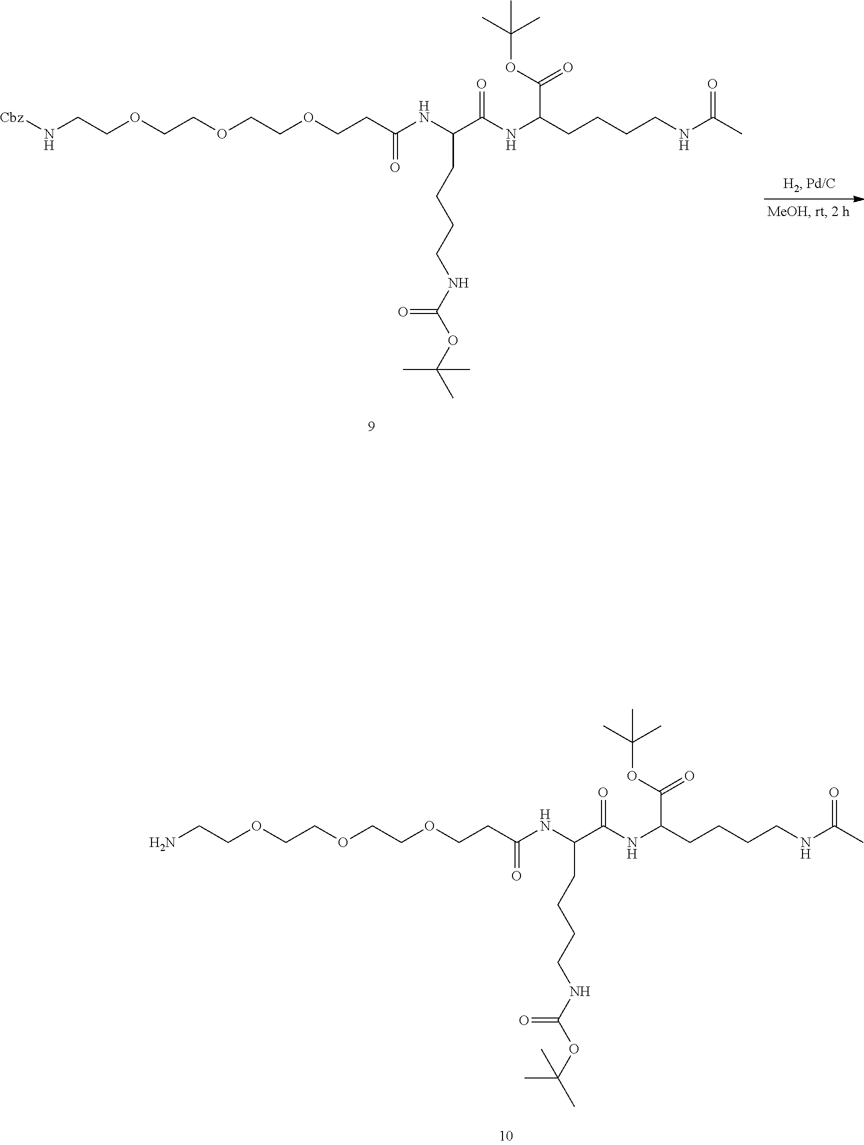 [SMILES: CC(=O)NCCCCC(NC(=O)C(CCCCNC(=O)OC(C)(C)C)NC(=O)CCOCCOCCOCCNC(=O)OCc1ccccc1)C(=O)OC(C)(C)C ; CC(=O)NCCCCC(NC(=O)C(CCCCNC(=O)OC(C)(C)C)NC(=O)CCOCCOCCOCCN)C(=O)OC(C)(C)C]

Palladium on carbon (10%, 0.03 g, wet, 67%) was suspended in a solution of tert-butyl 21-(4-acetamidobutyl)-18-(4-((tert-butoxycarbonyl)amino)butyl)-3,16,19-trioxo-1-phenyl-2,7,10,13-tetraoxa-4,17,20-triazadocosan-22-oate (130 mg, 0.160 mmol) in MeOH (3 ml). The suspension was stirred for 2 h at room temperature under H₂ at balloon pressure. The mixture was then filtered through celite, and the filtrate was evaporated to afford the target compound (106 mg, 98% yield) as a white solid. The crude product was used for the next step without further purification. LCMS: C₃₂H₆₁N₅O₁₀: m/z: 675.85, observed m/z=676.5 [M+H]⁺.

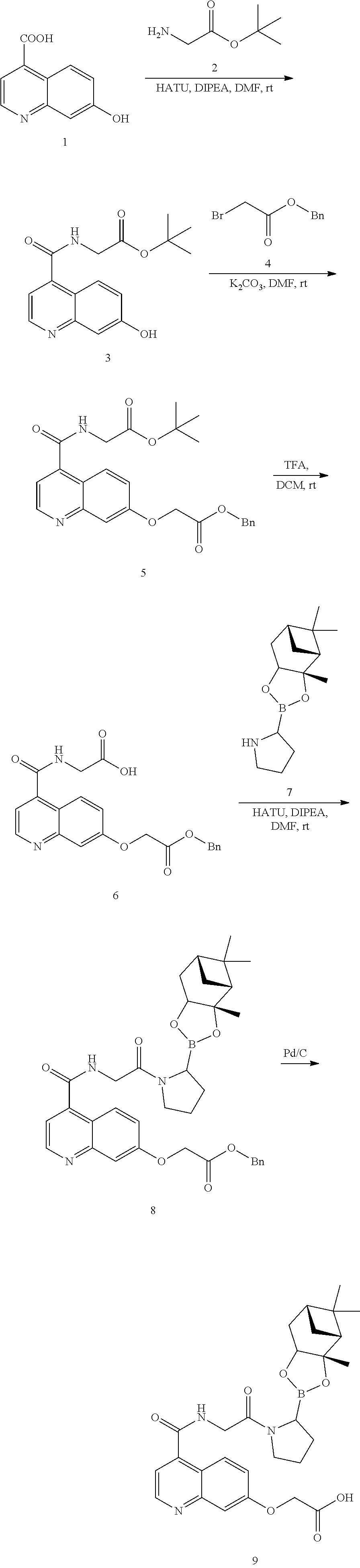

Procedure 76: Synthesis of tert-butyl [(7-hydroxy-4-quinolyl)carbonylamino]acetate

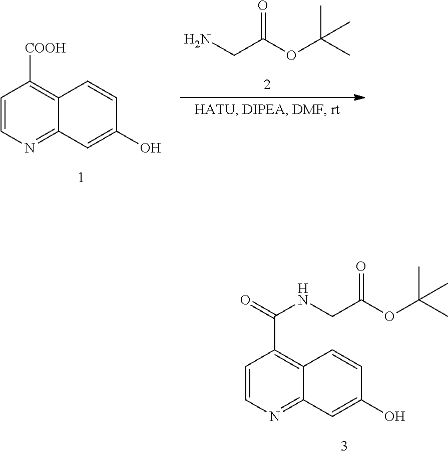

7-Hydroxy-4-quinolinecarboxylic acid (1.0 g, 5.3 mmol, 1 eq), glycine t-butyl ester hydrochloride (1.2 g, 5.78 mmol, 1.1 eq) and HATU (3.02 g, 7.95 mmol, 1.5 eq) in DMF (20 ml) was added DIPEA (2.2 ml, 2.5 eq) at 5° C. and stirred at room temperature for 2 h. Upon completion. H₂O was added and the product was extracted with n-butanol. The organic layer was separated, dried over Na₂SO₄ and evaporated under reduced pressure. The resulting residue was purified by flash chromatography (0-10% MeOH in DCM) to afford 3 (1.1 g, 67% yield) as an off-white powder. ESI-MS m/z calc. 302.324, found 303.0 [M+H]⁺.

Procedure 77: Synthesis of benzyl (4-{[(tert-butoxycarbonylmethyl)amino]carbonyl}-7-quinolyloxy)acet-ate

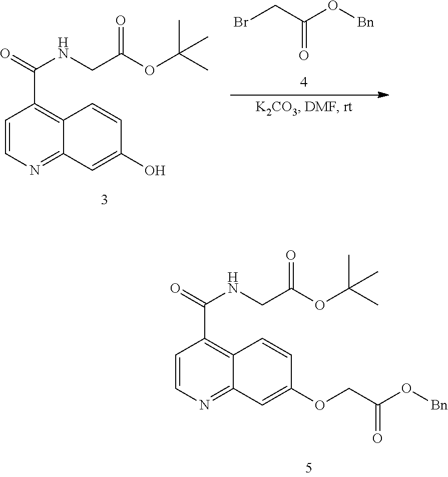

To a suspension of tert-butyl [(7-hydroxy-4-quinolyl)carbonylamino]acetate (1.1 g, 3.55 mmol, 1 eq) and K₂CO₃ (1.5 g, 10.7 mmol, 3 eq) in DMF (20 ml) was added benzyl bromoacetate (2.4 g, 10.7 mmol, 3 eq) at room temperature. The mixture was stirred for 16 h at 60° C. Upon completion, H₂O was added and the product was extracted with EtOAc. The organic layer was separated, dried over Na₂SO₄ and evaporated under reduced pressure. The resulting residue was purified by flash chromatography (0-80% ethyl acetate in hexane) to afford 5 (1 g, 61% yield) as an off-white powder. ESI-MS m/z calc. 450.482, found 451.0 [M+H]⁺.

Procedure 78: Synthesis of ({7-[(benzyloxycarbonyl)methoxy]-4-quinolyl}carbonylamino)acetic acid

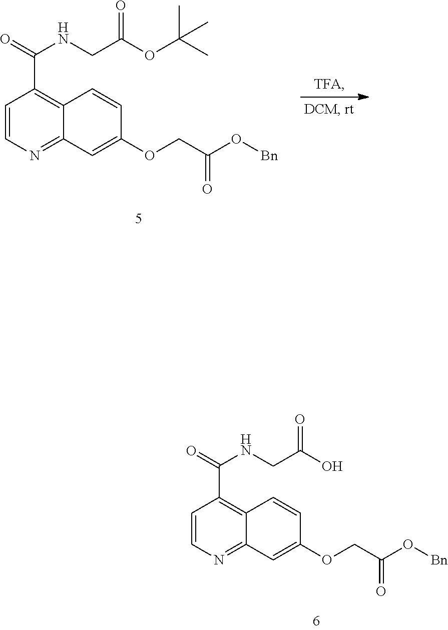 [SMILES: CC(C)(C)OC(=O)CNC(=O)c1ccnc2cc(OCC(=O)OCc3ccccc3)ccc12 ; O=C(O)CNC(=O)c1ccnc2cc(OCC(=O)OCc3ccccc3)ccc12]

To a solution of benzyl (4-{[(tert-butoxycarbonylmethyl)amino]carbonyl}-7-quinolyloxy)acetate (1 g, 2.2 mmol) in DCM was added 10 ml TFA/DCM (DCM:TFA, 1:2) at room temperature and the mixture was stirred for 4 h. Progress of the reaction was monitored by LCMS, and additional TFA solution was added as needed to drive it to completion. The solvents were evaporated under reduced pressure to afford 6 in quantitative yield as an off-white solid. ESI-MS m/z calc. 394.376, found 395.0 [M+H]⁺.

Procedure 79: Synthesis of benzyl (4-{[2-(2-{(1R,2S,8R)-2,9,9-trimethyl-3,5-dioxa-4-boratricyclo-[6.1.1.02,6]dec-4-yl}-1-pyrrolidinyl)-2-oxoethylamino]carbonyl}-7-quinolyloxy)acetate

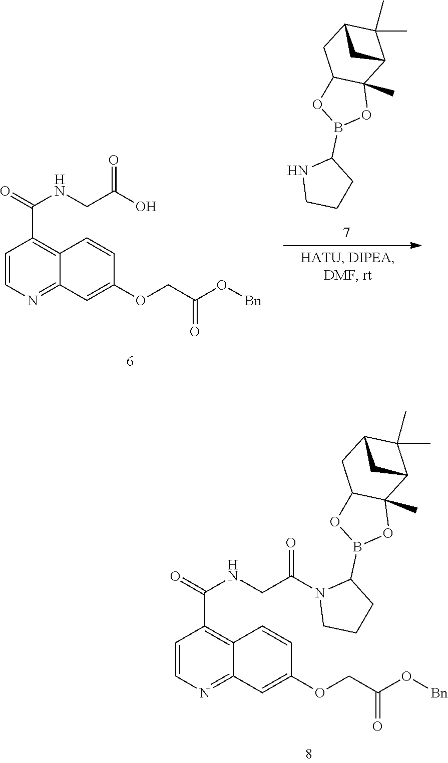 [SMILES: O=C(O)CNC(=O)c1ccnc2cc(OCC(=O)OCc3ccccc3)ccc12 ; CC1(C)[C@H]2CC3OB(C4CCCN4)O[C@@]3(C)[C@@H]1C2 ; CC1(C)[C@H]2CC3OB(C4CCCN4C(=O)CNC(=O)c4ccnc5cc(OCC(=O)OCc6ccccc6)ccc45)O[C@@]3(C)[C@@H]1C2]

To a solution of ({7-[(benzyloxycarbonyl)methoxy]-4-quinolyl}carbonylamino)acetic acid (0.87 g, 2.2 mmol, 1 eq), 2-{(1R,2R,8S)-2,9,9-trimethyltricyclo[6.1.1.02,6]dec-4-yl}pyrrolidine (0.6 g, 2.4 mmol, 1.1 eq) and HATU (1.25 g, 3.3 mmol, 1.5 eq) in DMF (5 ml) was added DIPEA (0.8 ml, 5.5 mmol, 2.5 eq) at 5° C. The mixture was stirred at room temperature for 2 h, H₂O was added, and the product was extracted with EtOAc. The organic layer was separated, dried over Na₂SO₄ and evaporated under reduced pressure. The resulting residue was purified by flash chromatography (0-10% MeOH in DCM) to afford 8 (1.1 g, 82% yield) as an off-white solid. ESI-MS m/z calc. 625.520, found 626.1 [M+H]⁺.

Procedure 80: Synthesis of (4-{[2-(2-{(1R,2S,8R)-2,9,9-Trimethyl-3,5-dioxa-4-boratricyclo[6.1.1.02,6]dec-4-yl}-1-pyrrolidinyl)-2-oxoethylamino]carbonyl}-7-quinolyloxy)acetic acid

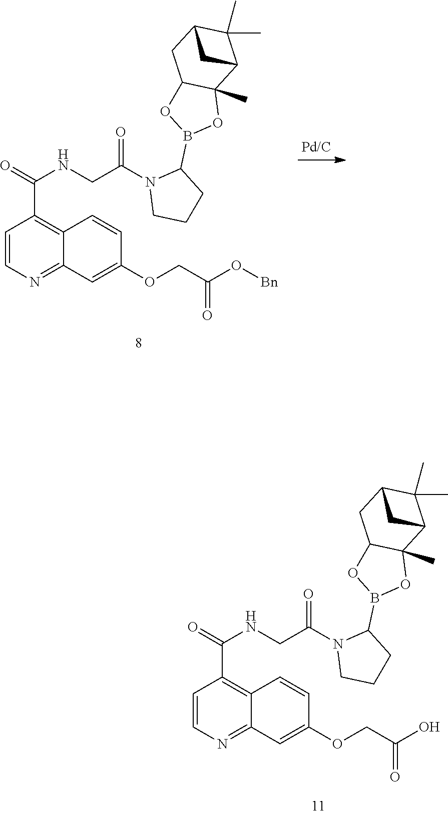 [SMILES: CC1(C)[C@H]2CC3OB(C4CCCN4C(=O)CNC(=O)c4ccnc5cc(OCC(=O)OCc6ccccc6)ccc45)O[C@@]3(C)[C@@H]1C2 ; CC1(C)[C@H]2CC3OB(C4CCCN4C(=O)CNC(=O)c4ccnc5cc(OCC(=O)O)ccc45)O[C@@]3(C)[C@@H]1C2]

Activated palladium on carbon (5%, 0.2 g) was suspended in a solution of 8 (1.1 g, 1.8 mmol) in MeOH (50 mL). The suspension was stirred for 1 h at room temperature under H₂ atmosphere (40 psi). The mixture was filtered through celite, and the filtrate was concentrated under reduced pressure to afford 11 in quantitative yield. (950 mg, 99% yield). ESI-MS m/z calc. 535.398, found 536.1 [M+H]⁺.

### Synthesis of RTX-1389R (16)

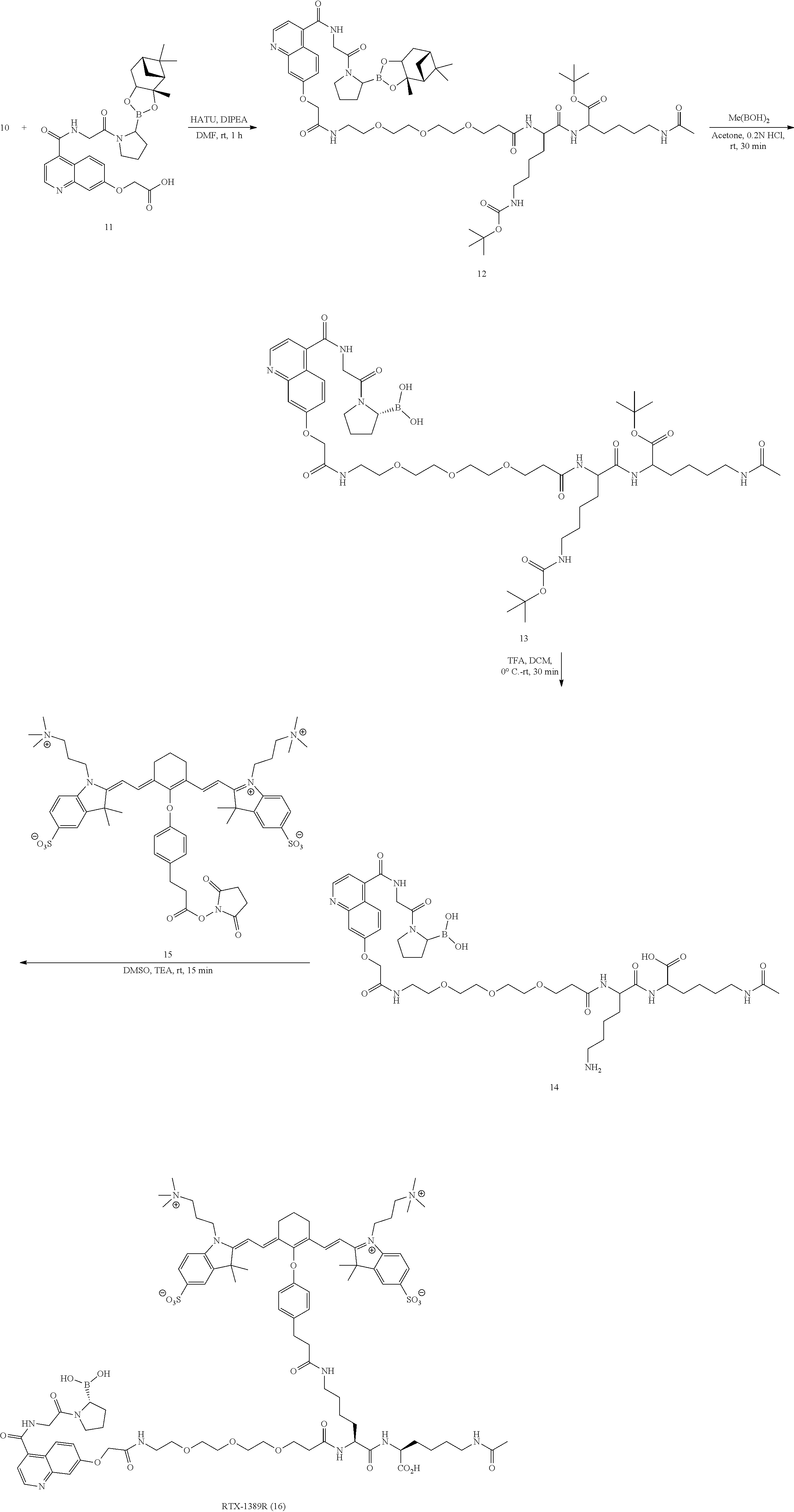

Procedure 81: Synthesis of tert-butyl 20-(4-acetamidobutyl)-17-(4-((tert-butoxycarbonyl)amino)butyl)-2,15,18-trioxo-1-((4-((2-oxo-2-(2-((3aS,4R,6R)-3a,5,5-trimethylhexahydro-4,6-methanobenzo[d][1,3,2]dioxaborol-2-yl)pyrrolidin-1-yl)ethyl)carbamoyl)quinolin-7-yl)oxy)-6,9,12-trioxa-3,16,19-triazahenicosan-21-oate (12):

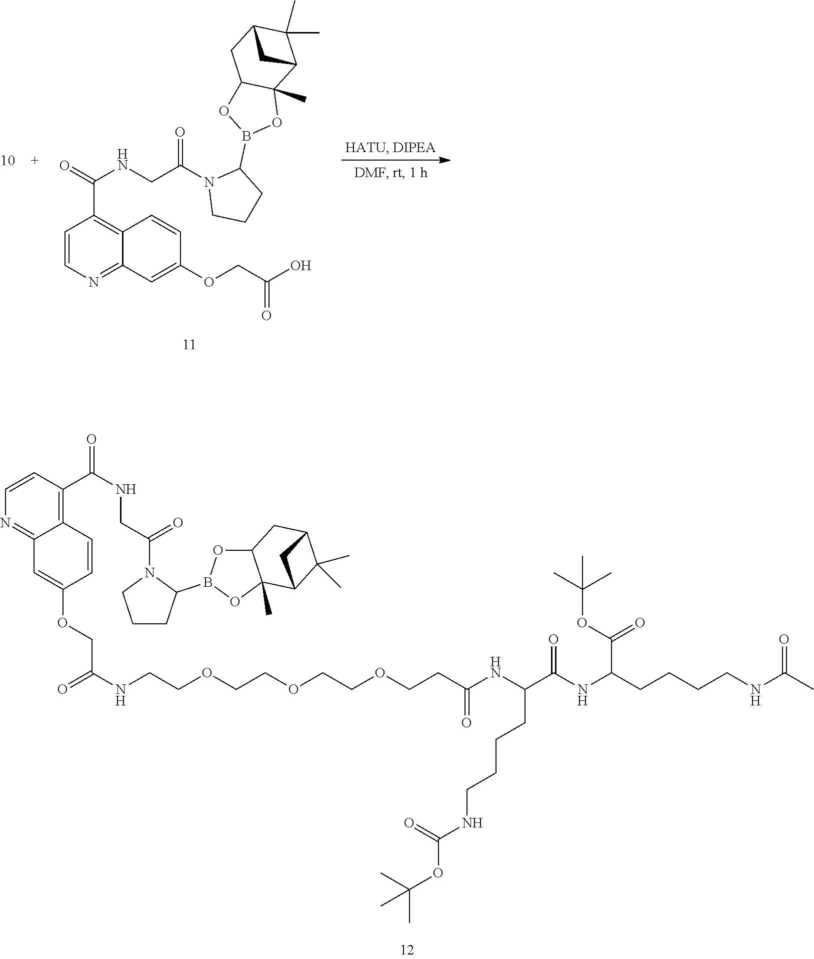 [SMILES: CC1(C)[C@H]2CC3OB(C4CCCN4C(=O)CNC(=O)c4ccnc5cc(OCC(=O)O)ccc45)O[C@@]3(C)[C@@H]1C2 ; CC(=O)NCCCCC(NC(=O)C(CCCCNC(=O)OC(C)(C)C)NC(=O)CCOCCOCCOCCNC(=O)COc1ccc2c(C(=O)NCC(=O)N3CCCC3B3OC4C[C@H]5C[C@H](C5(C)C)[C@]4(C)O3)ccnc2c1)C(=O)OC(C)(C)C]

To a solution of tert-butyl 17-(4-acetamidobutyl)-1-amino-14-(4-((tert-butoxycarbonyl)amino)butyl)-12,15-dioxo-3,6,9-trioxa-13,16-diazaoctadecan-18-oate (86 mg, 0.127 mmol) and 2-((4-((2-oxo-2-(2-((3aS,4R,6R)-3a,5,5-trimethylhexahydro-4,6-methanobenzo[d][1,3,2]dioxaborol-2-yl)pyrrolidin-1-yl)ethyl)carbamoyl)quinolin-7-yl)oxy)acetic acid (68 mg, 0.127 mmol) in 2 ml of DMF was added HATU (53 mg, 0.140 mmol) and DIEA (0.11 ml, 0.637 mmol) and the reaction mixture was stirred at room temperature for 1 h. The reaction progress was monitored by LCMS. DMF was removed under reduced pressure. The crude product was purified by flash chromatography (MeOH/DCM, eluted at 3-4% MeOH in DCM) to afford the product (75 mg, 50% yield) as colorless liquid. LCMS: C₆₀H₉₃BN₈O₁₆: m/z: 1193.24, observed m/z=1194.0 [M+H]⁺.

Procedure 82: Synthesis of (1-(2-(7-((20-(tert-butoxycarbonyl)-17-(4-((tert-butoxycarbonyl)amino)butyl)-2,15,18,26-tetraoxo-6,9,12-trioxa-3,16,19,25-tetraazaheptacosyl)oxy)quinoline-4-carbox-amido)acetyl)pyrrolidin-2-yl)boronic acid (13):

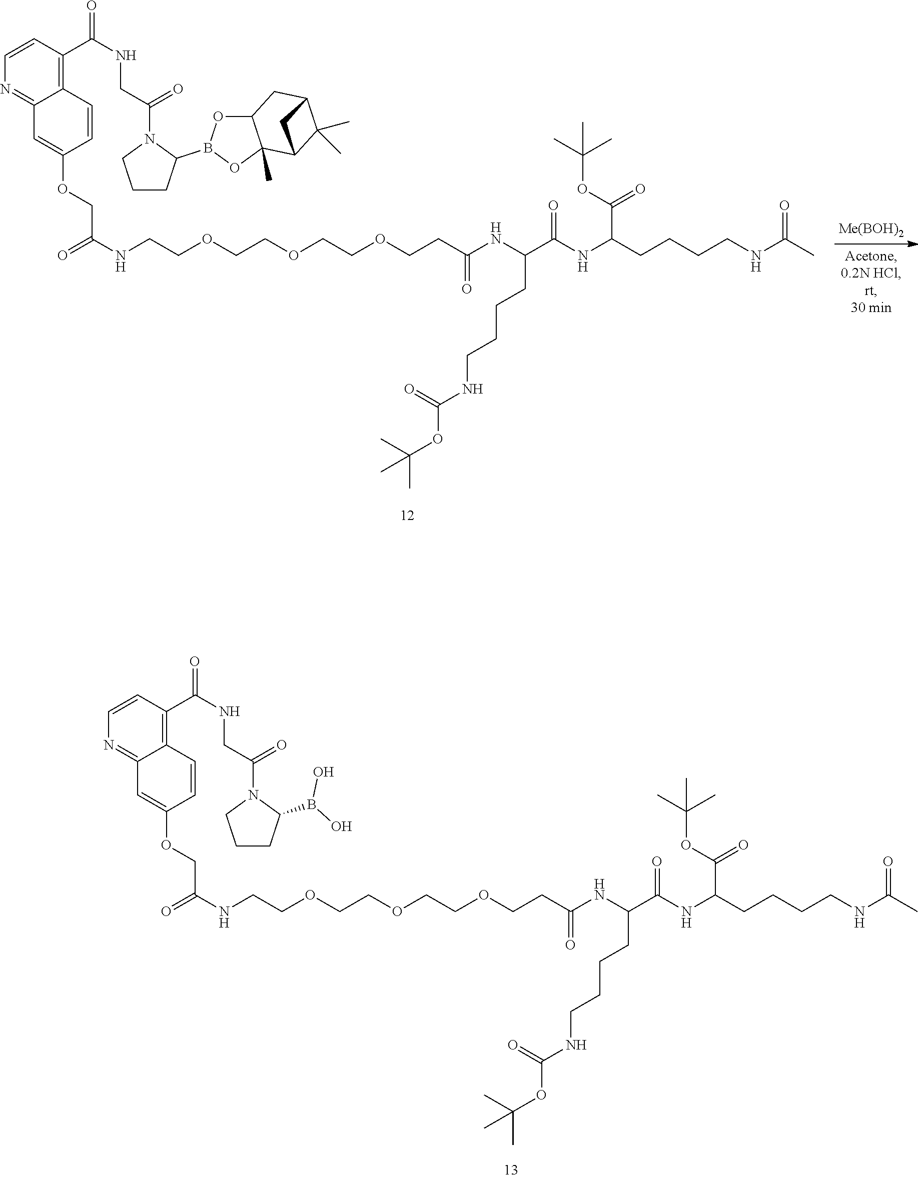 [SMILES: CC(=O)NCCCCC(NC(=O)C(CCCCNC(=O)OC(C)(C)C)NC(=O)CCOCCOCCOCCNC(=O)COc1ccc2c(C(=O)NCC(=O)N3CCCC3B3OC4C[C@H]5C[C@H](C5(C)C)[C@]4(C)O3)ccnc2c1)C(=O)OC(C)(C)C ; CC(=O)NCCCCC(NC(=O)C(CCCCNC(=O)OC(C)(C)C)NC(=O)CCOCCOCCOCCNC(=O)COc1ccc2c(C(=O)NCC(=O)N3CCC[C@H]3B(O)O)ccnc2c1)C(=O)OC(C)(C)C]

To a solution of tert-butyl 20-(4-acetamidobutyl)-17-(4-((tert-butoxycarbonyl)amino)butyl)-2,15,18-trioxo-1-((4-((2-oxo-2-(2-((3aS,4R,6R)-3a,5,5-trimethylhexahydro-4,6-methanobenzo[d][1,3,2]dioxabor-ol-2-yl)-pyrrolidin-1-yl)ethyl)carbamoyl)quinolin-7-yl)oxy)-6,9,12-trioxa-3,16,19-triazahenicosan-21-oate (65 mg, 0.054 mmol) and MeB(OH)₂ (16 mg, 0.272 mmol) in acetone (1 ml), was added 0.2N HCl (1 ml) at rt and the reaction mixture was stirred at ambient temperature for 30 min. The solvent was evaporated, and the residue dried in vacuo to obtain the product (84 mg, 100%) as a colorless liquid. The crude product was used for the next step without further purification. LCMS: C₅₀H₇₉BN₈O₁₆: m/z: 1059.02, observed m/z=1059.8 [M+H]⁺.

Procedure 83: Synthesis of 20-(4-acetamidobutyl)-17-(4-aminobutyl)-1-((4-((2-(2-boronopyrrolidin-1-yl)-2-oxoethyl)carbamoyl)quinolin-7-yl)oxy)-2,15,18-trioxo-6,9,12-trioxa-3,16,19-triazahenicosan-21-oic acid (14):

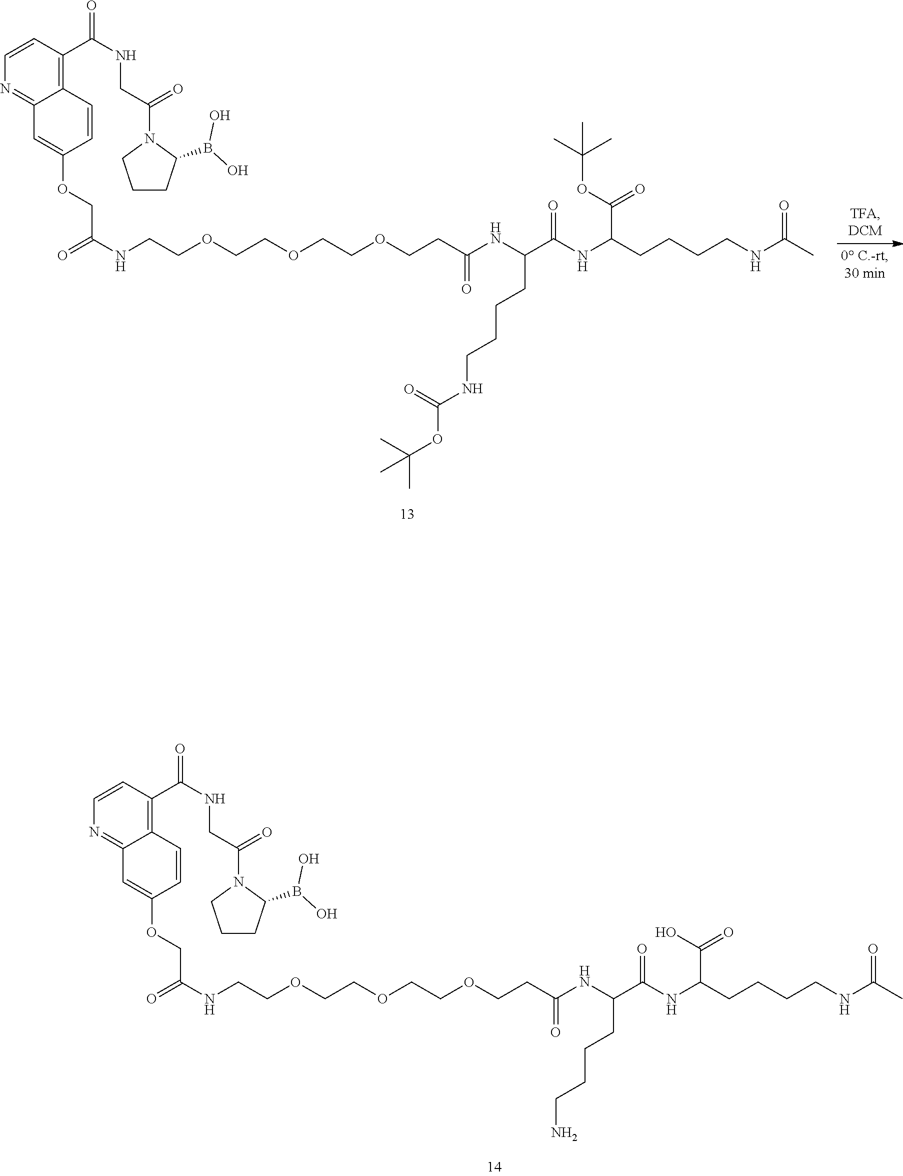 [SMILES: CC(=O)NCCCCC(NC(=O)C(CCCCNC(=O)OC(C)(C)C)NC(=O)CCOCCOCCOCCNC(=O)COc1ccc2c(C(=O)NCC(=O)N3CCC[C@H]3B(O)O)ccnc2c1)C(=O)OC(C)(C)C ; CC(=O)NCCCCC(NC(=O)C(CCCCN)NC(=O)CCOCCOCCOCCNC(=O)COc1ccc2c(C(=O)NCC(=O)N3CCC[C@H]3B(O)O)ccnc2c1)C(=O)O]

To a solution of (1-(2-(7-((20-(tert-butoxycarbonyl)-17-(4-((tert-butoxycarbonyl)amino)butyl)-2,15,18,26-tetraoxo-6,9,12-trioxa-3,16,19,25-tetraazaheptacosyl)oxy)quinoline-4-carboxamido)acetyl)pyrrolidin-2-yl)boronic acid (84 mg, 0.079 mmol) in DCM (1.5 ml), was added TFA (0.5 ml) at 0° C. and the reaction mixture was stirred at ambient temperature for 30 min. The solvents were evaporated, and the crude product was subjected to HPLC purification. Desired fractions were combined and lyophilized to obtain the product (28 mg, 48%) as an off-white solid. LCMS: C₄₁H₆₃BN₈O₁₄: m/z: 902.80, observed m/z=885.5 [M-OH]⁺.

Procedure 84: Synthesis of 2-((E)-2-((E)-2-(4-((17S)-17-(((S)-5-acetamido-1-carboxypentyl)carbamoyl)-1-((4-((2-(2-boronopyrrolidin-1-yl)-2-oxoethyl)carbamoyl)quinolin-7-yl)oxy)-2,15,23-trioxo-6,9,12-trioxa-3,16,22-triazapentacosan-25-yl)phenoxy)-3-((E)-2-(3,3-dimethyl-5-sulfonato-1-(3-(trimethylammonio)propyl)indolin-2-ylidene)ethylidene)cyclohex-1-en-1-yl)vinyl)-3,3-dimethyl-1-(3-(trimethylammonio)propyl)-3H-indol-1-ium-5-sulfonate

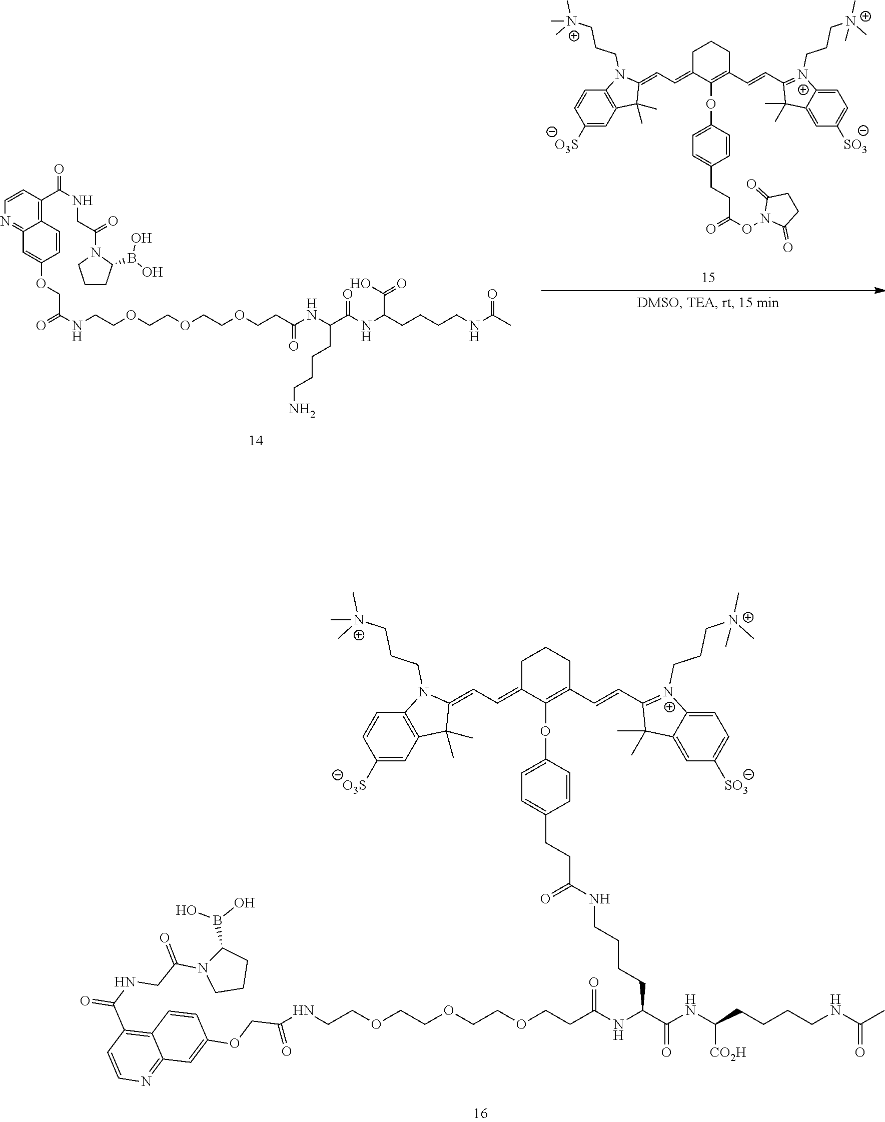

To a solution of 20-(4-acetamidobutyl)-17-(4-aminobutyl)-1-((4-((2-(2-boronopyrrolidin-1-yl)-2-oxo-ethyl)carbamoyl)quinolin-7-yl)oxy)-2,15,18-trioxo-6,9,12-trioxa-3,16,19-triazahenicosan-21-oic acid (5 mg, 0.0055 mmol) and 2-((E)-2-((E)-3-((E)-2-(3,3-dimethyl-5-sulfonato-1-(3-(trimethylammonio)-propyl)indolin-2-ylidene)ethylidene)-2-(4-(3-((2,5-dioxopyrrolidin-1-yl)oxy)-3-oxopropyl)phenoxy)cycl-ohex-1-en-1-yl)vinyl)-3,3-dimethyl-1-(3-(trimethylammonio)propyl)-3H-indol-1-ium-5-sulfonate (5.8 mg, 0.0055 mmol) in DMSO (0.2 ml) was added TEA (1.7 mg, 0.0166 mmol), and the reaction mixture was stirred at ambient temperature for 15 min. Full conversion was detected by LCMS. The crude product was subjected to HPLC purification. Desired fractions were combined and lyophilized to obtain the product (3 mg, 30%) as dark blue solid. LCMS: C₉₂H₁₂₈BN₁₂O₂₂S₂⁺: m/z: 1829.01, observed m/z=906.1 [M-OH/2]⁺.

### Synthesis of RTX-1403

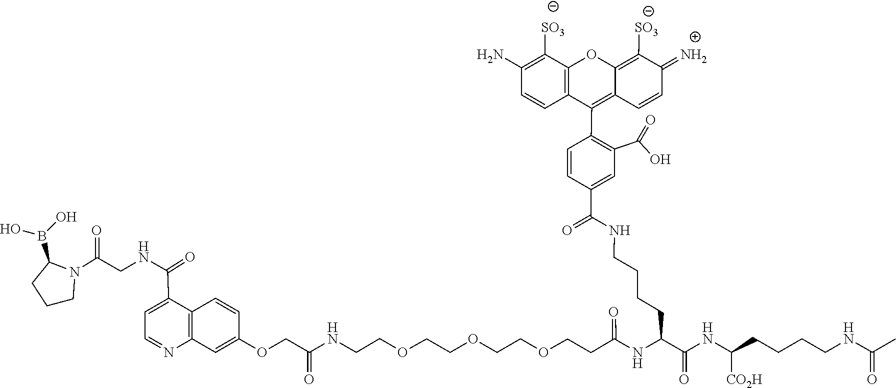

Prepared according to Example 10 using Alexa Fluor 488 in lieu of 2-((E)-2-((E)-3-((E)-2-(3,3-dimethyl-5-sulfonato-1-(3-(trimethylammonio)-propyl)indolin-2-ylidene)ethylidene)-2-(4-(3-((2,5-dioxopyrrolidin-1-yl)oxy)-3-oxopropyl)phenoxy)cycl-ohex-1-en-1-yl)vinyl)-3,3-dimethyl-1-(3-(trimethylammonio)propyl)-3H-indol-1-ium-5-sulfonate in procedure 84.

### Synthesis of RTX-1399R

Compounds of the invention with Macropa chelators can be prepared according to procedures disclosed in PCT/US2019/062479, the entire teachings of which are incorporated herein by reference.

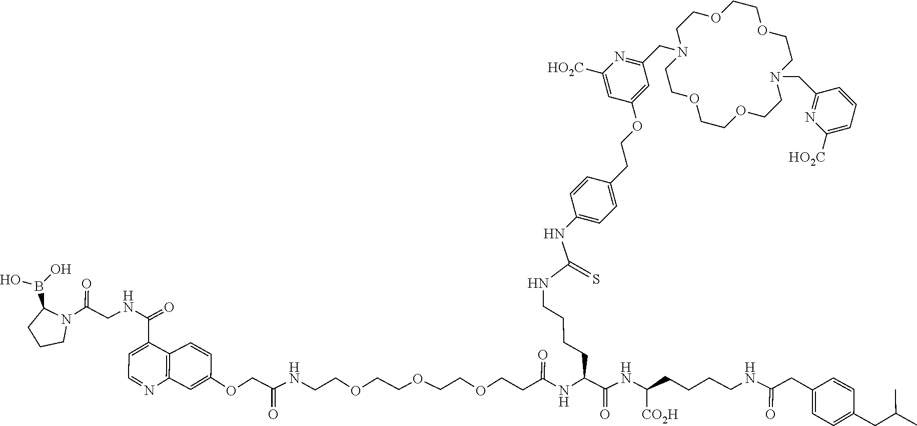 [SMILES: CC(C)Cc1ccc(CC(=O)NCCCC[C@H](NC(=O)[C@H](CCCCNC(=S)Nc2ccc(CCOc3cc(CN4CCOCCOCCN(Cc5cccc(C(=O)O)n5)CCOCCOCC4)nc(C(=O)O)c3)cc2)NC(=O)CCOCCOCCOCCNC(=O)COc2ccc3c(C(=O)NCC(=O)N4CCC[C@H]4B(O)O)ccnc3c2)C(=O)O)cc1]

Prepared according to Example 10 with the following changes:
Procedure 40 was used in lieu of Procedure 70.
In Procedure 40, 2-(4-isobutylphenyl)acetic acid was used in lieu of hexanoic acid.
The Macropa chelator was chelator was prepared and installed in place of the fluorophore using Procedures 85 and 86.

Procedure 85: Synthesis of 6-((16-((6-carboxypyridin-2-yl)methyl)-1,4,10,13-tetraoxa-7,16-diazacyclooctadecan-7-yl)methyl)-4-(4-isothiocyanatophenethoxy)picolinic acid (16):

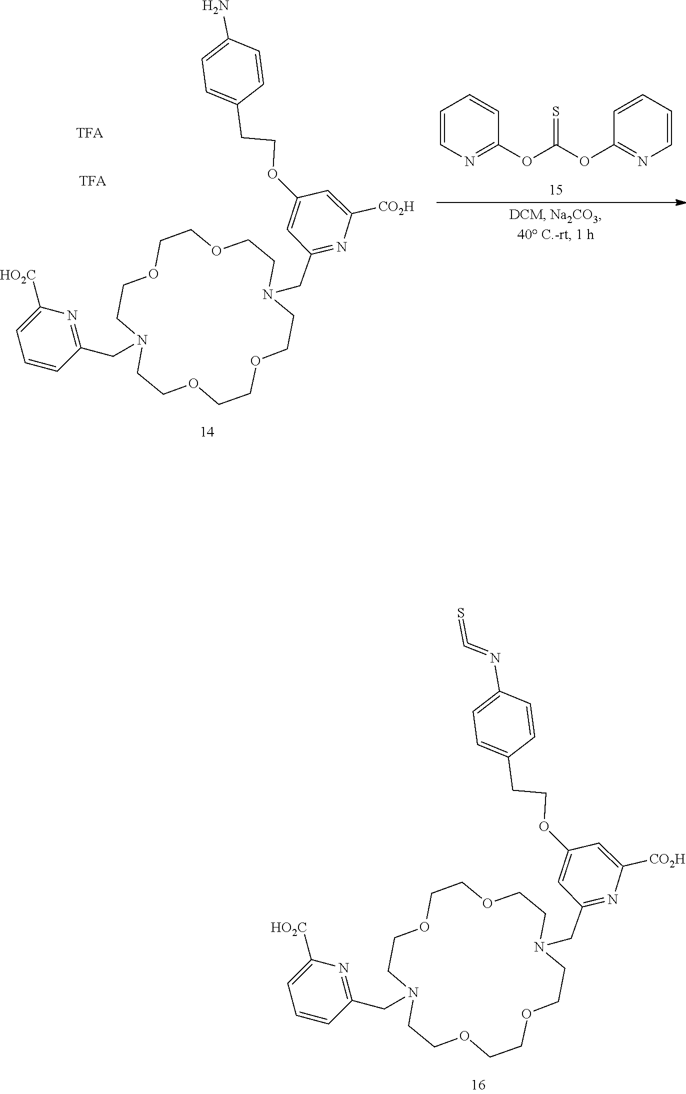 [SMILES: Nc1ccc(CCOc2cc(CN3CCOCCOCCN(Cc4cccc(C(=O)O)n4)CCOCCOCC3)nc(C(=O)O)c2)cc1 ; S=C(Oc1ccccn1)Oc1ccccn1 ; O=C(O)c1cccc(CN2CCOCCOCCN(Cc3cc(OCCc4ccc(N=C=S)cc4)cc(C(=O)O)n3)CCOCCOCC2)n1]

To a solution of 4-(4-aminophenethoxy)-6-((16-((6-carboxypyridin-2-yl)methyl)-1,4,10,13-tetraoxa-7,16-diazacyclooctadecan-7-yl)methyl)picolinic acid 2×TFA (120 mg, 0.14 mmol) in DCM (3.5 ml), was added Na₂CO₃ (44 mg, 0.42 mmol). The reaction mixture was stirred at 40° C. for 15 min (until a homogeneous solution was obtained). The reaction mixture was cooled to rt and O,O-di(pyridin-2-yl) carbonothioate (36 mg, 0.15 mmol) in 0.5 ml DCM was added to the reaction mixture. After LCMS analysis indicated consumption of the amine, the solids were removed by filtration and volatiles were removed under reduced pressure obtained the product 16 (100 mg, 100%) as a yellow color oil. LCMS: C₃₅H₄₃N₅O₉S: m/z: 709.81, observed m/z=710.4 [M+H]⁺.

Procedure 86: Synthesis of 4-(4-(3-((S)-1-((4-((2-((R)-2-boronopyrrolidin-1-yl)-2-oxoethyl)carbamoyl)quinolin-7-yl)oxy)-17-(((S)-1-carboxy-5-(2-(4-isobutylphenyl)acetamido)pentyl)carbamoyl)-2,15-dioxo-6,9,12-trioxa-3,16-diazahenicosan-21-yl)thioureido)phenethoxy)-6-((16-((6-carboxypyridin-2-yl)methyl)-1,4,10,13-tetraoxa-7,16-diazacyclooctadecan-7-yl)methyl)picolinic acid):

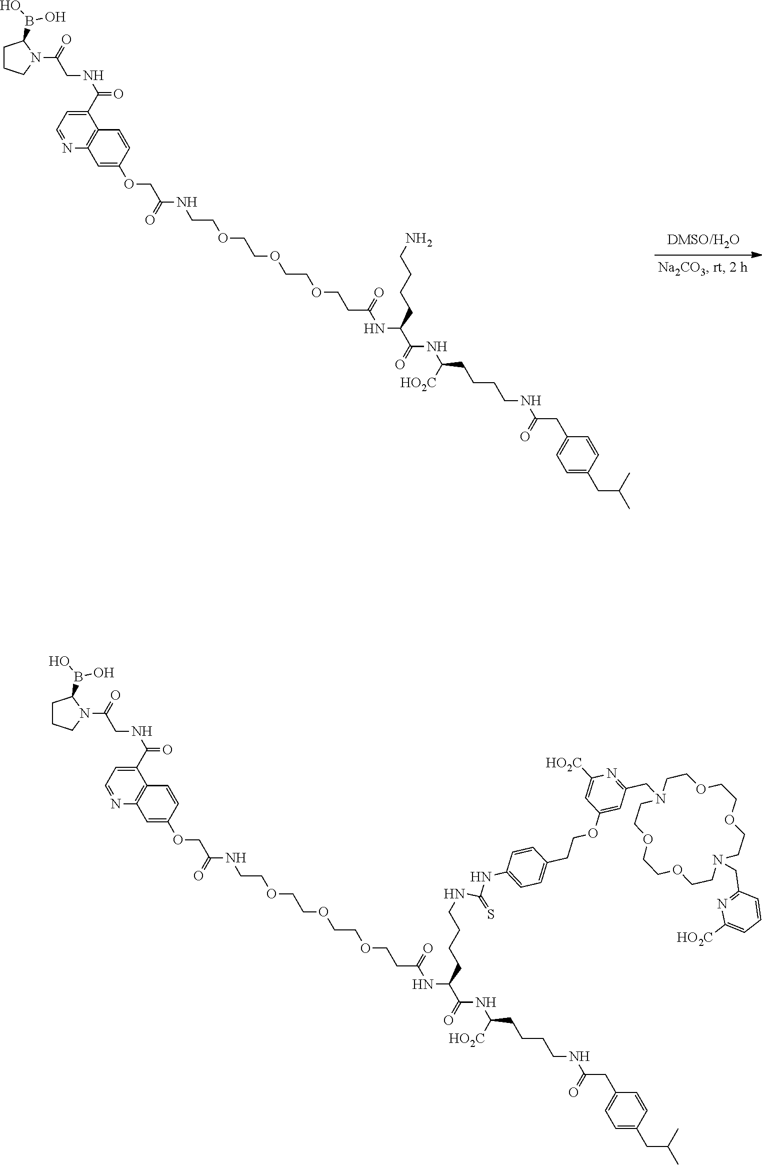 [SMILES: CC(C)Cc1ccc(CC(=O)NCCCC[C@H](NC(=O)[C@H](CCCCN)NC(=O)CCOCCOCCOCCNC(=O)COc2ccc3c(C(=O)NCC(=O)N4CCC[C@H]4B(O)O)ccnc3c2)C(=O)O)cc1 ; CC(C)Cc1ccc(CC(=O)NCCCC[C@H](NC(=O)[C@H](CCCCNC(=S)Nc2ccc(CCOc3cc(CN4CCOCCOCCN(Cc5cccc(C(=O)O)n5)CCOCCOCC4)nc(C(=O)O)c3)cc2)NC(=O)CCOCCOCCOCCNC(=O)COc2ccc3c(C(=O)NCC(=O)N4CCC[C@H]4B(O)O)ccnc3c2)C(=O)O)cc1]

To a solution of N²-((1-((4-((2-((R)-2-boronopyrrolidin-1-yl)-2-oxoethyl)carbamoyl)quinolin-7-yl)oxy)-2-oxo-6,9,12-trioxa-3-azapentadecan-15-oyl)-L-lysyl)-N⁶-(2-(4-isobutylphenyl)acetyl)-L-lysine (21 mg, 0.023 mmol) in DMSO:H₂O (0.5 ml:0.5 ml), were added Na₂CO₃ (8 mg, 0.069 mmol) and 6-((16-((6-carboxypyridin-2-yl)methyl)-1,4,10,13-tetraoxa-7,16-diazacyclooctadecan-7-yl)methyl)-4-(4-isothiocyanatophenethoxy)picolinic acid (20 mg, 0.027 mmol, dissolved in 0.5 ml DMSO), and stirred the reaction mixture at rt for 2 h. After LCMS analysis indicated complete consumption of the amine, the solids were removed by filtration and the crude product was submitted for HPLC purification. Desired fractions were combined and lyophilized to obtain the product (28 mg, 76%) as an off-white solid. LCMS: C₈₆H₁₁₈BN₁₃O₂₃S: m/z: 1743.83, observed m/z=855.6.

### Example 11—Radiolabeling Methods and Results

General procedure: 20 μg of precursor (unless otherwise noted) are combined with the indicated radioisotopes The radioisotopes are either eluted from a generator (for 68Ga) or obtained commercially in HCl solutions that were buffered with varying amounts of 3 N NaOAc to obtain a final pH of 4-6. Reactions with a C18 Sep-Pak Lite cartridge as needed for additional purity and/or reformulation for injection. Labeling results are shown in Table 1.

TABLE 1
Results for representative labeling reactions

| | | Precursor | T | Time | | | RCY | RCP | |
|---|---|---|---|---|---|---|---|---|---|
| Compound | Isotope | (μg) | (° C.) | (min) | Buffer | n | (%) | (%) | Purification |
| RTX- | ⁶⁸Ga | 20 | 90 | 10 | 3N | 1 | 54.6 | 99.9 | C-18 Lite |
| 1309S | | | | | NaOAc | | | | |
| RTX- | ⁶⁸Ga | 20 | 90 | 10 | 3N | 1 | 63.6 | 99.9 | C-18 Lite |
| 1316S | | | | | NaOAc | | | | |
| RTX- | ⁶⁸Ga | 20 | 90 | 10 | 3N | 2 | 76.2 | 99.9 | C-18 Lite |
| 1317S | | | | | NaOAc | | | | |
| RTX- | ⁶⁸Ga | 20 | 90 | 10 | 3N | 1 | 67.4 | 99.9 | C-18 Lite |
| 1318S | | | | | NaOAc | | | | |
| RTX- | ⁶⁸Ga | 20 | 90 | 10 | 3N | 1 | 73.2 | 96.1 | C-18 Lite |
| 1339S | | | | | NaOAc | | | | |
| RTX- | ⁶⁴Cu | 20 | 90 | 10 | 3N | 2 | 98.8 | 99.9 | C-18 Lite |
| 1339S | | | | | NaOAc | | | | |
| RTX- | ⁶⁸Ga | 20 | 90 | 10 | 3N | 1 | 74.1 | 96.7 | C-18 Lite |
| 1340S | | | | | NaOAc | | | | |
| RTX- | ⁶⁴Cu | 20 | 90 | 10 | 3N | 2 | 99.0 | 99.9 | C-18 Lite |
| 1340S | | | | | NaOAc | | | | |
| RTX- | ⁶⁸Ga | 20 | 90 | 10 | 3N | 2 | 88.5 | 98.0 | None |
| 1350S | | | | | NaOAc | | | | |
| RTX- | ⁶⁸Ga | 20 | 90 | 10 | 3N | 1 | 88.0 | 97.5 | None |
| 1351S | | | | | NaOAc | | | | |
| RTX- | ⁶⁸Ga | 20 | 90 | 10 | 3N | 1 | 88.9 | 98.4 | None |
| 1352S | | | | | NaOAc | | | | |
| RTX- | ⁶⁸Ga | 20 | 90 | 10 | 3N | 1 | 86.6 | 95.9 | None |
| 1353S | | | | | NaOAc | | | | |
| RTX- | ⁶⁸Ga | 20 | 90 | 10 | 3N | 1 | 88.6 | 98.1 | None |
| 1354S | | | | | NaOAc | | | | |
| RTX- | ¹⁷⁷Lu | 5 | 90 | 10 | 3N | 3 | 99.9 | 99.9 | None |
| 1354S | | | | | NaOAc | | | | |
| RTX- | ⁶⁸Ga | 20 | 90 | 10 | 3N | 2 | 85.1 | 94.1 | None |
| 1356R | | | | | NaOAc | | | | |
| RTX- | ⁶⁸Ga | 20 | 90 | 10 | 3N | 2 | 86.4 | 92.3 | None |
| 1357R | | | | | NaOAc | | | | |
| RTX- | ⁶⁸Ga | 20 | 90 | 10 | 3N | 2 | 83.2 | 92.2 | None |
| 1359R | | | | | NaOAc | | | | |
| RTX- | ¹⁷⁷Lu | 5 | 90 | 10 | 3N | 3 | 96.0 | 96.1 | None |
| 1359R | | | | | NaOAc | | | | |
| RTX- | ⁶⁸Ga | 20 | 90 | 10 | 3N | 9 | 95.3 | 99.9 | C-18 Lite |
| 1363S | | | | | NaOAc | | | | |
| RTX- | ⁶⁴Cu | 20 | 90 | 10 | 3N | 7 | 99.5 | 99.9 | C-18 Lite |
| 1363S | | | | | NaOAc | | | | |

### Example 12—Compounds of the Invention Bind to FAP with High Affinity

Protease reactions were assembled in 384 well plates (Greiner) in a total volume of 20 uL as described below.

Recombinant proteins were pre-diluted in assay buffer comprising of 100 mM HEPES, pH 7.5, 0.1% BSA, 0.01% Triton X-100, 1 mM DTT, and dispensed into 384 well plate (10 uL per well). Test compounds were serially pre-diluted in DMSO and added to the assay wells by acoustic dispensing (Labcyte Echo 550). Control samples (0%-inhibition in the absence of inhibitor, DMSO only) and 100%-inhibition (in the absence of enzyme) were assembled in replicates of four and used to calculate the %-inhibition in the presence of compounds. Concentration of DMSO was equalized to 1% in all samples.

Compounds were pre-incubated with enzymes for 15 minutes. Human FAP was obtained from Enzo, catalogue number BML-SE409-0010. The reactions are initiated by addition of 10 uL of 2×FAM-labeled substrate peptide (FAM-GPRPFNYLAKK-NH2) prepared in the same assay buffer. Final concentration of enzymes were 0.5 nM. Final concentration of substrate peptides was 1 uM.

The reactions were allowed to proceed at room temperature. Incubation time was three hours for human FAP, 0.5 hours for mouse FAP. After incubation, kinase reactions were quenched by addition of 50 uL of termination buffer: assay buffer supplemented with reference inhibitor at 100×IC₅₀.

Terminated plates were analyzed using a microfluidic electrophoresis instrument (Caliper LabChip® 3000, Caliper Life Sciences/Perkin Elmer). A change in relative intensity of the peptide substrate and cleaved product was the parameter measured. Activity in each test sample was determined as the product to sum ratio (PSR):P/(S+P), where P is the peak height of the product, and S is the peak height of the substrate. Percent inhibition (Pᵢₙₕ) was determined using the following equation: Pᵢₙₕ=(PSR_{0% inh}−PSR_{compound})/(PSR_{0% inh}−PSR_{100% inh})*100, in which: PSR_{compound} is the product/sum ratio in the presence of compound, PSR_{0% inh} is the product/sum ratio in the absence of compound and the PSR_{100% inh} is the product/sum ratio in the absence of the enzyme. To determine IC₅₀ of compounds (50%-inhibition) the %-inh data (Pᵢₙₕ versus compound concentration) are fitted by a 4 parameter sigmoid dose-response model using XLfit software (IDBS). These values are compiled in Table 2 and grouped, where A represents an IC₅₀<0.1 nM; B is an IC₅₀ from 0.1 to 0.5 nM; C is an IC₅₀ from 0.5 to 5.0 nM; D is an IC₅₀ from 5.0 to 100 nM; E an IC₅₀>100 nM.

TABLE 2
Results for Binding Affinity Studies and Mass Characterization Data

| | IC₅₀ | | | | IC₅₀ | | |
|---|---|---|---|---|---|---|---|
| Compound | (nm) | MS Calc (Found) | | Compound | (nM) | MS Calc (Found) | |
| RTX-1309R | C | 1636.58 | (1638.5) | RTX-1334S | E | 550.22 | (565.3) |
| RTX-1312R | C | 1333.60 | (1335.3) | RTX-1335S | | 1086.52 | (1088) |
| RTX-1312S | A | 1333.60 | (1335.3) | RTX-1336S | | 1275.55 | (1128.2) |
| RTX-1313R | D | 1283.59 | (1285.2) | RTX-1337S | | 1129.51 | (1131.2) |
| RTX-1314 | E | 1332.61 | (1352.8) | RTX-1338S | | 1318.54 | (1171.4) |
| RTX-1315 | E | 1350.62 | (1335.3) | RTX-1339S | B | 1168.58 | (1170.2) |
| RTX-1316R | C | 1465.70 | (1468.6) | RTX-1340S | B | 1196.61 | (1198.5) |
| RTX-1316S | A | 1465.70 | (1467.4) | RTX-1341S | A | 1808.87 | (1810.8) |
| RTX-1317R | C | 1361.63 | (1363.6) | RTX-1342S | A | 1493.73 | (748.8) |
| RTX-1317S | B | 1361.63 | (1363.6) | RTX-1343S | A | 1058.49 | (1116.2) |
| RTX-1318R | C | 1389.66 | (1391.2) | RTX-1350S | A | 1353.72 | (1355.2) |
| RTX-1318S | A | 1389.66 | (1392.4) | RTX-1351S | A | 1429.75 | (1431.4) |
| RTX-1319R | B | 1352.61 | (667.8) | RTX-1352S | A | 1429.75 | (1852.9961) |
| RTX-1320R | B | 1408.68 | (1374.3) | RTX-1353S | B | 1269.62 | |
| RTX-1321R | B | 1380.64 | (1365.4) | RTX-1354S | A | 1401.72 | (1403.2) |
| RTX-1322R | B | 1484.71 | (1468) | RTX-1355R | B | 1372.73 | (1375) |
| RTX-1323R | D | 438.19 | | RTX-1356R | B | 1448.76 | (1432.6) |
| RTX-1323S | C | 438.19 | | RTX-1357R | A | 1872.01 | (928.9) |
| RTX-1324S | A | 1030.46 | (1032.2) | RTX-1358R | | 1288.64 | (1254.3) |
| RTX-1325S | C | 1073.45 | (1075.4) | RTX-1359R | A | 1420.73 | (1404.4) |
| RTX-1326S | C | 338.14 | (339.4) | RTX-1360S | A | 1325.69 | (1327.5) |
| RTX-1327S | B | 527.24 | (259.3) | RTX-1361R | A | 1344.70 | |
| RTX-1328S | C | 613.27 | (614.2) | RTX-1363S | A | 1224.64 | (1226.1) |
| RTX-1329S | B | 612.29 | (613.3) | RTX-1363S-Cu | A | 1285.55 | (1287.3) |
| RTX-1330S | B | 711.36 | (712.6) | RTX-1370S | | 1690.65 | (847.8) |
| RTX-1331S | B | 713.30 | (714.7) | RTX-1389R | | 1827.88 | (906.1) |
| RTX-1332S | B | 826.39 | (827.5) | RTX-1399R | B | 1743.83 | (855.6) |

| | | | | | |
|---|---|---|---|---|---|
| RTX-1333S | E | 461.17 | RTX-1403R | 1417.44 | (702) |

It has been found from the inhibition studies described above that the S stereoisomers are considerably more potent than the R stereoisomers. The inhibition studies above also revealed that extending the chain off of the quinolinyl core of

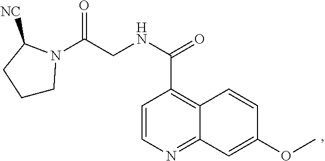 [SMILES: COc1ccc2c(C(=O)NCC(=O)N3CCC[C@H]3C#N)ccnc2c1]

which has an LC₅₀ of 1.5 nM, significantly improved activity. Specifically, the lengthening the chain piecemeal provided an improvement with each extension, and the addition of the final amino acid (lysine) provided an additional increase in activity.

Greater discrimination was observed between certain compounds of the invention and FAPI-46 in a surface plasmon resonance assay data (FAP-46 was measured to have an affinity of 255 pM, whereas certain disclosed compounds had an affinity of 20 pM or stronger). This was also reflected in the U87 bioD studies described below in comparison with FAPI-46, where certain compounds of the invention exhibited significantly greater retention in vivo

### Example 13—Radiolabeling of a FAP-Targeted Compound with ¹⁸F Direct Labelling

¹⁸F received from the manufacturer was loaded on to a Sep Pak QMA Light Plus cartridge and eluted into a glass reaction vial using a Cs₂CO₃/K₂₂₂ solution. Eluted ¹⁸F was azeotropically dried using acetonitrile (3×1 ml) at 95° C. under a stream of N₂. 2.5 mg of the FAP-targeted compound was dissolved in in 0.3 ml DMSO which was then added to the above vial containing the dried ¹⁸F. The vial was sealed and heated to 90° C. for 15 minutes. The vial was cooled and a solution of 1 N HCl was added to vial, which was then sealed and heated to 95° C. for 10 minutes. The reaction mixture was cooled and neutralized and then loaded on to a Semi Prep HPLC column for purification and formulated appropriately for use. Alternatively, a suitable FAP-targeted compound having a chelator can be used for ¹⁸F AlF labelling, wherein AlCl₃ stock in acetate buffer (22.5 μL, 45 nmol, 0.9 eq) was added to a ¹⁸F solution in sodium acetate (200 uL) and the reaction vial was left at room temperature for 5 min. From precursor stock, a FAP-targeted compound solution (50 nmol scale, 12.5 μL) was then added to the above vial. pH was corrected to about 4.0 by addition of 1% v/v Acetic acid in water (15 μL). Co-solvent, 200 μL of EtOH, was added and the reaction vial was sealed, then heated at 100° C. for 15 mins. Reaction mixture was diluted to 9.5 ml and loaded on to a tC18 Sep Pak cartridge. Product is eluted with 300 uL acidified EtOH and formulated for use.

### Example 14—Detection of Tumors in Murine Models with Compounds of the Invention

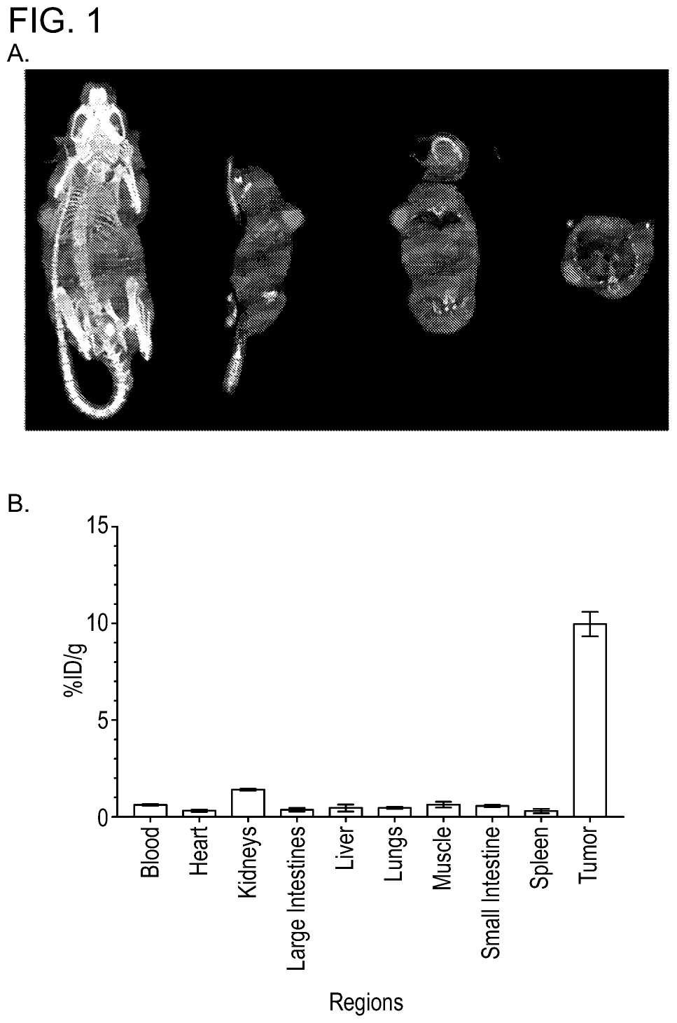

BALB/c nude mice were transplanted with approximately 10⁷ U-87 (human glioblastoma) cells and the tumors allowed to develop. 1 μg of an ¹⁸F-labeled FAP-targeted agent of the invention, RTX-1312S (prepared according to Example 13), was administered to the mice by intravenous injection (e.g., tail vein), which were then sacrificed after one hour. FIG. 1a illustrates tumor binding of the ¹⁸F-labeled FAP-targeted compound in mice. FIG. 1b shows the biodistribution of the compound in the various murine tissues.

### Example 15—Biodistribution Studies

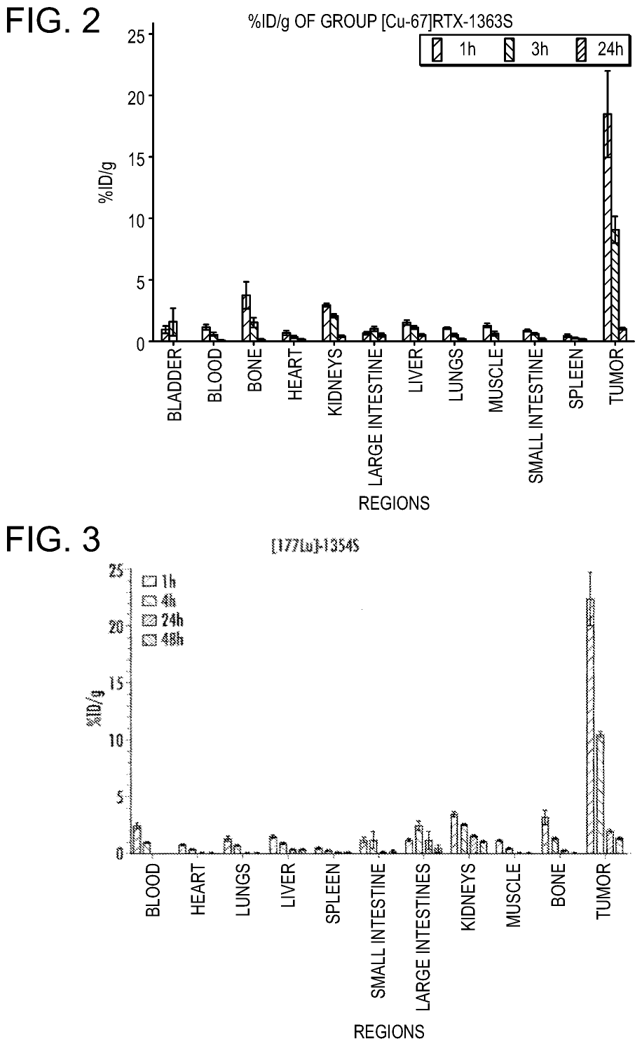

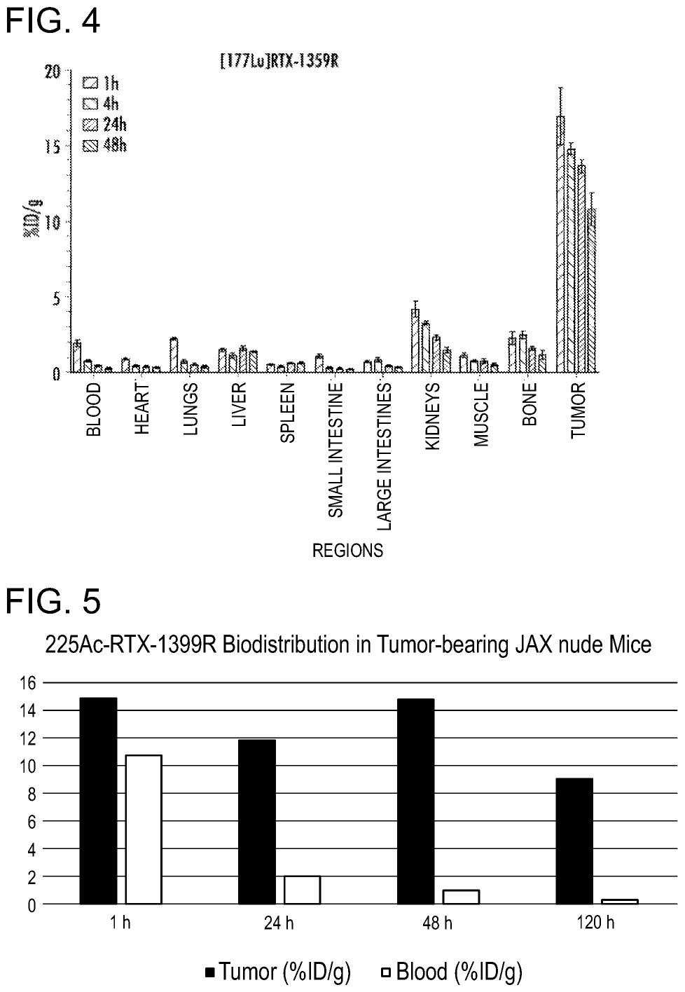

Female BALB/C nude mice were inoculated subcutaneously on the right shoulder with U87MG cells in 1:1 matrigel:PBS. When the tumors reached a volume of 150-500 mm³, 1 μg of radiolabeled ligand was administered intravenously (IV) via tail vein. At various time points post-injection, mice were humanely euthanized via exsanguination and tissue samples (bladder, blood, urine, bone (femur), heart, lungs, liver, both kidneys, small intestines (including contents), large intestines (including contents), muscle (quadriceps), tumor, and tail) were resected, weighed and counted with a gamma counter. The activity of each collected tissue was measured in units of counts per minute (CPM). Triplicate aliquots of the radiotracer were also assayed in the gamma counter to calculate a factor for converting counts to units of activity (μCi/CPM). Values were decay corrected to the time of injection and corrected for background radiation. The biodistribution of compounds [Cu-67]RTX-1363S, [177Lu]RTX-1354S, and [177Lu]1359R showed localization of the compounds within the tumor and minimal concentrations within other organs (See FIG. 2-4). This biodistribution demonstrated increased selectivity for the tumor over time demonstrating the high affinity for FAP.

### Example 16—Biodistribution Studies of [Ac-225]RTX-1399

Radiolabeling Procedure: 225Ac salt was dissolved in 600 μl of 1M NHOAc. 100 μl of the 225Ac stock solution was added to a reaction vial. 50 μl (50 μg) of precursor was dissolved in DMSO and then added to the reaction vial. 3 mL of normal saline (0.9%) was added and the reaction was shaken at 25° C. for 30 minutes. The radiolabeling yield and purity are measured by TLC using Macherey-Nagel Aluminum sheets (ALUGRAM SIL G/UV254, 4×8 cm spotted with 2-5 μl of solution and placed in a chamber of 50 mM EDTA in normal saline) and analyzed using a Packard Phosphorimager for quantification. 1 μg of radiolabeled compound was administered intravenously (IV) into tumor-bearing JAX nude mice. Over the course of 120 hours, it is evident that the target compound is localizing in the tumor and being filtered from the blood (See FIG. 5.)

### EQUIVALENTS

While certain embodiments have been illustrated and described a person with ordinary the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only and is not intended to be limiting. Thus, it is intended that the specification be considered as exemplary only with the breadth, scope and spirit of the present technology indicated only by the appended claims, definitions therein and any equivalents thereof.

All publications, patents, and other documents referred to in this specification are herein incorporated by reference in its entirety.

We claim:

1. A compound represented by the following structural formula: 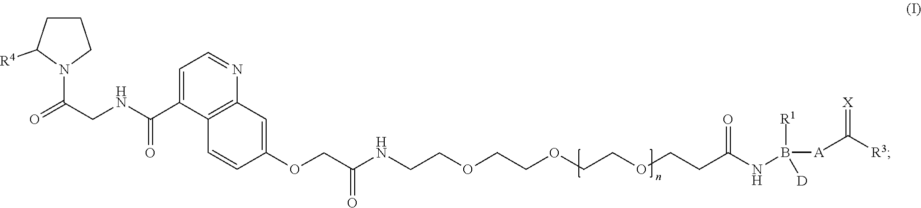
or a pharmaceutically acceptable salt thereof, wherein:
n is 0 or 1;
A is NH, O, S or CR⁶R⁷;
B comprises a branched, unbranched or cyclic aliphatic group of up to 30 carbon atoms optionally interrupted by up to 10 heteroatoms or a peptidyl chain of up to 20 amino acid residues, wherein B is optionally substituted with 1-5 groups selected from F, Cl, Br, I, ═O, OR⁶, OCOR⁶, COOR⁶, CN, ═NR⁶, NR⁶R⁷, ═S, and SR⁶, provided that B comprises at least 3 atoms in a chain between group D and the group A;
D is selected from the group consisting of OPO₃H₂, PO₃H₂, OSO₃H, SO₃H and COOH or a C₁-C₄ alkyl ester thereof;
X is O or S;
R¹ is a chelating group, an optical dye or fluorophore, a cytotoxic agent, an immune stimulant, or a benzoyl group optionally substituted by one or more groups represented by R⁵;
R³ is C₁-C₈ alkyl or C₁-C₄ aralkyl, wherein:
the alkyl and aryl portions of the aralkyl are each optionally and independently substituted with F, Cl, Br, I, branched, unbranched or cyclic C₁-C₆ aliphatic group, OR⁶, OCOR⁶, COOR⁶, CHO, COR⁶, CH₂OR⁶, NR⁶R⁷, CH₂NR⁶ R⁷, SR⁶, ═O, ═S and ═NH;
R⁴ is CN or B(OH)_{2;} and
each R⁵ is independently selected from halo, cyano, halomethyl, N⁺(CH₃)₃W⁻ wherein W⁻ is a pharmaceutically acceptable anion; and
R⁶ and R⁷ are independently selected from the group consisting of H or a C₁-C₆ alkyl.

2. The compound of claim 1 or a pharmaceutically acceptable salt thereof where B is a branched, unbranched or cyclic aliphatic group of up to 30 carbon atoms optionally interrupted by up to 10 heteroatoms or a peptidyl chain of up to 20 amino acid residues (for example 3-20 carbon atoms optionally interrupted by up to 6 heteroatoms or up to 5 amino acid residues), wherein B is optionally substituted with 1-5 groups selected from F, Cl, Br, I, ═O, OR⁶, OCOR⁶, COOR⁶, CN, ═NR⁶, NR⁶R⁷, ═S, and SR⁶, provided that B comprises at least 3 atoms in a chain between group D and the group A.

3. The compound of claim 1 or 2 represented by the following structural formula: 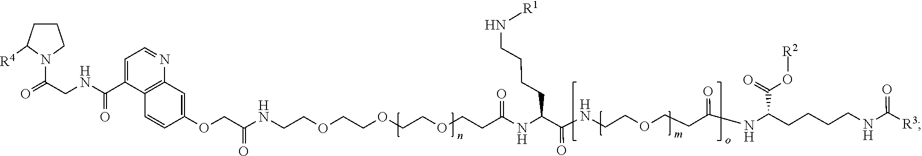
or a pharmaceutically acceptable salt thereof, wherein m is an integer from 0 to 12; o is 0 or 1; and R² is H or C₁-C₄ alkyl.

4. The compound of claim 3 represented by the following structural formula: 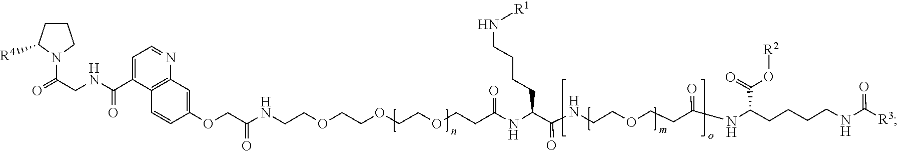
or a pharmaceutically acceptable salt thereof.

5. The compound of claim 3 represented by the following structural formula: 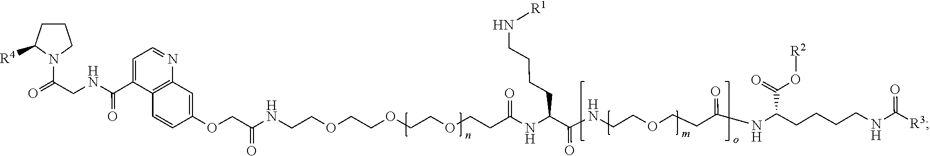
or a pharmaceutically acceptable salt thereof.

6. The compound of any one of claims 1-5, or a pharmaceutically acceptable salt thereof, wherein R³ is C₁-C₈ alkyl or C₁-C₄ aralkyl optionally substituted with C₁-C₄ alkyl.

7. The compound of any one of claims 1-6, or a pharmaceutically acceptable salt thereof, wherein R³ is methyl, propyl, pentyl, heptyl, (4-isobutylphenyl)methyl, (4-isobutylphenyl)propyl.

8. The compound of any one of claims 3 to 5, or a pharmaceutically acceptable salt thereof, wherein o is 1 and m is 3 to 12.

9. The compound of claim 6, or a pharmaceutically acceptable salt thereof, wherein m is 8.

10. The compound of any one of claims 3 to 5, or a pharmaceutically acceptable salt thereof, wherein o is 0.

11. The compound of any one of claims 1 to 10, or a pharmaceutically acceptable salt thereof. wherein n is 1.

12. The compound of any one of claims 1 to 11, or a pharmaceutically acceptable salt thereof, wherein R¹ is a fluorophore or an optical dye.

13. The compound of claim 12, or a pharmaceutically acceptable salt thereof, wherein the fluorophore is 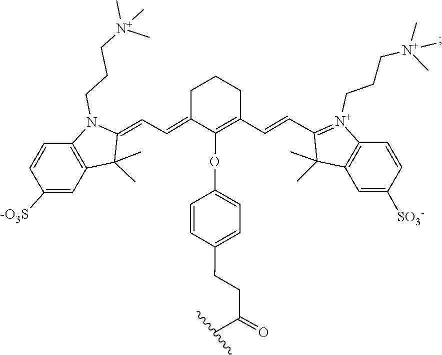
and the optical dye is selected from the group consisting of: a carbocyanin, indocarbocyanin, oxacarbocyanin, thiacarbocyanin, merocyanin, polymethine, coumarin, rhodamine, xanthene, fluorescein, Borodipyrromethane (BODIPY), VivoTag-680, VivoTag-S750, AlexaFluor dyes (e.g., AlexaFluor660, AlexaFluor680, AlexaFluor700, AlexaFluor750, AlexaFluor790) and DylightFluor dyes.

14. The compound of any one of claims 1 to 11 or a pharmaceutically acceptable salt thereof, wherein R¹ is a chelating group that is the residue of a chelating agent.

15. The compound of claim 14 or a pharmaceutically acceptable salt thereof, wherein the chelating group is the residue of a chelating agent selected from 1,4,7-triazacyclononane-1,4,7-triacetic acid (NOTA), p-SCN-Bn-NOTA, 1,4,7,10-tetraazacyclododecane-1,4,7,10-tetraacetic acid (DOTA), p-SCN-Bn-DOTA (also known as 2B-DOTA-NCS), PIP-DOTA, diethylenetriaminepentaacetic acid (DTPA), PIP-DTPA, AZEP-DTPA, ethylenediamine tetraacetic acid (EDTA), triethylenetetraamine-N,N,N′,N″,N′″,N′″-hexa-acetic acid (TTHA), 7-[2-(bis-carboxymethylamino)-ethyl]-4,10-bis-carboxymethyl-1,4,7,10-tetraaza-cyclododec-1-yl-acetic acid (DEPA), 2,2′,2″-(10-(2-(bis(carboxymethyl)amino)-5-(4-isothiocyanatophenyl) pentyl)-1,4,7,10-tetraazacyclododecane-1,4,7-triyl)triacetic acid (3p-C-DEPA-NCS), NETA, {4-carboxymethyl-7-[2-(carboxymethylamino)-ethyl]-perhydro-1,4,7-triazonin-1-yl}-acetic acid (NPTA), diacetylpyridinebis(benzoylhydrazone), 1,4,7,10,13,16-hexaazacyclooctadecane N,N′,N″,N′″,N″″,N′″″-hexaaceticacid (HEHA), octadentate terephthalamide ligands, 2,2′-(4-(2-(bis(carboxymethyl)amino)-5-(4-isothiocyanatophenyl)pentyl)-10-(2-(bis(carboxymethyl)amino)ethyl)-1,4,7,10-tetraazacyclododecane-1,7-diyl)diacetic acid, N,N′-bis[(6-carboxy-2-pyridil)methyl]-4,13-diaza-18-crown-6 (H2macropa), 6-((16-((6-carboxypyridin-2-yl)methyl)-1,4,10,13-tetraoxa-7,16-diazacyclooctadecan-7-yl)methyl)-4-isocyanatopicolinic acid (macropa-NCO), 6-((16-((6-carboxypyridin-2-yl)methyl)-1,4,10,13-tetraoxa-7,16-diazacyclooctadecan-7-yl)methyl)-4-isothiocyanatopicolinic acid (macropa-NCS), 3,9-carboxymethyl-6-(2-methoxy-5-isothiocyanatophenyl)carboxymethyl-3,6,9,15-tetraazabicyclo-[9.3.1]pentadeca-1(15), 11,13-triene and 2-[4,7,10-tris(2-amino-2-oxoethyl)-1,4,7,10-tetrazacyclododec-1-yl]acetamide (TCMC or DOTAM).

16. The compound of claim 14 or a pharmaceutically acceptable sat thereof, wherein the residue of the chelating agent is the residue of macropa-NCS or macropa-NCO.

17. The compound of claim 14 or a pharmaceutically acceptable salt thereof, wherein the residue of a chelating agent is the residue of p-SCN-Bn-NOTA, p-SCN-Bn-DOTA, NOTA or DOTA,

18. The compound of any one of claims 1-11 or 14 or a pharmaceutically acceptable salt thereof, wherein the chelating group is the residue of a siderophore.

19. The compound of any one of claims 1 to 11 or a pharmaceutically acceptable salt thereof, wherein:
R¹ is a benzoyl group optionally substituted by one or more groups represented by R⁵;
each R⁵ is independently selected from halo, cyano, halomethyl, N⁺(CH₃)₃W⁻; and
W⁻ is a pharmaceutically acceptable anion.

20. The compound of claim 19 or a pharmaceutically acceptable salt thereof, wherein each R⁵ is independently selected from fluoro, cyano, trifluoromethyl, N⁺(CH₃)₃W⁻.

21. The compound of claim 19 or 20, or a pharmaceutically acceptable salt thereof, wherein the halo or fluoro group represented by R⁵ is ¹⁸F.

22. The compound of any one of claims 1-21 or a pharmaceutically acceptable salt thereof, wherein R² is H and R⁴ is CN.

23. The compound of claim 1 or pharmaceutically acceptable salt thereof, represented by a structural formula selected from:

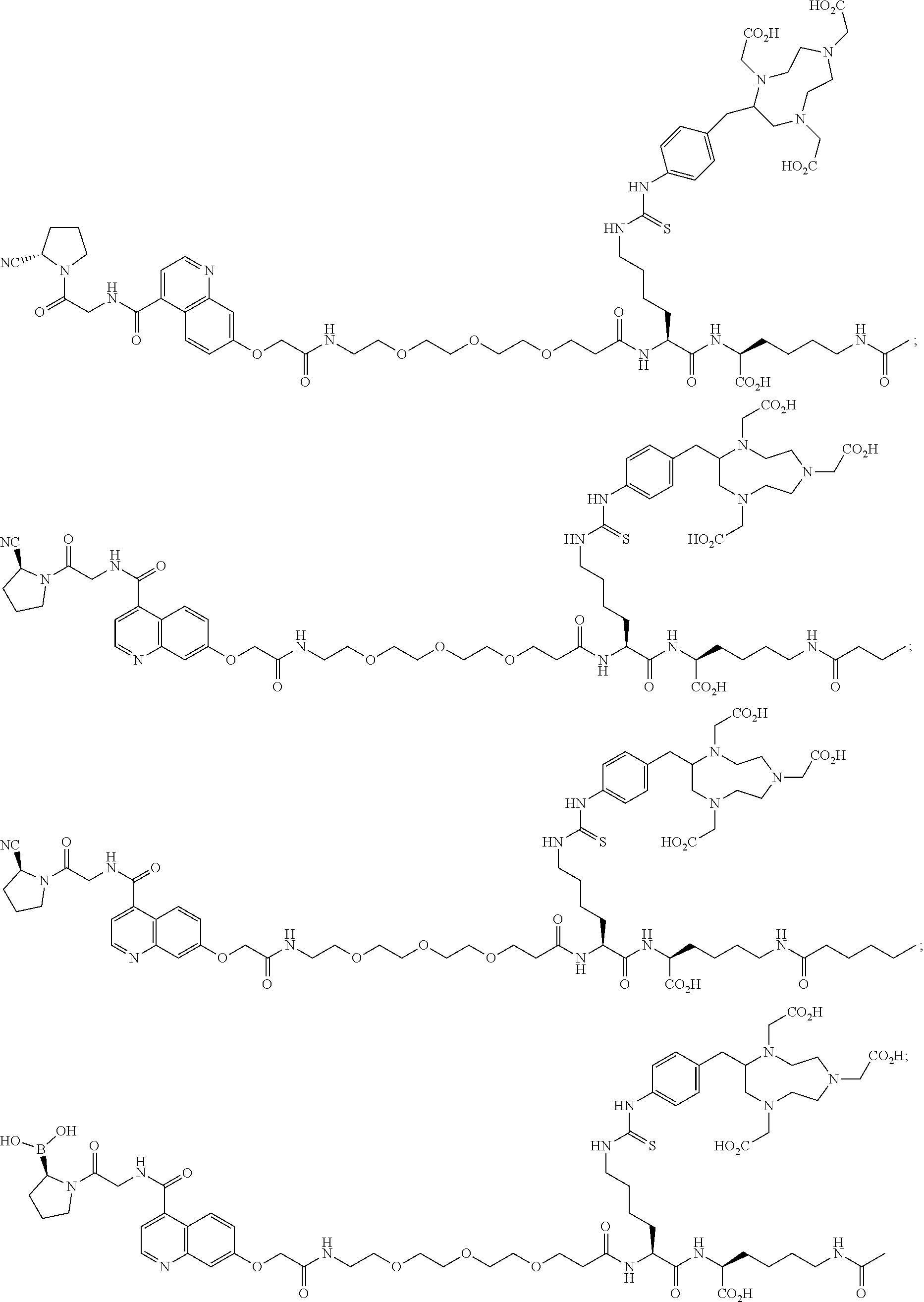

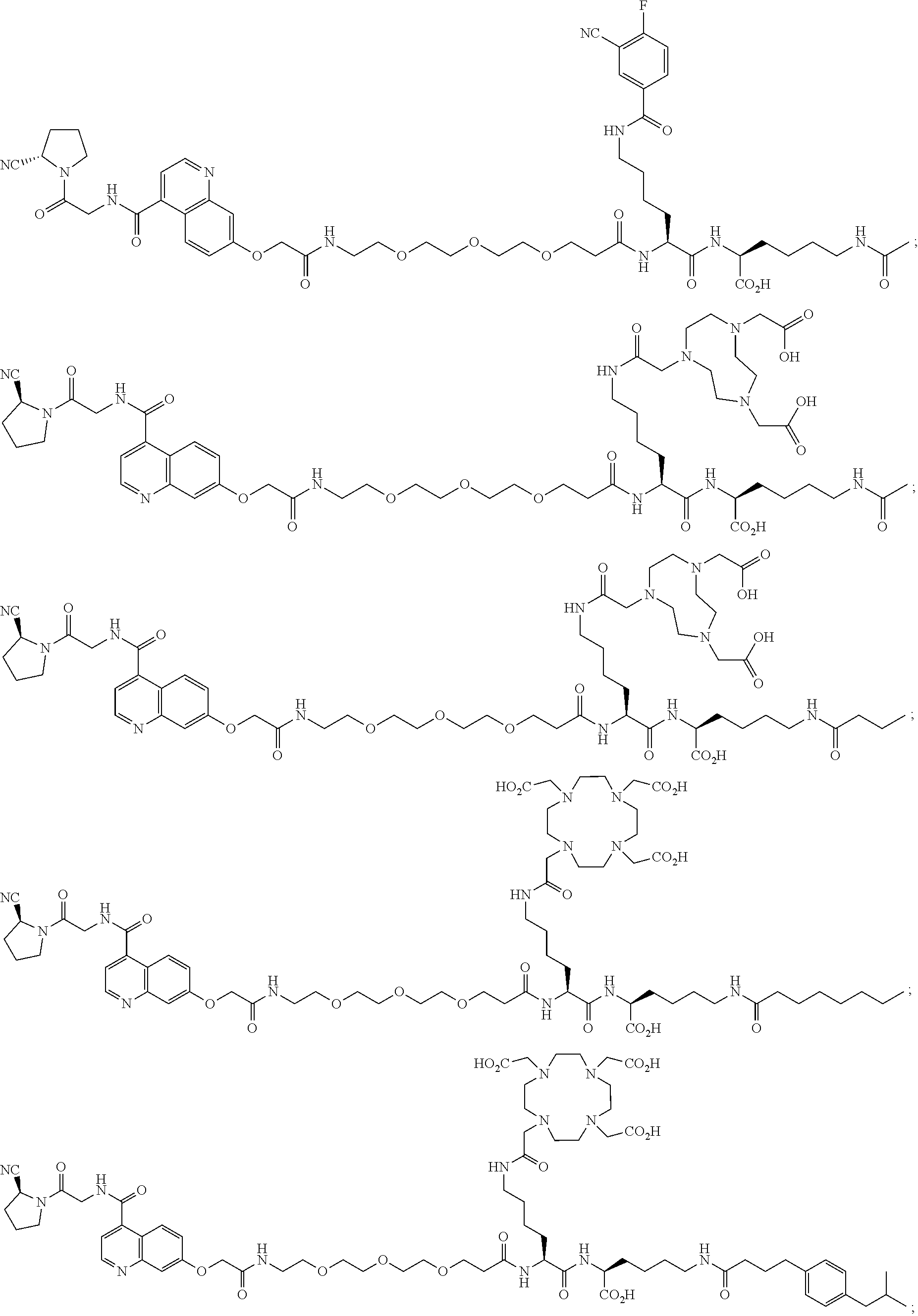

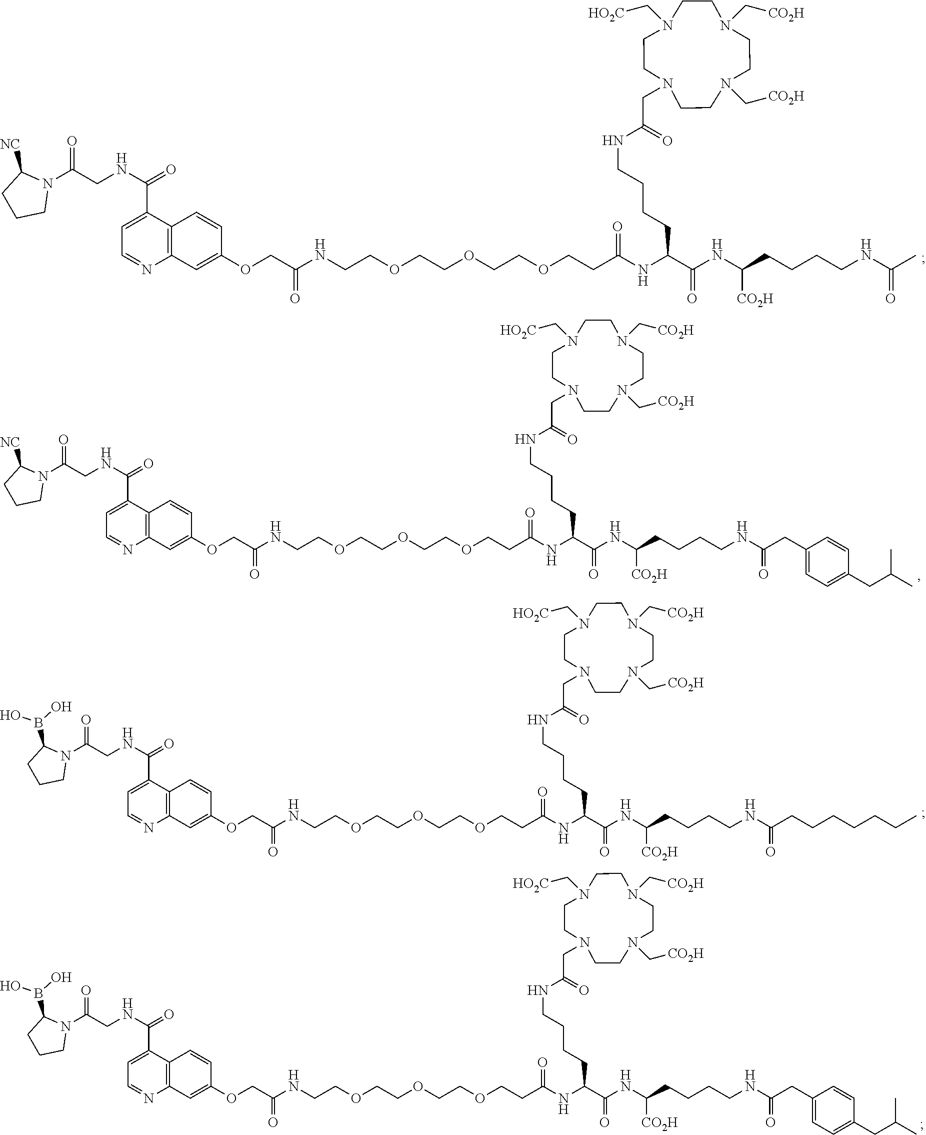

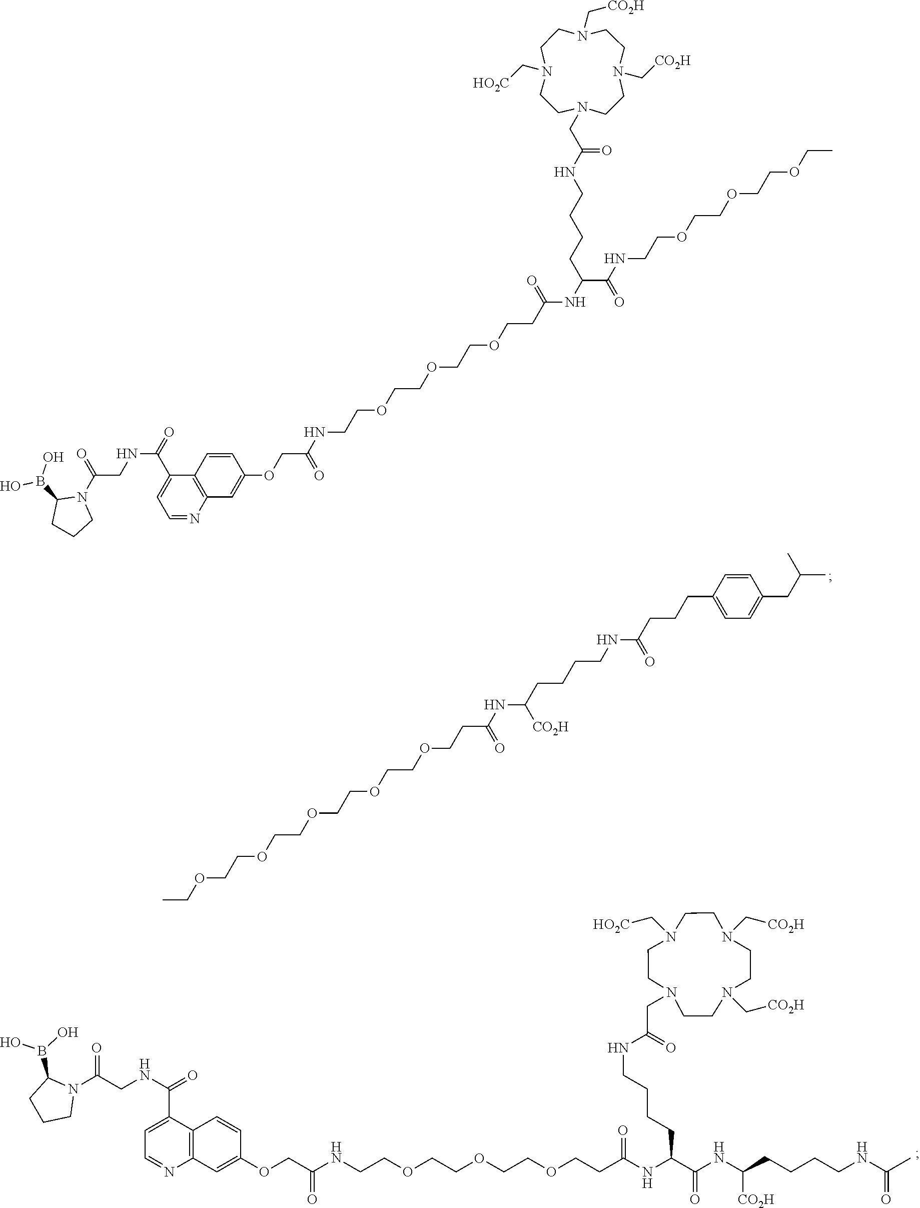

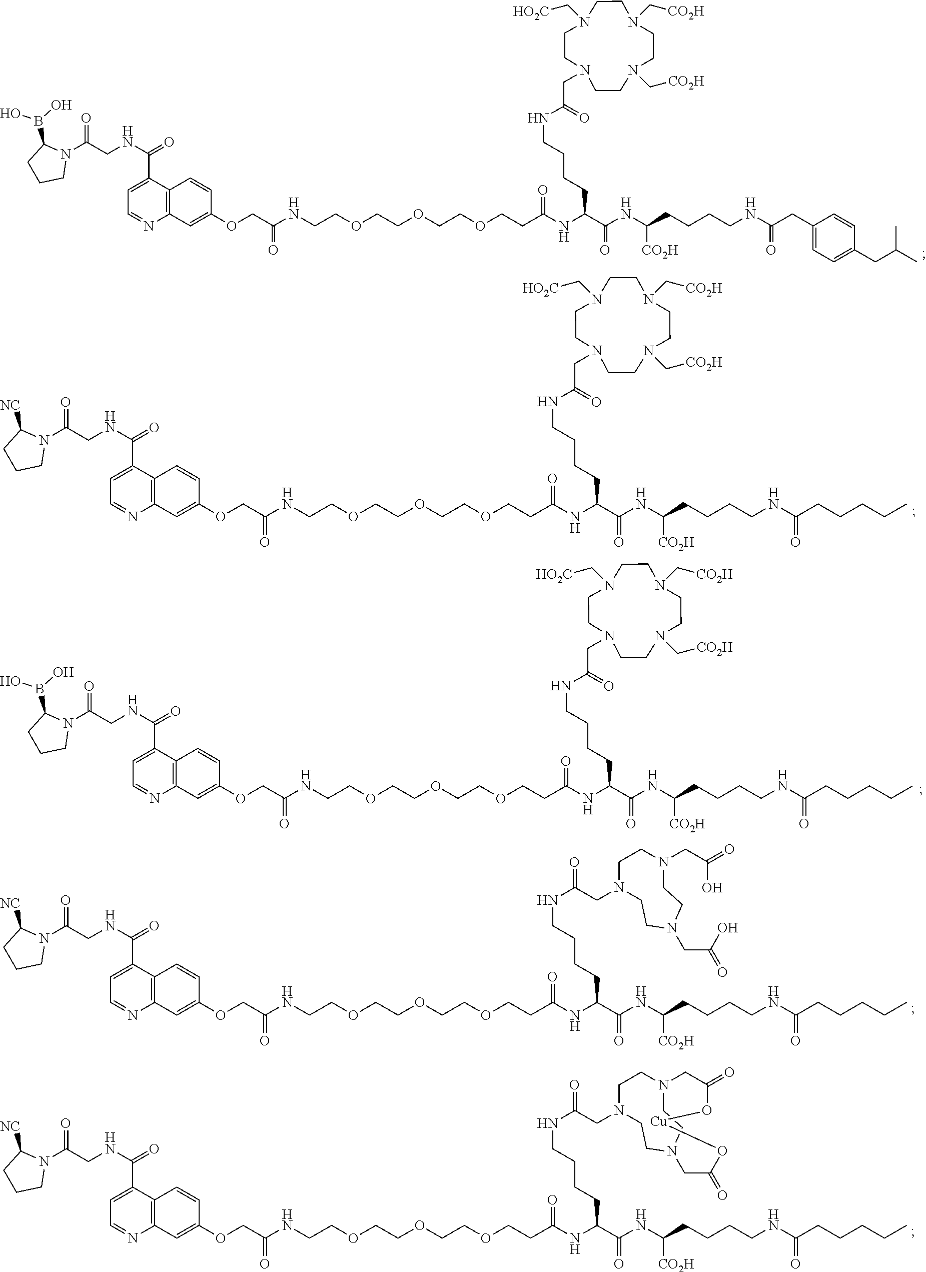

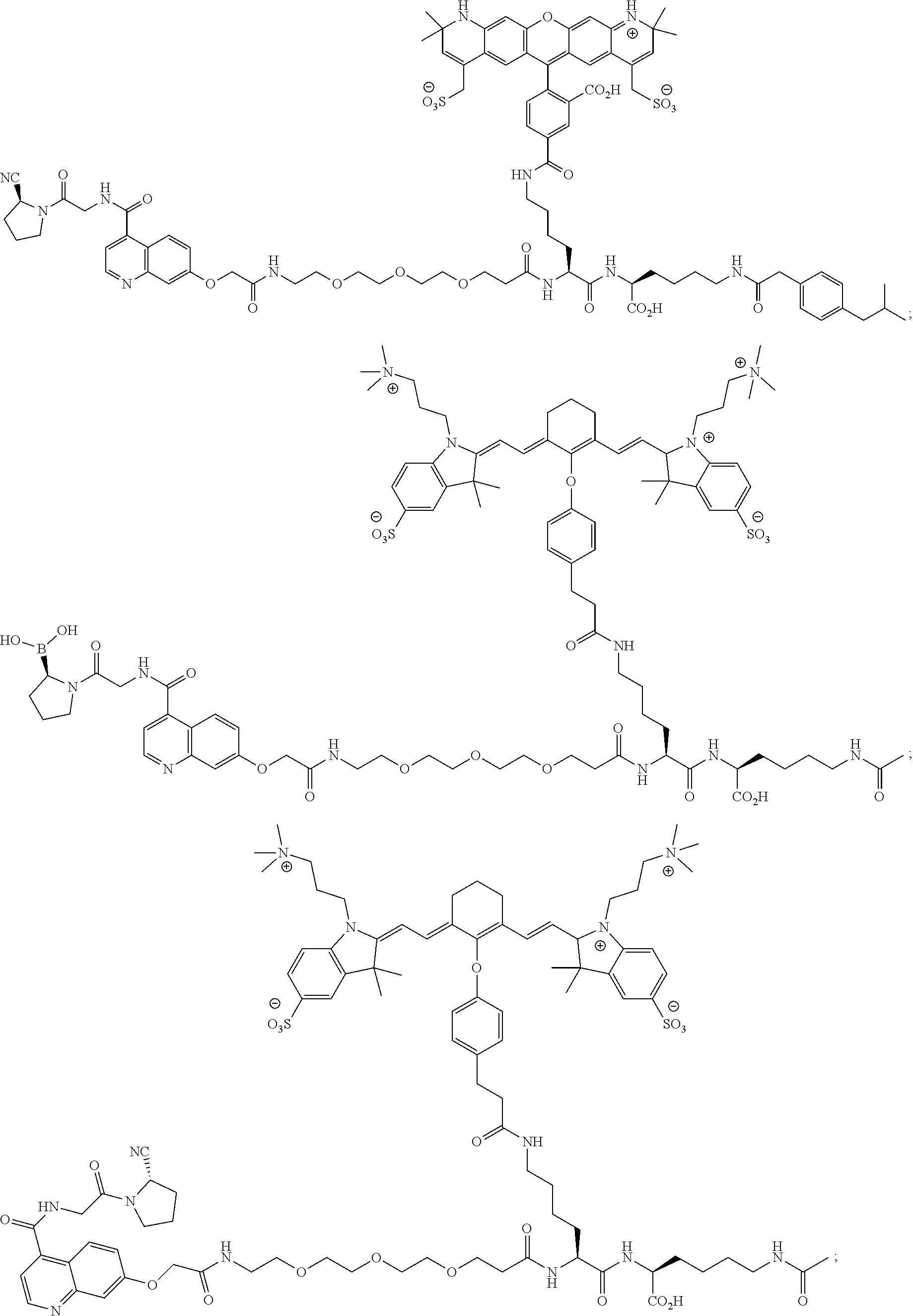

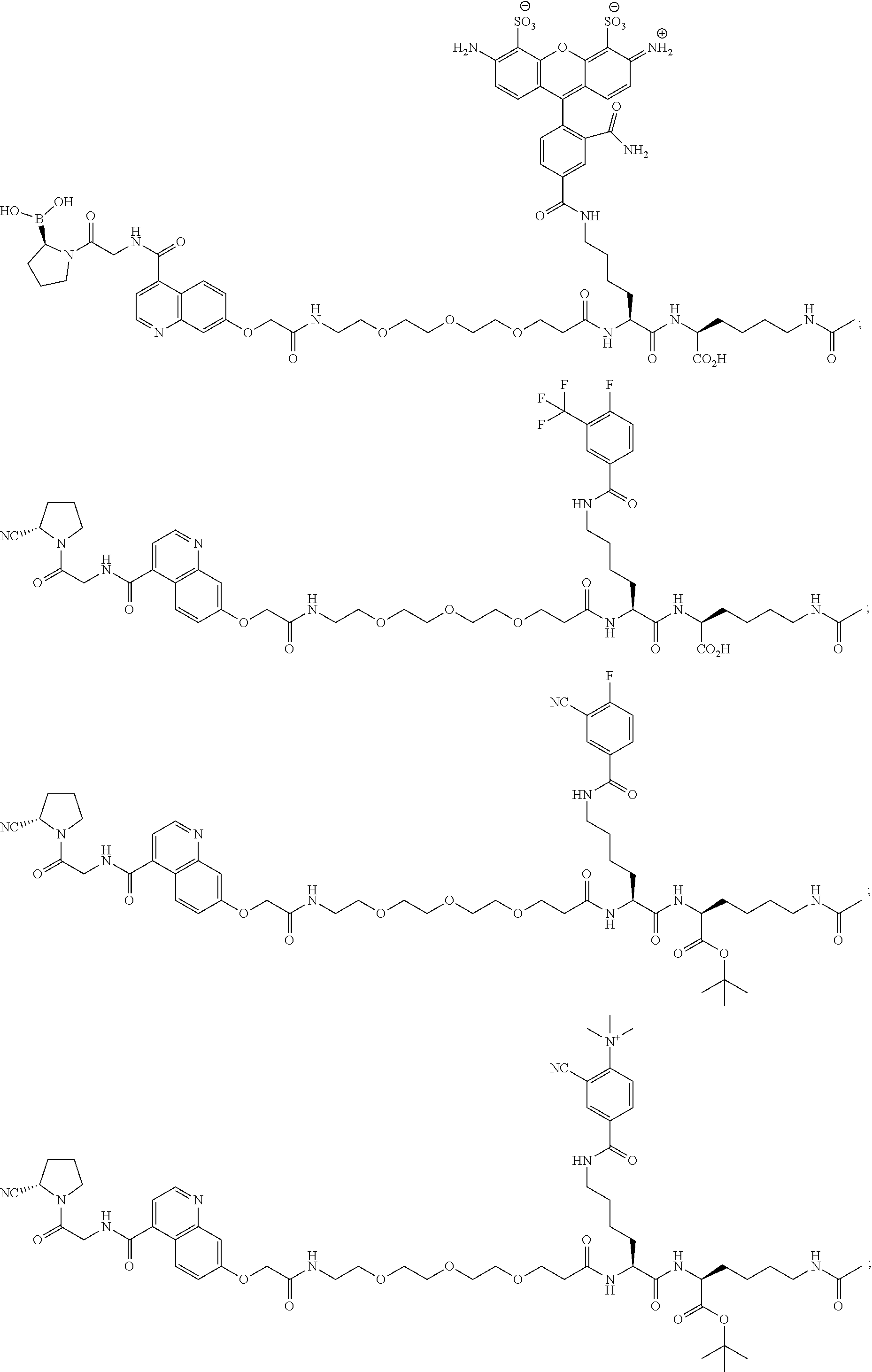

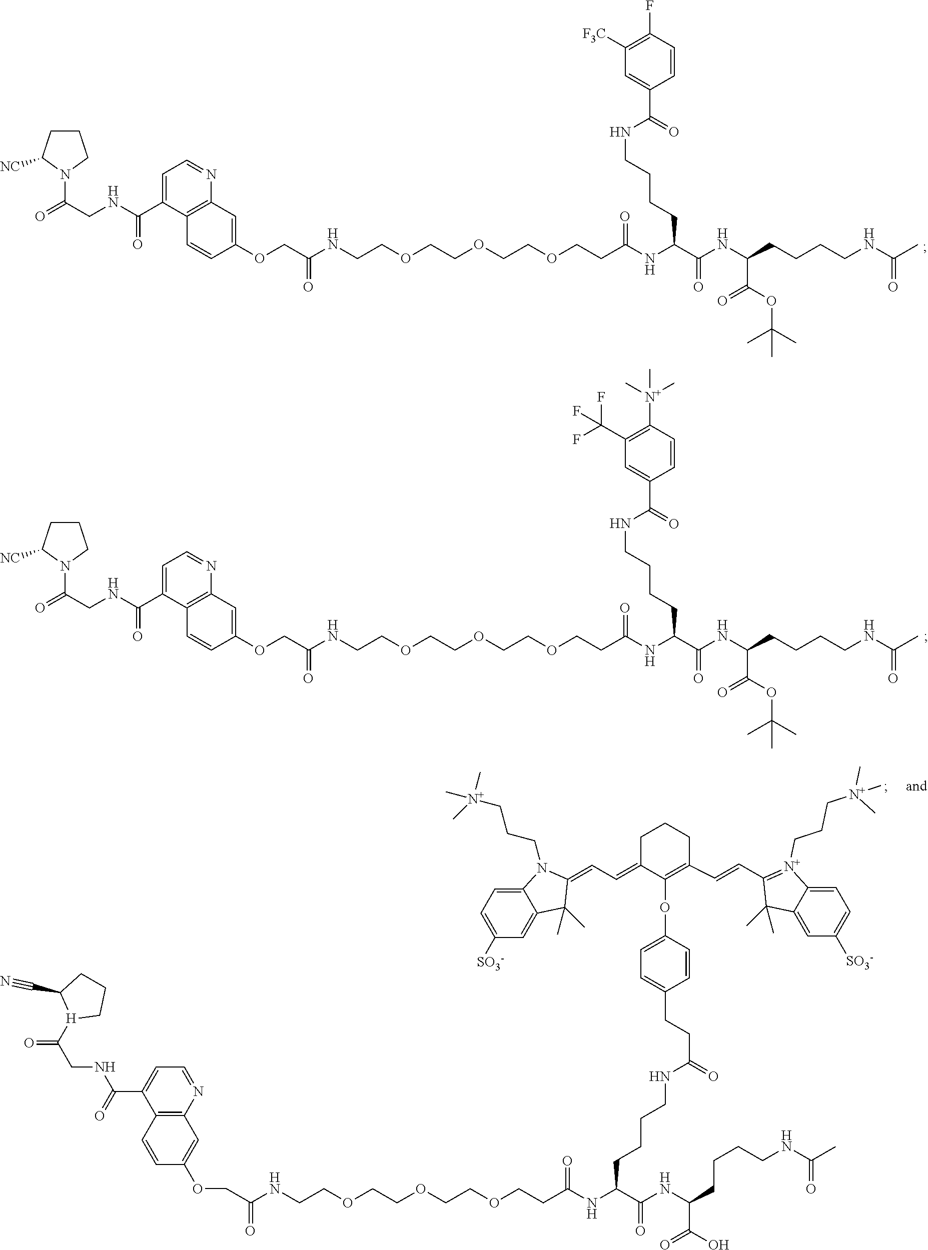

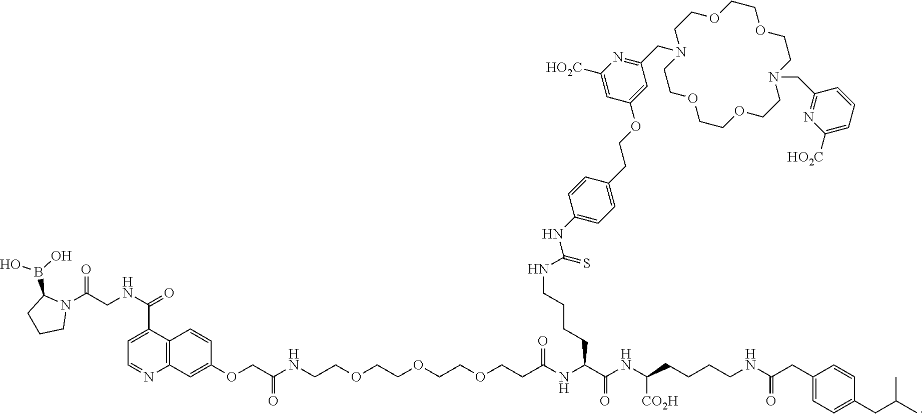 [SMILES: CC(C)Cc1ccc(CC(=O)NCCCC[C@H](NC(=O)[C@H](CCCCNC(=S)Nc2ccc(CCOc3cc(CN4CCOCCOCCN(Cc5cccc(C(=O)O)n5)CCOCCOCC4)nc(C(=O)O)c3)cc2)NC(=O)CCOCCOCCOCCNC(=O)COc2ccc3c(C(=O)NCC(=O)N4CCC[C@H]4B(O)O)ccnc3c2)C(=O)O)cc1]

24. The compound of any one of claims 1 to 11, 14-18 or 23, or a pharmaceutically acceptable salt thereof, wherein the residue of the chelating agent is chelated with a radionuclide.

25. The compound of claim 24 or a pharmaceutically acceptable salt thereof, wherein the radionuclide is selected from ¹⁷⁷Lu, ¹⁷⁵Lu, ⁴⁵Sc, ⁶⁴Cu, ⁶⁷Cu, ⁶⁸Cu, ⁶⁶Ga, ⁶⁷Ga, ⁶⁸Ga, ⁶⁹Ga, ⁷¹Ga, ⁹⁰Y, ⁸⁹Y, ⁸⁶Y, ⁸⁹Zr, ⁹⁰Y, ^{99m}Tc, ¹¹¹In, ¹¹³In, ¹¹⁵In, ¹³⁹La, ¹³⁴Ce, ¹³⁶Ce, ¹³⁸Ce, ¹⁴⁰Ce, ¹⁴²Ce, ¹⁵¹Eu, ¹⁵³Eu, ¹⁵²Dy, ¹⁴⁹Tb, ¹⁵⁹Tb, ¹⁵⁴Gd, ¹⁵⁵Gd, ¹⁵⁶Gd, ¹⁵⁷Gd, ¹⁵⁸Gd, ¹⁶⁰Gd, ¹⁸⁸Re, ¹⁸⁶Re, ²¹³Bi, ²¹¹At, ²¹⁷At, ²²⁷Th, ²²⁶Th, ²²⁵Ac, ²³³Ra, ¹⁵²Dy, ²¹³Bi, ²¹²Bi, ²¹¹Bi, ²⁰³Pb, ²¹²Pb, ²⁵⁵Fm, and uranium-230.

26. The compound of claim 24 or a pharmaceutically acceptable salt thereof, wherein the radionuclide is an alpha-emitting radionuclide such as ²²⁵Ac, ²³³Ra, and ²¹²Pb.

27. The compound of claim 24 or a pharmaceutically acceptable salt thereof, wherein the radionuclide is an Auger electron emitting radionuclide or a beta-emitting radionuclide such as ¹⁷⁷Lu, ⁹⁰Y, and ⁶⁷Cu.

28. The compound of claim 16 or a pharmaceutically acceptable salt thereof, wherein the residue of macropa-NCS or macropa-NCO is chelated with ²²⁵Ac.

29. The compound of claim 1 or a pharmaceutically acceptable salt thereof, wherein the compound is represented by the following structural formula:

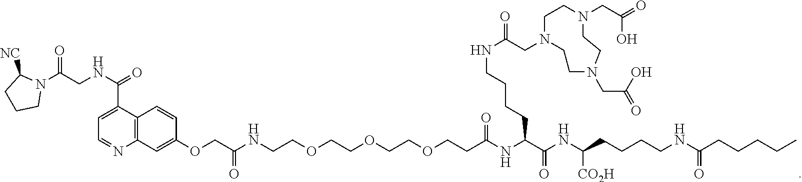 [SMILES: CCCCCC(=O)NCCCC[C@H](NC(=O)[C@H](CCCCNC(=O)CN1CCN(CC(=O)O)CCN(CC(=O)O)CC1)NC(=O)CCOCCOCCOCCNC(=O)COc1ccc2c(C(=O)NCC(=O)N3CCC[C@H]3C#N)ccnc2c1)C(=O)O]

30. The compound of claim 1 or a pharmaceutically acceptable salt thereof, wherein the compound is represented by the following structural formula: 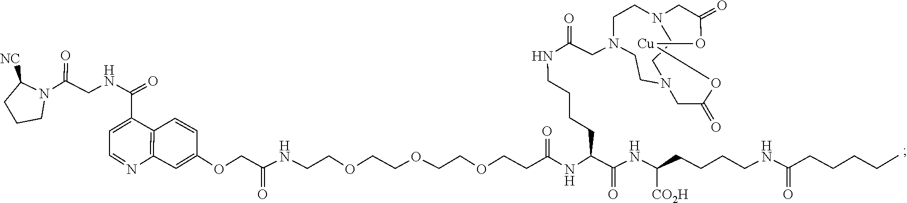
wherein Cu is ⁶⁴Cu.

31. A pharmaceutical composition comprising: i) the compound of any one of claims 1-28 or a pharmaceutically acceptable salt thereof; and ii) a pharmaceutically acceptable carrier or diluent.

32. A method of treating diseased tissue in a subject, wherein the diseased tissue expresses fibroblast activation protein alpha, comprising administering an effective amount of the compound or pharmaceutically acceptable salt of any one of claims 24-28 or the pharmaceutical composition of claim 29 to the subject and wherein the radionuclide is a therapeutic radionuclide.

33. The method of claim 30, the diseased tissue is a cancer.

34. The method of claim 31, wherein the cancer is pancreatic cancer, liver cancer, gall bladder cancer, neuroblastoma, breast cancer, ovarian cancer, esophageal cancer, kidney cancer, prostate cancer, colorectal cancer, soft tissue sarcoma, bone sarcoma or melanoma.

35. The method of claim 31, wherein the diseased tissue is fibrotic.

36. A method of imaging a region in a subject having or suspected of having diseased tissue which expresses fibroblast activation protein alpha or fibrotic tissue, comprising:
a. administering to the subject a diagnostically effective amount of a compound or pharmaceutically acceptable salt thereof of any one of claims 12-13, 19-22 or 24-28 or the pharmaceutical composition of claim 29 and wherein the radionuclide is a diagnostic radionuclide;
b. exposing the region in the subject to an imaging device; and
c. obtaining an image of the diseased tissue in the region.

37. The method of claim 33, wherein the region has or is suspected of having diseased tissue that includes a primary cancer or a metastasis of the cancer.

38. The method of claim 33, wherein the region has or is suspected of having diseased tissue that includes fibrotic tissue.

39. A method of imaging tumors, the method comprising:
a. contacting the tumor and/or surrounding tissue with a compound or pharmaceutically acceptable salt thereof of any one of claims 12-13 in an amount sufficient to bind to the tumor;
b. irradiating the tumor and/or surrounding tissue at a wavelength absorbed by the compound;
c. and detecting a signal from the compound, thereby imaging the tumor and/or surrounding tissue.

40. A method of treating diseased tissue, comprising:
a. administering to a subject, a compound disclosed herein, or a pharmaceutically acceptable salt thereof, or a pharmaceutical composition thereof, in an amount effective to contact and bind to the diseased tissue;
b. using the compound as a fiducial, irradiating the region of the bound compound with one or more doses of external beam radiation,
thereby treating the diseased tissue with radiation.

41. The method of claim 38, wherein the compound comprises a chelating group having a radionuclide that emits gamma-rays or positrons, or an optical dye or a fluorophore, or other detectible radiation.

42. A method of treating diseased tissue, comprising: administering to a subject, a compound disclosed herein, or a pharmaceutically acceptable salt thereof, or a pharmaceutical composition thereof, in an amount effective to contact and bind to the diseased tissue; and using the compound as a fiducial for guided surgery applications, to resect the region of the diseased tissue thereby excising the diseased tissue.

43. The method of claim 39, wherein the compound comprises a chelating group having a radionuclide that emits gamma-rays or positrons, or an optical dye or a fluorophore, or other detectible radiation.
